SEQUENCE LISTING (18 sequences)

SEQ ID NO: 1 - DNA, 378 bp, synthetic construct
  source 1..378: mol_type = other DNA
atgatggctgcagttcagctcttagggcttttgctgctctgcctccgagccatgagatgtgacatccagatgacccagtctccttcacacctgtcagcatctgtgggagacagagtcactctcagctgcaaagtaagtcagaatatttacaagtacttaaactggtatcagcaaaaacttggagaagctcccaaactcctgatatattatacaagctttttgcaaacgggcatcccgtcaaggttcagtggcagtggatctggtacagattacacactcaccatcagcagcctgcagcctgaagatgttgccacatatttctgccagaagtattatagcgggtggacgttcggtggaggcaccaagctggaattgaaa

SEQ ID NO: 2 - DNA, 408 bp, synthetic construct
  source 1..408: mol_type = other DNA
atgggatggagccagatcattctctttctggtggcagcaactacatgtgtccactcccaggtacagctacagcaatcagggactgaactggtgaagcctgggtcctcagtgaaaatttcctgcaaggcttctggcgacaccttcaccagtgactatatgcactggataaggcagcagcctggaagtggccttgagtggattgggtggatttatcctggaaatggtaatactaagtacaatcaaaagttcgatgggaaggcaacactcactgcagacaaatcctccagcacagcctatttgcagctcagcctcctgacatctgaggactatgcagtctatttctgtgcaagacagacggaggggtactttgattactggggccaaggagtcatggtcacagtctcctca

SEQ ID NO: 3 - protein, 478 aa, Homo sapiens
MIRKKRKRSAPPGPCRSHGPRPATAPAPPPSPEPTRPAWTGMGLRAAPSSAAAAAAEVEQRRSPGLCPPPLELLLLLLFSLGLLHAGDCQQPAQCRIQKCTTDFVSLTSHLNSAVDGFDSEFCKALRAYAGCTQRTSKACRGNLVYHSAVLGISDLMSQRNCSKDGPTSSTNPEVTHDPCNYHSHAGAREHRRGDQNPPSYLFCGLFGDPHLRTFKDNFQTCKVEGAWPLIDNNYLSVQVTNVPVVPGSSATATNKITIIFKAHHECTDQKVYQAVTDDLPAAFVDGTTSGGDSDAKSLRIVERESGHYVEMHARYIGTTVFVRQVGRYLTLAIRMPEDLAMSYEESQDLQLCVNGCPLSERIDDGQGQVSAILGHSLPRTSLVQAWPGYTLETANTQCHEKMPVKDIYFQSCVFDLLTTGDANFTAAAHSALEDVEALHPRKERWHIFPSSGNGTPRGGSDLSVSLGLTCLILIVFL

SEQ ID NO: 4 - protein, 437 aa, Homo sapiens
MGLRAAPSSAAAAAAEVEQRRSPGLCPPPLELLLLLLFSLGLLHAGDCQQPAQCRIQKCTTDFVSLTSHLNSAVDGFDSEFCKALRAYAGCTQRTSKACRGNLVYHSAVLGISDLMSQRNCSKDGPTSSTNPEVTHDPCNYHSHAGAREHRRGDQNPPSYLFCGLFGDPHLRTFKDNFQTCKVEGAWPLIDNNYLSVQVTNVPVVPGSSATATNKITIIFKAHHECTDQKVYQAVTDDLPAAFVDGTTSGGDSDAKSLRIVERESGHYVEMHARYIGTTVFVRQVGRYLTLAIRMPEDLAMSYEESQDLQLCVNGCPLSERIDDGQGQVSAILGHSLPRTSLVQAWPGYTLETANTQCHEKMPVKDIYFQSCVFDLLTTGDANFTAAAHSALEDVEALHPRKERWHIFPSSGNGTPRGGSDLSVSLGLTCLILIVFL

SEQ ID NO: 5 - protein, 477 aa, Homo sapiens
MMKKRKRSAPPGPCRSHGPRPATAPAPPPSPEPTRPAWTGMGLRAAPSSAAAAAAEVEQRRSPGLCPPPLELLLLLLFSLGLLHAGDCQQPAQCRIQKCTTDFVSLTSHLNSAVDGFDSEFCKALRAYAGCTQRTSKACRGNLVYHSAVLGISDLMSQRNCSKDGPTSSTNPEVTHDPCNYHSHAGAREHRRGDQNPPSYLFCGLFGDPHLRTFKDNFQTCKVEGAWPLIDNNYLSVQVTNVPVVPGSSATATNKITIIFKAHHECTDQKVYQAVTDDLPAAFVDGTTSGGDSDAKSLRIVERESGHYVEMHARYIGTTVFVRQVGRYLTLAIRMPEDLAMSYEESQDLQLCVNGCPLSERIDDGQGQVSAILGHSLPRTSLVQAWPGYTLETANTQCHEKMPVKDIYFQSCVFDLLTTGDANFTAAAHSALEDVEALHPRKERWHIFPSSGNGTPRGGSDLSVSLGLTCLILIVFL

SEQ ID NO: 6 - protein, 396 aa, Homo sapiens
MPPNCDCQQPAQCRIQKCTTDFVSLTSHLNSAVDGFDSEFCKALRAYAGCTQRTSKACRGNLVYHSAVLGISDLMSQRNCSKDGPTSSTNPEVTHDPCNYHSHAGAREHRRGDQNPPSYLFCGLFGDPHLRTFKDNFQTCKVEGAWPLIDNNYLSVQVTNVPVVPGSSATATNKITIIFKAHHECTDQKVYQAVTDDLPAAFVDGTTSGGDSDAKSLRIVERESGHYVEMHARYIGTTVFVRQVGRYLTLAIRMPEDLAMSYEESQDLQLCVNGCPLSERIDDGQGQVSAILGHSLPRTSLVQAWPGYTLETANTQCHEKMPVKDIYFQSCVFDLLTTGDANFTAAAHSALEDVEALHPRKERWHIFPSSGNGTPRGGSDLSVSLGLTCLILIVFL

SEQ ID NO: 7 - protein, 539 aa, Homo sapiens
MPVGRLHWFPEINGQEAHYLRALASGSQMLFAPAVLSGPACSEVQGPPDPAEPPPGAKGLRMIRKKRKRSAPPGPCRSHGPRPATAPAPPPSPEPTRPAWTGMGLRAAPSSAAAAAAEVEQRRSPGLCPPPLELLLLLLFSLGLLHAGDCQQPAQCRIQKCTTDFVSLTSHLNSAVDGFDSEFCKALRAYAGCTQRTSKACRGNLVYHSAVLGISDLMSQRNCSKDGPTSSTNPEVTHDPCNYHSHAGAREHRRGDQNPPSYLFCGLFGDPHLRTFKDNFQTCKVEGAWPLIDNNYLSVQVTNVPVVPGSSATATNKITIIFKAHHECTDQKVYQAVTDDLPAAFVDGTTSGGDSDAKSLRIVERESGHYVEMHARYIGTTVFVRQVGRYLTLAIRMPEDLAMSYEESQDLQLCVNGCPLSERIDDGQGQVSAILGHSLPRTSLVQAWPGYTLETANTQCHEKMPVKDIYFQSCVFDLLTTGDANFTAAAHSALEDVEALHPRKERWHIFPSSGNGTPRGGSDLSVSLGLTCLILIVFL

SEQ ID NO: 8 - RNA, 4480 nt, Homo sapiens
  source 1..4480: mol_type = mRNA
gtaacacacttgctctgggccttaggtgtctctttaaaccctctctcccctctttatttccaaatctttatctttggctttcttaagctgtaaaggcacactggcaccttaggtggtcaggtaagatcgctggattccttatttcggcgaaagtccagcggaccattcccgccactgtacccattttagaaacacttggaagacccttaaggatcatttacggagagaaaggtcctgcgtgttccgaagttcaggggccccctgatcccgctgagccccctccaggagcgaaagggttaagaatgataaggaagaagaggaagcgaagcgcgccccccggcccatgccgcagccacgggcccagacccgccacggcgcccgcgccgccgccctcgccggagcccacgagacctgcatggacgggcatgggcttgagagcagcaccttccagcgccgccgctgccgccgccgaggttgagcagcgccgcagccccgggctctgccccccgccgctggagctgctgctgctgctgctgttcagcctcgggctgctccacgcaggtgactgccaacagccagcccaatgtcgaatccagaaatgcaccacggacttcgtgtccctgacttctcacctgaactctgccgttgacggctttgactctgagttttgcaaggccttgcgtgcctatgctggctgcacccagcgaacttcaaaagcctgccgtggcaacctggtataccattctgccgtgttgggtatcagtgacctcatgagccagaggaattgttccaaggatggacccacatcctctaccaaccccgaagtgacccatgatccttgcaactatcacagccacgctggagccagggaacacaggagaggggaccagaaccctcccagttaccttttttgtggcttgtttggagatcctcacctcagaactttcaaggataacttccaaacatgcaaagtagaaggggcctggccactcatagataataattatctttcagttcaagtgacaaacgtacctgtggtccctggatccagtgctactgctacaaataagatcactattatcttcaaagcccaccatgagtgtacagatcagaaagtctaccaagctgtgacagatgacctgccggccgcctttgtggatggcaccaccagtggtggggacagcgatgccaagagcctgcgtatcgtggaaagggagagtggccactatgtggagatgcacgcccgctatatagggaccacagtgtttgtgcggcaggtgggtcgctacctgacccttgccatccgtatgcctgaagacctggccatgtcctacgaggagagccaggacctgcagctgtgcgtgaacggctgccccctgagtgaacgcatcgatgacgggcagggccaggtgtctgccatcctgggacacagcctgcctcgcacctccttggtgcaggcctggcctggctacacactggagactgccaacactcaatgccatgagaagatgccagtgaaggacatctatttccagtcctgtgtcttcgacctgctcaccactggtgatgccaactttactgccgcagcccacagtgccttggaggatgtggaggccctgcacccaaggaaggaacgctggcacattttccccagcagtggcaatgggactccccgtggaggcagtgatttgtctgtcagtctaggactcacctgcttgatccttatcgtgtttttgtaggggttgtcttttgttttggttttttattttttgtctataacaaaattttaaaatatatattgtcataatatattgagtaaaagagtatatatgtatataccatgtatatgacaggatgtttgtcctgggacacccaccagattgtacatactgtgtttggctgttttcacatatgttggatgtagtgttctttgattgtatcaattttgttttgcagttctgtgaaatgttttataatgtccctgcccagggacctgttagaaagcactttattttttatatattaaatatttatgtgtgtgcttggttgatatgtatagtacatatacacagacatccatatgcagcgtttcctttgaaggtgaccagttgtttgtagctattcttggctgtaccttcctgccctttcccattgctactgatttgccacggtgtgcagcttttactcgccaccttccggtggagctgcctcgttcctttgaactatgccctcacccttctgccctcacttgatttgaaagggtcgttaactctcccttacaggtgctttgactcttaaacgctgatcttaagaagctctcttcatctaagagctgttactttttcagaagggggggtattattggtattctgattactctcaattctaattgttatatatttgagcccatacagtgtattaggttgaaccatagaaactgctattctcgtaggtcaaaagggtctagtgatggaagttttgtagataagtaccaggcatctcagtaactcctagactttttctcatcccatgccccgttttaaattgtcagttttccctctgactcttctgtgttaaaacatgaaactataaatttagtaattatcatgccttgctctttttaatctatatgactgatgcaagcccctcttcttaaccgtttcttggctttgagcccagaaacacagctctccctgtctccaactccagtaagccctcctcagcctcaccttacgaatccaaagaactggggtttgttaggttctttctctaatgtagaggcccagatcccatcacaaagtttttcattcttccttgtccaccatgatcttcatcacagtctttgatatgtctgcatgcaaagtggaacagagttgggcggcaatgacagaagagcttccttggcctgactcggtgtgcggccacttcggcactgcttaatccagatattcttgttaactaagcattgtgcttcccaggtggtctgaagtcaggtactctctctctcaacacctgtagttgaatatgatttggtcagttgctcgttgtaacttggagaaattcctataaagtaagatctccttgcctcttccatccattgttggcacccccttgcaaaaggaaaagaacagcaaaagtcaggagcagtaatctgagaaagttaactccaggataggtaggtttctattgttatagctagatgtaaatctttagttccaagaagtgatagagtttctgctttaataatttgttgataagtttacataaacagaaataaaagatactatctttaccgtagtagttcaggccaagattatgcttagttttagttctccaggtagttacttttgccatgtcctattgatcagtgacactgccagaggcccataccggcaagaggaagaggacgtcattttgtaaagtttaacttcttagcgaactgatgtgccacccagtcacagagtggagttgtgaattcatgtagaggtggcaaacctctaccttgtgttgatgagagaataatcttgggcagtctgggaaaataaggaaggcatctccttcttactcatggagattcaactatagagagttgaaacctaaacccgccttccttttatagaagctggactagagacggactgaccatcagctctgaactgtggctttttttgttcacctatgatgccatgtaccaaattcagaagctatcgttaataatttgttttataattgagtagtacaagcgaggaaaaaatacggaggataaccactatttttgtgcaaataatatgaaagtgaagtaaaagcaatagaagaaatttctataggatctgggtttagagtgtgtatcattaataaatatacctttgctcttttcagggaaaataacaaccacccttactgatagttgggaaaagaagattgggttattttgccatatcatttagctggaagtgacatttaaaagcaccctgcatcactagtaatagtgtattttgctattctgcccttgtaatcggtgtccctgtaaaacaatccccacagattactttcagaaatagatgtatttctctacgtaagggccaggtttattttctccttttttgagatttctagaaaaaatgctgcttgcacatgttggttcttgaaaccttagctagaagaatttcaggtcataccaacatgtggataggctatagctgttcagaggtctcctgggggagcttaaaacgggggaaacactggttttcacagatgctccacatggctgtctttaaaagactcaaaacttttttttgtcctctttgttatgcttggaagctcccccccccccaacagtgtgtcgagtctttgcaaagaaacctttagatgtggttcatagatatatgaatacgtatctgtgtaaaacagtgagtgtgcagtgtgtaaatactttaaattattatgctagaaaaataaagttacataccttgctgtggaa

SEQ ID NO: 9 - RNA, 4684 nt, Homo sapiens
  source 1..4684: mol_type = mRNA
attctggagtcggagaactgggagagcggcccccgaggcggagcctccctcccgccccgagtcgcgctgccccccacctgggggagaggggcgggcgccgcgctgccttccctccgcgcctcggccgcgtggcttgcggcttattttcccagctggcaagcgtcgcgctgcagacaagggaatgcctgtgggaaggctgcactggtttcctgagataaatggacaggaagcgcattacctccgcgcgctggcaagcggctcccaaatgctttttgcacctgccgttttgagcggtgagagcagcggcggagcccgcagggattgcgggggcccgttgtctacgagcgggtgatgagccccccgcaagccccgggctctctccctccgcgagtgcattgctcacgccaagctttgggactcttctctcacacccaggtcctgcgtgttccgaagttcaggggccccctgatcccgctgagccccctccaggagcgaaagggttaagaatgataaggaagaagaggaagcgaagcgcgccccccggcccatgccgcagccacgggcccagacccgccacggcgcccgcgccgccgccctcgccggagcccacgagacctgcatggacgggcatgggcttgagagcagcaccttccagcgccgccgctgccgccgccgaggttgagcagcgccgcagccccgggctctgccccccgccgctggagctgctgctgctgctgctgttcagcctcgggctgctccacgcaggtgactgccaacagccagcccaatgtcgaatccagaaatgcaccacggacttcgtgtccctgacttctcacctgaactctgccgttgacggctttgactctgagttttgcaaggccttgcgtgcctatgctggctgcacccagcgaacttcaaaagcctgccgtggcaacctggtataccattctgccgtgttgggtatcagtgacctcatgagccagaggaattgttccaaggatggacccacatcctctaccaaccccgaagtgacccatgatccttgcaactatcacagccacgctggagccagggaacacaggagaggggaccagaaccctcccagttaccttttttgtggcttgtttggagatcctcacctcagaactttcaaggataacttccaaacatgcaaagtagaaggggcctggccactcatagataataattatctttcagttcaagtgacaaacgtacctgtggtccctggatccagtgctactgctacaaataagatcactattatcttcaaagcccaccatgagtgtacagatcagaaagtctaccaagctgtgacagatgacctgccggccgcctttgtggatggcaccaccagtggtggggacagcgatgccaagagcctgcgtatcgtggaaagggagagtggccactatgtggagatgcacgcccgctatatagggaccacagtgtttgtgcggcaggtgggtcgctacctgacccttgccatccgtatgcctgaagacctggccatgtcctacgaggagagccaggacctgcagctgtgcgtgaacggctgccccctgagtgaacgcatcgatgacgggcagggccaggtgtctgccatcctgggacacagcctgcctcgcacctccttggtgcaggcctggcctggctacacactggagactgccaacactcaatgccatgagaagatgccagtgaaggacatctatttccagtcctgtgtcttcgacctgctcaccactggtgatgccaactttactgccgcagcccacagtgccttggaggatgtggaggccctgcacccaaggaaggaacgctggcacattttccccagcagtggcaatgggactccccgtggaggcagtgatttgtctgtcagtctaggactcacctgcttgatccttatcgtgtttttgtaggggttgtcttttgttttggttttttattttttgtctataacaaaattttaaaatatatattgtcataatatattgagtaaaagagtatatatgtatataccatgtatatgacaggatgtttgtcctgggacacccaccagattgtacatactgtgtttggctgttttcacatatgttggatgtagtgttctttgattgtatcaattttgttttgcagttctgtgaaatgttttataatgtccctgcccagggacctgttagaaagcactttattttttatatattaaatatttatgtgtgtgcttggttgatatgtatagtacatatacacagacatccatatgcagcgtttcctttgaaggtgaccagttgtttgtagctattcttggctgtaccttcctgccctttcccattgctactgatttgccacggtgtgcagcttttactcgccaccttccggtggagctgcctcgttcctttgaactatgccctcacccttctgccctcacttgatttgaaagggtcgttaactctcccttacaggtgctttgactcttaaacgctgatcttaagaagctctcttcatctaagagctgttactttttcagaagggggggtattattggtattctgattactctcaattctaattgttatatatttgagcccatacagtgtattaggttgaaccatagaaactgctattctcgtaggtcaaaagggtctagtgatggaagttttgtagataagtaccaggcatctcagtaactcctagactttttctcatcccatgccccgttttaaattgtcagttttccctctgactcttctgtgttaaaacatgaaactataaatttagtaattatcatgccttgctctttttaatctatatgactgatgcaagcccctcttcttaaccgtttcttggctttgagcccagaaacacagctctccctgtctccaactccagtaagccctcctcagcctcaccttacgaatccaaagaactggggtttgttaggttctttctctaatgtagaggcccagatcccatcacaaagtttttcattcttccttgtccaccatgatcttcatcacagtctttgatatgtctgcatgcaaagtggaacagagttgggcggcaatgacagaagagcttccttggcctgactcggtgtgcggccacttcggcactgcttaatccagatattcttgttaactaagcattgtgcttcccaggtggtctgaagtcaggtactctctctctcaacacctgtagttgaatatgatttggtcagttgctcgttgtaacttggagaaattcctataaagtaagatctccttgcctcttccatccattgttggcacccccttgcaaaaggaaaagaacagcaaaagtcaggagcagtaatctgagaaagttaactccaggataggtaggtttctattgttatagctagatgtaaatctttagttccaagaagtgatagagtttctgctttaataatttgttgataagtttacataaacagaaataaaagatactatctttaccgtagtagttcaggccaagattatgcttagttttagttctccaggtagttacttttgccatgtcctattgatcagtgacactgccagaggcccataccggcaagaggaagaggacgtcattttgtaaagtttaacttcttagcgaactgatgtgccacccagtcacagagtggagttgtgaattcatgtagaggtggcaaacctctaccttgtgttgatgagagaataatcttgggcagtctgggaaaataaggaaggcatctccttcttactcatggagattcaactatagagagttgaaacctaaacccgccttccttttatagaagctggactagagacggactgaccatcagctctgaactgtggctttttttgttcacctatgatgccatgtaccaaattcagaagctatcgttaataatttgttttataattgagtagtacaagcgaggaaaaaatacggaggataaccactatttttgtgcaaataatatgaaagtgaagtaaaagcaatagaagaaatttctataggatctgggtttagagtgtgtatcattaataaatatacctttgctcttttcagggaaaataacaaccacccttactgatagttgggaaaagaagattgggttattttgccatatcatttagctggaagtgacatttaaaagcaccctgcatcactagtaatagtgtattttgctattctgcccttgtaatcggtgtccctgtaaaacaatccccacagattactttcagaaatagatgtatttctctacgtaagggccaggtttattttctccttttttgagatttctagaaaaaatgctgcttgcacatgttggttcttgaaaccttagctagaagaatttcaggtcataccaacatgtggataggctatagctgttcagaggtctcctgggggagcttaaaacgggggaaacactggttttcacagatgctccacatggctgtctttaaaagactcaaaacttttttttgtcctctttgttatgcttggaagctcccccccccccaacagtgtgtcgagtctttgcaaagaaacctttagatgtggttcatagatatatgaatacgtatctgtgtaaaacagtgagtgtgcagtgtgtaaatactttaaattattatgctagaaaaataaagttacataccttgctgtggaa

SEQ ID NO: 10 - RNA, 4542 nt, Homo sapiens
  source 1..4542: mol_type = mRNA
attctggagtcggagaactgggagagcggcccccgaggcggagcctccctcccgccccgagtcgcgctgccccccacctgggggagaggggcgggcgccgcgctgccttccctccgcgcctcggccgcgtggcttgcggcttattttcccagctggcaagcgtcgcgctgcagacaagggaatgcctgtgggaaggctgcactggtttcctgagataaatggacaggaagcgcattacctccgcgcgctggcaagcggctcccaaatgctttttgcacctgccgttttgagcggtcctgcgtgttccgaagttcaggggccccctgatcccgctgagccccctccaggagcgaaagggttaagaatgataaggaagaagaggaagcgaagcgcgccccccggcccatgccgcagccacgggcccagacccgccacggcgcccgcgccgccgccctcgccggagcccacgagacctgcatggacgggcatgggcttgagagcagcaccttccagcgccgccgctgccgccgccgaggttgagcagcgccgcagccccgggctctgccccccgccgctggagctgctgctgctgctgctgttcagcctcgggctgctccacgcaggtgactgccaacagccagcccaatgtcgaatccagaaatgcaccacggacttcgtgtccctgacttctcacctgaactctgccgttgacggctttgactctgagttttgcaaggccttgcgtgcctatgctggctgcacccagcgaacttcaaaagcctgccgtggcaacctggtataccattctgccgtgttgggtatcagtgacctcatgagccagaggaattgttccaaggatggacccacatcctctaccaaccccgaagtgacccatgatccttgcaactatcacagccacgctggagccagggaacacaggagaggggaccagaaccctcccagttaccttttttgtggcttgtttggagatcctcacctcagaactttcaaggataacttccaaacatgcaaagtagaaggggcctggccactcatagataataattatctttcagttcaagtgacaaacgtacctgtggtccctggatccagtgctactgctacaaataagatcactattatcttcaaagcccaccatgagtgtacagatcagaaagtctaccaagctgtgacagatgacctgccggccgcctttgtggatggcaccaccagtggtggggacagcgatgccaagagcctgcgtatcgtggaaagggagagtggccactatgtggagatgcacgcccgctatatagggaccacagtgtttgtgcggcaggtgggtcgctacctgacccttgccatccgtatgcctgaagacctggccatgtcctacgaggagagccaggacctgcagctgtgcgtgaacggctgccccctgagtgaacgcatcgatgacgggcagggccaggtgtctgccatcctgggacacagcctgcctcgcacctccttggtgcaggcctggcctggctacacactggagactgccaacactcaatgccatgagaagatgccagtgaaggacatctatttccagtcctgtgtcttcgacctgctcaccactggtgatgccaactttactgccgcagcccacagtgccttggaggatgtggaggccctgcacccaaggaaggaacgctggcacattttccccagcagtggcaatgggactccccgtggaggcagtgatttgtctgtcagtctaggactcacctgcttgatccttatcgtgtttttgtaggggttgtcttttgttttggttttttattttttgtctataacaaaattttaaaatatatattgtcataatatattgagtaaaagagtatatatgtatataccatgtatatgacaggatgtttgtcctgggacacccaccagattgtacatactgtgtttggctgttttcacatatgttggatgtagtgttctttgattgtatcaattttgttttgcagttctgtgaaatgttttataatgtccctgcccagggacctgttagaaagcactttattttttatatattaaatatttatgtgtgtgcttggttgatatgtatagtacatatacacagacatccatatgcagcgtttcctttgaaggtgaccagttgtttgtagctattcttggctgtaccttcctgccctttcccattgctactgatttgccacggtgtgcagcttttactcgccaccttccggtggagctgcctcgttcctttgaactatgccctcacccttctgccctcacttgatttgaaagggtcgttaactctcccttacaggtgctttgactcttaaacgctgatcttaagaagctctcttcatctaagagctgttactttttcagaagggggggtattattggtattctgattactctcaattctaattgttatatatttgagcccatacagtgtattaggttgaaccatagaaactgctattctcgtaggtcaaaagggtctagtgatggaagttttgtagataagtaccaggcatctcagtaactcctagactttttctcatcccatgccccgttttaaattgtcagttttccctctgactcttctgtgttaaaacatgaaactataaatttagtaattatcatgccttgctctttttaatctatatgactgatgcaagcccctcttcttaaccgtttcttggctttgagcccagaaacacagctctccctgtctccaactccagtaagccctcctcagcctcaccttacgaatccaaagaactggggtttgttaggttctttctctaatgtagaggcccagatcccatcacaaagtttttcattcttccttgtccaccatgatcttcatcacagtctttgatatgtctgcatgcaaagtggaacagagttgggcggcaatgacagaagagcttccttggcctgactcggtgtgcggccacttcggcactgcttaatccagatattcttgttaactaagcattgtgcttcccaggtggtctgaagtcaggtactctctctctcaacacctgtagttgaatatgatttggtcagttgctcgttgtaacttggagaaattcctataaagtaagatctccttgcctcttccatccattgttggcacccccttgcaaaaggaaaagaacagcaaaagtcaggagcagtaatctgagaaagttaactccaggataggtaggtttctattgttatagctagatgtaaatctttagttccaagaagtgatagagtttctgctttaataatttgttgataagtttacataaacagaaataaaagatactatctttaccgtagtagttcaggccaagattatgcttagttttagttctccaggtagttacttttgccatgtcctattgatcagtgacactgccagaggcccataccggcaagaggaagaggacgtcattttgtaaagtttaacttcttagcgaactgatgtgccacccagtcacagagtggagttgtgaattcatgtagaggtggcaaacctctaccttgtgttgatgagagaataatcttgggcagtctgggaaaataaggaaggcatctccttcttactcatggagattcaactatagagagttgaaacctaaacccgccttccttttatagaagctggactagagacggactgaccatcagctctgaactgtggctttttttgttcacctatgatgccatgtaccaaattcagaagctatcgttaataatttgttttataattgagtagtacaagcgaggaaaaaatacggaggataaccactatttttgtgcaaataatatgaaagtgaagtaaaagcaatagaagaaatttctataggatctgggtttagagtgtgtatcattaataaatatacctttgctcttttcagggaaaataacaaccacccttactgatagttgggaaaagaagattgggttattttgccatatcatttagctggaagtgacatttaaaagcaccctgcatcactagtaatagtgtattttgctattctgcccttgtaatcggtgtccctgtaaaacaatccccacagattactttcagaaatagatgtatttctctacgtaagggccaggtttattttctccttttttgagatttctagaaaaaatgctgcttgcacatgttggttcttgaaaccttagctagaagaatttcaggtcataccaacatgtggataggctatagctgttcagaggtctcctgggggagcttaaaacgggggaaacactggttttcacagatgctccacatggctgtctttaaaagactcaaaacttttttttgtcctctttgttatgcttggaagctcccccccccccaacagtgtgtcgagtctttgcaaagaaacctttagatgtggttcatagatatatgaatacgtatctgtgtaaaacagtgagtgtgcagtgtgtaaatactttaaattattatgctagaaaaataaagttacataccttgctgtggaa

SEQ ID NO: 11 - RNA, 4439 nt, Homo sapiens
  source 1..4439: mol_type = mRNA
attctggagtcggagaactgggagagcggcccccgaggcggagcctccctcccgccccgagtcgcgctgccccccacctgggggagaggggcgggcgccgcgctgccttccctccgcgcctcggccgcgtggcttgcggcttattttcccagctggcaagcgtcgcgctgcagacaagggaatgcctgtggtcctgcgtgttccgaagttcaggggccccctgatcccgctgagccccctccaggagcgaaagggttaagaatgataaggaagaagaggaagcgaagcgcgccccccggcccatgccgcagccacgggcccagacccgccacggcgcccgcgccgccgccctcgccggagcccacgagacctgcatggacgggcatgggcttgagagcagcaccttccagcgccgccgctgccgccgccgaggttgagcagcgccgcagccccgggctctgccccccgccgctggagctgctgctgctgctgctgttcagcctcgggctgctccacgcaggtgactgccaacagccagcccaatgtcgaatccagaaatgcaccacggacttcgtgtccctgacttctcacctgaactctgccgttgacggctttgactctgagttttgcaaggccttgcgtgcctatgctggctgcacccagcgaacttcaaaagcctgccgtggcaacctggtataccattctgccgtgttgggtatcagtgacctcatgagccagaggaattgttccaaggatggacccacatcctctaccaaccccgaagtgacccatgatccttgcaactatcacagccacgctggagccagggaacacaggagaggggaccagaaccctcccagttaccttttttgtggcttgtttggagatcctcacctcagaactttcaaggataacttccaaacatgcaaagtagaaggggcctggccactcatagataataattatctttcagttcaagtgacaaacgtacctgtggtccctggatccagtgctactgctacaaataagatcactattatcttcaaagcccaccatgagtgtacagatcagaaagtctaccaagctgtgacagatgacctgccggccgcctttgtggatggcaccaccagtggtggggacagcgatgccaagagcctgcgtatcgtggaaagggagagtggccactatgtggagatgcacgcccgctatatagggaccacagtgtttgtgcggcaggtgggtcgctacctgacccttgccatccgtatgcctgaagacctggccatgtcctacgaggagagccaggacctgcagctgtgcgtgaacggctgccccctgagtgaacgcatcgatgacgggcagggccaggtgtctgccatcctgggacacagcctgcctcgcacctccttggtgcaggcctggcctggctacacactggagactgccaacactcaatgccatgagaagatgccagtgaaggacatctatttccagtcctgtgtcttcgacctgctcaccactggtgatgccaactttactgccgcagcccacagtgccttggaggatgtggaggccctgcacccaaggaaggaacgctggcacattttccccagcagtggcaatgggactccccgtggaggcagtgatttgtctgtcagtctaggactcacctgcttgatccttatcgtgtttttgtaggggttgtcttttgttttggttttttattttttgtctataacaaaattttaaaatatatattgtcataatatattgagtaaaagagtatatatgtatataccatgtatatgacaggatgtttgtcctgggacacccaccagattgtacatactgtgtttggctgttttcacatatgttggatgtagtgttctttgattgtatcaattttgttttgcagttctgtgaaatgttttataatgtccctgcccagggacctgttagaaagcactttattttttatatattaaatatttatgtgtgtgcttggttgatatgtatagtacatatacacagacatccatatgcagcgtttcctttgaaggtgaccagttgtttgtagctattcttggctgtaccttcctgccctttcccattgctactgatttgccacggtgtgcagcttttactcgccaccttccggtggagctgcctcgttcctttgaactatgccctcacccttctgccctcacttgatttgaaagggtcgttaactctcccttacaggtgctttgactcttaaacgctgatcttaagaagctctcttcatctaagagctgttactttttcagaagggggggtattattggtattctgattactctcaattctaattgttatatatttgagcccatacagtgtattaggttgaaccatagaaactgctattctcgtaggtcaaaagggtctagtgatggaagttttgtagataagtaccaggcatctcagtaactcctagactttttctcatcccatgccccgttttaaattgtcagttttccctctgactcttctgtgttaaaacatgaaactataaatttagtaattatcatgccttgctctttttaatctatatgactgatgcaagcccctcttcttaaccgtttcttggctttgagcccagaaacacagctctccctgtctccaactccagtaagccctcctcagcctcaccttacgaatccaaagaactggggtttgttaggttctttctctaatgtagaggcccagatcccatcacaaagtttttcattcttccttgtccaccatgatcttcatcacagtctttgatatgtctgcatgcaaagtggaacagagttgggcggcaatgacagaagagcttccttggcctgactcggtgtgcggccacttcggcactgcttaatccagatattcttgttaactaagcattgtgcttcccaggtggtctgaagtcaggtactctctctctcaacacctgtagttgaatatgatttggtcagttgctcgttgtaacttggagaaattcctataaagtaagatctccttgcctcttccatccattgttggcacccccttgcaaaaggaaaagaacagcaaaagtcaggagcagtaatctgagaaagttaactccaggataggtaggtttctattgttatagctagatgtaaatctttagttccaagaagtgatagagtttctgctttaataatttgttgataagtttacataaacagaaataaaagatactatctttaccgtagtagttcaggccaagattatgcttagttttagttctccaggtagttacttttgccatgtcctattgatcagtgacactgccagaggcccataccggcaagaggaagaggacgtcattttgtaaagtttaacttcttagcgaactgatgtgccacccagtcacagagtggagttgtgaattcatgtagaggtggcaaacctctaccttgtgttgatgagagaataatcttgggcagtctgggaaaataaggaaggcatctccttcttactcatggagattcaactatagagagttgaaacctaaacccgccttccttttatagaagctggactagagacggactgaccatcagctctgaactgtggctttttttgttcacctatgatgccatgtaccaaattcagaagctatcgttaataatttgttttataattgagtagtacaagcgaggaaaaaatacggaggataaccactatttttgtgcaaataatatgaaagtgaagtaaaagcaatagaagaaatttctataggatctgggtttagagtgtgtatcattaataaatatacctttgctcttttcagggaaaataacaaccacccttactgatagttgggaaaagaagattgggttattttgccatatcatttagctggaagtgacatttaaaagcaccctgcatcactagtaatagtgtattttgctattctgcccttgtaatcggtgtccctgtaaaacaatccccacagattactttcagaaatagatgtatttctctacgtaagggccaggtttattttctccttttttgagatttctagaaaaaatgctgcttgcacatgttggttcttgaaaccttagctagaagaatttcaggtcataccaacatgtggataggctatagctgttcagaggtctcctgggggagcttaaaacgggggaaacactggttttcacagatgctccacatggctgtctttaaaagactcaaaacttttttttgtcctctttgttatgcttggaagctcccccccccccaacagtgtgtcgagtctttgcaaagaaacctttagatgtggttcatagatatatgaatacgtatctgtgtaaaacagtgagtgtgcagtgtgtaaatactttaaattattatgctagaaaaataaagttacataccttgctgtggaa

SEQ ID NO: 12 - RNA, 4436 nt, Homo sapiens
  source 1..4436: mol_type = mRNA
attctggagtcggagaactgggagagcggcccccgaggcggagcctccctcccgccccgagtcgcgctgccccccacctgggggagaggggcgggcgccgcgctgccttccctccgcgcctcggccgcgtggcttgcggcttattttcccagctggcaagcgtcgcgctgcagacaagggaatgcctgtggtcctgcgtgttccgaagttcaggggccccctgatcccgctgagccccctccaggagcgaaagggttaagaatgatgaagaagaggaagcgaagcgcgccccccggcccatgccgcagccacgggcccagacccgccacggcgcccgcgccgccgccctcgccggagcccacgagacctgcatggacgggcatgggcttgagagcagcaccttccagcgccgccgctgccgccgccgaggttgagcagcgccgcagccccgggctctgccccccgccgctggagctgctgctgctgctgctgttcagcctcgggctgctccacgcaggtgactgccaacagccagcccaatgtcgaatccagaaatgcaccacggacttcgtgtccctgacttctcacctgaactctgccgttgacggctttgactctgagttttgcaaggccttgcgtgcctatgctggctgcacccagcgaacttcaaaagcctgccgtggcaacctggtataccattctgccgtgttgggtatcagtgacctcatgagccagaggaattgttccaaggatggacccacatcctctaccaaccccgaagtgacccatgatccttgcaactatcacagccacgctggagccagggaacacaggagaggggaccagaaccctcccagttaccttttttgtggcttgtttggagatcctcacctcagaactttcaaggataacttccaaacatgcaaagtagaaggggcctggccactcatagataataattatctttcagttcaagtgacaaacgtacctgtggtccctggatccagtgctactgctacaaataagatcactattatcttcaaagcccaccatgagtgtacagatcagaaagtctaccaagctgtgacagatgacctgccggccgcctttgtggatggcaccaccagtggtggggacagcgatgccaagagcctgcgtatcgtggaaagggagagtggccactatgtggagatgcacgcccgctatatagggaccacagtgtttgtgcggcaggtgggtcgctacctgacccttgccatccgtatgcctgaagacctggccatgtcctacgaggagagccaggacctgcagctgtgcgtgaacggctgccccctgagtgaacgcatcgatgacgggcagggccaggtgtctgccatcctgggacacagcctgcctcgcacctccttggtgcaggcctggcctggctacacactggagactgccaacactcaatgccatgagaagatgccagtgaaggacatctatttccagtcctgtgtcttcgacctgctcaccactggtgatgccaactttactgccgcagcccacagtgccttggaggatgtggaggccctgcacccaaggaaggaacgctggcacattttccccagcagtggcaatgggactccccgtggaggcagtgatttgtctgtcagtctaggactcacctgcttgatccttatcgtgtttttgtaggggttgtcttttgttttggttttttattttttgtctataacaaaattttaaaatatatattgtcataatatattgagtaaaagagtatatatgtatataccatgtatatgacaggatgtttgtcctgggacacccaccagattgtacatactgtgtttggctgttttcacatatgttggatgtagtgttctttgattgtatcaattttgttttgcagttctgtgaaatgttttataatgtccctgcccagggacctgttagaaagcactttattttttatatattaaatatttatgtgtgtgcttggttgatatgtatagtacatatacacagacatccatatgcagcgtttcctttgaaggtgaccagttgtttgtagctattcttggctgtaccttcctgccctttcccattgctactgatttgccacggtgtgcagcttttactcgccaccttccggtggagctgcctcgttcctttgaactatgccctcacccttctgccctcacttgatttgaaagggtcgttaactctcccttacaggtgctttgactcttaaacgctgatcttaagaagctctcttcatctaagagctgttactttttcagaagggggggtattattggtattctgattactctcaattctaattgttatatatttgagcccatacagtgtattaggttgaaccatagaaactgctattctcgtaggtcaaaagggtctagtgatggaagttttgtagataagtaccaggcatctcagtaactcctagactttttctcatcccatgccccgttttaaattgtcagttttccctctgactcttctgtgttaaaacatgaaactataaatttagtaattatcatgccttgctctttttaatctatatgactgatgcaagcccctcttcttaaccgtttcttggctttgagcccagaaacacagctctccctgtctccaactccagtaagccctcctcagcctcaccttacgaatccaaagaactggggtttgttaggttctttctctaatgtagaggcccagatcccatcacaaagtttttcattcttccttgtccaccatgatcttcatcacagtctttgatatgtctgcatgcaaagtggaacagagttgggcggcaatgacagaagagcttccttggcctgactcggtgtgcggccacttcggcactgcttaatccagatattcttgttaactaagcattgtgcttcccaggtggtctgaagtcaggtactctctctctcaacacctgtagttgaatatgatttggtcagttgctcgttgtaacttggagaaattcctataaagtaagatctccttgcctcttccatccattgttggcacccccttgcaaaaggaaaagaacagcaaaagtcaggagcagtaatctgagaaagttaactccaggataggtaggtttctattgttatagctagatgtaaatctttagttccaagaagtgatagagtttctgctttaataatttgttgataagtttacataaacagaaataaaagatactatctttaccgtagtagttcaggccaagattatgcttagttttagttctccaggtagttacttttgccatgtcctattgatcagtgacactgccagaggcccataccggcaagaggaagaggacgtcattttgtaaagtttaacttcttagcgaactgatgtgccacccagtcacagagtggagttgtgaattcatgtagaggtggcaaacctctaccttgtgttgatgagagaataatcttgggcagtctgggaaaataaggaaggcatctccttcttactcatggagattcaactatagagagttgaaacctaaacccgccttccttttatagaagctggactagagacggactgaccatcagctctgaactgtggctttttttgttcacctatgatgccatgtaccaaattcagaagctatcgttaataatttgttttataattgagtagtacaagcgaggaaaaaatacggaggataaccactatttttgtgcaaataatatgaaagtgaagtaaaagcaatagaagaaatttctataggatctgggtttagagtgtgtatcattaataaatatacctttgctcttttcagggaaaataacaaccacccttactgatagttgggaaaagaagattgggttattttgccatatcatttagctggaagtgacatttaaaagcaccctgcatcactagtaatagtgtattttgctattctgcccttgtaatcggtgtccctgtaaaacaatccccacagattactttcagaaatagatgtatttctctacgtaagggccaggtttattttctccttttttgagatttctagaaaaaatgctgcttgcacatgttggttcttgaaaccttagctagaagaatttcaggtcataccaacatgtggataggctatagctgttcagaggtctcctgggggagcttaaaacgggggaaacactggttttcacagatgctccacatggctgtctttaaaagactcaaaacttttttttgtcctctttgttatgcttggaagctcccccccccccaacagtgtgtcgagtctttgcaaagaaacctttagatgtggttcatagatatatgaatacgtatctgtgtaaaacagtgagtgtgcagtgtgtaaatactttaaattattatgctagaaaaataaagttacataccttgctgtggaa

SEQ ID NO: 13 - RNA, 8167 nt, Homo sapiens
  source 1..8167: mol_type = mRNA
gccttccctccgcgcctcggccgcgtggcttgcggcttattttcccagctggcaagcgtcgcgctgcagacaagggaatgcctgtggtgagttgtttattttgttgaagtttgcggtgggcccgggggacgtggggggcgagcggcaggcagcggccgggactgttttgtttaggtctccatccacctgtggggtataaatctgccagcctcaaggcggcgggggaccggagtgagaaagctctgacgctcccaaaagccccttttcgcaacttacagatctaggaccacatttgattctccacgcctggaaccggctagatcctagtttacgctcaccccgccctccagcctcccatcaacgcttcccgcacccgctgtgtgcagggctggcgctccgcagagcaaacagcgcccacgacccagggacagctggccaccgaagtccctccccggcgcccttccccgttccttgaggataggggagctgaggttttcatgtgaagggcgcccatgaaattcgtgggaacttgttctggcgtcttctggagtggagttctttcaggggtttcgggtctgcccgggactacaaaatgggatcctgggttggctggagtttcccagactaggctggagagaggtcggctgttggctcgcgggagagcccagaggaaccccttgggcgatggcagtcaagaccggcggctgcgtttgcctggaagaaaagcctaaatacattagcgagctggtaaagcttttaaggccttcttgggagcgagtggctggctaatgagaggttaattttgtttcgaagggaaggctgcactggtttcctgagataaatggacaggaagcgcattacctccgcgcgctggcaagcggctcccaaatgctttttgcacctgccgttttgagcggtgagagcagcggcggagcccgcagggattgcgggggcccgttgtctacgagcgggtgatgagccccccgcaagccccgggctctctccctccgcgagtgcattgctcacgccaagctttgggactcttctctcacacccaggtcctgcgtgttccgaagttcaggggccccctgatcccgctgagccccctccaggagcgaaagggttaagaatgatgtaagtacaggcacttgatttctcaagtcgcccgcttggccctcttccaagttgtactttccttattttctctcctttcctttccttactgttctaacgctttttaataaactttcactcttgctctgaaaaaaaaaatgatgtaagtacaaaccacgagctgcttgacgctgagctgttggactccgggctatttcagccgctgctttgggtacacagtggccggggagcagccggggagcccttactctagaaaggattgcacaaatcgcaagcttaggtgatctgttaagcagtcagagattctcctgttagatttctggaaatggagacatattaactggagaacagttaaaaacatcacgtataggctcaggaaatgaaactgcatcttgtttagaatccattagtgtttctggcatgtgttttaattctgtccacttgcacgttcatgccgtagtatgctttgctggattctccggatgtactgttcaggtctcctccttcctttctgaaaacaaagagaaagggagaaagagtaccagtggcaagaaacagggtggatgtgtgaattacacaagtcatcagccacttcatgattttttttctgattaaaacataaaacttttataagtcaccgatttttttgtttgtgttctagctccctccccgatttttctttgtacaatataaagtcgtttgtaacttgcagctcacgggcttgaggagtggctgcggtataagcgactattcactacgctcttgctttccaagccaattcctgatcttgtgaacagtacgaatttacccgcgagtgtgtcaactgacctatgtcttgaaaataggaaccaccttgcggtttacttgtgaaaattagcattttcctctagcggctgaacacttatgcagcaataaaaagtgaattgttaggcttataaatctcgagcttgtcaaattttacaacagaggagcaataaagcaccttatagagatgaagtttttcctctgaagaacagttccgtttaacgacaccaggatttgcaatgactcgaggacgccctctattgataggaaaatctagagcacccgtgtacttatggaacaggatatagggggaggggagtaaaaaatcatagaaagtcacgagtggaaagacccaaaccaaacagaaattattgatccttaactattgagtatatgctttgaagtatgatcaataaagtgagatgctgcttttataggggtcgaattaaatcttgggttaagagaaaaaaatgtagtctttgttagccaaatactttgaaaatcagtattatgtttgattacttttttaagatgcttcttagtatgtcatgaactgtatctaattatatgaagtaattgcaagatagtaaatttttgctctaataatacttagtccgcagcattatctaaaaatgtcatataacttcctccccaaaagtcatttctccaggcaactgtgaagctattattaagcgtattatggatatacacaggcttggattatattgtggatagtgaaaggtgtgtttaccccttaagtcatcatagtatgtaacaagtaagtaatgaaaagttacagtaaaaacagataggcatccttcattagggacctagtggtttgggcaattcaaaatgtgtctaataagtgagctggtgagactgaaggtttgcccctcaattatcctagtccattgattagctcatttaaacatgtgtggaatatcagcatgtccgtggttgataagaaattcctaattagagattctgagtaacatagcgggtacagttgccaactacgatttggatgttagtggattttaaatggcaaccctgtgctgtttggggaaaggcaaaggcgagggcggtatcaagtgtctctcagccttgaggtcattcttgagattagataccacttcacacctaccgataacaaattggaatctatcatcagtctcataactgcttttctgtctccacttggcagcctctttctttgtagacaattgtctttttcctcactttcggacccatccgatcataaaaggagaaagtgagctgccattgaattaatccgtagaaatttgtggaaaatttaaataatctgtagaaatttgtgaaaaagccactgagcaacaacaaatcgtggcataattacactgcataaaaacccaccactctcattaattacactgtaaccgaacgttgaagtttgcccactcttagacctctccttgccgctttcaggaagggaaatgtcaaactagaacacctgagcctcgcacagctgccagcgaattccggcggcagttcgcgggtaccttcccagcccatcctatctgctccggaccgcgggtgacattcagatttccaacaccgccccgagcttgctttaccagacaccctcaaacgactcggaacgcggttccgcagatgtgattggctgaaaggggacctgccgcgcggcgctctgtaaaagcgcctgcttcctccaaggaggtggagctgaagctggcagggccagggcagggaggggtaattaaattaaaaaaaaaacttggagggaagatgaatcggatcacattaattcaaggagaggtcaaaggccggtacctttgtaacgccggcgagagtaagacttgcggtcgcccatttctgttgggcgcgaggaaacagtaaatcgcggaagggggtagagggggaaggaaagggagcgcgaggcagcatgcggcgcaggagagaggacttggagtcgatggacagggaactcgaattacactgagcgggcgggggagagggccctctgggtcctgcgattggcaggctgagtcgggctgacaaggaagaagaggaagcgaagcgcgccccccggcccatgccgcagccacgggcccagacccgccacggcgcccgcgccgccgccctcgccggagcccacgagacctgcatggacgggcatgggcttgagagcagcaccttccagcgccgccgctgccgccgccgaggttgagcagcgccgcagccccgggctctgccccccgccgctggagctgctgctgctgctgctgttcagcctcgggctgctccacgcaggtgactgccaacagccagcccaatgtcgaatccagaaatgcaccacggacttcgtgtccctgacttctcacctgaactctgccgttgacggctttgactctgagttttgcaaggccttgcgtgcctatgctggctgcacccagcgaacttcaaaagcctgccgtggcaacctggtataccattctgccgtgttgggtatcagtgacctcatgagccagaggaattgttccaaggatggacccacatcctctaccaaccccgaagtgacccatgatccttgcaactatcacagccacgctggagccagggaacacaggagaggggaccagaaccctcccagttaccttttttgtggcttgtttggagatcctcacctcagaactttcaaggataacttccaaacatgcaaagtagaaggggcctggccactcatagataataattatctttcagttcaagtgacaaacgtacctgtggtccctggatccagtgctactgctacaaataagatcactattatcttcaaagcccaccatgagtgtacagatcagaaagtctaccaagctgtgacagatgacctgccggccgcctttgtggatggcaccaccagtggtggggacagcgatgccaagagcctgcgtatcgtggaaagggagagtggccactatgtggagatgcacgcccgctatatagggaccacagtgtttgtgcggcaggtgggtcgctacctgacccttgccatccgtatgcctgaagacctggccatgtcctacgaggagagccaggacctgcagctgtgcgtgaacggctgccccctgagtgaacgcatcgatgacgggcagggccaggtgtctgccatcctgggacacagcctgcctcgcacctccttggtgcaggcctggcctggctacacactggagactgccaacactcaatgccatgagaagatgccagtgaaggacatctatttccagtcctgtgtcttcgacctgctcaccactggtgatgccaactttactgccgcagcccacagtgccttggaggatgtggaggccctgcacccaaggaaggaacgctggcacattttccccagcagtggcaatgggactccccgtggaggcagtgatttgtctgtcagtctaggactcacctgcttgatccttatcgtgtttttgtaggggttgtcttttgttttggttttttattttttgtctataacaaaattttaaaatatatattgtcataatatattgagtaaaagagtatatatgtatataccatgtatatgacaggatgtttgtcctgggacacccaccagattgtacatactgtgtttggctgttttcacatatgttggatgtagtgttctttgattgtatcaattttgttttgcagttctgtgaaatgttttataatgtccctgcccagggacctgttagaaagcactttattttttatatattaaatatttatgtgtgtgcttggttgatatgtatagtacatatacacagacatccatatgcagcgtttcctttgaaggtgaccagttgtttgtagctattcttggctgtaccttcctgccctttcccattgctactgatttgccacggtgtgcagcttttactcgccaccttccggtggagctgcctcgttcctttgaactatgccctcacccttctgccctcacttgatttgaaagggtcgttaactctcccttacaggtgctttgactcttaaacgctgatcttaagaagctctcttcatctaagagctgttactttttcagaagggggggtattattggtattctgattactctcaattctaattgttatatatttgagcccatacagtgtattaggttgaaccatagaaactgctattctcgtaggtcaaaagggtctagtgatggaagttttgtagataagtaccaggcatctcagtaactcctagactttttctcatcccatgccccgttttaaattgtcagttttccctctgactcttctgtgttaaaacatgaaactataaatttagtaattatcatgccttgctctttttaatctatatgactgatgcaagcccctcttcttaaccgtttcttggctttgagcccagaaacacagctctccctgtctccaactccagtaagccctcctcagcctcaccttacgaatccaaagaactggggtttgttaggttctttctctaatgtagaggcccagatcccatcacaaagtttttcattcttccttgtccaccatgatcttcatcacagtctttgatatgtctgcatgcaaagtggaacagagttgggcggcaatgacagaagagcttccttggcctgactcggtgtgcggccacttcggcactgcttaatccagatattcttgttaactaagcattgtgcttcccaggtggtctgaagtcaggtactctctctctcaacacctgtagttgaatatgatttggtcagttgctcgttgtaacttggagaaattcctataaagtaagatctccttgcctcttccatccattgttggcacccccttgcaaaaggaaaagaacagcaaaagtcaggagcagtaatctgagaaagttaactccaggataggtaggtttctattgttatagctagatgtaaatctttagttccaagaagtgatagagtttctgctttaataatttgttgataagtttacataaacagaaataaaagatactatctttaccgtagtagttcaggccaagattatgcttagttttagttctccaggtagttacttttgccatgtcctattgatcagtgacactgccagaggcccataccggcaagaggaagaggacgtcattttgtaaagtttaacttcttagcgaactgatgtgccacccagtcacagagtggagttgtgaattcatgtagaggtggcaaacctctaccttgtgttgatgagagaataatcttgggcagtctgggaaaataaggaaggcatctccttcttactcatggagattcaactatagagagttgaaacctaaacccgccttccttttatagaagctggactagagacggactgaccatcagctctgaactgtggctttttttgttcacctatgatgccatgtaccaaattcagaagctatcgttaataatttgttttataattgagtagtacaagcgaggaaaaaatacggaggataaccactatttttgtgcaaataatatgaaagtgaagtaaaagcaatagaagaaatttctataggatctgggtttagagtgtgtatcattaataaatatacctttgctcttttcagggaaaataacaaccacccttactgatagttgggaaaagaagattgggttattttgccatatcatttagctggaagtgacatttaaaagcaccctgcatcactagtaatagtgtattttgctattctgcccttgtaatcggtgtccctgtaaaacaatccccacagattactttcagaaatagatgtatttctctacgtaagggccaggtttattttctccttttttgagatttctagaaaaaatgctgcttgcacatgttggttcttgaaaccttagctagaagaatttcaggtcataccaacatgtggataggctatagctgttcagaggtctcctgggggagcttaaaacgggggaaacactggttttcacagatgctccacatggctgtctttaaaagactcaaaacttttttttgtcctctttgttatgcttggaagctcccccccccccaacagtgtgtcgagtctttgcaaagaaacctttagatgtggttcatagatatatgaatacgtatctgtgtaaaacagtgagtgtgcagtgtgtaaatactttaaattattatgctagaaaaataaagttacataccttgctgtggaa

SEQ ID NO: 14 - RNA, 4996 nt, Homo sapiens
  source 1..4996: mol_type = mRNA
gccttccctccgcgcctcggccgcgtggcttgcggcttattttcccagctggcaagcgtcgcgctgcagacaagggaatgcctgtggtgagttgtttattttgttgaagtttgcggtgggcccgggggacgtggggggcgagcggcaggcagcggccgggactgttttgtttaggtctccatccacctgtggggtataaatctgccagcctcaaggcggcgggggaccggagtgagaaagctctgacgctcccaaaagccccttttcgcaacttacagatctaggaccacatttgattctccacgcctggaaccggctagatcctagtttacgctcaccccgccctccagcctcccatcaacgcttcccgcacccgctgtgtgcagggctggcgctccgcagagcaaacagcgcccacgacccagggacagctggccaccgaagtccctccccggcgcccttccccgttccttgaggataggggagctgaggttttcatgtgaagggcgcccatgaaattcgtgggaacttgttctggcgtcttctggagtggagttctttcaggggtttcgggtctgcccgggactacaaaatgggatcctgggttggctggagtttcccagactaggctggagagaggtcggctgttggctcgcgggagagcccagaggaaccccttgggcgatggcagtcaagaccggcggctgcgtttgcctggaagaaaagcctaaatacattagcgagctggtcctgcgtgttccgaagttcaggggccccctgatcccgctgagccccctccaggagcgaaagggttaagaatgataaggaagaagaggaagcgaagcgcgccccccggcccatgccgcagccacgggcccagacccgccacggcgcccgcgccgccgccctcgccggagcccacgagacctgcatggacgggcatgggcttgagagcagcaccttccagcgccgccgctgccgccgccgaggttgagcagcgccgcagccccgggctctgccccccgccgctggagctgctgctgctgctgctgttcagcctcgggctgctccacgcaggtgactgccaacagccagcccaatgtcgaatccagaaatgcaccacggacttcgtgtccctgacttctcacctgaactctgccgttgacggctttgactctgagttttgcaaggccttgcgtgcctatgctggctgcacccagcgaacttcaaaagcctgccgtggcaacctggtataccattctgccgtgttgggtatcagtgacctcatgagccagaggaattgttccaaggatggacccacatcctctaccaaccccgaagtgacccatgatccttgcaactatcacagccacgctggagccagggaacacaggagaggggaccagaaccctcccagttaccttttttgtggcttgtttggagatcctcacctcagaactttcaaggataacttccaaacatgcaaagtagaaggggcctggccactcatagataataattatctttcagttcaagtgacaaacgtacctgtggtccctggatccagtgctactgctacaaataagatcactattatcttcaaagcccaccatgagtgtacagatcagaaagtctaccaagctgtgacagatgacctgccggccgcctttgtggatggcaccaccagtggtggggacagcgatgccaagagcctgcgtatcgtggaaagggagagtggccactatgtggagatgcacgcccgctatatagggaccacagtgtttgtgcggcaggtgggtcgctacctgacccttgccatccgtatgcctgaagacctggccatgtcctacgaggagagccaggacctgcagctgtgcgtgaacggctgccccctgagtgaacgcatcgatgacgggcagggccaggtgtctgccatcctgggacacagcctgcctcgcacctccttggtgcaggcctggcctggctacacactggagactgccaacactcaatgccatgagaagatgccagtgaaggacatctatttccagtcctgtgtcttcgacctgctcaccactggtgatgccaactttactgccgcagcccacagtgccttggaggatgtggaggccctgcacccaaggaaggaacgctggcacattttccccagcagtggcaatgggactccccgtggaggcagtgatttgtctgtcagtctaggactcacctgcttgatccttatcgtgtttttgtaggggttgtcttttgttttggttttttattttttgtctataacaaaattttaaaatatatattgtcataatatattgagtaaaagagtatatatgtatataccatgtatatgacaggatgtttgtcctgggacacccaccagattgtacatactgtgtttggctgttttcacatatgttggatgtagtgttctttgattgtatcaattttgttttgcagttctgtgaaatgttttataatgtccctgcccagggacctgttagaaagcactttattttttatatattaaatatttatgtgtgtgcttggttgatatgtatagtacatatacacagacatccatatgcagcgtttcctttgaaggtgaccagttgtttgtagctattcttggctgtaccttcctgccctttcccattgctactgatttgccacggtgtgcagcttttactcgccaccttccggtggagctgcctcgttcctttgaactatgccctcacccttctgccctcacttgatttgaaagggtcgttaactctcccttacaggtgctttgactcttaaacgctgatcttaagaagctctcttcatctaagagctgttactttttcagaagggggggtattattggtattctgattactctcaattctaattgttatatatttgagcccatacagtgtattaggttgaaccatagaaactgctattctcgtaggtcaaaagggtctagtgatggaagttttgtagataagtaccaggcatctcagtaactcctagactttttctcatcccatgccccgttttaaattgtcagttttccctctgactcttctgtgttaaaacatgaaactataaatttagtaattatcatgccttgctctttttaatctatatgactgatgcaagcccctcttcttaaccgtttcttggctttgagcccagaaacacagctctccctgtctccaactccagtaagccctcctcagcctcaccttacgaatccaaagaactggggtttgttaggttctttctctaatgtagaggcccagatcccatcacaaagtttttcattcttccttgtccaccatgatcttcatcacagtctttgatatgtctgcatgcaaagtggaacagagttgggcggcaatgacagaagagcttccttggcctgactcggtgtgcggccacttcggcactgcttaatccagatattcttgttaactaagcattgtgcttcccaggtggtctgaagtcaggtactctctctctcaacacctgtagttgaatatgatttggtcagttgctcgttgtaacttggagaaattcctataaagtaagatctccttgcctcttccatccattgttggcacccccttgcaaaaggaaaagaacagcaaaagtcaggagcagtaatctgagaaagttaactccaggataggtaggtttctattgttatagctagatgtaaatctttagttccaagaagtgatagagtttctgctttaataatttgttgataagtttacataaacagaaataaaagatactatctttaccgtagtagttcaggccaagattatgcttagttttagttctccaggtagttacttttgccatgtcctattgatcagtgacactgccagaggcccataccggcaagaggaagaggacgtcattttgtaaagtttaacttcttagcgaactgatgtgccacccagtcacagagtggagttgtgaattcatgtagaggtggcaaacctctaccttgtgttgatgagagaataatcttgggcagtctgggaaaataaggaaggcatctccttcttactcatggagattcaactatagagagttgaaacctaaacccgccttccttttatagaagctggactagagacggactgaccatcagctctgaactgtggctttttttgttcacctatgatgccatgtaccaaattcagaagctatcgttaataatttgttttataattgagtagtacaagcgaggaaaaaatacggaggataaccactatttttgtgcaaataatatgaaagtgaagtaaaagcaatagaagaaatttctataggatctgggtttagagtgtgtatcattaataaatatacctttgctcttttcagggaaaataacaaccacccttactgatagttgggaaaagaagattgggttattttgccatatcatttagctggaagtgacatttaaaagcaccctgcatcactagtaatagtgtattttgctattctgcccttgtaatcggtgtccctgtaaaacaatccccacagattactttcagaaatagatgtatttctctacgtaagggccaggtttattttctccttttttgagatttctagaaaaaatgctgcttgcacatgttggttcttgaaaccttagctagaagaatttcaggtcataccaacatgtggataggctatagctgttcagaggtctcctgggggagcttaaaacgggggaaacactggttttcacagatgctccacatggctgtctttaaaagactcaaaacttttttttgtcctctttgttatgcttggaagctcccccccccccaacagtgtgtcgagtctttgcaaagaaacctttagatgtggttcatagatatatgaatacgtatctgtgtaaaacagtgagtgtgcagtgtgtaaatactttaaattattatgctagaaaaataaagttacataccttgctgtggaa

SEQ ID NO: 15 - RNA, 5459 nt, Homo sapiens
  source 1..5459: mol_type = mRNA
gcagagatatccgggccgccggtgggtggtcgctagggctgggccagcctcttggaggtccacgcccgccgagcccacgctggctggggccggggtgccggcgcgctcgggactcgtctcagcagtcgctcacggtctttgtgtcttctcttccgcccctttccctgcctgccgcctccggccgccacgatgcccctgcgccccgctgctgccgccgcggactggctgcgccggctgcgcgctgcttgctgcggcggtggtggcgccccatctgctacacgggcctgaagaaggaagaagaggaagcgaagcgcgccccccggcccatgccgcagccacgggcccagacccgccacggcgcccgcgccgccgccctcgccggagcccacgagacctgcatggacgggcatgggcttgagagcagcaccttccagcgccgccgctgccgccgccgaggttgagcagcgccgcagccccgggctctgccccccgccgctggagctgctgctgctgctgctgttcagcctcgggctgctccacgcaggtaggacgggagcgcgcgaggggagccagccccggggcctagggctttgttcccaaatagccccaggtctcgaaggcgctcgccggggcggaagggcggcgtggcgcaacgggaaaggcctcccgagcccctgacacgcacctctgtcgggctccgggccacagtaacgcagccacccaccgggcggccgccgggcgcagagacgagccttctctccaccctccccgccttcggctcggaccgctgacaaatctcccctccgacagccgcttatcaaacatttagcgggcgggggccgcgcgcccgagacaaatgttccggatgctttattgctctctgcgctggtgtttactgaaagtcaggctcggatcaggtttccgccgtcgcagccgcctcggagggagacaaggagccttgtgctgcttttctggagttgggggtggcggcgaggcgaaggaagtacaccagatggaaagatttttaccagattttgcaaacctgggagttaatctcgaagtaacgtcaacttttttgagcagggatttctttcgttttggcaaaagctgcgtttgttcagcgatcagcctcccgggaaaagctcgtgttttgtgactagccgggtgtcttcgggctccgtgaggggttgacagataaccatgctgccttctggaggtcggcgcttccccagccttgaggcgtatgttccaagggaagtaagcctgcaaatgagccttctaccatgtgatacctatttttctttttaaaaaatcgtttcatgaaaatccaggacgctcttcacactatttttttctaggaatttgcctagacaaagaattttttttaatgctaaggttgaaagggtcattttcctttagtcttgagaacaaacagcattagataagtttcttttccttccagaccttttttgtcctgtatatatgagacacttcacaacgttctggtctggagaatcctgaacttcgatgccacccaactgtgactgccaacagccagcccaatgtcgaatccagaaatgcaccacggacttcgtgtccctgacttctcacctgaactctgccgttgacggctttgactctgagttttgcaaggccttgcgtgcctatgctggctgcacccagcgaacttcaaaagcctgccgtggcaacctggtataccattctgccgtgttgggtatcagtgacctcatgagccagaggaattgttccaaggatggacccacatcctctaccaaccccgaagtgacccatgatccttgcaactatcacagccacgctggagccagggaacacaggagaggggaccagaaccctcccagttaccttttttgtggcttgtttggagatcctcacctcagaactttcaaggataacttccaaacatgcaaagtagaaggggcctggccactcatagataataattatctttcagttcaagtgacaaacgtacctgtggtccctggatccagtgctactgctacaaataagatcactattatcttcaaagcccaccatgagtgtacagatcagaaagtctaccaagctgtgacagatgacctgccggccgcctttgtggatggcaccaccagtggtggggacagcgatgccaagagcctgcgtatcgtggaaagggagagtggccactatgtggagatgcacgcccgctatatagggaccacagtgtttgtgcggcaggtgggtcgctacctgacccttgccatccgtatgcctgaagacctggccatgtcctacgaggagagccaggacctgcagctgtgcgtgaacggctgccccctgagtgaacgcatcgatgacgggcagggccaggtgtctgccatcctgggacacagcctgcctcgcacctccttggtgcaggcctggcctggctacacactggagactgccaacactcaatgccatgagaagatgccagtgaaggacatctatttccagtcctgtgtcttcgacctgctcaccactggtgatgccaactttactgccgcagcccacagtgccttggaggatgtggaggccctgcacccaaggaaggaacgctggcacattttccccagcagtggcaatgggactccccgtggaggcagtgatttgtctgtcagtctaggactcacctgcttgatccttatcgtgtttttgtaggggttgtcttttgttttggttttttattttttgtctataacaaaattttaaaatatatattgtcataatatattgagtaaaagagtatatatgtatataccatgtatatgacaggatgtttgtcctgggacacccaccagattgtacatactgtgtttggctgttttcacatatgttggatgtagtgttctttgattgtatcaattttgttttgcagttctgtgaaatgttttataatgtccctgcccagggacctgttagaaagcactttattttttatatattaaatatttatgtgtgtgcttggttgatatgtatagtacatatacacagacatccatatgcagcgtttcctttgaaggtgaccagttgtttgtagctattcttggctgtaccttcctgccctttcccattgctactgatttgccacggtgtgcagcttttactcgccaccttccggtggagctgcctcgttcctttgaactatgccctcacccttctgccctcacttgatttgaaagggtcgttaactctcccttacaggtgctttgactcttaaacgctgatcttaagaagctctcttcatctaagagctgttactttttcagaagggggggtattattggtattctgattactctcaattctaattgttatatatttgagcccatacagtgtattaggttgaaccatagaaactgctattctcgtaggtcaaaagggtctagtgatggaagttttgtagataagtaccaggcatctcagtaactcctagactttttctcatcccatgccccgttttaaattgtcagttttccctctgactcttctgtgttaaaacatgaaactataaatttagtaattatcatgccttgctctttttaatctatatgactgatgcaagcccctcttcttaaccgtttcttggctttgagcccagaaacacagctctccctgtctccaactccagtaagccctcctcagcctcaccttacgaatccaaagaactggggtttgttaggttctttctctaatgtagaggcccagatcccatcacaaagtttttcattcttccttgtccaccatgatcttcatcacagtctttgatatgtctgcatgcaaagtggaacagagttgggcggcaatgacagaagagcttccttggcctgactcggtgtgcggccacttcggcactgcttaatccagatattcttgttaactaagcattgtgcttcccaggtggtctgaagtcaggtactctctctctcaacacctgtagttgaatatgatttggtcagttgctcgttgtaacttggagaaattcctataaagtaagatctccttgcctcttccatccattgttggcacccccttgcaaaaggaaaagaacagcaaaagtcaggagcagtaatctgagaaagttaactccaggataggtaggtttctattgttatagctagatgtaaatctttagttccaagaagtgatagagtttctgctttaataatttgttgataagtttacataaacagaaataaaagatactatctttaccgtagtagttcaggccaagattatgcttagttttagttctccaggtagttacttttgccatgtcctattgatcagtgacactgccagaggcccataccggcaagaggaagaggacgtcattttgtaaagtttaacttcttagcgaactgatgtgccacccagtcacagagtggagttgtgaattcatgtagaggtggcaaacctctaccttgtgttgatgagagaataatcttgggcagtctgggaaaataaggaaggcatctccttcttactcatggagattcaactatagagagttgaaacctaaacccgccttccttttatagaagctggactagagacggactgaccatcagctctgaactgtggctttttttgttcacctatgatgccatgtaccaaattcagaagctatcgttaataatttgttttataattgagtagtacaagcgaggaaaaaatacggaggataaccactatttttgtgcaaataatatgaaagtgaagtaaaagcaatagaagaaatttctataggatctgggtttagagtgtgtatcattaataaatatacctttgctcttttcagggaaaataacaaccacccttactgatagttgggaaaagaagattgggttattttgccatatcatttagctggaagtgacatttaaaagcaccctgcatcactagtaatagtgtattttgctattctgcccttgtaatcggtgtccctgtaaaacaatccccacagattactttcagaaatagatgtatttctctacgtaagggccaggtttattttctccttttttgagatttctagaaaaaatgctgcttgcacatgttggttcttgaaaccttagctagaagaatttcaggtcataccaacatgtggataggctatagctgttcagaggtctcctgggggagcttaaaacgggggaaacactggttttcacagatgctccacatggctgtctttaaaagactcaaaacttttttttgtcctctttgttatgcttggaagctcccccccccccaacagtgtgtcgagtctttgcaaagaaacctttagatgtggttcatagatatatgaatacgtatctgtgtaaaacagtgagtgtgcagtgtgtaaatactttaaattattatgctagaaaaataaagttacataccttgctgtggaa

SEQ ID NO: 16 - RNA, 4463 nt, Homo sapiens
  source 1..4463: mol_type = mRNA
gcagagatatccgggccgccggtgggtggtcgctagggctgggccagcctcttggaggtccacgcccgccgagcccacgctggctggggccggggtgccggcgcgctcgggactcgtctcagcagtcgctcacggtctttgtgtcttctcttccgcccctttccctgcctgccgcctccggccgccacgatgcccctgcgccccgctgctgccgccgcggactggctgcgccggctgcgcgctgcttgctgcggcggtggtggcgccccatctgctacacgggcctgaagaaggaagaagaggaagcgaagcgcgccccccggcccatgccgcagccacgggcccagacccgccacggcgcccgcgccgccgccctcgccggagcccacgagacctgcatggacgggcatgggcttgagagcagcaccttccagcgccgccgctgccgccgccgaggttgagcagcgccgcagccccgggctctgccccccgccgctggagctgctgctgctgctgctgttcagcctcgggctgctccacgcaggtgactgccaacagccagcccaatgtcgaatccagaaatgcaccacggacttcgtgtccctgacttctcacctgaactctgccgttgacggctttgactctgagttttgcaaggccttgcgtgcctatgctggctgcacccagcgaacttcaaaagcctgccgtggcaacctggtataccattctgccgtgttgggtatcagtgacctcatgagccagaggaattgttccaaggatggacccacatcctctaccaaccccgaagtgacccatgatccttgcaactatcacagccacgctggagccagggaacacaggagaggggaccagaaccctcccagttaccttttttgtggcttgtttggagatcctcacctcagaactttcaaggataacttccaaacatgcaaagtagaaggggcctggccactcatagataataattatctttcagttcaagtgacaaacgtacctgtggtccctggatccagtgctactgctacaaataagatcactattatcttcaaagcccaccatgagtgtacagatcagaaagtctaccaagctgtgacagatgacctgccggccgcctttgtggatggcaccaccagtggtggggacagcgatgccaagagcctgcgtatcgtggaaagggagagtggccactatgtggagatgcacgcccgctatatagggaccacagtgtttgtgcggcaggtgggtcgctacctgacccttgccatccgtatgcctgaagacctggccatgtcctacgaggagagccaggacctgcagctgtgcgtgaacggctgccccctgagtgaacgcatcgatgacgggcagggccaggtgtctgccatcctgggacacagcctgcctcgcacctccttggtgcaggcctggcctggctacacactggagactgccaacactcaatgccatgagaagatgccagtgaaggacatctatttccagtcctgtgtcttcgacctgctcaccactggtgatgccaactttactgccgcagcccacagtgccttggaggatgtggaggccctgcacccaaggaaggaacgctggcacattttccccagcagtggcaatgggactccccgtggaggcagtgatttgtctgtcagtctaggactcacctgcttgatccttatcgtgtttttgtaggggttgtcttttgttttggttttttattttttgtctataacaaaattttaaaatatatattgtcataatatattgagtaaaagagtatatatgtatataccatgtatatgacaggatgtttgtcctgggacacccaccagattgtacatactgtgtttggctgttttcacatatgttggatgtagtgttctttgattgtatcaattttgttttgcagttctgtgaaatgttttataatgtccctgcccagggacctgttagaaagcactttattttttatatattaaatatttatgtgtgtgcttggttgatatgtatagtacatatacacagacatccatatgcagcgtttcctttgaaggtgaccagttgtttgtagctattcttggctgtaccttcctgccctttcccattgctactgatttgccacggtgtgcagcttttactcgccaccttccggtggagctgcctcgttcctttgaactatgccctcacccttctgccctcacttgatttgaaagggtcgttaactctcccttacaggtgctttgactcttaaacgctgatcttaagaagctctcttcatctaagagctgttactttttcagaagggggggtattattggtattctgattactctcaattctaattgttatatatttgagcccatacagtgtattaggttgaaccatagaaactgctattctcgtaggtcaaaagggtctagtgatggaagttttgtagataagtaccaggcatctcagtaactcctagactttttctcatcccatgccccgttttaaattgtcagttttccctctgactcttctgtgttaaaacatgaaactataaatttagtaattatcatgccttgctctttttaatctatatgactgatgcaagcccctcttcttaaccgtttcttggctttgagcccagaaacacagctctccctgtctccaactccagtaagccctcctcagcctcaccttacgaatccaaagaactggggtttgttaggttctttctctaatgtagaggcccagatcccatcacaaagtttttcattcttccttgtccaccatgatcttcatcacagtctttgatatgtctgcatgcaaagtggaacagagttgggcggcaatgacagaagagcttccttggcctgactcggtgtgcggccacttcggcactgcttaatccagatattcttgttaactaagcattgtgcttcccaggtggtctgaagtcaggtactctctctctcaacacctgtagttgaatatgatttggtcagttgctcgttgtaacttggagaaattcctataaagtaagatctccttgcctcttccatccattgttggcacccccttgcaaaaggaaaagaacagcaaaagtcaggagcagtaatctgagaaagttaactccaggataggtaggtttctattgttatagctagatgtaaatctttagttccaagaagtgatagagtttctgctttaataatttgttgataagtttacataaacagaaataaaagatactatctttaccgtagtagttcaggccaagattatgcttagttttagttctccaggtagttacttttgccatgtcctattgatcagtgacactgccagaggcccataccggcaagaggaagaggacgtcattttgtaaagtttaacttcttagcgaactgatgtgccacccagtcacagagtggagttgtgaattcatgtagaggtggcaaacctctaccttgtgttgatgagagaataatcttgggcagtctgggaaaataaggaaggcatctccttcttactcatggagattcaactatagagagttgaaacctaaacccgccttccttttatagaagctggactagagacggactgaccatcagctctgaactgtggctttttttgttcacctatgatgccatgtaccaaattcagaagctatcgttaataatttgttttataattgagtagtacaagcgaggaaaaaatacggaggataaccactatttttgtgcaaataatatgaaagtgaagtaaaagcaatagaagaaatttctataggatctgggtttagagtgtgtatcattaataaatatacctttgctcttttcagggaaaataacaaccacccttactgatagttgggaaaagaagattgggttattttgccatatcatttagctggaagtgacatttaaaagcaccctgcatcactagtaatagtgtattttgctattctgcccttgtaatcggtgtccctgtaaaacaatccccacagattactttcagaaatagatgtatttctctacgtaagggccaggtttattttctccttttttgagatttctagaaaaaatgctgcttgcacatgttggttcttgaaaccttagctagaagaatttcaggtcataccaacatgtggataggctatagctgttcagaggtctcctgggggagcttaaaacgggggaaacactggttttcacagatgctccacatggctgtctttaaaagactcaaaacttttttttgtcctctttgttatgcttggaagctcccccccccccaacagtgtgtcgagtctttgcaaagaaacctttagatgtggttcatagatatatgaatacgtatctgtgtaaaacagtgagtgtgcagtgtgtaaatactttaaattattatgctagaaaaataaagttacataccttgctgtggaa

SEQ ID NO: 17 - DNA, 27863 bp, Homo sapiens
  source 1..27863: mol_type = genomic DNA
gtaacacacttgctctgggccttaggtgtctctttaaaccctctctcccctctttatttccaaatctttatctttggctttcttaagctgtaaaggcacactggcaccttaggtggtcaggtaagatcgctggattccttatttcggcgaaagtccagcggaccattcccgccactgtacccattttagaaacacttggaagacccttaaggatcatttacggagagaaaggtgtgcattctgcggctgccggcgggcagctagagaacgtggcggggggcagcgcatctggcgccttggccggggcgcggagtgggaacagcccgcacgcgtccgctgccgccctgcctggcgacgagtggcacaatcgcccaccaggaatcggctggtgccgcgggtgggcgaaggcagcgccgccggcccggagggcggcgcggagggtggcgctggcggcgtgaaaggcgtctttgtgcaagattgagtgtggtggggggaaggcgaagagagaactgatccaggacctggacgcgggggtctgcggcgcccctacgcccccacgcccccagccgtccgcagtgccggaatgagccggatagcgccggcagggggcagtgtggcgatataggggttgccggggggagaggagggtctctgttcacctctatttgaggctgactggggagaaagctgcctggaggaagctgctggggtgcggggggctgagggatttggctccccgccctctcccctagatggcggagaaatcgggctagctggaagggcgcagctcctctcccagagctcattctggagtcggagaactgggagagcggcccccgaggcggagcctccctcccgccccgagtcgcgctgccccccacctgggggagaggggcgggcgccgcgctgccttccctccgcgcctcggccgcgtggcttgcggcttattttcccagctggcaagcgtcgcgctgcagacaagggaatgcctgtggtgagttgtttattttgttgaagtttgcggtgggcccgggggacgtggggggcgagcggcaggcagcggccgggactgttttgtttaggtctccatccacctgtggggtataaatctgccagcctcaaggcggcgggggaccggagtgagaaagctctgacgctcccaaaagccccttttcgcaacttacagatctaggaccacatttgattctccacgcctggaaccggctagatcctagtttacgctcaccccgccctccagcctcccatcaacgcttcccgcacccgctgtgtgcagggctggcgctccgcagagcaaacagcgcccacgacccagggacagctggccaccgaagtccctccccggcgcccttccccgttccttgaggataggggagctgaggttttcatgtgaagggcgcccatgaaattcgtgggaacttgttctggcgtcttctggagtggagttctttcaggggtttcgggtctgcccgggactacaaaatgggatcctgggttggctggagtttcccagactaggctggagagaggtcggctgttggctcgcgggagagcccagaggaaccccttgggcgatggcagtcaagaccggcggctgcgtttgcctggaagaaaagcctaaatacattagcgagctggtaaagcttttaaggccttcttgggagcgagtggctggctaatgagaggttaattttgtttcgaagggaaggctgcactggtttcctgagataaatggacaggaagcgcattacctccgcgcgctggcaagcggctcccaaatgctttttgcacctgccgttttgagcggtgagagcagcggcggagcccgcagggattgcgggggcccgttgtctacgagcgggtgatgagccccccgcaagccccgggctctctccctccgcgagtgcattgctcacgccaagctttgggactcttctctcacacccaggtcctgcgtgttccgaagttcaggggccccctgatcccgctgagccccctccaggagcgaaagggttaagaatgatgtaagtacaggcacttgatttctcaagtcgcccgcttggccctcttccaagttgtactttccttattttctctcctttcctttccttactgttctaacgctttttaataaactttcactcttgctctgaaaaaaaaaatgatgtaagtacaaaccacgagctgcttgacgctgagctgttggactccgggctatttcagccgctgctttgggtacacagtggccggggagcagccggggagcccttactctagaaaggattgcacaaatcgcaagcttaggtgatctgttaagcagtcagagattctcctgttagatttctggaaatggagacatattaactggagaacagttaaaaacatcacgtataggctcaggaaatgaaactgcatcttgtttagaatccattagtgtttctggcatgtgttttaattctgtccacttgcacgttcatgccgtagtatgctttgctggattctccggatgtactgttcaggtctcctccttcctttctgaaaacaaagagaaagggagaaagagtaccagtggcaagaaacagggtggatgtgtgaattacacaagtcatcagccacttcatgattttttttctgattaaaacataaaacttttataagtcaccgatttttttgtttgtgttctagctccctccccgatttttctttgtacaatataaagtcgtttgtaacttgcagctcacgggcttgaggagtggctgcggtataagcgactattcactacgctcttgctttccaagccaattcctgatcttgtgaacagtacgaatttacccgcgagtgtgtcaactgacctatgtcttgaaaataggaaccaccttgcggtttacttgtgaaaattagcattttcctctagcggctgaacacttatgcagcaataaaaagtgaattgttaggcttataaatctcgagcttgtcaaattttacaacagaggagcaataaagcaccttatagagatgaagtttttcctctgaagaacagttccgtttaacgacaccaggatttgcaatgactcgaggacgccctctattgataggaaaatctagagcacccgtgtacttatggaacaggatatagggggaggggagtaaaaaatcatagaaagtcacgagtggaaagacccaaaccaaacagaaattattgatccttaactattgagtatatgctttgaagtatgatcaataaagtgagatgctgcttttataggggtcgaattaaatcttgggttaagagaaaaaaatgtagtctttgttagccaaatactttgaaaatcagtattatgtttgattacttttttaagatgcttcttagtatgtcatgaactgtatctaattatatgaagtaattgcaagatagtaaatttttgctctaataatacttagtccgcagcattatctaaaaatgtcatataacttcctccccaaaagtcatttctccaggcaactgtgaagctattattaagcgtattatggatatacacaggcttggattatattgtggatagtgaaaggtgtgtttaccccttaagtcatcatagtatgtaacaagtaagtaatgaaaagttacagtaaaaacagataggcatccttcattagggacctagtggtttgggcaattcaaaatgtgtctaataagtgagctggtgagactgaaggtttgcccctcaattatcctagtccattgattagctcatttaaacatgtgtggaatatcagcatgtccgtggttgataagaaattcctaattagagattctgagtaacatagcgggtacagttgccaactacgatttggatgttagtggattttaaatggcaaccctgtgctgtttggggaaaggcaaaggcgagggcggtatcaagtgtctctcagccttgaggtcattcttgagattagataccacttcacacctaccgataacaaattggaatctatcatcagtctcataactgcttttctgtctccacttggcagcctctttctttgtagacaattgtctttttcctcactttcggacccatccgatcataaaaggagaaagtgagctgccattgaattaatccgtagaaatttgtggaaaatttaaataatctgtagaaatttgtgaaaaagccactgagcaacaacaaatcgtggcataattacactgcataaaaacccaccactctcattaattacactgtaaccgaacgttgaagtttgcccactcttagacctctccttgccgctttcaggaagggaaatgtcaaactagaacacctgagcctcgcacagctgccagcgaattccggcggcagttcgcgggtaccttcccagcccatcctatctgctccggaccgcgggtgacattcagatttccaacaccgccccgagcttgctttaccagacaccctcaaacgactcggaacgcggttccgcagatgtgattggctgaaaggggacctgccgcgcggcgctctgtaaaagcgcctgcttcctccaaggaggtggagctgaagctggcagggccagggcagggaggggtaattaaattaaaaaaaaaacttggagggaagatgaatcggatcacattaattcaaggagaggtcaaaggccggtacctttgtaacgccggcgagagtaagacttgcggtcgcccatttctgttgggcgcgaggaaacagtaaatcgcggaagggggtagagggggaaggaaagggagcgcgaggcagcatgcggcgcaggagagaggacttggagtcgatggacagggaactcgaattacactgagcgggcgggggagagggccctctgggtcctgcgattggcaggctgagtcgggctgacgtgcggtcacaaccaatggggaggaagggagggacacggcgggggattatggcgtcgtggattggcgggcacagggcggggcgggacggcagtgcgggactgagattgatcgaccgcgggggctgccgcgcagagatatccgggccgccggtgggtggtcgctagggctgggccagcctcttggaggtccacgcccgccgagcccacgctggctggggccggggtgccggcgcgctcgggactcgtctcagcagtcgctcacggtctttgtgtcttctcttccgcccctttccctgcctgccgcctccggccgccacgatgcccctgcgccccgctgctgccgccgcggactggctgcgccggctgcgcgctgcttgctgcggcggtggtggcgccccatctgctacacgggcctgaagaaggaagaagaggaagcgaagcgcgccccccggcccatgccgcagccacgggcccagacccgccacggcgcccgcgccgccgccctcgccggagcccacgagacctgcatggacgggcatgggcttgagagcagcaccttccagcgccgccgctgccgccgccgaggttgagcagcgccgcagccccgggctctgccccccgccgctggagctgctgctgctgctgctgttcagcctcgggctgctccacgcaggtaggacgggagcgcgcgaggggagccagccccggggcctagggctttgttcccaaatagccccaggtctcgaaggcgctcgccggggcggaagggcggcgtggcgcaacgggaaaggcctcccgagcccctgacacgcacctctgtcgggctccgggccacagtaacgcagccacccaccgggcggccgccgggcgcagagacgagccttctctccaccctccccgccttcggctcggaccgctgacaaatctcccctccgacagccgcttatcaaacatttagcgggcgggggccgcgcgcccgagacaaatgttccggatgctttattgctctctgcgctggtgtttactgaaagtcaggctcggatcaggtttccgccgtcgcagccgcctcggagggagacaaggagccttgtgctgcttttctggagttgggggtggcggcgaggcgaaggaagtacaccagatggaaagatttttaccagattttgcaaacctgggagttaatctcgaagtaacgtcaacttttttgagcagggatttctttcgttttggcaaaagctgcgtttgttcagcgatcagcctcccgggaaaagctcgtgttttgtgactagccgggtgtcttcgggctccgtgaggggttgacagataaccatgctgccttctggaggtcggcgcttccccagccttgaggcgtatgttccaagggaagtaagcctgcaaatgagccttctaccatgtgatacctatttttctttttaaaaaatcgtttcatgaaaatccaggacgctcttcacactatttttttctaggaatttgcctagacaaagaattttttttaatgctaaggttgaaagggtcattttcctttagtcttgagaacaaacagcattagataagtttcttttccttccagaccttttttgtcctgtatatatgagacacttcacaacgttctggtctggagaatcctgaacttcgatgccacccaactgtgcggagtaatttttacttattaaaccatacccgccatgctaaaaatcagggaggtcttttaagtaatgggggggaaaatgtgacatctgaggagacttaatttactttgcgtagctttgcattatgatgtaatagctgctttttaacaccgacaattagggaaaagacggcttaataaaaaagtgtaatagaaagttttaggatgattacggctttcaattagtaacctaagaaccttagaaaatagtgtcaatctgcatgtagttacgtggaaaatttttcttgacatgttaaaagatatattcaggagtgtttgtgcgaaaaatatgtgtccataataccaaaatgtaagtttcatgaaatggaaattgtgctaggtgggattgtgtcccccttggctgcttggatattgtttcccatagttgccataggatcctgtgacagaattggaatgtggtccctgaaggtttctttgctagcccgtgaaaggcatactaagttggcttaaaggtttagaaaagtagggtgtcttcgttcaagaatttggaatgtgaagctcccagtatcttcatgtgtcctcatttaagtcaccattagattcagcccaacaatgtggtaagcgatttctatggacaacttgttggagtgaagcgattaatcctgttaaagaaaagaacgctccttttgagagatttctttgaagagtggtgctatttaaatgcccgggttgtttctcagataagggaacccagcaagagtgccagtgctctttgcagatggactactatatgttttatgcctggatccttatcatctgcctcagcaccccatatggctccacatttcacttaattcagcttcagaatacctcatttcttttattataaactggtgattgtgctgcagattcacacgtgtagtcctggtgttaaagaaactgtcttgaatggtgaggtctttatgtctgattcattcaaaaatgatgaaatgaaagctctagacccacaaatgctttagcatgttcagtaactggtgtgaaaatactgtcacactgatcacagataatctgacccaaaaatagagaaatctgtagctgcagagttttaattctgcccttggggaatgaaaagagtatgaaaagagtacctttagttgctttattttattttattttttttaagcaaacctctgcaattcctgaatagagatccaatatgggagttacttgcttactcatacacttcatggccttcttggctacatccttgagattgtttctgctccgcattgtcttggcattttgacaggaggcttactttaccgcctataacaggcaaagtgtgcgttgtcgttataaattcatgaagcctggatatttctgggtgtatgctacaaattagtcattatatagaagcccacaaggcagctgaaaatgctgacccaatcgcactttctatttagccctcttcgctcccatcatctctgtagcagaaggctgttggaagccttagccaaagcaaaagaaccccgggagatctgtttgatttgagatttgtttaagcagtcagcacctacctgctatgaaagagggaaaggtttaggccgggcgctgtggctcacgcctgtaatcccagcactttgggagactgaggcgggcagatcacctgaggttgggagttcaagacctgcctgaccaacatggagaaaccctgtctctactaaaaatacaaaattagccgggtgtggtggcgcgtgcctgtaatcccagctactagggaggctgaggtaggagaatcgcttgaacctgggaggcggaggttgcagtgagccgagatcgcgccactgctctccagcctgggcaacaagagcaagactccgtctcaaaaaaaaaaaaaaggaaaggaaaaggtttggggggttgcaaggaaggtatgaaaagggagctgggaatctaaattccacttttctttccttctgaatggttccatctgcatttccatactgttcgtttgatattttatgactatttgaaataattataaagaaacgtatgctcggatagattttatagaccataaaaagaacaaataggcactgcaagtttagcgggaagttcttgtgctgtcatttatccgtagtcttaagagctcttcattactgagaccttggccccatatttattttcttgctttctgtgtatctgattttaaaagaaaaaaaatgcaatagctacttaagtctttttttcagccatcaagtactgtcattgcagttaggaaataagaggagacgtgacctgtgatggaagaacaacaagttttgagggaaggcaccacatcctggcttaatgatgctagctgttttatgattctgccagaacaagccctttaatatccctttgccttggatttcccatctgcaaaatggctttaacgccttttggagttgtttagttggttaatttctgttttggtttgatttgagttctagtgaaaaatagctgaaaattatttgatctgctttggaaagagaaattcagtaaatactggagctacttttgtcagctttggaatgagcaaacataaagtagattccacacaaagatgtttttgtaactttttttttgaaagtatgatttgatgattgctgtaaatccatagctccaacatattaaaatgttttaatctttgactcaggttaaagtgaataccaagcctagactatatagtattttcccctttttgacccaaacacactttctctgtatttagccctcttccctcccatcatctctgtagcaaaatgctgttggaagccttagccaaagcagaagaacccaagaagatttgaaatttaaaatttaaaatttaaaattttgcaaatttaaaagtggtgacagagttgtcttcagaagttgtcttctcccacctgtttctcccagccaaagccagaaacagagcttacaagctactaaattcagatttaagagcagtttactagtgaaaatgttaaaatagctttctaagacaatattaggctaaattttaagtctttatcagatggagttccctaaatcttgatatcatcctgaagtttattgcatccacatcttgaaataattatatatttaataataatctactggccctgaattttttcccccatccagaatacaaatcataagatatttactgtctggaaaagaatacaaagttcgtcttagaatagcttcctcatttctcagatgaggaccctcacatttaaagagattaagagacgtctccatagtttaaagacctataaagattttatgtaacacaaataatgtgttacagttcatttatataaaatttgcagttgatgacaaagaacagtgtctcttttcactaagtctgttgtttccattttatggaacagaatattaaattatttggggacacacaactgacctagtattggtagtttttgtgctttaggagattttataaatttttgtttgagaaactagtggtaaaaagcatcatttggaaattgatttttttaaaactaatagggcagtgatactttggtttttcataatgtttatatgaattgtttttatttagtgtgcttttacttgtgatctgcttgggagctaggatcctagggtaatggagatagacagtaaggaaaaagtaaatatatagcttggtcaagtgaatcttgtttttttttttcttagagcatttacattttgtagaaattaggaaattgtgtcagtttatttacagtttcttggtaaacgtgcctcaggagaagtagaatgacagtacctggatttttttttttcttattctgtagccatacaggtaaatcaagtaaaggtgttttaactaattatactataatttaggattttacaccttcctgaagtggggaggttttaaccatggttttgtgcctagtgctgcttttccaatgctttctgaaggaagttatgcaattttggaaaatcaaaacaatgttcatgtttttggcgtaaccttagagtaatctttttcaaaacctatgaacaaatagcaacatttataaaatattttatttactattcccttgtaatctcaacctgtgatcactggactagactcatgttcactcacagtggctcatgctttaaagttgtaagttaactaattgcatgcagtttttcttccagtggcttagtttgagaaagacacaggaacacaatgaaaccctgtatctgaaggcttcttaaaataggagtacagtgtataaaatagaacaaacaattcccaaagaacaatgtgattttctcatgtagctcaggaagttatgaagagtttacaaatgtttggtcttatcttctttcccataggtgactgccaacagccagcccaatgtcgaatccagaaatgcaccacggacttcgtgtccctgacttctcacctgaactctgccgttgacggctttgactctgagttttgcaaggccttgcgtgcctatgctggctgcacccagcgaacttcaaaagcctgccgtggcaacctggtataccattctgccgtgttgggtatcagtgacctcatgagccagaggaattgttccaaggatggacccacatcctctaccaaccccgaagtgacccatgatccttgcaactatcacagccacgctggagccagggaacacaggagaggggaccagaaccctcccagttaccttttttgtggcttgtttggagatcctcacctcagaactttcaaggataacttccaaacatgcaaagtagaaggggcctggccactcatagataataattatctttcagttcaagtgacaaacgtacctgtggtccctggatccagtgctactgctacaaataaggcaagtataccttcttttttccctctccctactcaactttcaaaagatgtttgattcttcagataacttttgaaatgtgctataaagggcctagtttaaaaggaacttcttttgaaaagcaattaacagttgataaagggttaaataaaaattatctagtaaggaatttcttattggaatgtaaacgtggttctaattttaaatagacagtgatataaagaataaaaagtaaacagtgaaattgagttctccagggaaaaggcagacctgtttagtaaaaaaaggatgcttttttcagtgatgtctttttttgagtgcatatgtgtgtgactcttgaagaaatccatgttcagatttatcagatgattgaagtgggtgttctgaataaagaaagctgtgaggcctgaggcagtgacgtatcaggaaacatattttattggagatttggaagctatagtaaaacataatggcaataagccaacttcccagtggtaaacccacagtggtggtttagttactaacctcttgatgaccgaggaggttaataattggatattgcagagcagcaatatgtaacctgtgtgtaatctcagggcctcaggttaacagtttcagtcagaagctaagagaaacactgacaaaatttagcttaccatgactagctgccagtttatgtggtctgtgttcgtttgttttaaagaaactaatactgtacagagatctttgacaccaacttcaaggcaccagtgtttagtgcaagcaggtagagaggacctatgaactaggtttctaattggcagtcagtgctaattcagccattagtaagtaaagtcacttttccactaaaatagtgtcccgactaaccacctccttccgtgtcttttccaaccacaaaaccacacacatagaccatctcatcttactttatttcttgcatttattgtatccaaaaaaattggtttagttttttaatgtaattgacaagactcaagcttttgaaatacttacgactgttctcttgattccaggacaccctcccctgtgtctaaacatgtgtggtatactttttaaataaattaaagcttattttacttggcattttgtggagggaattttacaacacctcgggagatatctgcagacaccctggggtgtcacaaatttaagactagaagtcattgccctaatcaaagacgactgttacactggtctagacatcttaggggaactccaaaatgtttatacttcaactctcagtaaaagctcaagcttgtcttttgccttaccaaattccatatctcaaaataaacttttctccctctggtttttgtatgtactcaaatcatttttttataaatatctcagaatatgtatttgctataaattatatataaagttatagaattatgaagttataagagaaacgatggccccaaatccatcattactgcagagtcatatggcacatgaaagctgggaatagtctttgggtaactgaaagaaggcagtccattgagaatggaaaaagtcatcaattgtgcaaataattgaggacttggcagagtgaccttcgaacaaaactatagagattaatcagagcttgtaaatggggaaatactcattctcttcacaagaagcagtattcaaattttattcttcatgccttccatccggtcatgtcacttctgttattcagtctttcagaattctattgttactgattatggtgaaaatatgtgggggataaccttatgaatattgctatatatatggaacagtttacactgaagtatttaagatcattttatttcagttgaattctgttgtaatttttagcaaataaataaaaaggacaatcttgttttctccctcagtttggaataaggatcaaaggttttacccattcaaaaaaagttggagaaagtggtcctctgacataaattcaagtagatgatgagaatgcttggtctttaattgcactattggaaggtattttcccacatctggtgaccatgctgttcagcagcaaactattctgtagcctcacttcacaacctctaggagttgttgggtttggttccccatcccccttctctttgtaactctctccaaagccccataagcatctggaaggttgctcttgaagacctgtctcaatactagtatgacgtgtgctggtgaaaccaaagaaaggcctaaatcttacaatacgttccttttacccagccaggaccaaaataaaacctttaaaaaccttcagcacaaaaaagtggtggtgataaaaataaaaacaataagaggcataaaagtgcttataaaataagttctaaacaagatctgaaattttcccttaatgtctaccaccatactttctttaaaaaatcttttgaaaaattttttcatggaaatcccagctttactgagaatttaagcaagtgcatagaccagttgttgggtaacctagtactgtgccttgtagataccagatcatccttgtgtggtcaagtgtttttaataacccacgtgctcagtgccactagattgaaagactgatgtcattggctagctggccagctgtgatactgaaattttcggaaacatccattcatttcgctccctttaacactgcactgaagcactagaccccagggtattttgctcaatgaggctactaagacagcttctcagtcctgggaacctggcctcttgagcaagccgtcttcaagcccaactttgtgagctctgcctgttcctaggtaccccccttttccttatcacaaaaagacaaagtgggggaaaaatctaaacaccatagaatgaaatgaagtcacccggggccatatattagctaagccttttctttttctctacatggcaagcagtgagttcaaggcagacatgcctgcggtgcttaatgatgttgcctcatctcccctggtctactgttttcttccccaccatggccctctcctccccttccttgggaaaggtagggtttgttttcctgttggtcatggtgtgcacaggtatgctttctcaaggcctctgtgtttttagttttgccctgtgcctgcctccctctctttggaggaacctgtgccagttggctaccttgccaggtagatgtgtttctgaaggggaagccagggctggtttttcatctctccaccccgggggctgcaggggtgtctggcacacacacatacctcggctgacatccacatcagctctgggccatcttccccacctttggctgtgagaggcggctacaggcatgtaggcactcagaatgagatgagagggacaggctctgaactggcagagttagctacactgagaagaaaggcaggttttgttttgcaggaggaagtagcccacccttcacaggctccagagagggcacatgtcagtagttgtcctgtgcctcacacactcaagataaaccagatgatctgttttgaggatgcttgacttcttccatgttatttttgccaacctctcagtctcaactcttgttcattcattctgtggatctttatatctctgtttaagtgggttcaataagaatctgtatatgccatgatcacagaaggtgattggggaataaatttttttatgaaagattctgcactttgcaaatgtattcccaatagcatatattaaaattgccagtctccagggaattcaaaagtattcctttttcaaaaacaattctgaaattcatttattattaagttgcatacctaaacaaacaatggaaatgtttttagtaattatttttgtacaagtagtttgaatgccttcattttatttttccttttgttttaatgtggttgtatcctttttcaccactcttgctttcttattctatccttccacctcagtaacacaaaataaaaagtaggagtggagactggggatgacaacaggtcctactgcagatcttctgctacacattttactgtgcctcacccttgagtgagagaaggtagtttaccttaaggcgtttgaatttgggaaagggctgtgctcatcgtacttgaccaagcaagatgactgttaggcagatggacagatgctctgaggtgcctatcttgtacgtttttggtgctgccaacggccaccagctcctggcaatcctatgggcagcactcatcgcctgtccaaagcatagtagcatacccatcatgcattatggatagtctaaattccatacttacccctgaatttcaaattctgtagctactttcttgggaattcttgaaaatttccgctggttgaagatactagagatgtagaaagacttagtttggtttgcttttggttaatgtcttcttcaaactctgtaacctacagttttccattgtaagaaacaccattaccttgtaggctcctagagcatgaggtaggtctgctttctggtgccccttagcagggagttattgttctacaacagcatgtgggcatgtaaaccagtgttctgggtcattctttgttggttacaaaatcagaaccagacatgcctgcattgcaacagctttcagttgtcattttaagtatatctctgcataaggtttggaaatctgttctctgttgaccccagacctagtacatttattgtagctaagtttttgcaacccattatgtttttagcatctttatggaaaccatgtgagctacatagtgtttatcattatttttctggactaagggatatggttcttccattgataggtctgtgttttgaatctatggacactgttcttgcatgcagtgttataaagagggggacgcatggtatttgtgggggaaaaatagaccattttccctccacaaggaaactgcaactctcttggtgatacattgtaactaattcttataatctgagatcgctctttatttttgttacaattacattgttgcatgggtaattcttggcatctctttgagatttgctccttattgtgaccctccttgctttatgaagatgctaagaagttctgactggttgaggcatattgggaacagatggaaaggattttgcagttacacctccctgcttaatgtaattatttccctgataatcattctctgataaatgtatggttaatttggtgctgcatttgaaacagaaggtactaaagggcccactgtgggagatgtagaggtgaatgcagcctttgcctcaatctaagcacataactaaaatttgcagcagttggtgggaaagcacggggtttgtgtgtaaacccagcattttcagggttcttttcacattgacaagctggttccaattgatgttattcatttagcaagttggcaggggtgtcaagaagagccagaagagtgggctgctacaaaggcctgtcccacgctaacacccctccttgccaaatcattgtctttactcccgggatttggcacagtctctttccacctcccacagtaagaatgctcacctgcatctgtatttcctcatctgcatacaacctagcagggcctagaaccctggctcccacttttgaaagactgctgatctttgatgttataccaaaaagctttctagaatttgactgtcatagtcgccagttcttaattaaattcattttccagagcctcatctcatctttaaaattctcataaaagcatatgttagctatttttacctccctgaatttttttattatagaaacaatattgaaacattaaaagccaggttttcaaaagagcagaaatcagctgtagttccaggtggcagtctaattatttgtccttttctatttatcatgcagctgttgtgcatactggattataagatcttaggcccaggaagtactcaactttgctttatatcatttcagtaaattgcctggaggtgaaaaggtttccaagtggatgtggggaaaggaagcagagaaaaatggggaaattgctagctgtgtaatgaacctatgtctgaaaaatcattttgtaagataaatgagctacctgtttattctttatgctatacttttcaaagaagatttgcctttgtttcattcatacacattaaaaattctgggtaatgagtcttttaccatctttctaatctccctgactgtgccactccttctgtcccagatgtggtttggaaggaggaagagaccttcagaatgccccccatttgtaagtgaaattaacccagcgcctgtccctgtgtgagccaggcaagattccgtgagggtgctatggggctctgctgctttaaaacaaatccacactcttccatagtttaaaccagaggcatgaggccaagtctgatacacagctgagagttagaacttgtttttctgtcacatttccaaatgtctgcctggttaaattttctttgagtcaacattttgaggggaaggggtgagaaggaagcattggatgtttggtcctttttttgttggcttctattaatgctaaccagtttctgctcgtgtggaatgtggcatataaaaagttacctacattgttgaacaatgaaaactgctaccttatccattttgtttttatttgttttgactgcagtagaacttgctcttccactgtttcgaattttgtgttcttgtgggtggggtaacttaaggacttttcatactgacaaggaagctggccaacttgtcagtaagtaataaaagaaaagctaacatacatctaagttacagactttaactggacagccttgcagtgaaatcacagtgctattgggtctagaaagaagtttaggtgaaactggttctatggagagaggagttttttatgccccagttaactgtttactttaagagttggagccaaaagaacatgggggttctttcatgttgggacacagcatgtatactgtgaataggatgatatgacaccatctaaaactcacttctcatctgctctgcatgtgctcctgaaatgggggtcagtttccatttaagacatgtgtgtgcatgcacagatgtacatgcatgtgcttttacatggtatcgaggtcttcccctcaccctaccaaggaaaatgcagacataaagatattgttttgtaggatttcggttaactcacttcttgaaatgcatggtaaccattctaaccacttcattcctgagttctcatgggtcttttctttttcagagtgtgagtgtattgtgaagatcttcaagcccttttactttttttgctcacctcccaaagctgaaaaatgatcaaagagcctgtggttttctaatcccatcggtttggagaacagctgtgctgaacctaggcccagagcagggcctcgggtctgtagtcctgccacctctctctaccaggagagggaatgcctgaagggaagacctgctaggacagattcggctgagagttactgccaccagccctggctctcacctttcagaatatttttagcctctagtctgaagcaaggtggcagcctaggcctataatactgtccacacaggcctgccttcatatctgggcatatagtgagttcctacacttgctggcctcctcacaatgtggagtccgaacacaacagacaagctaagggagaggttccatttggagattttggagtgaccaagacttacaggacaaaggcctttcctcatgctcaccctaatacaaggaagttggcgaatttgcctgtctctgtggctatttatgcttccagcattcactgtcttaattgtacagaaattactcttgggaagcagcatttttgtgcctgttccttttatgtgtcttcctgggccagcttccctttcattctcgttttagccactgagcttgatgctgcttctctagaccaccctagctgccttcagctctgcgtgtccttcccctctggtttggtttcttcattctctatctgctgccaagtaccccttcagcagccaaggcagcccctgtttttgcatgtatttattacctacggcaacactgaagaataagtttggtccattccatttctctggatcctgtaagtgctccaagctctgtggaaaaagacatgaggatagagaaactgatacctctttttaaggtagctgattttttagtctcattcaagccctaaaaggagtccacagtatgagttttaatctgccactcacctcgtaaacacaggtactgtcttactgttagtatcttgtcctagtccaataaaattgtttaacaaaatgtatttttgtggatcttaagttgagcttcctgtagatatttgttttataagatatttgatatgtaggctgagtggtgattgtaaaattacatcccagttcattgagttcatagcttcctaacagtttttttcataatttttaaaggaaaattatcaatgacatcaagtattttctagtccattattttttcttaaatatggcatatataaaaactctcctgggttttgcatgaaattgttcagaaagagttattctaaatcaatatacctcttttcccccctcgagagagagacagagttctccccagtttttatgtaaccaggggacagttgccaccccgcgtggacagagtacctctgctctggccaaaatccacacttcagaattaccctggcgggtagggaggagtgggaccagggaagacacttgaaggaaggtgctttcaaaggcctctacttcctttaggtgcttcagagcatcctagtctctgacttcgtaacatttagaaaaattgacccttggaagtaaaatttgggttttttaggaatggagagagagcatgtatatgtttttctcctttgaaacatgtgctcttgttggaatggtttcttttttgttcactggggaagactgtagaggctgcatgctgttaaccatgtgttaagagtaaaaggggctgtgagcaccgtgggcttattctcccttagagtcttctatttttcaacctatcattgcacccagatgatatggcaaagtagacacatcagctaattatatgaaataaaattcatcgacttctttgacactgcagaattttaaccaggtgcatatcaaaattatgttttcatgtgtttcaagctagtttctgtgcctcttcagtttagtttactgaccagtctagagtccagagatgagagctcctaaagtctggctctcatgttttgaggattccagaaaagtcctacactcacagtgccaaatggcctgattaaacacagataaatgtttcttccaaaatatattacgcacaagtagtcaaggtgcacagccatttgagaagtctgctgttttaaaacctgtttgtggcattttgcaaggctgctgtgctttggatagatatctctgtacctcttggcgaggcagtacctgtgtatttccctctttgttggctacctaattttcatttaatatgttttttttttttctcccggtggaaaggagaggcaggtgcctcttaaaattatttcacagcaaaccttcgtaagttcttattgcctatttcatgtagaaaggttgatttgtagctgctgtctttctcttcctaataaaccttgtgggtggtgaagatctaggagagtgaaacattaaaaatgaatgaagagaactgaagtagtttgtctaaggtgggggttgagaggggctgggtggaggaggtttaacaaaaggcattgatgttttaaatgcacagaaatgccatctatcaataataaatggtgtggcatagaacagatgtgccaaattgagttatgacagcatatatattagttcatagaggggaaaagtaagtagcatagctaacgtttttaaattatctgtgagcttttgaaatacaattttatgtattcagtttttactttctagtggctgctagctggaaatattaaaagtttggtgttttttaaaattccaattaaattgtgcttgtttctaaagtaagaattaagaatgaataaattttgcttttctgaagaggaagacagtctgaaatgaatttcttgcccttcattggctgagtacttgccttttaaaagctcatgttgtaattgttatgaggaatgaaagtgcattataaaacttaagtcatgtgaaactattacccagtgttttctaaatttacattgattatatctctaggtacctgctcagtgcatatttaaactacaagggactactcaggaaacccactcatgctttacaatcaattcacccccctcagcattttgattcaagggataattctacacatatgctctgtgtgagaattaccttgcttggttgggaaacaacaagagaaatatccacttgtctcacagcaaatcactactagtaaattggaaagacaaatcttttaggttaactaggatattttgtaggtgtagatactagagcagaaaacaagttttgagcttctagataggtaggagttattgttaatgtcactggcatagcagaggacaatactgtgggaggtgtttgaaatgaatcctgagttcctgagttggaattggtgtgcttaatgaaaaaaacagccgtggtgagacatatggaaccattccactcatctcaatatggacccttgcttgctttttggaaacttgtttggaatcggttatctttgctttgttggcccaacacaagtaaatcctctttgtttttcctaaggtgaacaaaaaagtagttcttgcagataatatcaagttggtttaaattttaggtttgttagactagaaagacgaattatttagccgtcattgaagagtttaatctttgagtagatgttacaggtgaaaatgagcatagtgattatataaaaagtctattactaactcttattttagccatctgtattgttgactttcaactttttcaggtgcaaaattttcttgaatcaatcttttcatctccaagggatcccagcatgccttaaaaaaaaacacttgtcaacggattttagagccaccaaataaatctattttatggtcccgttagatatctagctcaaaacccccattcactctgaaaggtcctatggttggatgcacaacaaatttcctgaaatcatctctgatgcacctcctttgggttgttaagaaggtgttgaaatgtgagaaggctggtagtgaaatcaagttgtggctcagggtaattaggaagggagaaaacccagcattttcatagtacattgtctggcttttagttgcagctgcacacagcggatatgaatttaaccggttgctacagtgcagctggttcagatctggctttataactggctgaagtaaaatgaaatggctggaaaattccagtcaagtaacaccagtgggaacaagtggaattaaatagtgtgagcaaatgctggccgagttggagaaaaacttaagacataaacttgagggtggtggtgaggacttcgggtgggcaggcggggggaggggggtgcattttctggtctttttcacaagggtctattgtttcgattagtttccttgcaggaggtagaaggtttcctccatcccgagtaattcatagagtgtttggcttgtctaccttcctcctgactgaagtcacctgatactttttgttttttcaagaaggaagagagaaccctgttgcctcagttactagcaatgatacaattctcaaaatctggtctttttttgtttctttgaaatagtttctccatgttgtgtgacacagcagcccctgtcttatcatagttgtcttccctccaccacctgtaccagagatgttggatatgttggaggtgaaggtgtgcaaggtttttaactaactgttctaattaaaggattctgcaggaaagaacatgggtttacaaaagagaagcttttgtattattagtaattttttttctttgatgaatttatgtgcttagtttggagaatcgagagttggctgggaaaagatttctgaggagttaagggactctggtgctgtttgggaaaacatttgctgtaactaggtttagggtattcagtctctgccagtgcttaggagctatatttgccaggtgatttccctgtcgcagggattagccagtgatgtgaaacatttgattaagggactaatagaaatctttcctggggtacatgagtggtggctgttactggggtgccccctcctcaactctttctctctgtatttcgagtaagtttgtagtggtgagtcatgggatacagttttttaatcctatgaagaatcttggtaggaagcaaagttctgttaggtatattgttaattaagccctttaaagtttctgcataataactcagaaacgagcctttttcttttctcagaactatgccatgactcatccactttggaagcagacaagatataaattatcaataaaaattgcccataatctggcctggcacagtggctcatgcctgtaatcccagcactttgggagatgatggtgggaggatcgcttgaggccaggagttcaagaccagcctgggcaacgtagtgaggaacccccgacctccaccatcccccccgccccccaccaaccccctcacttcaaagttttttaaatcagccacgcatggtggtatatgcccttagtcctgtgtactcaggaggctgaagtgggaaagtcctttgagcccaggagttgaacgttgcagtgagctatgaatgtgccaccgcactctagcctgggcaagagagctgaccctgtcacttaaaaaaaaaaaaaaaaaaaaaccgatattctgatagcaacactgctcacttgctcactctttctcttaggaaagtaaaaaagttttgtcttgatgcacagatttatttaaactttagttctaaatatataggactttaaaaaatattttctgaaagtgatatccatttttgaggtgcaagcagttgggtccattttggagattccgccttaagccattcagcttcaaaatggcggggagctcttgctacagagggttaccccgttctgcttccttcccattctgttaaaccttgtacatgctccttcccacagatcactattatcttcaaagcccaccatgagtgtacagatcagaaagtctaccaagctgtgacagatgacctgccggccgcctttgtggatggcaccaccagtggtggggacagcgatgccaagagcctgcgtatcgtggaaagggagagtggccactatgtggagatgcacgcccgctatatagggaccacagtgtttgtgcggcaggtgggtcgctacctgacccttgccatccgtatgcctgaagacctggccatgtcctacgaggagagccaggacctgcagctgtgcgtgaacggctgccccctgagtgaacgcatcgatgacgggcagggccaggtgtctgccatcctgggacacagcctgcctcgcacctccttggtgcaggcctggcctggctacacactggagactgccaacactcaatgccatgagaagatgccagtgaaggacatctatttccagtcctgtgtcttcgacctgctcaccactggtgatgccaactttactgccgcagcccacagtgccttggaggatgtggaggccctgcacccaaggaaggaacgctggcacattttccccagcagtggcaatgggactccccgtggaggcagtgatttgtctgtcagtctaggactcacctgcttgatccttatcgtgtttttgtaggggttgtcttttgttttggttttttattttttgtctataacaaaattttaaaatatatattgtcataatatattgagtaaaagagtatatatgtatataccatgtatatgacaggatgtttgtcctgggacacccaccagattgtacatactgtgtttggctgttttcacatatgttggatgtagtgttctttgattgtatcaattttgttttgcagttctgtgaaatgttttataatgtccctgcccagggacctgttagaaagcactttattttttatatattaaatatttatgtgtgtgcttggttgatatgtatagtacatatacacagacatccatatgcagcgtttcctttgaaggtgaccagttgtttgtagctattcttggctgtaccttcctgccctttcccattgctactgatttgccacggtgtgcagcttttactcgccaccttccggtggagctgcctcgttcctttgaactatgccctcacccttctgccctcacttgatttgaaagggtcgttaactctcccttacaggtgctttgactcttaaacgctgatcttaagaagctctcttcatctaagagctgttactttttcagaagggggggtattattggtattctgattactctcaattctaattgttatatatttgagcccatacagtgtattaggttgaaccatagaaactgctattctcgtaggtcaaaagggtctagtgatggaagttttgtagataagtaccaggcatctcagtaactcctagactttttctcatcccatgccccgttttaaattgtcagttttccctctgactcttctgtgttaaaacatgaaactataaatttagtaattatcatgccttgctctttttaatctatatgactgatgcaagcccctcttcttaaccgtttcttggctttgagcccagaaacacagctctccctgtctccaactccagtaagccctcctcagcctcaccttacgaatccaaagaactggggtttgttaggttctttctctaatgtagaggcccagatcccatcacaaagtttttcattcttccttgtccaccatgatcttcatcacagtctttgatatgtctgcatgcaaagtggaacagagttgggcggcaatgacagaagagcttccttggcctgactcggtgtgcggccacttcggcactgcttaatccagatattcttgttaactaagcattgtgcttcccaggtggtctgaagtcaggtactctctctctcaacacctgtagttgaatatgatttggtcagttgctcgttgtaacttggagaaattcctataaagtaagatctccttgcctcttccatccattgttggcacccccttgcaaaaggaaaagaacagcaaaagtcaggagcagtaatctgagaaagttaactccaggataggtaggtttctattgttatagctagatgtaaatctttagttccaagaagtgatagagtttctgctttaataatttgttgataagtttacataaacagaaataaaagatactatctttaccgtagtagttcaggccaagattatgcttagttttagttctccaggtagttacttttgccatgtcctattgatcagtgacactgccagaggcccataccggcaagaggaagaggacgtcattttgtaaagtttaacttcttagcgaactgatgtgccacccagtcacagagtggagttgtgaattcatgtagaggtggcaaacctctaccttgtgttgatgagagaataatcttgggcagtctgggaaaataaggaaggcatctccttcttactcatggagattcaactatagagagttgaaacctaaacccgccttccttttatagaagctggactagagacggactgaccatcagctctgaactgtggctttttttgttcacctatgatgccatgtaccaaattcagaagctatcgttaataatttgttttataattgagtagtacaagcgaggaaaaaatacggaggataaccactatttttgtgcaaataatatgaaagtgaagtaaaagcaatagaagaaatttctataggatctgggtttagagtgtgtatcattaataaatatacctttgctcttttcagggaaaataacaaccacccttactgatagttgggaaaagaagattgggttattttgccatatcatttagctggaagtgacatttaaaagcaccctgcatcactagtaatagtgtattttgctattctgcccttgtaatcggtgtccctgtaaaacaatccccacagattactttcagaaatagatgtatttctctacgtaagggccaggtttattttctccttttttgagatttctagaaaaaatgctgcttgcacatgttggttcttgaaaccttagctagaagaatttcaggtcataccaacatgtggataggctatagctgttcagaggtctcctgggggagcttaaaacgggggaaacactggttttcacagatgctccacatggctgtctttaaaagactcaaaacttttttttgtcctctttgttatgcttggaagctcccccccccccaacagtgtgtcgagtctttgcaaagaaacctttagatgtggttcatagatatatgaatacgtatctgtgtaaaacagtgagtgtgcagtgtgtaaatactttaaattattatgctagaaaaataaagttacataccttgctgtggaa

SEQ ID NO: 18 - DNA, 27865 bp, Homo sapiens
  source 1..27865: mol_type = genomic DNA
gtaacacacttgctctgggccttaggtgtctctttaaaccctctctcccctctttatttccaaatctttatctttggctttcttaagctgtaaaggcacactggcaccttaggtggtcaggtaagatcgctggattccttatttcggcgaaagtccagcggaccattcccgccactgtacccattttagaaacacttggaagacccttaaggatcatttacggagagaaaggtgtgcattctgcggctgccggcgggcagctagagaacgtggcggggggcagcgcatctggcgccttggccggggcgcggagtgggaacagcccgcacgcgtccgctgccgccctgcctggcgacgagtggcacaatcgcccaccaggaatcggctggtgccgcgggtgggcgaaggcagcgccgccggcccggagggcggcgcggagggtggcgctggcggcgtgaaaggcgtctttgtgcaagattgagtgtggtggggggaaggcgaagagagaactgatccaggacctggacgcgggggtctgcggcgcccctacgcccccacgcccccagccgtccgcagtgccggaatgagccggatagcgccggcagggggcagtgtggcgatataggggttgccggggggaggggagggtctctgttcacctctatttgaggctgactggggagaaagctgcctggaggaagctgctggggtgcggggggctgagggatttggctccccgccctctcccctagatggcggagaaatcgggctagctggaagggcgcagctcctctcccagagctcattctggagtcggagaactgggagagcggcccccgaggcggagcctccctcccgccccgagtcgcgctgccccccacctgggggagaggggcgggcgccgcgctgccttccctccgcgcctcggccgcgtggcttgcggcttattttcccagctggcaagcgtcgcgctgcagacaagggaatgcctgtggtgagttgtttattttgttgaagtttgcggtgggcccgggggacgtggggggcgagcggcaggcagcggccgggactgttttgtttaggtctccatccacctgtgtggtataaatctgccagcctcaaggcggcgggggaccggagtgagaaagctctgacgctcccaaaagccccttttcgcaacttacagatctaggaccacatttgattctccacgcctggaaccggctagatcctagtttacgctcaccccgccctccagcctcccatcaacgcttcccgcacccgctgtgtgcagggctggcgctccgcagagcaaacagcgcccacgacccagggacagctggccaccgaagtccctccccggcgcccttccccgttccttgaggataggggagctgaggttttcatgtgaagggcgcccatgaaattcgtgggaacttgttctggcgtcttctggagtggagttctttcaggggtttcgggtctgcccgggactacaaaatgggatcctgggttggctggagtttcccagactaggctggagagaggtcggctgttggctcgcgggagagcccagaggaaccccttgggcgatggcagtcaagaccggcggctgcgtttgcctggaagaaaagcctaaatacattagcgagctggtaaagcttttaaggccttcttgggagcgagtggatggctaatgagaggttaattttgtttcgaagggaaggctgcactggtttcctgagataaatggacaggaagcgcattacctccgcgcgctggcaagcggctcccaaatgctttttgcacctgccgttttgagcggtgagagcagcggcggagcccgcagggattgcgggggcccgttgtctacgagcgggtgatgagccccccgcaagccccgggctctctccctccgcgagtgcattgctcacgccaagctttgggactcttctctcacacccaggtcctgcgtgttccgaagttcaggggccccctgatcccgctgagccccctccaggagcgaaagggttaagaatgatgtaagtacaggcacttgatttctcaagtcgcccgcttggccctcttccaagttgtactttccttattttctctcctttcctttccttactgttctaacgctttttaataaactttcactcttgctctgaaaaaaaaaatgatgtaagtacaaaccacgagctgcttgacgctgagctgttggactccgggctatttcagccgctgctttgggtacacagtggccggggagcagccggggagcccttactctagaaaggattgcacaaatcgcaagcttaggtgatctgttaagcagtcagagattctcctgttagatttctggaaatggagacatattaactggagaacagttaaaaacatcacgtataggctcaggaaatgaaactgcatcttgtttagaatccattagtgtttctggcatgtgttttaattctgtccacttgcacgttcatgccgtagtatgctttgctggattctccggatgtactgttcaggtctcctccttcctttctgaaaacaaagagaaagggagaaagagtaccagtggcaagaaacagggtggatgtgtgaattacacaagtcatcagccacttcatgattttttttctgattaaaacataaaacttttataagtcaccgatttttttgtttgtgttctagctccctccccgatttttctttgtacaatataaagtcgtttgtaacttgcagctcacgggcttgaggagtggctgcggtataagcgactattcactacgctcttgctttccaagccaattcctgatcttgtgaacagtacgaatttacccgcgagtgtgtcaactgacctatgtcttgaaaataggaaccaccttgcggtttacttgtgaaaattagcattttcctctagcggctgaacacttatgcagcaataaaaagtgaattgttaggcttataaatctcgagcttgtcaaattttacaacagaggagcaataaagcaccttatagagatgaagtttttcctctgaagaacagttccgtttaacgacaccaggatttgcaatgactcgaggacgccctctattgataggaaaatctagagcacccgtgtacttatggaacaggatatagggggaggggagtaaaaaatcatagaaagtcacgagtggaaagacccaaaccaaacagaaattattgatccttaactattgagtatatgctttgaagtatgatcaataaagtgagatgctgcttttataggggtcgaattaaatcttgggttaagagaaaaaaatgtagtctttgttagccaaatactttgaaaatcagtattatgtttgattacttttttaagatgcttcttagtatgtcatgaactgtatctaattatatgaagtaattgcaagatagtaaatttttgctctaataatacttagtccgcagcattatctaaaaatgtcatataacttcctccccaaaagtcatttctccaggcaactgtgaagctattattaagcgtattatggatatacacaggcttggattatattgtggatagtgaaaggtgtgtttaccccttaagtcatcatagtatgtaacaagtaagtaatgaaaagttacagtaaaaacagataggcatccttcattagggacctagtggtttgggcaattcaaaatgtgtctaataagtgagctggtgagactgaaggtttgcccctcaattatcctagtccattgattagctcatttaaacatgtgtggaatatcagcatgtccgtggttgataagaaattcctaattagagattctgagtaacatagcgggtacagttgccaactacgatttggatgttagtggattttaaatggcaaccctgtgctgtttggggaaaggcaaaggcgagggcggtatcaagtgtctctcagccttgaggtcattcttgagattagataccacttcacacctaccgataacaaattggaatctatcatcagtctcataactgcttttctgtctccacttggcagcctctttctttgtagacaattgtctttttcctcactttcggacccatccgatcataaaaggagaaagtgagctgccattgaattaatccgtagaaatttgtggaaaatttaaataatctgtagaaatttgtgaaaaagccactgagcaacaacaaatcgtggcataattacactgcataaaaacccaccactctcattaattacactgtaaccgaacgttgaagtttgcccactcttagacctctccttgccgctttcaggaagggaaatgtcaaactagaacacctgagcctcgcacagctgccagcgaattccggcggcagttcgcgggtaccttcccagcccatcctatctgctccggaccgcgggtgacattcagatttccaacaccgccccgagcttgctttaccagacaccctcaaacgactcggaacgcggttccgcagatgtgattggctgaaaggggacctgccgcgcggcgctctgtaaaagcgcctgcttcctccaaggaggtggagctgaagctggcagggccagggcagggaggggtaattaaattaaaaaaaaaacttggagggaagatgaatcggatcacattaattcaaggagaggtcaaaggccggtacctttgtaacgccggcgagagtaagacttgcggtcgcccatttctgttgggcgcgaggaaacagtaaatcgcggaagggggtagagggggaaggaaagggagcgcgaggcagcatgcggcgcaggagagaggacttggagtcgatggacagggaactcgaattacactgagcgggcgggggagagggccctctgggtcctgcgattggcaggctgagtcgggctgacgtgcggtcacaaccaatggggaggaagggagggacacggcgggggattatggcgtcgtggattggcgggcacagggcggggcgggacggcagtgcgggactgagattgatcgaccgcgggggctgccgcgcagagatatccgggccgccggtgggtggtcgctagggctgggccagcctcttggaggtccacgcccgccgagcccacgctggctggggccggggtgccggcgcgctcgggactcgtctcagcagtcgctcacggtctttgtgtcttctcttccgcccctttccctgcctgccgcctccggccgccacgatgcccctgcgccccgctgctgccgccgcggactggctgcgccggctgcgcgctgcttgctgcggcggtggtggcgccccatctgctacacgggcctgaagaaggaagaagaggaagcgaagcgcgccccccggcccatgccgcagccacgggcccagacccgccacggcgcccgcgccgccgccctcgccggagcccacgagacctgcatggacgggcatgggcttgagagcagcaccttccagcgccgccgctgccgccgccgaggttgaacagcgccgccgccccgggctctgccccccgccgctggagctgctgctgctgctgctgttcagcctcgggctgctccacgcaggtaggacgggagcgcgcgaggggagccagccccggggcctagggctttgttcccaaatagccccaggtctcgaaggcgctcgccggggcggaagggcggcgtggcgcaacgggaaaggcctcccgagcccctgacacgcacctctgtcgggctccgggccaccgtaacgcagccacccaccgggcggccgccgggcgcagagacgagccttctctccaccctccccgccttcggctcggaccgctgacaaatctcccctccgacagccgcttatcaaacatttagcgggcgggggccgcgcgcccgagacaaatgttccggatgctttattgctctctgcgctggtgtttactgaaagtcaggctcggatcaggtttccgccgtcgcagccgcctcggagggagacaaggagccttgtgctgcttttctggagttgggggtggcggcgaggcgaaggaagtacaccagatggaaagatttttaccagattttgcaaacctgggagttaatctcgaagtaacgtcaacttttttgagcagggatttctttcgttttggcaaaagctgcgtttgttcagcgatcagcctcccgggaaaagctcgtgttttgtgactagccgggtgtcttcgggctccgtgaggggttgacagataaccatgctgccttctggaggtcggcgcttccccagccttgaggcgtatgttccaagggaagtaagcctgcaaatgagccttctaccatgtgatacctatttttctttttaaaaaatcgtttcatgaaaatccaggacgctcttcacactatttttttctaggaatttgcctagacaaagaattttttttaatgctaaggttgaaagggtcattttcctttagtcttgagaacaaacagcattagataagtttcttttccttccagaccttttttgtcctgtatatatgagacacttcacaacgttctggtctggagaatcctgaacttcgatgccacccaactgtgcggagtaatttttacttattaaaccatacccgccatgctaaaaatcagggaggtcttttaagtaatgggggggaaaatgtgacatctgaggagacttaatttactttgcgtagctttgcattatgatgtaatagctgctttttaacaccgacaattagggaaaagacggcttaataaaaaagtgtaatagaaagttttaggatgattacggctttcaattagtaacctaagaaccttagaaaatagtgtcaatctgcatgtagttacgtggaaaatttttcttgacatgttaaaagatatattcaggagtgtttgtgcgaaaaatatgtgtccataataccaaaatgtaagtttcatgaaatggaaattgtgctaggtgggattgtgtcccccttggctgcttggatattgtttcccatagttgccataggatcctgtgacagaattggaatgtggtccctgaaggtttctttgctagcccgtgaaaggcatactaagttggcttaaaggtttagaaaagtagggtgtcttcgttcaagaatttggaatgtgaagctcccagtatcttcatgtgtcctcatttaagtcaccattagattcagcccaacaatgtggtaagcgatttctatggacaacttgttggagtgaagcgattaatcctgttaaagaaaagaacgctccttttgagagatttctttgaagagtggtgctatttaaatgcccgggttgtttctcagataagggaacccagcaagagtgccagtgctctttgcagatggactactatatgttttatgcctggatccttatcatctgcctcagcaccccatatggctccacatttcacttaattcagcttcagaatacctcatttcttttattataaactggtgattgtgctgcagattcacacgtgtagtcctggtgttaaagaaactgtcttgaatggtgaggtctttatgtctgattcattcaaaaatgatgaaatgaaagctctagacccacaaatgctttagcatgttcagtaactggtgtgataatactgtcacactgatcacagataatctgacccaaaaatagagaaatctgtagctgcagagttttaattctgcccttggggaatgaaaagagtatgaaaagagtacctttagttgctttattttattttattttttttaagcaaacctctgcaattcctgaatagagatccaatatgggagttacttgcttactcatacacttcatggccttcttggctacatccttgagattgtttctgctccgcattgtcttggcattttgacaggaggcttactttaccgcctataacaggcaaagtgtgcgttgtcgttataaattcatgaagcctggatatttctgggtgtatgctacaaattagtcattatatagaagcccacaaggcagctgaaaatgctgacccaatcgcactttctatttagccctcttcgctcccatcatctctgtagcagaaggctgttggaagccttagccaaagcaaaagaaccccgggagatctgtttgatttgagatttgtttaagcagtcagcacctacctgctatgaaagagggaaaggtttaggccgggcgctgtggctcacgcctgtaatcccagcactttgggagactgaggcgggcagatcacctgaggttgggagttcaagacctgcctgaccaacatggagaaaccctgtctctactaaaaatacaaaattagccgggtgtggtggcgcgtgcctgtaatcccagctactagggaggctgaggtaggagaatcgcttgaacctgggaggcggaggttgcagtgagccgagatcgcgccactgctctccagcctgggcaacaagagcaagactccgtctcaaaaaaaaaaaaaaggaaaggaaaaggtttggggggttgcaaggaaggtatgaaaagggagctgggaatctaaattccacttttctttccttctgaatggttccatctgcatttccatactgttcgtttgatattttatgactatttgaaataattataaagaaacgtatgctcggatagattttatagaccataaaaagaacaaataggcactgcaagtttagcgggaagttcttgtgctgtcatttatccgtagtcttaagagctcttcattactgagaccttggccccatatttattttcttgctttctgtgtatctgattttaaaagaaaaaaaatgcaatagctacttaagtctttttttcagccatcaagtactgtcattgcagttaggaaataagaggagacgtgacctgtgatggaagaacaacaagttttgagggaaggcaccacatcctggcttaatgatgctagctgttttatgattctgccagaacaagccctttaatatccctttgccttggatttcccatctgcaaaatggctttaacgccttttggagttgtttagttggttaatttctgttttggtttgatttgagttctagtgaaaaatagctgaaaattatttgatctgctttggaaagagaaattcagtaaatactggagctacttttgtcagctttggaatgagcaaacataaagtagattccacacaaagatgtttttgtaactttttttttgaaagtatgatttgatgattgctgtaaatccatagctccaacatattaaaatgttttaatctttgactcaggttaaagtgaataccaagcctagactatatagtattttcccctttttgacccaaacacactttctctgtatttagccctcttccctcccatcatctctgtagcaaaatgctgttggaagccttagccaaagcagaagaacccaagaagatttgaaatttaaaatttaaaatttaaaattttgcaaatttaaaagtggtgacagagttgtcttcagaagttgtcttctcccacctgtttctcccagccaaagccagaaacagagcttacaagctactaaattcagatttaagagcagtttactagtgaaaatgttaaaatagctttctaagacaatattaggctaaattttaagtctttatcagatggagttccctaaatcttgatatcatcctgaagtttattgcatccacatcttgaaataattatatatttaataataatctactggccctgaattttttcccccatccagaatacaaatcataagatatttactgtctggaaaagaatacaaagttcgtcttagaatagctttctcatttctcagatgaggaccctcacatttaaagagattaagagacgtctccatagtttaaagacctataaagattttatgtaacacaaataatgtgttacagttcatttatataaaatttgcagttgatgacaaagaacagtgtctcttttcactaagtctgttgtttccattttatggaacagaatattaaattatttggggacacacaactgacctagtattggtagtttttgtgctttaggagattttataaatttttgtttgagaaactagtggtaaaaagcatcatttggaaattgatttttttaaaactaatagggcagtgatactttggtttttcataatgtttatatgaattgtttttatttagtgtgcttttacttgtgatctgcttgggagctaggatcctagggtaatggagatagacagtaaggaaaaagtaaatatatagcttggtcaagtgaatcttgtttttttttttcttagagcatttacattttgtagaaattaggaaattgtgtcagtttatttacagtttcttggtaaacgtgcctcaggagaagtagaatgacagtacctggatttttttttttcttattctgtagccatacaggtaaatcaagtaaaggtgttttaactaattatactataatttaggattttacaccttcctgaagtggggaggttttaaccatggttttgtgcctagtgctgcttttccaatgctttctgaaggaagttatgcaattttggaaaatcaaaacaatgttcatgtttttggcgtaaccttagagtaatctttttcaaaacctatgaacaaatagcaacatttataaaatattttatttactattcccttgtaatctcaacctgtgatcactggactagactcatgttcactcacagtggctcatgctttaaagttgtaagttaactaattgcatgcagtttttcttccagtggcttagtttgagaaagacacaggaacacaatgaaaccctgtatctgaaggcttcttaaaataggagtacagtgtataaaatagaacaaacaattcccaaagaacaatgtgattttctcatgtagctcaggaagttatgaagagtttacaaatgtttggtcttatcttctttcccataggtgactgccaacagccagcccaatgtcgaatccagaaatgcaccacggacttcgtgtccctgacttctcacctgaactctgccgttgacggctttgactctgagttttgcaaggccttgcgtgcctatgctggctgcacccagcgaacttcaaaagcctgccgtggcaacctggtataccattctgccgtgttgggtatcagtgacctcatgagccagaggaattgttccaaggatggacccacatcctctaccaaccccgaagtgacccatgatccttgcaactatcacagccacgctggagccagggaacacaggagaggggaccagaaccctcccagttaccttttttgtggcttgtttggagatcctcacctcagaactttcaaggataacttccaaacatgcaaagtagaaggggcctggccactcatagataataattatctttcagttcaagtgacaaacgtacctgtggtccctggatccagtgctactgctacaaataaggcaagtataccttcttttttccctctccctactcagctttcaaaagatgtttgattcttcagataacttttgaaatgtgctataaagggcctagtttaaaaggaacttcttttgaaaagcaattaacagttgataaaggattaaataaaaattatctagtaaggaatttcttattggaatgtaaacgtggttctaattttaaatagacagtgatataaagaataaaaagtaaacagtgaaattgagttctccagggaaaaggcagacctgtttagtaaaaaaaggatgcttttttcagtgatgtctttttttgagtgcatatgtgtgtgactcttgaagaaatccatgttcagatttatcagatgattgaagtgggtgttctgaataaagaaagctgtgaggcctgaggcagtgacgtatcaggaaacatattttattggagatttggaagctatagtaaaacataatggcaataagccaacttcccagtggtaaacccacagtggtggtttagttactaacctcttgatgaccgaggaggttaataattggatattgcagagcagcaatatgtaacctgtgtgtaatctcagggcctcaggttaacagtttcagtcagaagctaagagaaacactgacaaaatttagcttaccatgactagctgccagtttatgtggtctgtgttcgtttgttttaaagaaactaatactgtacagagatctttgacaccaacttcaaggcaccagtgtttagtgcaagcaggtagagaggacctatgaactaggtttctaattggcagtcagtgctaattcagccattagtaagtaaagtcacttttccactaaaatagtgtcccgactaaccacctccttccgtgtcttttccaaccacaaaaccacacacatagaccatctcatcttactttatttcttgcatttattgtatccaaaaaaattggtttagttttttaatgtaattgacaagactcaagcttttgaaatacttacgactgttctcttgattccaggacaccctcccctgtgtctaaacatgtgtggtatactttttaaataaattaaagcttattttacttggcattttgtggagggaattttacaacacctcgggagatatctgcagacaccctggggtgtcacaaatttaagactagaagtcattgccctaatcaaagacgactgttacactggtctagacatcttaggggaactccaaaatgtttatacttcaactctcagtaaaagctcaagcttgtcttttgccttaccaaattccatatctcaaaataaacttttctccctctggtttttgtatgtactcaaatcatttttttataaatatctcagaatatgtatttgctataaattatatataaagttatagaattatgaagttataagagaaacgatggccccaaatccatcattactgcagagtcatatggcacatgaaagctgggaatagtctttgggtaactgaaagaaggcagtccattgagaatggaaaaagtcatcaattgtgcaaataattgaggacttggcagagtgaccttcgaacaaaactatagagattaatcagagcttgtaaatggggaaatactcattctcttcacaagaagcagtattcaaattttattcttcatgccttccatccggtcatgtcacttctgttattcagtctttcagaattctattgttactgattatggtgaaaatatgtgggggataaccttatgaatattgctatatatatggaacagtttacactgaagtatttaagatcattttatttcagttgaattctgttgtaatttttagcaaataaataaaaaggacaatcttgttttctccctcagtttggaataaggatcaaaggttttacccattcaaaaaaagttggagaaagtggtcctctgacataaattcaagtagatgatgagaatgcttggtctttaattgcactattggaaggtattttcccacatctggtgaccatgctgttcagcagcaaactattctgtagcctcacttcacaacctctaggagttgttgggtttggttccccatcccccttctctttgtaactctctccaaagccccataagcatctggaaggttgctcttgaagacctgtctcaatactagtatgacgtgtgctggtgaaaccaaagaaaggcctaaatcttacaatacgttccttttacccagccaggaccaaaataaaacctttaaaaaccttcagcacaaaaaagtggtggtgataaaaataaaaacaataagaggcataaaagtgcttataaaataagttctaaacaagatctgaaattttcccttaatgtctaccaccatactttctttaaaaaatcttttgaaaaattttttcatggaaatcccagctttactgagaatttaagcaagtgcatagaccagttgttgggtaacctagtactgtgccttgtagataccagatcatccttgtgtggtcaagtgtttttaataacccacgtgctcagtgccactagattgaaagactgatgtcattggctagctggccagctgtgatactgaaattttcggaaacatccattcatttcgctccctttaacactgcactgaagcactagaccccagggtattttgctcaatgaggctactaagacagcttctcagtcctgggaacctggcctcttgagcaagccgtcttcaagcccaactttgtgagctctgcctgttcctaggtaccccccttttccttatcacaaaaagacaaagtgggggaaaaatctaaacaccatagaatgaaatgaagtcacccggggccatatattagctaagccttttctttttctctacatggcaagcagtgagttcaaggcagacatgcctgcggtgcttaatgatgttgcctcatctcccctggtctactgttttcttccccaccatggccctctcctccccttccttgggaaaggtagggtttgttttcctgttggtcatggtgtgcacaggtatgctttctcaaggcctctgtgtttttagttttgccctgtgcctgcctccctctctttggaggaacctgtgccagttggctaccttgccaggtagatgtgtttctgaaggggaagccagggctggtttttcatctctccaccccgggggctgcaggggtgtctggcacacacacatacctcggctgacatccacatcagctctgggccatcttccccacctttggctgtgagaggcggctacaggcatgtaggcactcagaatgagatgagagggacaggctctgaactggcagagttagctacactgagaagaaaggcaggttttgttttgcaggaggaagtagcccacccttcacaggctccagagagggcacatgtcagtagttgtcctgtgcctcacacactcaagataaaccagatgatctgttttgaggatgcttgacttcttccatgttatttttgccaacctctcagtctcaactcttgttcattcattctgtggatctttatatctctgtttaagtgggttcaataagaatctgtatatgccatgatcacagaaggtgattggggaataaatttttttatgaaagattctgcactttgcaaatgtattcccaatagcatatattaaaattgccagtctccagggaattcaaaagtattcctttttcaaaaacaattctgaaattcatttattattaagttgcatacctaaacaaacaatggaaatgtttttagtaattatttttgtacaagtagtttgaatgccttcattttatttttccttttgttttaatgtggttgtatcctttttcaccactcttgctttcttattctatccttccacctcagtaacacaaaataaaaagtaggagtggagactggggatgacaacaggtcctactgcagatcttctgctacacattttactgtgcctcacccttgagtgagagaaggtagtttaccttaaggcgtttgaatttgggaaagggctgtgctcatcgtacttgaccaagcaagatgactgttaggcagatgggcagatgctctgaggtgcctatcttgtacgtttttggtgctgccaacggccaccagctcctggcaatcctatgggcagcactcatcgcctgtccaaagcatagtagcatacccatcatgcattatggatagtctaaattccatacttacccctgaatttcaaattctgtagctactttcttgggaattcttgaaaatttccgctggttgaagatactagagatgtagaaagacttagtttggtttgcttttggttaatgtcttcttcaaactctgtaacctacagttttccattgtaagaaacaccattaccttgtaggctcctagagcatgaggtaggtctgctttctggtgccccttagcagggagttattgttctacaacagcatgtgggcatgtaaaccagtgttctgggtcattctttgttggttacaaaatcagaaccagacatgcctgcattgcaacagctttcagttgtcattttaagtatatctctgcataaggtttggaaatctgttctctgttgaccccagacctagtacatttattgtagctaagtttttgcaacccattatgtttttagcatctttatggaaaccatgtgagctacatagtgtttatcattatttttctggactaagggatatggttcttccattgataggtctgtgttttgaatctatggacactgttcttgcatgcagtgttataaagagggggacgcatggtatttgtgggggaaaaatagaccattttccctccacaaggaaactgcaactctcttggtgatacattgtaactaattcttataatctgagatcgctctttatttttgttacaattacattgttgcatgggtaattcttggcatctctttgagatttgctccttattgtgaccctccttgctttatgaagatgctaagaagttctgactggttgaggcatattgggaacagatggaaaggattttgcagttacacctccctgcttaatgtaattatttccctgataatcattctctgataaatgtatggttaatttggtgctgcatttgaaacagaaggtactaaagggcccactgtgggagatgtagaggtgaatgcagcctttgcctcaatctaagcacataactaaaatttgcagcagttggtgggaaagcacggggtttgtgtgtaaacccagcattttcagggttcttttcacattgacaagctggttccaattgatgttattcatttagcaagttggcaggggtgtcaagaagagccagaagagtgggctgctacaaaggcctgtcccacgctaacacccctccttgccaaatcattgtctttactcccgggatttggcacagtctctttccacctcccacagtaagaatgctcacctgcatctgtatttcctcatctgcatacaacctagcagggcctagaaccctggctcccacttttgaaagactgctgatctttgatgttataccaaaaagctttctagaatttgactgtcatagtcgccagttcttaattaaattcattttccagagcctcatctcatctttaaaattctcataaaagcatatgttagctatttttacctccctgaatttttttattatagaaacaatattgaaacattaaaagccaggttttcaaaagagcagaaatcagctgtagttccaggtggcagtctaattatttgtccttttctatttatcatgcagctgttgtgcatactggattataagatcttaggcccaggaagtactcaactttgctttatatcatttcagtaaattgcctggaggtgaaaaggtttccaagtggatgtggggaaaggaagcagagaaaaatggggaaattgctagctgtgtaatgaacctatgtctgaaaaatcattttgtaagataaatgagctacctgtttattctttatgctatacttttcaaagaagatttgcctttgtttcattcatacacattaaaaattctgggtaatgagtcttttaccatctttctaatctccctgactgtgccactccttctgtcccagatgtggtttggaaggaggaagagaccttcagaatgccccccatttgtaagtgaaattaacccagcgcctgtccctgtgtgagccaggcaagattccgtgagggtgctatggggctctgctgctttaaaacaaatccacactcttccatagtttaaaccagaggcatgaggccaagtctgatacacagctgagagttagaacttgtttttctgtcacatttccaaatgtctgcctggttaaattttctttgagtcaacattttgaggggaaggggtgagaaggaagcattggatgtttggtcctttttttgttggcttctattaatgctaaccagtttctgctcgtgtggaatgtggcatataaaaagttacctacattgttgaacaatgaaaactgctaccttatccattttgtttttatttgttttgactgcagtagaacttgctcttccactgtttcgaattttgtgttcttgtgggtggggtaacttaaggacttttcatactgacaaggaagctggccaacttgtcagtaagtaataaaagaaaagctaacatacatctaagttacagactttaactggacagccttgcagtgaaatcacagtgctattgggtctagaaagaagtttaggtgaaactggttctatggagagaggagttttttatgccccagttaactgtttactttaagagttggagccaaaagaacatgggggttctttcatgttgggacacagcatgtatactgtgaataggatgatatgacaccatctaaaactcacttctcatctgctctgcatgtgctcctgaaatgggggtcagtttccatttaagacatgtgtgtgcatgcacagatgtacatgcatgtgcttttacatggtatcgaggtcttcccctcaccctaccaaggaaaatgcagacataaagatattgttttgtaggatttcggttaactcacttcttgaaatgcatggtaaccattctaaccacttcattcctgagttctcatgggtcttttctttttcagagtgtgagtgtattgtgaagatcttcaagcccttttactttttttgctcacctcccaaagctgaaaaatgatcaaagagcctgtggttttctaatcccatcggtttggagaacagctgtgctgaacctaggcccagagcagggcctcgggtctgtagtcctgccacctctctctaccaggagagggaatgcctgaagggaagacctgctaggacagattcggctgagagttactgccaccagccctggctctcacctttcagaatatttttagcctctagtctgaagcaaggtggcagcctaggcctataatactgtccacacaggcctgccttcatatctgggcatatagtgagttcctacacttgctggcctcctcacaatgtggagtccgaacacaacagacaagctaagggagaggttccatttggagattttggagtgaccaagacttacaggacaaaggcctttcctcatgctcaccctaatacaaggaagttggcgaatttgcctgtctctgtggctatttatgcttccagcattcactgtcttaattgtacagaaattactcttgggaagcagcatttttgtgcctgttccttttatgtgtcttcctgggccagcttccctttcattctcgttttagccactgagcttgatgctgcttctctagaccaccctagctgccttcagctctgcgtgtccttcccctctggtttggtttcttcattctctatctgctgccaagtaccccttcagcagccaaggcagcccctgtttttgcatgtatttattacctacggcaacactgaagaataagtttggtccattccatttctctggatcctgtaagtgctccaagctctgtggaaaaagacatgaggatagagaaactgatacctctttttaaggtagctgattttttagtctcattcaagccctaaaaggagtccacagtatgagttttaatctgccactcacctcgtaaacacaggtactgtcttactgttagtatcttgtcctagtccaataaaattgtttaacaaaatgtatttttgtggatcttaagttgagcttcctgtagatatttgttttataagatatttgatatgtaggctgagtggtgattgtaaaattacatcccagttcattgagttcatagcttcctaacagtttttttcataatttttaaaggaaaattatcaatgacatcaagtattttctagtccattattttttcttaaatatggcatatataaaaactctcctgggttttgcatgaaattgttcagaaagagttattctaaatcaatatacctcttttcccccctcgagagagagacagagttctccccagtttttatgtaaccaggggacagttgccaccccgcgtggacagagtacctctgctctggccaaaatccacacttcagaattaccctggcgggtagggaggagtgggaccagggaagacacttgaaggaaggtgctttcaaaggcctctacttcctttaggtgcttcagagcatcctagtctctgacttcgtaacatttagaaaaattgacccttggaagtaaaatttgggttttttaggaatggagagagagcatgtatatgtttttctcctttgaaacatgtgctcttgttggaatggtttcttttttgttcactggggaagactgtagaggctgcatgctgttaaccatgtgttaagagtaaaaggggctgtgagcaccgtgggcttattctcccttagagtcttctatttttcaacctatcattgcacccagatgatatggcaaagtagacacatcagctaattatatgaaataaaattcatcgacttctttgacactgcagaattttaaccaggtgcatatcaaaattatgttttcatgtgtttcaagctagtttctgtgcctcttcagtttagtttactgaccagtctagagtccagagatgagagctcctaaagtctggctctcatgttttgaggattccagaaaagtcctacactcacagtgccaaatggcctgattaaacacagataaatgtttcttccaaaatatattacgcacaagtagtcaaggtgcacagccatttgagaagtctgctgttttaaaacctgtttgtggcattttgcaaggctgctgtgctttggatagatatctctgtacctcttggcgaggcagtacctgtgtatttccctctttgttggctacctaattttcatttaatatgttttttttttttctcccggtggaaaggagaggcaggtgcctcttaaaattatttcacagcaaaccttcgtaagttcttattgcctatttcatgtagaaaggttgatttgtagctgctgtctttctcttcctaataaaccttgtgggtggtgaagatctaggagagtgaaacattaaaaatgaatgaagagaactgaagtagtttgtctaaggtgggggttgagaggggctgggtggaggaggtttaacaaaaggcattgatgttttaaatgcacagaaatgccatctatcaataataaatggtgtggcatagaacagatgtgccaaattgagttatgacagcatatagattagttcatagaggggaaaagtaagtagcatagctaacgtttttaaattatctgtgagcttttgaaatacaattttatgtattcagtttttactttctagtggctgctagctggaaatattaaaagtttggtgttttttaaaattccaattaaattgtgcttgtttctaaagtaagaattaagaatgaataaattttgcttttctgaagaggaagacagtctgaaatgaatttcttgcccttcattggctgagtacttgccttttaaaagctcatgttgtaattgttatgaggaatgaaagtgcattataaaacttaagtcatgtgaaactattacccagtgttttctaaatttacattgattatatctctaggtacctgctcagtgcatatttaaactacaagggactactcaggaaacccactcatgctttacaatcaattcacccccctcagcattttgattcaagggataattctacacatatgctctgtgtgagaattaccttgcttggttgggaaacaacaagagaaatatccacttgtctcacagcaaatcactactagtaaattggaaagacaaatcttttaggttaactaggatattttgtaggtgtagatactagagcagaaaacaagttttgagcttctagataggtaggagttattgttaatgtcactggcatagcagaggacaatactgtgggaggtgtttgaaatgaatcctgagttcctgagttggaattggtgtgcttaatgaaaaaaacagccgtggtgagacatatggaaccattccactcatctcaatatggacccttgcttgctttttggaaacttgtttggaatcggttatctttgctttgttggcccaacacaagtaaatcctctttgtttttcctaaggtgaacaaaaaagtagttcttgcagataatatcaagttggtttaaattttaggtttgttagactagaaagacgaattatttagccgtcattgaagagtttaatctttgagtagatgttacaggtgaaaatgagcatagtgattatataaaaagtctattactaactcttattttagccatctgtattgttgactttcaactttttcaggtgcaaaattttcttgaatcaatcttttcatctccaagggatcccagcatgccttaaaaaaaaacacttgtcaacggattttagagccaccaaataaatctattttatggtcccgttagatatctagctcaaaacccccattcactctgaaaggtcctatggttggatgcacaacaaatttcctgaaatcatctctgatgcacctcctttgggttgttaagaaggtgttgaaatgtgagaaggctggtagtgaaatcaagttgtggctcagggtaattaggaagggagaaaacccagcattttcatagtacattgtctggcttttagttgcagctgcacacagcggatatgaatttaaccggttgctacagtgcagctggttcagatctggctttataactggctgaagtaaaatgaaatggctggaaaattccagtcaagtaacaccagtgggaacaagtggaattaaatagtgtgagcaaatgctggccgagttggagaaaaacttaagacataaacttgagggtggtggtgaggacttcgggtgggcaggcggggggaggggggtgcattttctggtctttttcacaagggtctattgtttcgattagtttccttgcaggaggtagaaggtttcctccatcccgagtaattcatagagtgtttggcttgtctaccttcctcctgactgaagtcacctgatactttttgttttttcaagaaggaagagagaaccctgttgcctcagttactagcaatgatacaattctcaaaatctggtctttttttgtttctttgaaatagtttctccatgttgtgtgacacagcagcccctgtcttatcatagttgtcttccctccaccacctgtaccagagatgttggatatgttggaggtgaaggtgtgcaaggtttttaactaactgttctaattaaaggattctgcaggaaagaacatgggtttacaaaagagaagcttttgtattattagtaattttttttctttgatgaatttatgtgcttagtttggagaatcgagagttggctgggaaaagatttctgaggagttaagggactctggtgctgtttgggaaaacatttgctgtaactaggtttagggtattcagtctctgccagtgcttaggagctatatttgccaggtgatttccctgtcgcagggattagccagtgatgtgaaacatttgattaagggactaatagaaatctttcctggggtacatgagtggtggctgttactggggtgccccctcctcaactctttctctctgtatttcgagtaagtttgtagtggtgagtcatgggatacagttttttaatcctatgaagaatcttggtaggaagcaaagttctgttaggtatattgttaattaagccctttaaagtttctgcataataactcagaaacgagcctttttcttttctcagaactatgccatgactcatccactttggaagcagacaagatataaattatcaataaaaattgcccataatctggcctggcacagtggctcatgcctgtaatcccagcactttgggagatgatggtgggaggatcgcttgaggccaggagttcaagaccagcctgggcaacgtagtgaggaacccccgacctccaccatcccccccgccccccaccaaccccctcacttcaaagttttttaaatcagccacgcatggtggtatatgcccttagtcctgtgtactcaggaggctgaagtgggaaagtcctttgagcccaggagttgaacgttgcagtgagctatgaatgtgccaccgcactctagcctgggcaagagagctgaccctgtcacttaaaaaaaaaaaaaaaaaaaaaccgatattctgatagcaacactgctcacttgctcactctttctcttaggaaagtaaaaaagttttgtcttgatgcacagatttatttaaactttagttctaaatatataggactttaaaaaatattttctgaaagtgatatccatttttgaggtgcaagcagttgggtccattttggagattccgccttaagccattcagcttcaaaatggcggggagctcttgctacagagggttaccccgttctgcttccttcccattctgttaaaccttgtacatgctccttcccacagatcactattatcttcaaagcccaccatgagtgtacagatcagaaagtctaccaagctgtgacagatgacctgccggccgcctttgtggatggcaccaccagtggtggggacagcgatgccaagagcctgcgtatcgtggaaagggagagtggccactatgtggagatgcacgcccgctatatagggaccacagtgtttgtgcggcaggtgggtcgctacctgacccttgccatccgtatgcctgaagacctggccatgtcctacgaggagagccaggacctgcagctgtgcgtgaacggctgccccctgagtgaacgcatcgatgacgggcagggccaggtgtctgccatcctgggacacagcctgcctcgcacctccttggtgcaggcctggcctggctacacactggagactgccaacactcaatgccatgagaagatgccagtgaaggacatctatttccagtcctgtgtcttcgacctgctcaccactggtgatgccaactttactgccgcagcccacagtgccttggaggatgtggaggccctgcacccaaggaaggaacgctggcacattttccccagcagtggcaatgggactccccgtggaggcagtgatttgtctgtcagtctaggactcacctgcttgatccttatcgtgtttttgtaggggttgtcttttgttttggttttttattttttgtctataacaaaattttaaaatatatattgtcataatatattgagtaaaagagtatatatgtatataccatgtatatgacaggatgtttgtcctgggacacccaccagattgtacatactgtgtttggctgttttcacatatgttggatgtagtgttctttgattgtatcaattttgttttgcagttctgtgaaatgttttataatgtccctgcccagggacctgttagaaagcactttattttttatatattaaatatttatgtgtgtgcttggttgatatgtatagtacatatacacagacatccatatgcagcgtttcctttgaaggtgaccagttgtttgtagctattcttggctgtaccttcctgccctttcccattgctactgatttgccacggtgtgcagcttttactcgccaccttccggtggagctgcctcgttcctttgaactatgccctcacccttctgccctcacttgatttgaaagggtcgttaactctcccttacaggtgctttgactcttaaacgctgatcttaagaagctctcttcatctaagagctgttactttttcagaagggggggtattattggtattctgattactctcaattctaattgttatatatttgagcccatacagtgtattaggttgaaccatagaaactgctattctcgtaggtcaaaagggtctagtgatggaagttttgtagataagtaccaggcatctcagtaactcctagactttttctcatcccatgccccgttttaaattgtcagttttccctctgactcttctgtgttaaaacatgaaactataaatttagtaattatcatgccttgctctttttaatctatatgactgatgcaagcccctcttcttaaccgtttcttggctttgagcccagaaacacagctctccctgtctccaactccagtaagccctcctcagcctcaccttacgaatccaaagaactggggtttgttaggttctttctctaatgtagaggcccagatcccatcacaaagtttttcattcttccttgtccacgatgatcttcatcacagtctttgatatgtctgcatgcaaagtggaacagagttgggcggcaatgacagaagagcttccttggcctgactcggtgtgcggccacttcggcactgcttaatccagatattcttgttaactaagcattgtgcttcccaggtggtctgaagtcaggtactctctctctcaacacctgtagttgaatatgatttggtcagttgctcgttgtaacttggagaaattcctataaagtaagatctccttgcctcttccatccattgttggcacccccttgcaaaaggaaaagaacagcaaaagtcaggagcagtaatctgagaaagttaactccaggataggtaggtttctattgttatagctagatgtaaatctttagttccaagaagtgatagagtttctgctttaataatttgttgataagtttacataaacagaaataaaagatactatctttaccgtagtagttcaggccaagattatgcttagttttagttctccaggtagttacttttgccatgtcctattgatcagtgacactgccagaggcccataccggcaagaggaagaggacgtcattttgtaaagtttaacttcttagcgaactgatgtgccacccagtcacagagtggagttgtgaattcatgtagaggtggcaaacctctaccttgtgttgatgagagaataatcttgggcagtctgggaaaataaggaaggcatctccttcttactcatggagattcaactatagagagttgaaacctaaacccgccttccttttatagaagctggactagagacggactgaccatcagctctgaactgtggctttttttgttcacctatgatgccatgtaccaaattcagaagctatcgttaataatttgttttataattgagtagtacaagcgaggaaaaaatacggaggataaccactatttttgtgcaaatagtatgaaagtgaagtaaaagcaatagaagaaatttctataggatctgggtttagagtgtgtatcattaataaatatacctttgctcttttcagggaaaataacaaccacccttactgatagttgggaaaagaagattgggttattttgccatatcatttagctggaagtgacatttaaaagcaccctgcatcactagtaatagtgtattttgctattctgcccttgtaatcggtgtccctgtaaaacaatccccacagattactttcagaaatagatgtatttctctacgtaagggccaggtttattttctccttttttgagatttctagaaaaaatgctgcttgcacatgttggttcttgaaaccttagctagaagaatttcaggtcataccaacatgtggataggctatagctgttcagaggtctcctgggggagcttaaaacgggggaaacactggttttcacagatgctccacatggctgtctttaaaagactcaaaacttttttttgtcctctttgttatgcttggaagctccccccccccccccacagtgtgtcgagtctttgcaaagaaacctttagatgtggttcatagatatatgaatacgtatctgtgtaaaacagtgagtgtgcagtgtgtaaatactttaaattattatgctagaaaaataaagttacataccttgctgtggaa

(19) United States
(12) Patent Application Publication
(10) Pub. No.: US 2025/0230412 A1
(43) Pub. Date: Jul. 17, 2025

(54) METHODS AND COMPOSITIONS FOR TREATING CANCER

(71) Applicants: President and Fellows of Harvard College, Cambridge, MA (US); Dana-Farber Cancer Institute, Inc., Boston, MA (US)

(72) Inventors: Arlene H. Sharpe, Brookline, MA (US); Dennis L. Kasper, Brookline, MA (US); Joon Seok Park, Brookline, MA (US); Francesca S. Gazzaniga, Jamaica Plain, MA (US); Gordon J. Freeman, Brookline, MA (US)

(21) Appl. No.: 18/834,697

(22) PCT Filed: Feb. 1, 2023
(86) PCT No.: PCT/US2023/012139

§ 371 Date: Jul. 31, 2024

Related U.S. Application Data
(60) Provisional application No. 63/305,427, filed on Feb. 1, 2022.

(51) Int. Cl. C12N 5/0783; A61K 40/11; A61K 40/31; A61K 45/06; A61P 35/00; C12N 15/113

(52) U.S. Cl. CPC C12N 5/0638; A61K 40/11; A61K 40/31; A61K 45/06; A61P 35/00; C12N 15/1135

(57) ABSTRACT

Provided herein are methods and compositions for treating cancer or a tumor in a subject by administering to the subject T cells with reduced RGMb expression or activity and an immune checkpoint inhibitor such as a PD-1 or PD-L1 inhibitor.

### RELATED APPLICATIONS

This application is a § 371 national-stage application based on PCT/US2023/012139, filed Feb. 1, 2023, which claims the benefit of priority to U.S. Provisional Application Ser. No. 63/305,427, filed on Feb. 1, 2022, which is incorporated herein in its entirety by this reference.

### STATEMENT AS TO FEDERALLY SPONSORED RESEARCH

This invention was made with government support under CA247072 and AI056299 awarded by National Institutes of Health (NIH). The government has certain rights in this invention.

### REFERENCE TO A SEQUENCE LISTING XML

This application contains a Sequence Listing which has been submitted electronically in XML format. The Sequence Listing XML is incorporated herein by reference. Said XML file, created on Mar. 26, 2025, is named HMV-31001_SL.xml and is 120,931 bytes in size.

### BACKGROUND

T cell therapies have emerged as a promising approach in treating various diseases, especially in the context of immunosuppression. With the increasing prevalence of immunocompromised conditions, there is a critical need for new T cell therapies to improve patient outcomes. For example, recent studies have shown the efficacy of T cell therapies in treating cancer, autoimmune diseases, and infections (e.g. T Cell Therapies for Cancer, Chen et al., Nature Reviews Cancer, 2020; T Cell Therapy for Autoimmune Diseases, Kivity et al., Nature Reviews Immunology, 2020). These findings highlight the importance of continued research and development in the field of T cell therapies, to address the growing demand for treatments that can effectively modulate the immune system.

The first generations of T cell therapies have shown promise in treating various diseases, but their rapid immunosuppression and limited sustained effect highlights the need for less immunosuppressable T cell constructs. These early T cell therapies often get subject to immunosuppression mechanisms developed by the tumor. To address these limitations, there is a growing need for T cell therapies that are less susceptible to immunosuppression, and can maintain their therapeutic effect over a longer period of time. Such therapies could have a significant impact on patient outcomes, by improving the safety and efficacy of T cell-based treatments.

On the other hand, immune checkpoint blockade, or immunotherapy, is a novel therapeutic approach that reinvigorates tumor-specific T cells to efficiently kill cancer cells by blocking inhibitory pathways in T cells including CTLA-4 and PD-1. In recent years, antibodies against immune checkpoint molecules have attracted attention as new therapeutic agents for cancer. CTLA-4 and PD-1 inhibitors promote the activation of T cells by inhibiting a molecule that suppresses the activation and function of T cells, and enhances the antitumor response of the T cells. In a treatment with an immune checkpoint inhibitor, cancer is eliminated by activating the immune state of the living body.

Despite the clinical success of immune checkpoint blockade-based drugs, a significant fraction of cancer patients do not respond to the therapy. Thus, novel therapies for cancer are needed.

### SUMMARY

The combination of immune checkpoint inhibitors with T cell therapy is relevant and needed, as it has the potential to enhance the efficacy of T cell-based treatments and overcome the limitations posed by immunosuppression. Immune checkpoint inhibitors are drugs that target specific proteins that regulate the immune system, and can enhance the ability of T cells to identify and destroy cancer cells. By combining these drugs with T cell therapy, it may be possible to improve the efficacy of T cell-based treatments, and target both the cancer cells and the immunosuppressive factors that can limit therapeutic success. Recent studies have demonstrated the potential of combining immune checkpoint inhibitors with T cell therapy, especially in the context of cancer treatment, but with significant challenges so far (“Combination of genetically engineered T cells and immune checkpoint blockade for the treatment of cancer”, Rafaela Rossetti et al., Immunotherapy Advances, Volume 2, Issue 1, 2022), due to the inherent properties of T cells being still largely immunosuppressed.

Therefore, current invention has the potential to revolutionize the field of T cell therapies, by enabling the development of treatments that can effectively modulate the immune system, with reduced risk of immunosuppression and improved patient outcomes, as stand-alone and also in combination with immune checkpoint inhibitors.

In some aspects, provided herein are populations of T cells (e.g., modified T cells) that comprise decreased expression or activity of RGMb. Decreased expression or activity of RGMb can be achieved, for example, by contacting the T cells with an agent disclosed herein (such as RGMb agent) as well as engineering the T cells to express reduced levels of RGMb (such as through gene knock-out or knock down). The T cells may comprise cytotoxic T cells (CTLs). In some embodiments, the T cells are autologous. In some embodiments, the T cells are allogenic.

In some embodiments, the T cells have been modified ex vivo to inhibit the activity or expression of RGMb, wherein modifying comprises contacting the T cells with an RGMb agent that decreases the expression of or inhibits or disrupts the activity of RGMb in the T cells. The RGMb agent may be, for example, an antibody that specifically binds to an RGMb peptide, an RNA inhibitory molecule, a gRNA complementary to a portion of a RGMb gene.

In some embodiments, the T cells are CAR-T cells or TCR-T cells.

In some embodiments, the compositions comprising T cells provided herein further comprise an agent that specifically binds to PD-1 or PD-L1, such as an antibody that blocks PD-1 or an antibody that blocks PD-L1.

In some embodiments, the compositions comprising T cells provided herein further comprise an agent that specifically binds to PD-1 or PD-L1 and an RGMb agent disclosed herein, such as antibody RGMb agent.

Antibodies disclosed herein may be monoclonal and/or humanized. In some embodiments, the antibodies disclosed herein are bispecific.

In some embodiments, the antibody that blocks PD-1 is selected from cemiplimab (REGN2810), nivolumab (BMS-936558, MDX-1106, ONO-4538), pembrolizumab (MK-3475, SCH 900475), SHR1210, sintilimab (IBI308), spartalizumab (PDR001), tislelizumab (BGB-A317), pidilizumab, BCD-100, toripalimab (JS001), PF-06801591, AB122, AK105, AMG 404, BCD-100, BI 754091, F520, HLX10, HX008, JTX-4014, LZM009, MEDI0680, MGA012, Sym021, TSR-042, PSB205, MGD019, MGD013, AK104, XmAb20717, R07121661, and CX-188.

In some embodiments, the antibody that blocks PD-L1 is selected from atezolizumab (MPDL3280A, RG7446, RO5541267), durvalumab (MEDI4736, MEDI-4736), avelumab (MSB0010718C), FS118, BCD-135, BGB-A333, CBT-502, CK-301, CS1001, FAZ053, HLX20, KN035, MDX-1105, MSB2311, SHR-1316, TG-1501, ZKAB001, INBRX-105, MCLA-145, KN046, M7824, and LY3415244.

Also provided herein are methods of treating cancer. In some aspects, provided herein are methods of treating or preventing cancer in a subject, comprising: a) isolating a population of T cells ex vivo and b) contacting the T cells with an RGMb agent that decreases the expression of or inhibits or disrupts the activity of RGMb in the T cells.

In some embodiments, the methods further comprise administering to the subject the T cells conjointly with an agent that specifically binds to PD-1 or PD-L1 (such as an agent that specifically binds to PD-1 or PD-L1 disclosed herein).

In some embodiments, the methods further comprise administering to the subject the T cells conjointly with an agent that specifically binds to PD-1 or PD-L1 (such as an agent that specifically binds to PD-1 or PD-L1 disclosed herein) and an RGMb agent disclosed herein, such as antibody RGMb agent.

In some embodiments, the methods further comprise administering to the subject the T cells conjointly with an agent that specifically binds to PD-1 or PD-L1 (such as an agent that specifically binds to PD-1 or PD-L1 disclosed herein) and the RGMb agent, such as antibody RGMb agent.

In some embodiments, the RGMb agent is an antibody that specifically binds to an RGMb peptide. The RGMb agent may be an antibody that disrupts the binding between RGMb and PD-L2. The RGMb agent may be an RNA inhibitory molecule. In some embodiments, the RGMb agent is a gRNA comprising complementary to a portion of a RGMb gene.

In some aspects, provided herein are methods of treating or preventing cancer in a subject, comprising administering to the subject T cells, wherein the T cells comprise decreased expression or activity of RGMb.

In some aspects, provided herein are methods of treating or preventing cancer in a subject, comprising administering to the subject T cells conjointly with an agent that specifically binds to PD-1 or PD-L1, wherein the T cells comprise decreased expression or activity of RGMb.

In some embodiments, the methods further comprises administering to the subject the T cells conjointly with an agent that specifically binds to PD-1 or PD-L1 (such as an agent that specifically binds to PD-1 or PD-L1 disclosed herein) and the RGMb agent, such as antibody RGMb agent.

In some embodiments, the methods further comprise a step of modifying the T cells to inhibit the activity of RGMb, the expression of RGMb, or disrupt the activity of RGMb prior to administration to the subject.

As used herein, “modifying” comprises, but is not limited to, contacting the T cells with an RGMb agent that decreases the expression of or inhibits or disrupts the activity of RGMb in the T cells. In some embodiments, the RGMb agent is an antibody that specifically binds to an RGMb peptide. The RGMb agent may be an antibody that disrupts the binding between RGMb and PD-L2. The RGMb agent may be an RNA inhibitory molecule. In some embodiments, the RGMb agent is a gRNA comprising complementary to a portion of a RGMb gene. “Modifying” also includes engineering T cells to comprise reduced expression of RGMb. Additionally, “modifying” may also include obtaining a sample comprising T cells (e.g., modified or unmodified T cells), and sorting such T cells to isolate those that have decreased expression of RGMb compared to the original isolated sample.

In some embodiments, the T cells are CAR-T cells or TCR-T cells. The T cells may be allogenic or autologous.

In some embodiments, the compositions comprising T cells provided herein further comprise an agent that specifically binds to PD-1 or PD-L1, such as an antibody that blocks PD-1 or an antibody that blocks PD-L1.

Antibodies disclosed herein may be monoclonal and/or humanized. In some embodiments, the antibodies disclosed herein are bispecific.

The antibody that blocks PD-1 may be selected from cemiplimab (REGN2810), nivolumab (BMS-936558, MDX-1106, ONO-4538), pembrolizumab (MK-3475, SCH 900475), SHR1210, sintilimab (IBI308), spartalizumab (PDR001), tislelizumab (BGB-A317), pidilizumab, BCD-100, toripalimab (JS001), PF-06801591, AB122, AK105, AMG 404, BCD-100, BI 754091, F520, HLX10, HX008, JTX-4014, LZM009, MEDI0680, MGA012, Sym021, TSR-042, PSB205, MGD019, MGD013, AK104, XmAb20717, R07121661, and CX-188.

In some embodiments, the antibody that blocks PD-L1 is selected from atezolizumab (MPDL3280A, RG7446, RO5541267), durvalumab (MEDI4736, MEDI-4736), avelumab (MSB0010718C), FS118, BCD-135, BGB-A333, CBT-502, CK-301, CS1001, FAZ053, HLX20, KN035, MDX-1105, MSB2311, SHR-1316, TG-1501, ZKAB001, INBRX-105, MCLA-145, KN046, M7824, and LY3415244.

In some embodiments, the subject is nonresponsive to immune checkpoint inhibitor therapy, such as therapy that targets CTLA-4, VISTA, B7-H2, B7-H3, B7-H4, B7-H6, ICOS, HVEM, CD160, gp49B, PIR-B, KIR family receptors, TIM-1, TIM-3, TIM-4, LAG-3, BTLA, SIRPalpha (CD47), CD48, 2B4 (CD244), B7.1, B7.2, ILT-2, ILT-4, TIGIT, HHLA2, butyrophilins, or A2aR. In some embodiments, the subject is nonresponsive to anti-PD-1 therapy or anti-PD-L1 therapy.

In some aspects, provided herein are compositions comprising a population of modified T cells, wherein the T cells comprise a single chain variable fragment (scFv) specific for an RGMb peptide. In some embodiments, the composition further comprises an agent that specifically binds to PD-1 or PD-L1 (e.g., any agent that specifically binds to PD-1 or PD-L1 disclosed herein). In some aspects, provided herein are compositions comprising populations of modified T cells, wherein the T cells comprise a single chain variable fragment (scFv) specific for an RGMb peptide and a second single chain variable fragment (scFv) specific for a PD-1 or PD-L1 peptide. In some aspects, such compositions are administered to subject in methods of treating cancer in a subject.

The cancer may be lung cancer, a breast cancer, a colon cancer, a cervical cancer, a pancreatic cancer, a renal cancer, a stomach cancer, a GI cancer, a liver cancer, a bone cancer, a hematological cancer, a neural tissue cancer, a melanoma, a thyroid cancer, a ovarian cancer, a testicular cancer, a prostate cancer, a cervical cancer, a vaginal cancer, or a bladder cancer.

In some embodiments, the cancer comprises a tumor. The tumor may be an adenocarcinoma, an adrenal tumor, an anal tumor, a bile duct tumor, a bladder tumor, a bone tumor, a brain/CNS tumor, a breast tumor, a cervical tumor, a colorectal tumor, an endometrial tumor, an esophageal tumor, an Ewing tumor, an eye tumor, a gallbladder tumor, a gastrointestinal, a kidney tumor, a laryngeal or hypopharyngeal tumor, a liver tumor, a lung tumor, a mesothelioma tumor, a multiple myeloma tumor, a muscle tumor, a nasopharyngeal tumor, a neuroblastoma, an oral tumor, an osteosarcoma, an ovarian tumor, a pancreatic tumor, a penile tumor, a pituitary tumor, a primary tumor, a prostate tumor, a retinoblastoma, a Rhabdomyosarcoma, a Salivary gland tumor, a soft tissue sarcoma, a melanoma, a metastatic tumor, a basal cell carcinoma, a Merkel cell tumor, a testicular tumor, a thymus tumor, a thyroid tumor, a uterine tumor, a vaginal tumor, a vulvar tumor, or a Wilms tumor.

In some aspects, provided herein are methods of preparing a cancer therapy comprising T cells (e.g., modified T cells), comprising: isolating a population of T cells ex vivo and contacting the T cells with an RGMb agent that decreases the expression of or inhibits the activity of RGMb in the T cells.

The RGMb agent may be an antibody that disrupts the binding between RGMb and PD-L2. The RGMb agent may be an RNA inhibitory molecule. In some embodiments, the RGMb agent is a gRNA comprising complementary to a portion of a RGMb gene.

A therapeutic method is described herein to convert non-responders to anti-PD-1/anti-PD-L1 therapy to responders by immunotherapy comprising T cells (e.g., an adoptive immunotherapy). The invention described herein, is based, in part, on the novel and surprising discovery that deletion of RGMb in T cells, but not in macrophages and granulocytes, improved anti-tumor responses in subjects given anti-PD-L1. This disclosure demonstrates a method to convert non-responders to anti-PD-1/anti-PD-L1 therapy to responders by combination therapy with PD-1 and/or PD-L1 inhibitors and T cells (e.g., T cells with reduced/decreased expression or activity of RGMb).

Provided herein are methods of treating or preventing cancer in a subject by administering to the subject T cells (e.g., modified T cells disclosed herein) conjointly with an agent that specifically binds to PD-1 or PD-L1 and/or decreases the activity or expression of PD-1 or PD-L1, wherein the T cells comprise decreased expression or activity of RGMb. The T cells may be cytotoxic T cells (CTLs). The T cells may be chimeric antigen receptor (CAR)-T cells and/or engineered T-cell receptor (TCR) T cells. The T cells may be autologous or allogenic. In some embodiments, the T cells are selected for administration because of reduced activity or expression of RGMb (e.g., reduced expression or activity compared to means or medians of RGMb expression or activity in T cell populations). In some embodiments, the T cells have been modified to decrease or inhibit the activity of expression of RGMb. For example, modification of T cells disclosed herein to inhibit or decrease the expression or activity of RGMb can comprise gene editing, RNAi technology, peptide inhibitors, blocking antibodies, or any other technology to knock-out or knock-down RGMb activity or expression. As used herein an “RGMb agent” is an agent that decreases the expression of or inhibits or disrupts the activity of RGMb. The agent may be an antibody, such as an antibody that blocks RGMb or an antibody that disrupts the interaction between RGMb and its ligands. The agent may be an antibody, such as an antibody that disrupts the interaction between RGMb and PD-L2.

In some embodiments, the T cells are administered conjointly with an agent. The agent may be an agent that disrupts PD-1, disrupts PD-L1, or disrupts the interaction between PD-1 and PD-L1. An agent may be an antibody or antigen binding fragment thereof, peptide, small molecule, or an inhibitory nucleic acid.

The agent may be an antibody, such as an antibody that blocks PD-1, an antibody that blocks PD-L1, an antibody that disrupts the interaction between PD-1 and its ligands or an antibody that disrupts the interaction between PD-L1 and its ligands. The antibody may be a monoclonal antibody, a humanized antibody, and/or a bispecific antibody.

The antibody that blocks PD-1 may be selected from cemiplimab (REGN2810), nivolumab (BMS-936558, MDX-1106, ONO-4538), pembrolizumab (MK-3475, SCH 900475), SHR1210, sintilimab (IBI308), spartalizumab (PDR001), tislelizumab (BGB-A317), pidilizumab, BCD-100, toripalimab (JS001), PF-06801591, AB122, AK105, AMG 404, BCD-100, BI 754091, F520, HLX10, HX008, JTX-4014, LZM009, MEDI0680, MGA012, Sym021, TSR-042, PSB205, MGD019, MGD013, AK104, XmAb20717, R07121661, and CX-188.

The antibody that blocks PD-L1 may be selected from atezolizumab (MPDL3280A, RG7446, RO5541267), durvalumab (MEDI4736, MEDI-4736), avelumab (MSB0010718C), FS118, BCD-135, BGB-A333, CBT-502, CK-301, CS1001, FAZ053, HLX20, KN035, MDX-1105, MSB2311, SHR-1316, TG-1501, ZKAB001, INBRX-105, MCLA-145, KN046, M7824, and LY3415244.

The agent and or any composition disclosed herein may be administered systemically, orally, parenterally, or intravenously.

In some embodiments, the subject is nonresponsive to immune checkpoint inhibitor therapy. In some embodiments, the subject is nonresponsive to anti-PD-1 therapy or anti-PD-L1 therapy. In some embodiments, at least one antibiotic (e.g., at least two, at least three, at least four, at least five, at least six, at least seven, at least eight, at least nine or at least ten antibiotics) is administered to the subject within a period of time prior to administration of the T cells and/or agent. The at least one antibiotic may be Vancomycin, Neomycin, Metronidazole and/or Ampicillin. The period of time may be one day, one week, two weeks, three weeks, one month, two months, three months, four months, five months, six months, nine months, one year, two years, three years, four years or five years.

The cancer may be lung cancer, a breast cancer, a colon cancer, a cervical cancer, a pancreatic cancer, a renal cancer, a stomach cancer, a GI cancer, a liver cancer, a bone cancer, a hematological cancer, a neural tissue cancer, a melanoma, a thyroid cancer, a ovarian cancer, a testicular cancer, a prostate cancer, a cervical cancer, a vaginal cancer, or a bladder cancer.

The cancer may comprise a tumor, and the tumor may be an adenocarcinoma, an adrenal tumor, an anal tumor, a bile duct tumor, a bladder tumor, a bone tumor, a brain/CNS tumor, a breast tumor, a cervical tumor, a colorectal tumor, an endometrial tumor, an esophageal tumor, an Ewing tumor, an eye tumor, a gallbladder tumor, a gastrointestinal, a kidney tumor, a laryngeal or hypopharyngeal tumor, a liver tumor, a lung tumor, a mesothelioma tumor, a multiple myeloma tumor, a muscle tumor, a nasopharyngeal tumor, a neuroblastoma, an oral tumor, an osteosarcoma, an ovarian tumor, a pancreatic tumor, a penile tumor, a pituitary tumor, a primary tumor, a prostate tumor, a retinoblastoma, a Rhabdomyosarcoma, a salivary gland tumor, a soft tissue sarcoma, a melanoma, a metastatic tumor, a basal cell carcinoma, a Merkel cell tumor, a testicular tumor, a thymus tumor, a thyroid tumor, a uterine tumor, a vaginal tumor, a vulvar tumor, or a Wilms tumor.

In some embodiments, an additional agent is administered, such as a chemotherapeutic agent or an additional immune checkpoint inhibitor. The additional immune checkpoint inhibitor may comprise an antibody or agent specific for an immune checkpoint protein selected from CTLA-4, VISTA, B7-H2, B7-H3, B7-H4, B7-H6, ICOS, HVEM, CD160, gp49B, PIR-B, KIR family receptors, TIM-1, TIM-3, TIM-4, LAG-3, BTLA, SIRPalpha (CD47), CD48, 2B4 (CD244), B7.1, B7.2, ILT-2, ILT-4, TIGIT, HHLA2, butyrophilins, and A2aR.

### BRIEF DESCRIPTION OF THE DRAWINGS

This patent or application file contains at least one drawing executed in color. Copies of this patent or patent application publication with color drawings will be provided by the Office upon request and payment of necessary fee.

FIG. 1A-FIG. 11. RGMb on T cells regulates anti-tumor immunity in GF mice. FIG. 1 A shows the level of RGMb RNA expression in tumor-infiltrating CD4⁺ T cells, CD8⁺ T cells, CDT11⁺ MHC class II⁺ cells and CD11b+ cells sorted from MC38 tumors in GF and SPF mice on day 11 after implantation, and quantified by qPCR. FIG. 1B shows surface expression of RGMb protein on tumor-infiltrating leukocytes isolated from MC38 tumors on day 13 after implantation, measured by flow cytometry using monoclonal antibody (clone 9D3) against RGMb. Representative histograms of expression of RGMb on CD8⁺ T cells (upper left) and CD11c+MHC class II⁺ cells (lower left) in GF (black) and SPF (red) mice are shown and frequencies of RMGb⁺ cells within indicated cell populations quantified (right). n=4-5 mice per group. Data are representative from 2 different experiments. FIG. 1C shows RGMb expression on CD8+ T cells in MC38 tumors from GF and SPF mice treated with isotype and anti-PD-L1 on Day 11 pi (FIG. 1D-FIG. 1G) MC38 tumors were harvested from GF mice treated with indicated antibodies at day 18 pi and total numbers of (FIG. 1D) CD8⁺ T cells, (FIG. 1E) CD4⁺ T cells and (FIG. 1F) Treg cells in tumors were measured. FIG. 1G shows the ratio of CD8⁺ to Treg was calculated. MC38 tumor growth in (FIG. 1H) rgmb^{f/f} vs rgmb^{f/f}CD4-Cre or (FIG. 11) rgmb^{f/f} vs rgmb^{f/f} LysM-Cre mice treated with ABX and given anti-PD-L1. n=7-12 mice per group. Data are representative from 2 different experiments.

FIG. 2A-2D shows RGMb expression is modulated by the gut microbiota. Tumor draining lymph nodes from GF and SPF mice implanted with MC38 tumor cells subcutaneously were analyzed on day 11 after implantation. FIG. 2A shows relative mRNA expression and FIG. 2B-2D shows cell surface expression of RGMb protein in CD4+ T cells, CD8+ T cells, CD11c+ MHCII+ and CD11b+ cells. The levels of rgmb transcripts were normalized to expression of an internal control gene 18S rRNA. FIG. 2B shows frequencies of RGMb-expressing CD4+ T cells, CD8+T cells, CD1 lc+MHCII+ and CD11b+ cells were measured using 9D3 clone mAb. FIG. 2C-FIG. 2D shows geometric Mean Fluorescent Intensity (gMFI) of RGMb was assessed in indicated populations from tumor in FIG. 2C and tumor in FIG. 2D draining lymph nodes using anti-RGMb polyclonal antibody. n=4-5. Significance measured by unpaired Student's t-test. **** P<0.0001, *** P<0.001, ** P<0.01, *P<0.05.

FIG. 3A-3C shows RGMb is elevated on CD8⁺ T cells in tumors of mice colonized with stool from melanoma patients who did not respond to anti-PD-1. GF mice were colonized with stools from three patients who received anti-PD-1 therapy and responded or did not respond—Complete Responder (CR) or Non-Responder 1(NR1) or Non-Responder 2(NR2). The mice were injected subcutaneously with MC38 tumor cells and treated with rat IgG2b isotype control. Frequencies of RGMb-expressing tumor-infiltrating (FIG. 3A) CD8⁺ T cells, (FIG. 3B) CD11b⁺ MHC II⁺ cells, and (FIG. 3C) CD11c⁺ MHC II⁺ cells were examined on post-implantation day 29. n=5-6 Significance measured by one-way ANOVA. ** P<0.01, ns=not significant.

FIG. 4 shows RGMb and PD-L2 are not expressed on MC38 tumor cells. MC38 tumor cells expressing GFP were implanted in GF or SPF mice. The mice were treated with two doses of isotype control or anti-PD-L1 one week after tumor implantation, and tumors were harvested 13 days after tumor implantation. MC38-GFP cells were isolated and examined to measure PD-L2 (Upper), PD-L1 (Middle), and RGMb (Lower) expression by flow cytometry. Representative of 5 mice per group.

FIG. 5 shows RGMb disruption potentiates pro-inflammatory cytokine TNF-α, production by CD4⁺ tumor-infiltrating T cells. GF mice were implanted with MC38 tumor cells subcutaneously and treated with indicated antibodies as in FIG. 1A. Tumor-infiltrating CD4⁺ T cells were isolated on day 11 after tumor implantation and stimulated with PMA/Ionomycin for 5 hours. Frequencies of TNF-α, producing cells among CD4⁺ T cell population were measured by intracellular staining and flow cytometry. Significance measured by one-way ANOVA with Bonferroni's multiple comparisons. **** P<0.0001, *** P<0.001, ** P<0.01, *P<0.05.

FIG. 6A-FIG. 6C shows generation of RGMb conditional knockout mice. FIG. 6A shows strategies to generate RGMb conditional knockout mice. FIG. 6B shows validation of CD4-Cre mediated deletion of RGMb in peripheral naïve CD8 T cells by qPCR (FIG. 6C) Validation of LysM-Cre mediated deletion of RGMb in bone marrow derived macrophages by qPCR.

FIG. 7A-FIG. 7K shows RGMb on T cells regulates anti-tumor immunity in germ free (GF) mice. FIG. 7A shows levels of RGMb RNA expression in tumor-infiltrating CD4+ T cells, CD8+ T cells, CD11c+MHC class II+ cells and CD11b+ cells sorted from MC38 tumors in GF and SPF mice on day 11 after implantation, and quantified by qPCR. FIG. 7B shows surface expression of RGMb protein on tumor-infiltrating leukocytes isolated from MC38 tumors on day 13 after implantation, measured by flow cytometry using monoclonal antibody (clone 9D3) against RGMb. Representative histograms of expression of RGMb on CD8+ T cells (upper left) and CD11c+MHC class II+ cells (lower left) in GF (black) and SPF (red) mice are shown and frequencies of RGMb+ cells within indicated cell populations quantified (right). n=4-5 mice per group. Significance measured by unpaired Mann-Whitney test. Data are representative from 2 different experiments. FIG. 7C shows RGMb expression on CD8+ T cells in MC38 tumors from GF and SPF mice treated with isotype and anti-PD-L1 on Day 11 pi. (FIG. 7D-FIG. 7G) MC38 tumors were harvested from GF mice treated with indicated antibodies at day 18 pi and total numbers of (FIG. 7D) CD8+ T cells, (FIG. 7E) CD4+ T cells and (FIG. 7F) Treg cells in tumors were measured. FIG. 7G shows the ratio of CD8⁺ to Treg was calculated. Significance measured by non-parametric one-way ANOVA and Dunn's multiple comparisons test. MC38 tumor growth in (FIG. 7H) rgmbf/f vs rgmbf/f CD4-Cre or (FIG. 7I) rgmbf/f vs rgmbf/f LysM-Cre mice treated with ABX and given anti-PD-L1. n=7-12 mice per group. Significance at day 30 is shown and was measured by two-way ANOVA and Sidak's multiple comparisons test. Data are representative from 2 different experiments. WT BMDCs were co-cultured with WT or RGMb KO T-cells and CD8⁺ T-cells were analyzed by flow cytometry for (FIG. 7J) Mean Fluorescence Intensity (MFI) of CD44 and (FIG. 7K) frequency of cells expressing IFNγ, TNFα, and IL-2. Significance measured by unpaired Mann-Whitney test. **** P<0.0001, *** P<0.001, P<0.01, * P<0.05.

FIG. 8A-FIG. 8D shows RGMb KO vs WT T-cell activation in vitro. WT or RGMb KO CD8⁺ T-cells were co-cultured with WT BMDCs. CD8⁺ T cells were analyzed by flow cytometry for (FIG. 8A) Mean Fluorescence Intensity (MFI) of T-Bet (FIG. 8B) MFI of CD107a (FIG. 8C) MFI of Granzyme B and (FIG. 8D) proliferation measured by Cell Trace Violet. N=3 mice per group. Representative experiment of 3 experiments. Significance determined by unpaired Mann-Whitney test. *P<0.05.

FIG. 9 shows RGMb is associated with TCR signaling (Top) WT or RGMb KO CD8+ T cells were stimulated for 0, 2, 10 minutes by TCR crosslinking, and protein expression of phosphorylated Erk1/2, Erk, phospoyrlated AKT, pan-AKT, and beta-actin were measured by Western Blot. (Bottom) Relative mRNA expression levels of RGMb in naïve T cells and TCR-activated T cells.

### DETAILED DESCRIPTION

In some aspects, provided herein are populations of T cells (e.g., modified T cells) that comprise decreased expression or activity of RGMb.

In some aspects, provided herein are methods of treating or preventing cancer in a subject, comprising administering to the subject T cells (e.g., modified T cells) conjointly with an agent that specifically binds to PD-1 or PD-L1, wherein the T cells comprise decreased expression or activity of RGMb.

In some embodiments, the methods further comprise administering to the subject the T cells (e.g., modified T cells) conjointly with an agent that specifically binds to PD-1 or PD-L1 (such as an agent that specifically binds to PD-1 or PD-L1 disclosed herein) and the RGMb agent, such as an antibody RGMb agent.

Provided herein are methods of treating or preventing cancer in a subject, comprising administering to the subject T cells (e.g., modified T cells) conjointly with an agent that specifically binds to PD-1 or PD-L1, wherein the T cells comprise decreased expression or activity of RGMb. In some embodiments, the subject is non-responsive or otherwise categorized as a “non-responder” to immune checkpoint blockade (such as PD-1/PD-L1 checkpoint blockade).As used herein, the term “nonresponsive” or “non-responders” includes patients that are refractory or resistant to previous treatments (e.g., refractory or resistant to PD-1 and/or PD-L1 immune checkpoint blockade). Nonresponsive patients include any patient who was subjected to PD-1 or PD-L1 blockade therapy and did not exhibit parameters of cancer regression or slowing of tumor progression. Nonresponsive patients include any patient that has been deemed clinically nonresponsive to PD-1 or PD-L1 blockade, or otherwise exhibited a poor clinical response to PD-1 or PD-L1 blockade. In some embodiments, a nonresponsive patient includes patients that initially respond to PD-1 or PD-L1 blockade therapy but develop resistance to such therapies.

In some aspects, provided herein are compositions comprising a population of modified T cells, wherein the T cells comprise a single chain variable fragment (scFv) specific for an RGMb peptide. In some embodiments, the composition further comprises an agent that specifically binds to PD-1 or PD-L1 (e.g., any agent that specifically binds to PD-1 or PD-L1 disclosed herein). In some aspects, provided herein are compositions comprising populations of modified T cells, wherein the T cells comprise a single chain variable fragment (scFv) specific for an RGMb peptide and a second single chain variable fragment (scFv) specific for a PD-1 or PD-L1 peptide. In some aspects, such compositions are administered to a subject in methods of treating cancer in a subject.

In some aspects, provided herein are methods of preparing a cancer therapy comprising T cells, comprising: isolating a population of T cells ex vivo and contacting the T cells with an RGMb agent that decreases the expression of or inhibits the activity of RGMb in the T cells.

### Definitions

For convenience, certain terms employed in the specification, examples, and appended claims are collected here.

As used herein, the term “administering” means providing a pharmaceutical agent or composition to a subject, and includes, but is not limited to, administering by a medical professional and self-administering.

The term “agent” is used herein to denote a chemical compound, a small molecule, a mixture of chemical compounds and/or a biological macromolecule (such as an antibody, nucleic acid, a protein, or a peptide, such as a non-activating form of RGMb or a fusion protein). Agents may be identified as having a particular activity by screening assays described herein below. The activity of such agents may render them suitable as a “therapeutic agent” which is a biologically, physiologically, or pharmacologically active substance (or substances) that acts locally or systemically in a subject.

The term “amino acid” is intended to embrace all molecules, whether natural or synthetic, which include both an amino functionality and an acid functionality and capable of being included in a polymer of naturally-occurring amino acids. Exemplary amino acids include naturally-occurring amino acids; analogs, derivatives and congeners thereof; amino acid analogs having variant side chains; and all stereoisomers of any of any of the foregoing.

As used herein, the term “antibody” may refer to both an intact antibody and an antigen-binding fragment thereof. Intact antibodies are glycoproteins that include at least two heavy (H) chains and two light (L) chains inter-connected by disulfide bonds. Each heavy chain includes a heavy chain variable region (abbreviated herein as V_{H}) and a heavy chain constant region. Each light chain includes a light chain variable region (abbreviated herein as V_{L}) and a light chain constant region. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Clq) of the classical complement system. The term “antibody” includes, for example, monoclonal antibodies, polyclonal antibodies, chimeric antibodies, humanized antibodies, human antibodies, multi-specific antibodies (e.g., bispecific antibodies), single-chain antibodies and antigen-binding antibody fragments. An “isolated antibody,” as used herein, refers to an antibody which is substantially free of other antibodies having different antigenic specificities. An isolated antibody may, however, have some cross-reactivity to other, related antigens.

The terms “antigen-binding fragment” and “antigen-binding portion” of an antibody, as used herein, refers to one or more fragments of an antibody that retain the ability to bind to an antigen. Examples of binding fragments encompassed within the term “antigen-binding fragment” of an antibody include Fab, Fab′, F(ab′)₂, Fv, scFv, disulfide linked Fv, Fd, diabodies, single-chain antibodies, NANOBODIES®, isolated CDRH3, and other antibody fragments that retain at least a portion of the variable region of an intact antibody. These antibody fragments can be obtained using conventional recombinant and/or enzymatic techniques and can be screened for antigen binding in the same manner as intact antibodies.

The term “chimeric antigen receptor” (CAR) refers to molecules that combine a binding domain against a component present on the target cell, for example an antibody-based specificity for a desired antigen (e.g., a tumor antigen) with a T cell receptor-activating intracellular domain to generate a chimeric protein that exhibits a specific anti-target cellular immune activity. Generally, CARs can consist of an extracellular single chain antigen-binding domain (scFv) fused to the intracellular signaling domain of the T cell antigen receptor complex zeta chain, and have the ability, when expressed in T cells, to redirect antigen recognition based on the monoclonal antibody's specificity.

The terms “CDR”, and its plural “CDRs”, refer to a complementarity determining region (CDR) of an antibody or antibody fragment, which determine the binding character of an antibody or antibody fragment. In most instances, three CDRs are present in a light chain variable region (CDRL1, CDRL2 and CDRL3) and three CDRs are present in a heavy chain variable region (CDRH1, CDRH2 and CDRH3). CDRs contribute to the functional activity of an antibody molecule and are separated by amino acid sequences that comprise scaffolding or framework regions. Among the various CDRs, the CDR3 sequences, and particularly CDRH3, are the most diverse and therefore have the strongest contribution to antibody specificity. There are at least two techniques for determining CDRs: (1) an approach based on cross-species sequence variability (i.e., Kabat et al., Sequences of Proteins of Immunological Interest (National Institute of Health, Bethesda, Md. (1987), incorporated by reference in its entirety); and (2) an approach based on crystallographic studies of antigen-antibody complexes (Chothia et al., Nature, 342:877 (1989), incorporated by reference in its entirety).

A “guide RNA” or “gRNA” is an RNA molecule that binds to a Cas protein (e.g., Cas9 protein) and targets the Cas protein to a specific location within a target DNA. Guide RNAs can comprise two segments: a “DNA-targeting segment” and a “protein-binding segment.” “Segment” includes a section or region of a molecule, such as a contiguous stretch of nucleotides in an RNA. Some gRNAs, such as those for Cas9, can comprise two separate RNA molecules: an “activator-RNA” (e.g., tracrRNA) and a “targeter-RNA” (e.g., CRISPR RNA or crRNA). Other gRNAs are a single RNA molecule (single RNA polynucleotide), which can also be called a “single-molecule gRNA,” a “single-guide RNA,” or an “sgRNA.” See, e.g., WO 2013/176772, WO 2014/065596, WO 2014/089290, WO 2014/093622, WO 2014/099750, WO 2013/142578, and WO 2014/131833, each of which is herein incorporated by reference in its entirety for all purposes. For Cas9, for example, a single-guide RNA can comprise a crRNA fused to a tracrRNA (e.g., via a linker).

The term “guide RNA target sequence” as used herein refers specifically to the sequence on the non-complementary strand corresponding to (i.e., the reverse complement of) the sequence to which the guide RNA hybridizes on the complementary strand. That is, the guide RNA target sequence refers to the sequence on the non-complementary strand adjacent to the PAM (e.g., upstream or 5′ of the PAM in the case of Cas9). A guide RNA target sequence is equivalent to the DNA-targeting segment of a guide RNA, but with thymines instead of uracils. As one example, a guide RNA target sequence for an SpCas9 enzyme can refer to the sequence upstream of the 5′-NGG-3′ PAM on the non-complementary strand.

As used herein, the term “humanized antibody” refers to an antibody that has at least one CDR derived from a mammal other than a human, and a FR region and the constant region of a human antibody. A humanized antibody is useful as an effective component in a therapeutic agent since antigenicity of the humanized antibody in human body is lowered.

As used herein, the term “modified” refers to a modification of a nucleic acid sequence of a cell, including, but not limited to, deleting a coding or non-coding region or a portion thereof or inserting a coding region or a portion thereof. The term modified includes any treatment of a T cell which reduces or decreases the activity or expression of RGMb in the T cell, including, but not limited to, RNAi, peptide inhibitor, or antibody treatment. In some embodiments, the T cells are modified ex vivo prior to administration to the patient or subject.

As used herein, the term “monoclonal antibody” refers to an antibody obtained from a population of substantially homogeneous antibodies that specifically bind to the same epitope, i.e., the individual antibodies comprising the population are identical except for possible naturally occurring mutations that may be present in minor amounts. The modifier “monoclonal” indicates the character of the antibody as being obtained from a substantially homogeneous population of antibodies, and is not to be construed as requiring production of the antibody by any particular method.

The terms “polynucleotide”, and “nucleic acid” are used interchangeably. They refer to a polymeric form of nucleotides of any length, either deoxyribonucleotides or ribonucleotides, or analogs thereof. Polynucleotides may have any three-dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified, such as by conjugation with a labeling component. The term “recombinant” polynucleotide means a polynucleotide of genomic, cDNA, semisynthetic, or synthetic origin which either does not occur in nature or is linked to another polynucleotide in a non-natural arrangement.

The phrase “pharmaceutically-acceptable carrier” as used herein means a pharmaceutically-acceptable material, composition or vehicle, such as a liquid or solid filler, diluent, excipient, or solvent encapsulating material, involved in carrying or transporting the subject compound from one organ, or portion of the body, to another organ, or portion of the body.

As used herein, a therapeutic that “prevents” a disorder or condition refers to a compound that, when administered to a statistical sample prior to the onset of the disorder or condition, reduces the occurrence of the disorder or condition in the treated sample relative to an untreated control sample, or delays the onset or reduces the severity of one or more symptoms of the disorder or condition relative to the untreated control sample.

As used herein, “specific binding” refers to the ability of an antibody to bind to a predetermined antigen or the ability of a polypeptide to bind to its predetermined binding partner. Typically, an antibody or polypeptide specifically binds to its predetermined antigen or binding partner with an affinity corresponding to a K_{D} of about 10⁻⁷ M or less, and binds to the predetermined antigen/binding partner with an affinity (as expressed by K_{D}) that is at least 10 fold less, at least 100 fold less or at least 1000 fold less than its affinity for binding to a non-specific and unrelated antigen/binding partner (e.g., BSA, casein).

The term “small molecule” is a term of the art and includes molecules that are less than about 1000 molecular weight or less than about 500 molecular weight. In one embodiment, small molecules do not exclusively comprise peptide bonds. In another embodiment, small molecules are not oligomeric. Exemplary small molecule compounds which can be screened for activity include, but are not limited to, peptides, peptidomimetics, nucleic acids, carbohydrates, small organic molecules (e.g., polyketides) (Cane et al. (1998) Science 282:63), and natural product extract libraries. In another embodiment, the compounds are small, organic non-peptidic compounds. In a further embodiment, a small molecule is not biosynthetic.

As used herein, the term “subject” means a human or non-human animal selected for treatment or therapy.

As used herein, the term “T cell” includes, but is not limited to, any T cell type listed herein, including CD4+ T cells and CD8+T cells. The term “T cell” also includes both T helper 1 type T cells and T helper 2 type T cells. T cells express a cell surface receptor that recognizes a specific antigenic moiety on the surface of a target cell. The cell surface receptor may be a wild type or recombinant T cell receptor (TCR), a chimeric antigen receptor (CAR), or any other surface receptor capable of recognizing an antigenic moiety that is associated with the target cell. Typically, a TCR has two protein chains (alpha- and beta-chain), which bind to specific peptides presented by an MHC protein on the surface of certain cells. TCRs recognize peptides in the context of MHC molecules expressed on the surface of a target cell. TCRs also recognize cancer antigens presented directly on the surface of cancer cells.

The “tumor microenvironment” is an art-recognized term and refers to the cellular environment in which the tumor exists, and includes, for example, interstitial fluids surrounding the tumor, surrounding blood vessels, immune cells, other cells, fibroblasts, signaling molecules, and the extracellular matrix.

The phrases “therapeutically-effective amount” and “effective amount” as used herein means the amount of an agent which is effective for producing the desired therapeutic effect in at least a sub-population of cells in a subject at a reasonable benefit/risk ratio applicable to any medical treatment.

“Treating” a disease in a subject or “treating” a subject having a disease refers to subjecting the subject to a pharmaceutical treatment, e.g., the administration of a drug, such that at least one symptom of the disease is decreased or prevented from worsening.

### T-Cells

T cells described herein can be, for example, any T cell that expresses RGMb prior to modification.

Provided herein are compositions comprising at least one agent that specifically binds to PD-1 or PD-L1 and T cells that comprise decreased expression or activity of RGMb. In some embodiments, the T cells have been modified to inhibit the activity of RGMb, the expression of RGMb, or disrupt the activity of RGMb, wherein modifying comprises contacting the T cells ex vivo with an RGMb agent that decreases the expression of or inhibits or disrupts the activity of RGMb in the T cells. The RGMb agent may be an antibody that specifically binds to an RGMb peptide, such as a peptide listed in Table 1. In some embodiments, the RGMb agent is an RNA inhibitory molecule that targets an RGMb mRNA transcript, such as a transcript listed in Table 2. In some embodiments, the RGMb agent is a gRNA complementary for a portion of the RGMb genomic sequence, such as a genomic sequence listed in Table 3.

T cells, as described herein, can be modified following isolation using known methods, or the T cells can be activated and expanded (or differentiated in the case of progenitors) in vitro prior or after to being modified. In another embodiment, the T cells are genetically modified with the chimeric antigen receptors (e.g., transduced with a viral vector comprising a nucleic acid encoding a CAR) and then are activated and expanded in vitro. In some embodiments, the T cells are genetically modified with the engineered T cell receptors (e.g., transduced with a viral vector comprising a nucleic acid encoding a TCR) and then are activated and expanded in vitro. Methods for activating and expanding T cells are known in the art and are described, for example, in U.S. Pat. Nos. 6,905,874; 6,867,041; 6,797,514; WO2012079000. Generally, such methods include contacting PBMC or isolated T cells with a stimulatory agent and costimulatory agent, such as anti-CD3 and anti-CD28 antibodies, generally attached to a bead or other surface, in a culture medium with appropriate cytokines, such as IL-2 (e.g., recombinant human IL-2). Anti-CD3 and anti-CD28 antibodies attached to the same bead serve as a “surrogate” antigen presenting cell (APC). In other embodiments, the T cells may be activated and stimulated to proliferate with feeder cells and appropriate antibodies and cytokines using methods such as those described in U.S. Pat. Nos. 6,040,177; 5,827,642; and WO2012129514.

The T cell populations disclosed herein may comprise T helper cells. T helper cells (T_{H} cells) assist other white blood cells in immunologic processes, including maturation of B cells into plasma cells and memory B cells, and activation of cytotoxic T cells and macrophages. These cells are also known as CD4⁺ T cells because they express the CD4 glycoprotein on their surface. Helper T cells become activated when they are presented with peptide antigens by MHC class II molecules, which are expressed on the surface of antigen-presenting cells (APCs). Once activated, they divide rapidly and secrete small proteins called cytokines that regulate or assist in the active immune response. These cells can differentiate into one of several subtypes, including T_{H}1, T_{H}2, T_{H}3, T_{H}17, T_{H}9, or T^{FH}, which secrete different cytokines to facilitate a different type of immune response.

The T cell populations disclosed herein may comprise CTL cells. Cytotoxic T cells (Tc cells, or CTLs) destroy virally infected cells and tumor cells, and are also implicated in transplant rejection. These cells are also known as CD8⁺ T cells since they express the CD8 glycoprotein at their surface. These cells recognize their targets by binding to antigen associated with MHC class I molecules, which are present on the surface of all nucleated cells. Through IL-10, adenosine and other molecules secreted by regulatory T cells, the CD8⁺ cells can be inactivated to an anergic state, which prevents autoimmune diseases.

The T cell populations disclosed herein may comprise memory T cells. Memory T cells are a subset of antigen-specific T cells that persist long-term after an infection has resolved. They quickly expand to large numbers of effector T cells upon re-exposure to their cognate antigen, thus providing the immune system with “memory” against past infections. Memory cells may be either CD4⁺ or CD8⁺. Memory T cells typically express the cell surface protein CD45RO.

The T cell populations disclosed herein may comprise regulatory T cells. Regulatory T cells (T_{reg} cells), formerly known as suppressor T cells, are crucial for the maintenance of immunological tolerance. Their major role is to shut down T cell-mediated immunity toward the end of an immune reaction and to suppress auto-reactive T cells that escaped the process of negative selection in the thymus. Two major classes of CD4⁺ T_{reg} cells have been described—naturally occurring T_{reg} cells and adaptive T_{reg} cells.

The T cell populations disclosed herein may comprise Natural killer T (NKT) cells. Natural killer T (NKT) cells (not to be confused with natural killer (NK) cells) bridge the adaptive immune system with the innate immune system. Unlike conventional T cells that recognize peptide antigens presented by major histocompatibility complex (MHC) molecules, NKT cells recognize glycolipid antigen presented by a molecule called CD1d.

In some embodiments, the T cells comprise a mixture of CD4⁺ cells. In other embodiments, the T cells are enriched for one or more subsets based on cell surface expression. For example, in some cases, the T comprise are cytotoxic CD8⁺ T lymphocytes.

Natural-killer (NK) cells are CD56⁺CD3⁻ large granular lymphocytes that can kill virally infected and transformed cells, and constitute a critical cellular subset of the innate immune system (Godfrey J, et al. Leuk Lymphoma 2012 53:1666-1676). Unlike cytotoxic CD8⁺ T lymphocytes, NK cells launch cytotoxicity against tumor cells without the requirement for prior sensitization, and can eradicate MHC-I-negative cells (Narni-Mancinelli E, et al. Int Immunol 2011 23:427-431). NK cells are safer effector cells, as they may avoid the potentially lethal complications of cytokine storms (Morgan R A, et al. Mol Ther 2010 18:843-851), tumor lysis syndrome (Porter D L, et al. N Engl J Med 2011 365:725-733), and on-target, off-tumor effects.

In some aspects, provided herein are compositions comprising a population of modified T cells, wherein the T cells comprise a single chain variable fragment (scFv) specific for an RGMb peptide (e.g., an RGMB peptide or portion thereof listed in Table 1). In some embodiments, the composition further comprises an agent that specifically binds to PD-1 or PD-L1 (e.g., any agent that specifically binds to PD-1 or PD-L1 disclosed herein). In some aspects, provided herein are compositions comprising populations of modified T cells, wherein the T cells comprise a single chain variable fragment (scFv) specific for an RGMb peptide and a second single chain variable fragment (scFv) specific for a PD-1 or PD-L1 peptide. Any population of T cells disclosed herein may be used as a cancer treatment. A single chain variable fragment or “scFv” can refer to a single folded polypeptide comprising the VH and VL domains of an antibody linked through a linker molecule. In such a scFv, the VH and VL domains can be either in the VH-linker-VL or VL-linker-VH order. In addition to facilitate its production, a scFv fragment may contain a tag molecule linked to the scFv via a spacer. Therefore, in some embodiments, a scFv fragment thus comprises the VH and VL domains implicated in antigen recognizing but not the immunogenic constant domains of the corresponding antibody. Construction of scFvs can be accomplished by using the DNA segments encoding the antibody heavy (VH) and light (VL) variable chains specific for a peptide thats amplified from cDNA in hybridoma cells. scFvs can be expressed in T cells by transfection of vectors encoding scFvs. Therefore, included herein are nucleic acids encoding such a scFv or fragments thereof, expression vectors and host cells to produce such a scFv or fragment thereof, as well as therapeutic uses of such a scFv fragment.

In some embodiments, the invention concerns a single chain variable fragment (scFv) directed against an RGMb peptide comprising or consisting of an amino acid sequence having at least 95%, at least 96%, at least 97%, at least 98%, at least 98.5%, at least 99%, at least 99.5%, at least 99.6%, at least 99.7%, at least 99.8%, identity with a RGMb peptide or fragment thereof listed in Table 1.

Framework regions may be mutated to generate a chimeric, in particular humanized, scFv fragment directed against human RGMb, PD-1, or PD-L1. With regards to a reference antibody or antibody fragment of a given species, a corresponding “chimeric” antibody or antibody fragment in which framework regions have been replaced by corresponding framework regions of another species. More particularly, a “humanized” antibody or antibody fragment is an antibody or antibody fragment with identical CDR regions in which framework regions have been replaced by human framework regions. To generate such a humanized scFv fragment, a well known technology called CDR grafting may be used, which involves selecting the complementary determining regions (CDRs) from a donor scFv fragment, and grafting them onto a human scFv fragment framework of known three dimensional structure (see, e.g., WO98/45322 (24); WO 87/02671 (25); U.S. Pat. No. 5,859,205 (26); U.S. Pat. No. 5,585,089 (27); U.S. Pat. No. 4,816,567 (28); EP0173494 (29); and references 20-21 and 30-31) or performing database searches to identify potential candidates. In a typical method aided by computer modeling and comparison to human germline sequences, the antigen binding loops of the monoclonal antibody to be humanized are superimposed onto the best fitting frameworks. Such methods can also be used to humanize antibodies disclosed herein.

Such a host cell may be either prokaryotic or eukaryotic. Suitable prokaryotic host cells include gram-positive and gram-negative bacteria. Among gram-negative bacteria, a preferred host cell is represented by E. coli. For use in bacteria, the expression vector may preferably contain a leader sequence directing the expression of the scFv fragment into bacterial periplasm, corresponding to the space between the plasma membrane and the outer membrane of gram-negative bacteria or between the plasma membrane and the peptidoglycan layer (cell wall) of gram-positive bacteria. Suitable sequences directing the expression of a polypeptide to bacteria periplasm include ompA, ompF, ompT, LamB, β-lactamase, cp VIII from M13, pelB, malE or phoA signal peptides or leader sequences. In a particular embodiment, said leader sequence is pelB.

Alternatively, a eukaryotic cell may be used, in particular a mammalian cell. Indeed, this may permit directly generating a glycosylated scFv fragment. Mammalian cell lines available as hosts for expression are known in the art and include many immortalized cell lines available notably from the American Type Culture Collection (ATCC), including but not limited to, Chinese hamster ovary (CHO) cells, HeLa cells, baby hamster kidney (131-1K) cells, monkey kidney cells (COS), human hepatocellular carcinoma cells (e.g., Hep G2), and a number of other cell lines. As eukaryotic cells, yeast cells may also be used. To obtain a glycosylation pattern closest to a natural human glycosylation pattern, it may be resorted to human cell lines as host cells. Alternatively, Pichia pastoris yeast cells, a robust organism commonly used in fermentation processes which can be grown to high cell density in a chemically defined growth medium, have been modified by first eliminating endogenous yeast glycosylation pathways, while sequentially engineering into the organism a synthetic in vivo glycosylation pathway that enables the yeast to produce a complex human N-glycan, GlcNAc2Man3GlcNAc2, in vivo. Such modified yeast cells with a humanized glycosylation pathway are able to secrete a human glycoprotein with uniform complex N-glycosylation.

In some embodiments, the T cells are genetically modified to target an antigen, e.g., a cancer antigen. In some embodiments, the T cells are genetically modified with the chimeric antigen receptors (CAR). In some embodiments, the CAR comprises an antigen-binding domain, a transmembrane domain, and an intracellular domain. In one embodiment, the antigen-binding domain is selected from the group consisting of an antibody, an scFv, and a Fab. In one embodiment, the CAR further comprising a hinge domain. Exemplary hinge domains include a Fc fragment of an antibody, a hinge region of an antibody, a CH2 region of an antibody, a CH3 region of an antibody, an artificial hinge domain, a hinge comprising an amino acid sequence of CD8, or any combination thereof. In certain embodiments, the transmembrane domain is selected from the group consisting of an artificial hydrophobic sequence and transmembrane domain of a type I transmembrane protein, an alpha, beta, or zeta chain of a T cell receptor, CD28, CD3 epsilon, CD45, CD4, CD5, CD8, CD9, CD16, CD22, CD33, CD37, CD64, CD80, CD86, CD134, CD137, and CD154. In other embodiments, the intracellular domain comprises at least one co-stimulatory domain selected from the group consisting of co-stimulatory domains of proteins in the TNFR superfamily, CD28, 4-1BB (CD137), OX40 (CD134), PD-1, CD7, LIGHT, CD83L, DAP10, DAP12, CD27, CD2, CD5, ICAM-1, LFA-1, Lck, TNFR-I, TNFR-II, Fas, CD30, CD40, ICOS, NKG2C, and B7-H3. In some embodiments, the intracellular domain comprises an intracellular domain selected from the group consisting of cytoplasmic signaling domains of a human CD3 zeta chain, FcγRIII, FcsRI, a cytoplasmic tail of an Fc receptor, an immunoreceptor tyrosine-based activation motif (ITAM) bearing cytoplasmic receptors, TCR zeta, FcR gamma, FcR beta, CD3 gamma, CD3 delta, CD3 epsilon, CD5, CD22, CD79a, CD79b, and CD66d.

In some aspects, provided herein are methods comprising transfecting the T-cells with a DNA encoding a chimeric T-cell receptor (CAR) and, in some cases, a transposase. Methods of transfecting of cells are well known in the art, but in certain aspects, highly efficient transfections methods such as electroporation can be employed. For example, nucleic acids may be introduced into cells using a nucleofection apparatus.

Further aspects of the embodiments concern transfecting cells with an expression vector encoding a CAR. A wide range of CAR constructs and expression vectors for the same are known in the art and are further detailed herein. For example, in some aspects, the CAR expression vector is a DNA expression vector such as a plasmid, linear expression vector or an episome. In some aspects, the vector comprises additional sequences, such as sequence that facilitate expression of the CAR, such a promoter, enhancer, poly-A signal, and/or one or more introns. In preferred aspects, the CAR coding sequence is flanked by transposon sequences, such that the presence of a transposase allows the coding sequence to integrate into the genome of the transfected cell.

As detailed supra, in certain aspects, cells are further transfected with a transposase that facilitates integration of a CAR coding sequence into the genome of the transfected cells. In some aspects, the transposase is provided as DNA expression vector.

However, in some aspects, the transposase is provided as an expressible RNA or a protein such that long-term expression of the transposase does not occur in the transgenic cells. For example, in some aspects, the transposase is provided as an mRNA (e.g., an mRNA comprising a cap and poly-A tail). Any transposase system may be used in accordance with the embodiments. However, in some aspects, the transposase is salmonid-type Tel-like transposase (SB). For example, the transposase can be the so called “Sleeping beauty” transposase, see e.g., U.S. Pat. No. 6,489,458, incorporated herein by reference. In certain aspects, the transposase is an engineered enzyme with increased enzymatic activity. Some specific examples of transposases include, without limitation, SB10, SB 11 or SB100× transposase (see, e.g., Mates et al., 2009, incorporated herein by reference). For example, a method can involve electroporation of cells with a mRNA encoding a SB10, SB11 or SB100× transposase.

In still further aspects, a transgenic CAR cell of the embodiments further comprises an expression vector for expression of a membrane-bound cytokine that stimulates proliferation and/or survival of T-cells. In particular, CAR cells comprising such cytokines can proliferate and/or persist with little or no ex vivo culture with activating and propagating cells (AaPCs) or artificial antigen presenting cells (aAPCs) due the simulation provided by the cytokine expression. Likewise, such CAR cells can proliferate in vivo even when large amounts of antigen recognized by the CAR is not present (e.g., as in the case of a cancer patient in remission or a patient with minimal residual disease). In some aspects, the CAR cells comprise a DNA or RNA expression vector for expression of a Cγ cytokine and elements (e.g., a transmembrane domain) to provide surface expression of the cytokine. For example, the CAR cells can comprise membrane-bound versions of IL-7, IL-15 or IL-21. In some aspects, the cytokine is tethered to the membrane by fusion of the cytokine coding sequence with the receptor for the cytokine. For example, a cell can comprise a vector for expression of a IL-15-IL-15Ru fusion protein. In still further aspects, a vector encoding a membrane-bound Cγ cytokine is a DNA expression vector, such as vector integrated into the genome of the CAR cells or an extra-chromosomal vector (e.g., and episomal vector). In still further aspects, expression of the membrane-bound Cγ cytokine is under the control of an inducible promoter (e.g., a drug inducible promoter) such that the expression of the cytokine in the CAR cells (and thereby the proliferation of the CAR cells) can be controlled by inducing or suppressing promoter activity.

Aspects of the embodiments concern obtaining a sample from a patient comprising NKT cells, T-cells or T-cell progenitor cells. For example, in some cases, the sample is an umbilical cord blood sample, a peripheral blood sample (e.g., a mononuclear cell fraction) or a sample from the subject comprising pluripotent cells. In some aspects, a sample from the subject can be cultured to generate induced pluripotent stem (iPS) cells and these cells used to produce NKT cells or T-cells. Cell samples may be cultured directly from the subject or may be cryopreserved prior to use. In some aspects, obtaining a cell sample comprises collecting a cell sample. In other aspects, the sample is obtained by a third party. In still further aspects, a sample from a subject can be treated to purify or enrich the T-cells or T-cell progenitors in the sample. For example, the sample can be subjected to gradient purification, cell culture selection and/or cell sorting (e.g., via fluorescence-activated cell sorting (FACS)).

In still further aspects, a method of the embodiments comprises a step for enriching the cell population for CAR-expressing T-cells after transfection of the cells. For example, the enrichment step can comprise sorting of the cell (e.g., via FACS), for example, by using an antigen bound by the CAR or a CAR-binding antibody. In still further aspects, the enrichment step comprises depletion of the non-T-cells or depletion of cells that lack CAR expression. For example, CD56⁺ cells can be depleted from a culture population. In yet further aspects, a sample of CAR cells is preserved (or maintained in culture) when the cells are administered to the subject. For example, a sample may be cryopreserved for later expansion or analysis. In some embodiments, the exogenous receptor is a T cell receptor (TCR), e.g., wild-type TCR, a high affinity TCR, or a chimeric TCR. In certain embodiments, the exogenous TCR comprises at least one disulfide bond. In certain embodiments, the exogenous TCR comprises a TCR alpha chain and a TCR beta chain.

As used herein, the term “T cell receptor” or “TCR” refers to a complex of membrane proteins that participate in the activation of T cells in response to the presentation of antigen. The TCR is responsible for recognizing antigens bound to major histocompatibility complex molecules. TCR is composed of a heterodimer of an alpha (a) and beta (β) chain, although in some cells the TCR consists of gamma and delta (γ/δ) chains. TCRs may exist in alpha/beta and gamma/delta forms, which are structurally similar but have distinct anatomical locations and functions. Each chain is composed of two extracellular domains, a variable and constant domain. In some embodiments, the TCR may be modified on any cell comprising a TCR, including, for example, a helper T cell, a cytotoxic T cell, a memory T cell, or a regulatory T cell.

Provided herein are compositions and methods for modified immune cells or precursors thereof (e.g., modified T cells) comprising an exogenous (e.g., transgenic) T cell receptor (TCR). The present invention includes an isolated transgenic T cell receptor (TCR) for use in the modified T-cell disclosed herein. In another aspect, the invention includes an isolated nucleic acid encoding a transgenic T cell receptor (TCR) for use in the modified cell disclosed herein. In some embodiments, provided herein are genetically modified T cells comprising an exogeneous nucleic acid encoding a transgenic TCR. The TCR expression system may comprise, for example, a tetracycline (Tet)-On inducible gene expression system, wherein the Tet-On inducible gene expression system comprises a reverse Tet transactivator (Tet-On 3G) protein and at least one promoter fused downstream of at least one Tet-operator sequence (PTRE3GS Inducible Promoter) that drives the expression of a transgenic TCR in the presence of doxycycline. In one embodiment, provided herein are modified T cells comprising an exogenous TCR, wherein the exogenous TCR is expressed by a Tet-On inducible system, wherein the Tet-On inducible system comprises a promoter (e.g., constitutive promoter) operably linked to a nucleic acid encoding a reverse Tet transactivator (e.g., Tet-On 3G), and an inducible promoter operably linked to a nucleic acid encoding the exogenous TCR.

A T cell receptor is a complex of membrane proteins that participate in the activation of T cells in response to the presentation of antigen (e.g., a cancer antigen). Stimulation of the TCR is triggered by major histocompatibility complex molecules (MEW) on antigen presenting cells that present antigen peptides to the T cells and bind to the TCR complexes to induce a series of intracellular signaling cascades.

The TCR is generally composed of six different membrane bound chains that form the TCR heterodimer responsible for ligand recognition, and participate in the activation of T cells in response to an antigen. TCRs exist in alpha/beta and gamma/delta forms, which are structurally similar but have distinct anatomical locations and functions. An alpha/beta TCR comprises a TCR alpha chain and a TCR beta chain. T cells expressing a TCR comprising a TCR alpha chain and a TCR beta chain are commonly referred to as alpha/beta T cells. Gamma/delta TCRs comprise a TCR gamma chain and a TCR delta chain. T cells expressing a TCR comprising a TCR gamma chain and a TCR delta chain are commonly referred to as gamma/delta T cells. A TCR of the present disclosure is a TCR comprising a TCR alpha chain and a TCR beta chain. In one embodiment, the TCR comprises a TCR alpha and beta chain, such as the nucleic acid encoding the TCR comprises a nucleic acid encoding a TCR alpha and a TCR beta chain. In another embodiment, an alpha or beta chain or both comprises at least one N-deglycosylation.

Each chain is composed of two extracellular domains, a variable and constant domain. The TCR alpha chain and the TCR beta chain are each comprised of two extracellular domains, a variable region and a constant region. The TCR alpha chain variable region and the TCR beta chain variable region are required for the affinity of a TCR to a target antigen. Each variable region comprises three hypervariable or complementarity-determining regions (CDRs) which provide for binding to a target antigen. The constant region of the TCR alpha chain and the constant region of the TCR beta chain are proximal to the cell membrane. A TCR further comprises a transmembrane region and a short cytoplasmic tail. CD3 molecules are assembled together with the TCR heterodimer. CD3 molecules comprise a characteristic sequence motif for tyrosine phosphorylation, known as immunoreceptor tyrosine-based activation motifs (ITAMs). Proximal signaling events are mediated through the CD3 molecules, and accordingly, TCR-CD3 complex interaction plays an important role in mediating cell recognition events.

Each of the constant and variable domains of the TCR may include an intra-chain disulfide bond. In one embodiment, TCR comprises at least one disulfide bond. The variable domains include the highly polymorphic loops analogous to the complementarity determining regions (CDRs) of antibodies. The diversity of TCR sequences is generated via somatic rearrangement of linked variable (V), diversity (D), joining (J), and constant genes. Functional alpha and gamma chain polypeptides are formed by rearranged V-J-C regions, whereas beta and delta chains consist of V-D-J-C regions. The extracellular constant domain includes a membrane proximal region and an immunoglobulin region.

The TCR can include a wildtype TCR, a high affinity TCR, and/or a chimeric TCR. When the TCR is modified, it may have higher affinity for the target cell antigen than a wildtype TCR. A high affinity TCR may be the result of modifications to a wild-type TCR that confers a higher affinity for a target antigen (e.g., a cancer antigen) compared to the wild-type TCR. A high affinity TCR may be an affinity-matured TCR. Methods for modifying TCRs and/or the affinity-maturation of TCRs are known to those of skill in the art. Techniques for engineering and expressing TCRs include, but are not limited to, the production of TCR heterodimers which include the native disulphide bridge which connects the respective subunits (Garboczi, et al., (1996), Nature 384(6605): 134-41; Garboczi, et al., (1996), J Immunol 157(12): 5403-10; Chang et al., (1994), PNAS USA 91: 11408-11412; Davodeau et al., (1993), J. Biol. Chem. 268(21): 15455-15460; Golden et al., (1997), J. Imm. Meth. 206: 163-169; U.S. Pat. No. 6,080,840).

In embodiments where the TCR is a chimeric TCR, the TCR may include chimeric domains, such as the TCR comprises a co-stimulatory signaling domain at a C′ terminal of at least one of the chains. In other embodiment, the TCR may include a modified chain, such as a modified alpha or beta chain. Such modifications may include, but are not limited to, N-deglycosylation, altered domain (such as an engineered variable region to target a specific antigen or increase affinity), addition of one or more disulfide bonds, entire or fragment of a chain derived from a different species, and any combination thereof.

In some embodiments, the exogenous TCR is a full TCR or an antigen-binding portion or antigen-binding fragment thereof. In some embodiments, the TCR is an intact or full-length TCR, including TCRs in the up form or γδ form. In some embodiments, the TCR is an antigen-binding portion that is less than a full-length TCR but that binds to a specific peptide bound in an MHC molecule, such as binds to an MHC-peptide complex. In some cases, an antigen-binding portion or fragment of a TCR can contain only a portion of the structural domains of a full-length or intact TCR, but yet is able to bind the peptide epitope, such as MHC-peptide complex, to which the full TCR binds. In some cases, an antigen-binding portion contains the variable domains of a TCR, such as variable a chain and variable β chain of a TCR, sufficient to form a binding site for binding to a specific MHC-peptide complex. Generally, the variable chains of a TCR contain complementarity determining regions (CDRs) involved in recognition of the peptide, MHC and/or MHC-peptide complex.

In some embodiments, the variable domains of the TCR contain hypervariable loops, or CDRs, which generally are the primary contributors to antigen recognition and binding capabilities and specificity. In some embodiments, a CDR of a TCR or combination thereof forms all or substantially all of the antigen-binding site of a given TCR molecule. The various CDRs within a variable region of a TCR chain generally are separated by framework regions (FRs), which generally display less variability among TCR molecules as compared to the CDRs (see, e.g., Jores et al, Proc. Nat'l Acad. Sci. U.S.A. 87:9138, 1990; Chothia et al., EMBO J. 7:3745, 1988; see also Lefranc et al., Dev. Comp. Immunol. 27:55, 2003). In some embodiments, CDR3 is the main CDR responsible for antigen binding or specificity, or is the most important among the three CDRs on a given TCR variable region for antigen recognition, and/or for interaction with the processed peptide portion of the peptide-MHC complex. In some contexts, the CDR1 of the alpha chain can interact with the N-terminal part of certain antigenic peptides. In some contexts, CDR1 of the beta chain can interact with the C-terminal part of the peptide. In some contexts, CDR2 contributes most strongly to or is the primary CDR responsible for the interaction with or recognition of the MHC portion of the MHC-peptide complex. In some embodiments, the variable region of the β-chain can contain a further hypervariable region (CDR4 or HVR4), which generally is involved in superantigen binding and not antigen recognition (Kotb (1995) Clinical Microbiology Reviews, 8:411-426).

In some embodiments, a TCR contains a variable alpha domain (Va) and/or a variable beta domain (V) or antigen-binding fragments thereof. In some embodiments, the a-chain and/or n-chain of a TCR also can contain a constant domain, a transmembrane domain and/or a short cytoplasmic tail (see, e.g., Janeway et al., Immunobiology: The Immune System in Health and Disease, 3 Ed., Current Biology Publications, p. 4:33, 1997). In some embodiments, the a-chain constant domain is encoded by the TRAC gene (IMGT nomenclature) or is a variant thereof. In some embodiments, the β-chain constant region is encoded by TRBC1 or TRBC2 genes (IMGT nomenclature) or is a variant thereof. In some embodiments, the constant domain is adjacent to the cell membrane. For example, in some cases, the extracellular portion of the TCR formed by the two chains contains two membrane-proximal constant domains, and two membrane-distal variable domains, which variable domains each contain CDRs.

In some embodiments, the TCR may be a heterodimer of two chains a and β (or optionally γ and δ) that are linked, such as by a disulfide bond or disulfide bonds. In some embodiments, the constant domain of the TCR may contain short connecting sequences in which a cysteine residue forms a disulfide bond, thereby linking the two chains of the TCR. In some embodiments, a TCR may have an additional cysteine residue in each of the a and β chains, such that the TCR contains two disulfide bonds in the constant domains. In some embodiments, each of the constant and variable domains contain disulfide bonds formed by cysteine residues.

In some embodiments, the TCR for engineering cells as described is one generated from a known TCR sequence(s), such as sequences of vα,β chains, for which a substantially full-length coding sequence is readily available. Methods for obtaining full-length TCR sequences, including V chain sequences, from cell sources are well known. In some embodiments, nucleic acids encoding the TCR can be obtained from a variety of sources, such as by polymerase chain reaction (PCR) amplification of TCR-encoding nucleic acids within or isolated from a given cell or cells, or synthesis of publicly available TCR DNA sequences. In some embodiments, the TCR is obtained from a biological source, such as from cells such as from a T cell (e.g. cytotoxic T cell), T-cell hybridomas or other publicly available source. In some embodiments, the T-cells can be obtained from in vivo isolated cells. In some embodiments, the T-cells can be a cultured T-cell hybridoma or clone. In some embodiments, the TCR or antigen-binding portion thereof can be synthetically generated from knowledge of the sequence of the TCR. In some embodiments, a high-affinity T cell clone for a target antigen (e.g., a cancer antigen) is identified, isolated from a patient, and introduced into the cells. In some embodiments, the TCR clone for a target antigen has been generated in transgenic mice engineered with human immune system genes (e.g., the human leukocyte antigen system, or HLA). See, e.g., tumor antigens (see, e.g., Parkhurst et al. (2009) Clin Cancer Res. 15: 169-180 and Cohen et al. (2005) J Immunol. 175:5799-5808. In some embodiments, phage display is used to isolate TCRs against a target antigen (see, e.g., Varela-Rohena et al. (2008) Nat Med. 14: 1390-1395 and Li (2005) Nat Biotechnol. 23:349-354.

In some embodiments, the TCR or antigen-binding portion thereof is one that has been modified or engineered. In some embodiments, directed evolution methods are used to generate TCRs with altered properties, such as with higher affinity for a specific MHC-peptide complex. In some embodiments, directed evolution is achieved by display methods including, but not limited to, yeast display (Holler et al. (2003) Nat Immunol, 4, 55-62; Holler et al. (2000) Proc Natl Acad Sci USA, 97, 5387-92), phage display (Li et al. (2005) Nat Biotechnol, 23, 349-54), or T cell display (Chervin et al. (2008) J Immunol Methods, 339, 175-84). In some embodiments, display approaches involve engineering, or modifying, a known, parent or reference TCR. For example, in some cases, a wild-type TCR can be used as a template for producing mutagenized TCRs in which in one or more residues of the CDRs are mutated, and mutants with an desired altered property, such as higher affinity for a desired target antigen, are selected.

In some embodiments, an inducible TCR or CAR expression system provided herein comprises: a first nucleic acid comprising a constitutive promoter operably linked to a nucleic acid sequence encoding a transactivator protein; and a second nucleic acid comprising an inducible promoter operably linked to a nucleic acid sequencing encoding a TCR or CAR. The first nucleic acid and the second nucleic acid may reside on separate expression constructs. In an exemplary embodiment, the first nucleic acid and the second nucleic acid reside within the same expression construct. In an exemplary embodiment, the direction of the first nucleic acid is in reverse orientation of the second nucleic acid (i.e., the second nucleic acid is in reverse orientation to the first nucleic acid). In such an embodiment, the inducible expression system is said to be within a bidirectional expression construct.

### Therapeutic Methods

The T cells, agents and/or compositions described herein may be administered systemically, intravenously, intramuscularly, orally, or locally (e.g., delivered locally to a tumor). The compositions and/or agents disclosed herein may be delivered by any suitable route of administration, including orally, nasally, as by, for example, a spray, rectally, intravaginal, parenterally, intracisternally and topically, as by powders, ointments or drops, including buccally and sublingually. In certain embodiments, the agents and/or compositions are delivered generally (e.g., via oral or parenteral administration). In certain other embodiments, the compositions and/or agents are delivered locally through injection. The therapeutics described herein may be administered through conjunctive therapy. Conjunctive therapy includes sequential, simultaneous and separate, and/or co-administration of the compositions and/or agents in such a way that the therapeutic effects of the first agent administered have not entirely disappeared when the subsequent agent is administered. In certain embodiments, the additional agent may be co-formulated with the first and/or second agent or be formulated in a separate pharmaceutical composition. In some embodiments, the compositions and additional agents are administered at the same time or at different times (e.g., the compositions and additional agents are administered sequentially).

Typical subjects for treatment include persons afflicted with or suspected of having or being pre-disposed to a disease disclosed herein, or persons susceptible to, suffering from or that have suffered a disease disclosed herein. A subject may or may not have a genetic predisposition for a disease disclosed herein. As used herein, the phrase “conjoint administration” refers to any form of administration of two or more different agents (e.g., two different antibodies) such that the second agent is administered while the previously administered agent is still effective in the body. For example, the compositions disclosed herein can be administered either in the same formulation or in a separate formulation, either concomitantly or sequentially.

The pharmaceutical compositions disclosed herein may be delivered by any suitable route of administration, including orally, locally, and parenterally. In certain embodiments the pharmaceutical compositions are delivered generally (e.g., via oral or parenteral administration).

In certain aspects, agents and/or compositions disclosed herein may be administered at a dose sufficient to achieve the desired result.

Immune checkpoint inhibitor dosing may follow any dosing regime or schedule known in the art. For example, dosing can be determined by cancer type or cancer disease stage, as well as the characteristics of the afflicted patient, such as weight, sex, ethnicity, and/or sensitivity to medication. Exemplary dosing regimes and schedules can be found at https://packageinserts.bms.com/pi/pi_opdivo.pdf;
https://www.merck.com/product/usa/pi_circulars/k/keytruda/keytruda_pi.pdf;
https://www.accessdata.fda.gov/drugsatfda_docs/label/2018/761069s0021bl.pdf; or
https://www.accessdata.fda.gov/drugsatfda_docs/label/2017/761049s0001bl.pdf.

In some embodiments, administering an agent (e.g., the T cells or second agent) or composition to the subject comprises administering a bolus of the composition. The method may comprise administering the composition to the subject at least once per month, twice per month, three times per month. In certain embodiments, the method may comprise administering the composition at least once per week, at least once every two weeks, or once every three weeks. In some embodiments, the method may comprise administering the composition to the subject 1, 2, 3, 4, 5, 6, or 7 times per week.

The compositions and agents disclosed herein may be administered over any period of time effective to achieve the desired therapeutic response for a particular patient, composition, and mode of administration, without being toxic to the patient. The period of time may be at least 1 day, at least 10 days, at least 20 days, at least 30, days, at least 60 days, at least three months, at least six months, at least a year, at least three years, at least five years, or at least ten years. The dose may be administered when needed, sporadically, or at regular intervals. For example, the dose may be administered monthly, weekly, biweekly, triweekly, once a day, or twice a day. In certain embodiments, a dose of the composition is administered at regular intervals over a period of time. In some embodiments, a dose of the composition is administered at least once a week. In some embodiments, a dose of the composition is administered at least twice a week. In certain embodiments, a dose of the composition is administered at least three times a week. In some embodiments, a dose of the composition is administered at least once a day. In some embodiments, a dose of the composition is administered at least twice a day. In some embodiments, doses of the composition are administered for at least 1 week, for at least 2 weeks, for at least 3 weeks, for at least 4 weeks, for at least 1 month, for at least 2 months, for at least 3 months, for at least 4 months, for at least 5 months, for at least 6 months, for at least 1 year, for at least two years, at least three years, or at least five years.

The selected dosage level will depend upon a variety of factors including the activity of the particular agent employed, the route of administration, the time of administration, the rate of excretion or metabolism of the particular compound being employed, the duration of the treatment, other drugs, compounds and/or materials used in combination with the particular compound employed, the age, sex, weight, condition, general health and prior medical history of the patient being treated, and like factors well known in the medical arts.

A physician having ordinary skill in the art can readily determine and prescribe the effective amount of the pharmaceutical composition required. For example, the physician could prescribe and/or administer doses of the compounds employed in the pharmaceutical composition at levels lower than that required in order to achieve the desired therapeutic effect and gradually increase the dosage until the desired effect is achieved.

### Interfering Nucleic Acid Agents

Certain embodiments disclosed herein relate to modification of T cells to decrease of inhibit the expression or activity of RGMb (e.g., by administering an interfering RNA molecule, such as a interfering agent that targets an RGMb mRNA transcript, such as a transcript listed in Table 2). RGMb may be modulated in the T cells by interfering nucleic acids. Interfering nucleic acids generally include a sequence of cyclic subunits, each bearing a base-pairing moiety, linked by intersubunit linkages that allow the base-pairing moieties to hybridize to a target sequence in a nucleic acid (typically an RNA) by Watson-Crick base pairing, to form a nucleic acid:oligomer heteroduplex within the target sequence. Interfering RNA molecules include, but are not limited to, antisense molecules, siRNA molecules, single-stranded siRNA molecules, miRNA molecules and shRNA molecules.

Typically at least 17, 18, 19, 20, 21, 22 or 23 nucleotides of the complement of the target mRNA sequence are sufficient to mediate inhibition of a target transcript. Perfect complementarity is not necessary. In some embodiments, the interfering nucleic acid molecule is double-stranded RNA. The double-stranded RNA molecule may have a 2 nucleotide 3′ overhang. In some embodiments, the two RNA strands are connected via a hairpin structure, forming a shRNA molecule. shRNA molecules can contain hairpins derived from microRNA molecules. For example, an RNAi vector can be constructed by cloning the interfering RNA sequence into a pCAG-miR30 construct containing the hairpin from the miR30 miRNA. RNA interference molecules may include DNA residues, as well as RNA residues.

Interfering nucleic acid molecules provided herein can contain RNA bases, non-RNA bases or a mixture of RNA bases and non-RNA bases. For example, interfering nucleic acid molecules provided herein can be primarily composed of RNA bases but also contain DNA bases or non-naturally occurring nucleotides.

The interfering nucleic acids can employ a variety of oligonucleotide chemistries. Examples of oligonucleotide chemistries include, without limitation, peptide nucleic acid (PNA), linked nucleic acid (LNA), phosphorothioate, 2'O-Me-modified oligonucleotides, and morpholino chemistries, including combinations of any of the foregoing. In general, PNA and LNA chemistries can utilize shorter targeting sequences because of their relatively high target binding strength relative to 2'O-Me oligonucleotides. Phosphorothioate and 2'O-Me-modified chemistries are often combined to generate 2'O-Me-modified oligonucleotides having a phosphorothioate backbone. See, e.g., PCT Publication Nos. WO/2013/112053 and WO/2009/008725, incorporated by reference in their entireties.

Peptide nucleic acids (PNAs) are analogs of DNA in which the backbone is structurally homomorphous with a deoxyribose backbone, consisting of N-(2-aminoethyl) glycine units to which pyrimidine or purine bases are attached. PNAs containing natural pyrimidine and purine bases hybridize to complementary oligonucleotides obeying Watson-Crick base-pairing rules, and mimic DNA in terms of base pair recognition (Egholm, Buchardt et al. 1993). The backbone of PNAs is formed by peptide bonds rather than phosphodiester bonds, making them well-suited for antisense applications (see structure below). The backbone is uncharged, resulting in PNA/DNA or PNA/RNA duplexes that exhibit greater than normal thermal stability. PNAs are not recognized by nucleases or proteases.

Despite a radical structural change to the natural structure, PNAs are capable of sequence-specific binding in a helix form to DNA or RNA. Characteristics of PNAs include a high binding affinity to complementary DNA or RNA, a destabilizing effect caused by single-base mismatch, resistance to nucleases and proteases, hybridization with DNA or RNA independent of salt concentration and triplex formation with homopurine DNA. PANAGENE™. has developed its proprietary Bts PNA monomers (Bts; benzothiazole-2-sulfonyl group) and proprietary oligomerization process. The PNA oligomerization using Bts PNA monomers is composed of repetitive cycles of deprotection, coupling and capping. PNAs can be produced synthetically using any technique known in the art. See, e.g., U.S. Pat. Nos. 6,969,766, 7,211,668, 7,022,851, 7,125,994, 7,145,006 and 7,179,896. See also U.S. Pat. Nos. 5,539,082; 5,714,331; and 5,719,262 for the preparation of PNAs. Further teaching of PNA compounds can be found in Nielsen et al., Science, 254:1497-1500, 1991. Each of the foregoing is incorporated by reference in its entirety.

Interfering nucleic acids may also contain “locked nucleic acid” subunits (LNAs). “LNAs” are a member of a class of modifications called bridged nucleic acid (BNA). BNA is characterized by a covalent linkage that locks the conformation of the ribose ring in a C30-endo (northern) sugar pucker. For LNA, the bridge is composed of a methylene between the 2'-O and the 4′-C positions. LNA enhances backbone preorganization and base stacking to increase hybridization and thermal stability.

The structures of LNAs can be found, for example, in Wengel, et al., Chemical Communications (1998) 455; Tetrahedron (1998) 54:3607, and Accounts of Chem. Research (1999) 32:301); Obika, et al., Tetrahedron Letters (1997) 38:8735; (1998) 39:5401, and Bioorganic Medicinal Chemistry (2008) 16:9230. Compounds provided herein may incorporate one or more LNAs; in some cases, the compounds may be entirely composed of LNAs. Methods for the synthesis of individual LNA nucleoside subunits and their incorporation into oligonucleotides are described, for example, in U.S. Pat. Nos. 7,572,582, 7,569,575, 7,084,125, 7,060,809, 7,053,207, 7,034,133, 6,794,499, and 6,670,461, each of which is incorporated by reference in its entirety. Typical intersubunit linkers include phosphodiester and phosphorothioate moieties; alternatively, non-phosphorous containing linkers may be employed. One embodiment is an LNA containing compound where each LNA subunit is separated by a DNA subunit. Certain compounds are composed of alternating LNA and DNA subunits where the intersubunit linker is phosphorothioate.

“Phosphorothioates” (or S-oligos) are a variant of normal DNA in which one of the nonbridging oxygens is replaced by a sulfur. The sulfurization of the internucleotide bond reduces the action of endo-and exonucleases including 5′ to 3′ and 3′ to 5′ DNA POL 1 exonuclease, nucleases S1 and P1, RNases, serum nucleases and snake venom phosphodiesterase. Phosphorothioates are made by two principal routes: by the action of a solution of elemental sulfur in carbon disulfide on a hydrogen phosphonate, or by the method of sulfurizing phosphite triesters with either tetraethylthiuram disulfide (TETD) or 3H-1, 2-bensodithiol-3-one 1, 1-dioxide (BDTD) (see, e.g., Iyer et al., J. Org. Chem. 55, 4693-4699, 1990). The latter methods avoid the problem of elemental sulfur's insolubility in most organic solvents and the toxicity of carbon disulfide. The TETD and BDTD methods also yield higher purity phosphorothioates.

“2'O-Me oligonucleotides” molecules carry a methyl group at the 2'-OH residue of the ribose molecule. 2'-O-Me-RNAs show the same (or similar) behavior as DNA, but are protected against nuclease degradation. 2'-O-Me-RNAs can also be combined with phosphothioate oligonucleotides (PTOs) for further stabilization. 2'O-Me oligonucleotides (phosphodiester or phosphothioate) can be synthesized according to routine techniques in the art (see, e.g., Yoo et al., Nucleic Acids Res. 32:2008-16, 2004).

The interfering nucleic acids described herein may be contacted with a cell or administered to an organism (e.g., a human). Alternatively, constructs and/or vectors encoding the interfering RNA molecules may be contacted with or introduced into a cell or organism. In certain embodiments, a viral, retroviral or lentiviral vector is used. In some embodiments the vector is an adeno-associated virus.

Typically at least 17, 18, 19, 20, 21, 22 or 23 nucleotides of the complement of the target mRNA sequence are sufficient to mediate inhibition of a target transcript. Perfect complementarity is not necessary. In some embodiments, the interfering nucleic acids contain a 1, 2 or 3 nucleotide mismatch with the target sequence. The interfering nucleic acid molecule may have a 2 nucleotide 3′ overhang. If the interfering nucleic acid molecule is expressed in a cell from a construct, for example from a hairpin molecule or from an inverted repeat of the desired sequence, then the endogenous cellular machinery will create the overhangs. shRNA molecules can contain hairpins derived from microRNA molecules. For example, an RNAi vector can be constructed by cloning the interfering RNA sequence into a pCAG-miR30 construct containing the hairpin from the miR30 miRNA. RNA interference molecules may include DNA residues, as well as RNA residues.

In some embodiments, the interfering nucleic acid molecule is a siRNA molecule. Such siRNA molecules should include a region of sufficient homology to the target region, and be of sufficient length in terms of nucleotides, such that the siRNA molecule down-regulate target RNA. The term “ribonucleotide” or “nucleotide” can, in the case of a modified RNA or nucleotide surrogate, also refer to a modified nucleotide, or surrogate replacement moiety at one or more positions. It is not necessary that there be perfect complementarity between the siRNA molecule and the target, but the correspondence must be sufficient to enable the siRNA molecule to direct sequence-specific silencing, such as by RNAi cleavage of the target RNA. In some embodiments, the sense strand need only be sufficiently complementary with the antisense strand to maintain the overall double-strand character of the molecule.

In addition, an siRNA molecule may be modified or include nucleoside surrogates. Single stranded regions of an siRNA molecule may be modified or include nucleoside surrogates, e.g., the unpaired region or regions of a hairpin structure, e.g., a region which links two complementary regions, can have modifications or nucleoside surrogates. Modification to stabilize one or more 3′- or 5′-terminus of an siRNA molecule, e.g., against exonucleases, or to favor the antisense siRNA agent to enter into RISC are also useful. Modifications can include C3 (or C6, C7, C12) amino linkers, thiol linkers, carboxyl linkers, non-nucleotidic spacers (C3, C6, C9, C12, abasic, triethylene glycol, hexaethylene glycol), special biotin or fluorescein reagents that come as phosphoramidites and that have another DMT-protected hydroxyl group, allowing multiple couplings during RNA synthesis.

Each strand of an siRNA molecule can be equal to or less than 35, 30, 25, 24, 23, 22, 21, or 20 nucleotides in length. In some embodiments, the strand is at least 19 nucleotides in length. For example, each strand can be between 21 and 25 nucleotides in length. In some embodiments, siRNA agents have a duplex region of 17, 18, 19, 29, 21, 22, 23, 24, or 25 nucleotide pairs, and one or more overhangs, such as one or two 3′ overhangs, of 2-3 nucleotides.

A “small hairpin RNA” or “short hairpin RNA” or “shRNA” includes a short RNA sequence that makes a tight hairpin turn that can be used to silence gene expression via RNA interference. The shRNAs provided herein may be chemically synthesized or transcribed from a transcriptional cassette in a DNA plasmid. The shRNA hairpin structure is cleaved by the cellular machinery into siRNA, which is then bound to the RNA-induced silencing complex (RISC).

In some embodiments, shRNAs are about 15-60, 15-50, or 15-40 (duplex) nucleotides in length, about 15-30, 15-25, or 19-25 (duplex) nucleotides in length, or are about 20-24, 21-22, or 21-23 (duplex) nucleotides in length (e.g., each complementary sequence of the double-stranded shRNA is 15-60, 15-50, 15-40, 15-30, 15-25, or 19-25 nucleotides in length, or about 20-24, 21-22, or 21-23 nucleotides in length, and the double-stranded shRNA is about 15-60, 15-50, 15-40, 15-30, 15-25, or 19-25 base pairs in length, or about 18-22, 19-20, or 19-21 base pairs in length). shRNA duplexes may comprise 3′ overhangs of about 1 to about 4 nucleotides or about 2 to about 3 nucleotides on the antisense strand and/or 5′-phosphate termini on the sense strand. In some embodiments, the shRNA comprises a sense strand and/or antisense strand sequence of from about 15 to about 60 nucleotides in length (e.g., about 15-60, 15-55, 15-50, 15-45, 15-40, 15-35, 15-30, or 15-25 nucleotides in length), or from about 19 to about 40 nucleotides in length (e.g., about 19-40, 19-35, 19-30, or 19-25 nucleotides in length), or from about 19 to about 23 nucleotides in length (e.g., 19, 20, 21, 22, or 23 nucleotides in length).

Non-limiting examples of shRNA include a double-stranded polynucleotide molecule assembled from a single-stranded molecule, where the sense and antisense regions are linked by a nucleic acid-based or non-nucleic acid-based linker; and a double-stranded polynucleotide molecule with a hairpin secondary structure having self-complementary sense and antisense regions. In some embodiments, the sense and antisense strands of the shRNA are linked by a loop structure comprising from about 1 to about 25 nucleotides, from about 2 to about 20 nucleotides, from about 4 to about 15 nucleotides, from about 5 to about 12 nucleotides, or 1, 2, 3, 4, 5, 6, 7, 8, 9, 10, 11, 12, 13, 14, 15, 16, 17, 18, 19, 20, 21, 22, 23, 24, 25, or more nucleotides.

Additional embodiments related to the shRNAs, as well as methods of designing and synthesizing such shRNAs, are described in U.S. patent application publication number 2011/0071208, the disclosure of which is herein incorporated by reference in its entirety for all purposes.

In some embodiments, provided herein are micro RNAs (miRNAs). miRNAs represent a large group of small RNAs produced naturally in organisms, some of which regulate the expression of target genes. miRNAs are formed from an approximately 70 nucleotide single-stranded hairpin precursor transcript by Dicer. miRNAs are not translated into proteins, but instead bind to specific messenger RNAs, thereby blocking translation. In some instances, miRNAs base-pair imprecisely with their targets to inhibit translation.

In some embodiments, antisense oligonucleotide compounds are provided herein. In certain embodiments, the degree of complementarity between the target sequence and antisense targeting sequence is sufficient to form a stable duplex. The region of complementarity of the antisense oligonucleotides with the target RNA sequence may be as short as 8-11 bases, but can be 12-15 bases or more, e.g., 10-40 bases, 12-30 bases, 12-25 bases, 15-25 bases, 12-20 bases, or 15-20 bases, including all integers in between these ranges. An antisense oligonucleotide of about 14-15 bases is generally long enough to have a unique complementary sequence.

In certain embodiments, antisense oligonucleotides may be 100% complementary to the target sequence, or may include mismatches, e.g., to improve selective targeting of allele containing the disease-associated mutation, as long as a heteroduplex formed between the oligonucleotide and target sequence is sufficiently stable to withstand the action of cellular nucleases and other modes of degradation which may occur in vivo. Hence, certain oligonucleotides may have about or at least about 70% sequence complementarity, e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence complementarity, between the oligonucleotide and the target sequence. Oligonucleotide backbones that are less susceptible to cleavage by nucleases are discussed herein. Mismatches, if present, are typically less destabilizing toward the end regions of the hybrid duplex than in the middle. The number of mismatches allowed will depend on the length of the oligonucleotide, the percentage of G:C base pairs in the duplex, and the position of the mismatch(es) in the duplex, according to well understood principles of duplex stability.

Interfering nucleic acid molecules can be prepared, for example, by chemical synthesis, in vitro transcription, or digestion of long dsRNA by Rnase III or Dicer. These can be introduced into cells by transfection, electroporation, or other methods known in the art. See Hannon, G J, 2002, RNA Interference, Nature 418: 244-251; Bernstein E et al., 2002, The rest is silence. RNA 7: 1509-1521; Hutvagner G et al., RNAi: Nature abhors a double-strand. Curr. Opin. Genetics & Development 12: 225-232; Brummelkamp, 2002, A system for stable expression of short interfering RNAs in mammalian cells. Science 296: 550-553; Lee N S, Dohjima T, Bauer G, Li H, Li M-J, Ehsani A, Salvaterra P, and Rossi J. (2002). Expression of small interfering RNAs targeted against HIV-1 rev transcripts in human cells. Nature Biotechnol. 20:500-505; Miyagishi M, and Taira K. (2002). U6-promoter-driven siRNAs with four uridine 3′ overhangs efficiently suppress targeted gene expression in mammalian cells. Nature Biotechnol. 20:497-500; Paddison P J, Caudy A A, Bernstein E, Hannon G J, and Conklin D S. (2002). Short hairpin RNAs (shRNAs) induce sequence-specific silencing in mammalian cells. Genes & Dev. 16:948-958; Paul C P, Good P D, Winer I, and Engelke D R. (2002). Effective expression of small interfering RNA in human cells. Nature Biotechnol. 20:505-508; Sui G, Soohoo C, Affar E-B, Gay F, Shi Y, Forrester W C, and Shi Y. (2002). A DNA vector-based RNAi technology to suppress gene expression in mammalian cells. Proc. Natl. Acad. Sci. USA 99(6):5515-5520; Yu J-Y, DeRuiter SL, and Turner D L. (2002). RNA interference by expression of short-interfering RNAs and hairpin RNAs in mammalian cells. Proc. Natl. Acad. Sci. USA 99(9):6047-6052.

In the present methods, an interfering nucleic acid molecule or an interfering nucleic acid encoding polynucleotide can be administered to the subject, for example, as naked nucleic acid, in combination with a delivery reagent, and/or as a nucleic acid comprising sequences that express an interfering nucleic acid molecule. In some embodiments the nucleic acid comprising sequences that express the interfering nucleic acid molecules are delivered within vectors, e.g. plasmid, viral and bacterial vectors. Any nucleic acid delivery method known in the art can be used in the methods described herein. Suitable delivery reagents include, but are not limited to, e.g., the Mirus Transit TKO lipophilic reagent; lipofectin; lipofectamine; cellfectin; polycations (e.g., polylysine), atelocollagen, nanoplexes and liposomes. The use of atelocollagen as a delivery vehicle for nucleic acid molecules is described in Minakuchi et al. Nucleic Acids Res., 32(13):e109 (2004); Hanai et al. Ann NY Acad Sci., 1082:9-17 (2006); and Kawata et al. Mol Cancer Ther., 7(9):2904-12 (2008); each of which is incorporated herein in their entirety. Exemplary interfering nucleic acid delivery systems are provided in U.S. Pat. Nos. 8,283,461, 8,313,772, 8,501,930. 8,426,554, 8,268,798 and 8,324,366, each of which is hereby incorporated by reference in its entirety.

In some embodiments of the methods described herein, liposomes are used to deliver an inhibitory oligonucleotide to a subject. Liposomes suitable for use in the methods described herein can be formed from standard vesicle-forming lipids, which generally include neutral or negatively charged phospholipids and a sterol, such as cholesterol. The selection of lipids is generally guided by consideration of factors such as the desired liposome size and half-life of the liposomes in the blood stream. A variety of methods are known for preparing liposomes, for example, as described in Szoka et al. (1980), Ann. Rev. Biophys. Bioeng. 9:467; and U.S. Pat. Nos. 4,235,871, 4,501,728, 4,837,028, and 5,019,369, the entire disclosures of which are herein incorporated by reference.

The liposomes for use in the present methods can also be modified so as to avoid clearance by the mononuclear macrophage system (“MMS”) and reticuloendothelial system (“RES”). Such modified liposomes have opsonization-inhibition moieties on the surface or incorporated into the liposome structure.

Opsonization-inhibiting moieties for use in preparing the liposomes described herein are typically large hydrophilic polymers that are bound to the liposome membrane. As used herein, an opsonization inhibiting moiety is “bound” to a liposome membrane when it is chemically or physically attached to the membrane, e.g., by the intercalation of a lipid-soluble anchor into the membrane itself, or by binding directly to active groups of membrane lipids. These opsonization-inhibiting hydrophilic polymers form a protective surface layer that significantly decreases the uptake of the liposomes by the MMS and RES; e.g., as described in U.S. Pat. No. 4,920,016, the entire disclosure of which is herein incorporated by reference.

In some embodiments, opsonization inhibiting moieties suitable for modifying liposomes are water-soluble polymers with a number-average molecular weight from about 500 to about 40,000 daltons, or from about 2,000 to about 20,000 daltons. Such polymers include polyethylene glycol (PEG) or polypropylene glycol (PPG) derivatives; e.g., methoxy PEG or PPG, and PEG or PPG stearate; synthetic polymers such as polyacrylamide or poly N-vinyl pyrrolidone; linear, branched, or dendrimeric polyamidoamines; polyacrylic acids; polyalcohols, e.g., polyvinylalcohol and polyxylitol to which carboxylic or amino groups are chemically linked, as well as gangliosides, such as ganglioside GM1. Copolymers of PEG, methoxy PEG, or methoxy PPG, or derivatives thereof, are also suitable. In addition, the opsonization inhibiting polymer can be a block copolymer of PEG and either a polyamino acid, polysaccharide, polyamidoamine, polyethyleneamine, or polynucleotide. The opsonization inhibiting polymers can also be natural polysaccharides containing amino acids or carboxylic acids, e.g., galacturonic acid, glucuronic acid, mannuronic acid, hyaluronic acid, pectic acid, neuraminic acid, alginic acid, carrageenan; aminated polysaccharides or oligosaccharides (linear or branched); or carboxylated polysaccharides or oligosaccharides, e.g., reacted with derivatives of carbonic acids with resultant linking of carboxylic groups. In some embodiments, the opsonization-inhibiting moiety is a PEG, PPG, or derivatives thereof. Liposomes modified with PEG or PEG-derivatives are sometimes called “PEGylated liposomes.”

### CRISPR/Gene Editing

Certain embodiments disclosed herein relate to modifying T cells to reduce the expression or activity of RGMb through gene editing.

Deletion of DNA may be performed using gene therapy to knock-out or disrupt the target gene. As used herein, a “knock-out” can be a gene knock-down or the gene can be knocked out by a mutation such as, a point mutation, an insertion, a deletion, a frameshift, or a missense mutation by techniques known in the art, including, but not limited to, retroviral gene transfer. In some embodiments, the agent is a nuclease (e.g., a zinc finger nuclease or a TALEN). Zinc-finger nucleases (ZFNs) are artificial restriction enzymes generated by fusing a zinc finger DNA-binding domain to a DNA-cleavage domain. Zinc finger domains can be engineered to target desired DNA sequences, which enable zinc-finger nucleases to target unique sequence within a complex genome. By taking advantage of endogenous DNA repair machinery, these reagents can be used to precisely alter the genomes of higher organisms. Other technologies for genome customization that can be used to knock out genes are meganucleases and TAL effector nucleases (TALENs). A TALEN is composed of a TALE DNA binding domain for sequence-specific recognition fused to the catalytic domain of an endonuclease that introduces double-strand breaks (DSB). The DNA binding domain of a TALEN is capable of targeting with high precision a large recognition site (for instance, 17 bp). Meganucleases are sequence-specific endonucleases, naturally occurring “DNA scissors,” originating from a variety of single-celled organisms such as bacteria, yeast, algae and some plant organelles. Meganucleases have long recognition sites of between 12 and 30 base pairs. The recognition site of natural meganucleases can be modified in order to target native genomic DNA sequences (such as endogenous genes). The gene editing agent disclosed herein may comprise a DNA binding domain comprising a sequence that is complementary to a RGMb genomic sequence, such as a sequence listed in Table 3. The gene editing agent disclosed herein may target a RGMb genomic sequence, such as a sequence listed in Table 3.

In another embodiment, the agent comprises a CRISPR-Cas9 guided nuclease and/or a sgRNA (Wiedenheft et al., “RNA-Guided Genetic Silencing Systems in Bacteria and Archaea,” Nature 482:331-338 (2012); Zhang et al., “Multiplex Genome Engineering Using CRISPR/Cas Systems,” Science 339(6121): 819-23 (2013); and Gaj et al., “ZFN, TALEN, and CRISPR/Cas-based Methods for Genome Engineering,” Cell 31(7):397-405 (2013), which are hereby incorporated by reference in their entirety). Like the TALENs and ZFNs, CRISPR-Cas9 interference is a genetic technique which allows for sequence-specific control of gene expression in prokaryotic and eukaryotic cells by guided nuclease double-stranded DNA cleavage. It is based on the bacterial immune system—derived CRISPR (clustered regularly interspaced palindromic repeats) pathway. In some embodiments, the agent is an sgRNA. An sgRNA combines tracrRNA and crRNA, which are separate molecules in the native CRISPR/Cas9 system, into a single RNA construct, simplifying the components needed to use CRISPR/Cas9 for genome editing. In some embodiments, the crRNA of the sgRNA has complementarity to at least a portion of a gene that encodes RGMb (or a fragment thereof). In some embodiments, the sgRNA may target at least a portion of a gene that encodes RGMb protein. The gene editing agent disclosed herein may comprise a gRNA comprising a sequence that is complementary to a RGMb genomic sequence, such as a sequence listed in Table 3.

### Antibody Agents

In certain embodiments, the methods and compositions provided herein relate to antibodies and antigen binding fragments thereof that bind specifically to RGMb (i.e., to RGMb in T cells ex vivo), PD-1 or PD-L1. In some embodiments, the antibodies block the molecules disclosed herein. In some embodiments, the antibodies disrupt the molecules disclosed herein. Such antibodies can be polyclonal or monoclonal and can be, for example, murine, chimeric, humanized or fully human. The antibody may be bispecific.

In some embodiments, the antibody that disrupts or blocks RGMb is a human anti-RGMb antibody that is structurally related to 307.9D1, 307.8B2, 307.1H6, 307.9D3, or 307.5G1. In some embodiments, the antibody that disrupts or blocks RGMb comprises the heavy chain variable domain amino acid sequence encoded by SEQ ID NO: 2, as shown below, and the light chain variable domain amino acid sequence encoded by SEQ ID NO: 1, as shown below. In some embodiments, the antibody that disrupts or blocks RGMb is a bispecific antibody. Exemplary RGMb antibodies can be found in WO2022060986, hereby incorporated by reference in its entirety.

| | | |
|---|---|---|
| SEQ | atgatggctg cagttcagct cttagggctt | 9D1 |
| ID | ttgctgctct gcctccgagc catgagatgt | VL |
| NO: | gacatccaga tgacccagtc tccttcacac | |
| 1 | ctgtcagcat ctgtgggaga cagagtcact | |
| | ctcagctgca aagtaagtca gaatatttac | |
| | aagtacttaa actggtatca gcaaaaactt | |
| | ggagaagctc ccaaactcct gatatattat | |
| | acaagctttt tgcaaacggg catcccgtca | |
| | aggttcagtg gcagtggatc tggtacagat | |
| | tacacactca ccatcagcag cctgcagcct | |
| | gaagatgttg ccacatattt ctgccagaag | |
| | tattatagcg ggtggacgtt cggtggaggc | |
| | accaagctgg aattgaaa | |
| SEQ | atgggatgga gccagatcat tctctttctg | 9D1 |
| ID | gtggcagcaa ctacatgtgt ccactcccag | VH |
| NO: | gtacagctac agcaatcagg gactgaactg | |
| 2 | gtgaagcctg ggtcctcagt gaaaatttcc | |
| | tgcaaggctt ctggcgacac cttcaccagt | |
| | gactatatgc actggataag gcagcagcct | |
| | ggaagtggcc ttgagtggat tgggtggatt | |
| | tatcctggaa atggtaatac taagtacaat | |
| | caaaagttcg atgggaaggc aacactcact | |
| | gcagacaaat cctccagcac agcctatttg | |
| | cagctcagcc tcctgacatc tgaggactat | |
| | gcagtctatt tctgtgcaag acagacggag | |
| | gggtactttg attactgggg ccaaggagtc | |
| | atggtcacag tctcctca | |

Exemplary PD-L1 or PD-1 antibodies include, without limitation, cemiplimab (REGN2810), nivolumab (BMS-936558, MDX-1106, ONO-4538), pembrolizumab (MK-3475, SCH 900475), SHR1210, sintilimab (IBI308), spartalizumab (PDR001), tislelizumab (BGB-A317), pidilizumab, BCD-100, toripalimab (JS001), PF-06801591, AB122, AK105, AMG 404, BCD-100, BI 754091, F520, HLX10, HX008, JTX-4014, LZM009, MEDI0680, MGA012, Sym021, TSR-042, PSB205, MGD019, MGD013, AK104, XmAb20717, R07121661, CX-188, atezolizumab (MPDL3280A, RG7446, RO5541267), durvalumab (MEDI4736, MEDI-4736), avelumab (MSB0010718C), FS118, BCD-135, BGB-A333, CBT-502, CK-301, CS1001, FAZ053, HLX20, KN035, MDX-1105, MSB2311, SHR-1316, TG-1501, ZKAB001, INBRX-105, MCLA-145, KN046, M7824, and LY3415244.

Polyclonal antibodies can be prepared by immunizing a suitable subject (e.g., a mouse) with a polypeptide antigen (e.g., a polypeptide having a sequence of RGMb or a fragment thereof). The polypeptide antibody titer in the immunized subject can be monitored over time by standard techniques, such as with an enzyme linked immunosorbent assay (ELISA) using immobilized polypeptide. If desired, the antibody directed against the antigen can be isolated from the mammal (e.g., from the blood) and further purified by well-known techniques, such as protein A chromatography to obtain the IgG fraction.

At an appropriate time after immunization, e.g., when the antibody titers are highest, antibody-producing cells can be obtained from the subject and used to prepare monoclonal antibodies using standard techniques, such as the hybridoma technique originally described by Kohler and Milstein (1975) Nature 256:495-497) (see also Brown et al. (1981) J. Immunol. 127:539-46; Brown et al. (1980) J. Biol. Chem. 255:4980-83; Yeh et al. (1976) Proc. Natl. Acad. Sci. 76:2927-31; and Yeh et al. (1982) Int. J. Cancer 29:269-75), the more recent human B cell hybridoma technique (Kozbor et al. (1983) Immunol. Today 4:72), the EBV-hybridoma technique (Cole et al. (1985) Monoclonal Antibodies and Cancer Therapy, Alan R. Liss, Inc., pp. 77-96) or trioma techniques. The technology for producing monoclonal antibody hybridomas is well known (see generally Kenneth, R. H. in Monoclonal Antibodies: A New Dimension In Biological Analyses, Plenum Publishing Corp., New York, New York (1980); Lerner, E. A. (1981) Yale J. Biol. Med. 54:387-402; Gefter, M. L. et al. (1977) Somatic Cell Genet. 3:231-36). Briefly, an immortal cell line (typically a myeloma) is fused to lymphocytes (typically splenocytes) from a mammal immunized with an immunogen as described above, and the culture supernatants of the resulting hybridoma cells are screened to identify a hybridoma producing a monoclonal antibody that binds to the polypeptide antigen, preferably specifically.

As an alternative to preparing monoclonal antibody-secreting hybridomas, a monoclonal specific for a receptor or ligand provided herein can be identified and isolated by screening a recombinant combinatorial immunoglobulin library (e.g., an antibody phage display library or an antibody yeast display library) with the appropriate polypeptide to thereby isolate immunoglobulin library members that bind the polypeptide.

Additionally, recombinant antibodies specific for a receptor or ligand provided herein, such as chimeric or humanized monoclonal antibodies, can be made using standard recombinant DNA techniques. Such chimeric and humanized monoclonal antibodies can be produced by recombinant DNA techniques known in the art, for example using methods described in U.S. Pat. Nos. 4,816,567; 5,565,332; Better et al. (1988) Science 240:1041-1043; Liu et al. (1987) Proc. Natl. Acad. Sci. USA 84:3439-3443; Liu et al. (1987) J. Immunol. 139:3521-3526; Sun et al. (1987) Proc. Natl. Acad. Sci. 84:214-218; Nishimura et al. (1987) Cancer Res. 47:999-1005; Wood et al. (1985) Nature 314:446-449; and Shaw et al. (1988) J. Natl. Cancer Inst. 80:1553-1559); Morrison, S. L. (1985) Science 229:1202-1207; Oi et al. (1986) Biotechniques 4:214; Winter U.S. Pat. No. 5,225,539; Jones et al. (1986) Nature 321:552-525; Verhoeyan et al. (1988) Science 239:1534; and Beidler et al. (1988) J. Immunol. 141:4053-4060.

Human monoclonal antibodies specific for a receptor or ligand provided herein can be generated using transgenic or transchromosomal mice carrying parts of the human immune system rather than the mouse system. For example, “HuMAb mice” which contain a human immunoglobulin gene miniloci that encodes unrearranged human heavy (μ and γ) and κ light chain immunoglobulin sequences, together with targeted mutations that inactivate the endogenous μ and κ chain loci (Lonberg, N. et al. (1994) Nature 368(6474): 856 859). Accordingly, the mice exhibit reduced expression of mouse IgM or κ, and in response to immunization, the introduced human heavy and light chain transgenes undergo class switching and somatic mutation to generate high affinity human IgGκ monoclonal antibodies (Lonberg, N. et al. (1994), supra; reviewed in Lonberg, N. (1994) Handbook of Experimental Pharmacology 113:49 101; Lonberg, N. and Huszar, D. (1995) Intern. Rev. Immunol. Vol. 13: 65 93, and Harding, F. and Lonberg, N. (1995) Ann. N. Y Acad. Sci 764:536 546). The preparation of HuMAb mice is described in Taylor, L. et al. (1992) Nucleic Acids Research 20:6287 6295; Chen, J. et al. (1993) International Immunology 5: 647 656; Tuaillon et al. (1993) Proc. Natl. Acad. Sci USA 90:3720 3724; Choi et al. (1993) Nature Genetics 4:117 123; Chen, J. et al. (1993) EMBO J. 12: 821 830; Tuaillon et al. (1994) J. Immunol. 152:2912 2920; Lonberg et al., (1994) Nature 368(6474): 856 859; Lonberg, N. (1994) Handbook of Experimental Pharmacology 113:49 101; Taylor, L. et al. (1994) International Immunology 6: 579 591; Lonberg, N. and Huszar, D. (1995) Intern. Rev. Immunol. Vol. 13: 65 93; Harding, F. and Lonberg, N. (1995) Ann. N.Y. Acad. Sci 764:536 546; Fishwild, D. et al. (1996) Nature Biotechnology 14: 845 851. See further, U.S. Pat. Nos. 5,545,806; 5,569,825; 5,625,126; 5,633,425; 5,789,650; 5,877,397; 5,661,016; 5,814,318; 5,874,299; 5,770,429; and 5,545,807.

### Pharmaceutical Compositions

In certain embodiments, provided herein is a composition, e.g., a pharmaceutical composition, containing at least one agent and/or population of T cells described herein together with a pharmaceutically acceptable carrier. In one embodiment, the composition includes a combination of multiple (e.g., two or more) agents described herein.

In some embodiments, the composition comprises an antibody that disrupts or blocks RGMb. Such an antibody may be a human anti-RGMb antibody that is structurally related to 307.9D1, 307.8B2, 307.1H6, 307.9D3, or 307.5G1. In some embodiments, the antibody that disrupts or blocks RGMb comprises the heavy chain variable domain amino acid sequence encoded by SEQ ID NO: 2, as shown below, and the light chain variable domain amino acid sequence encoded by SEQ ID NO: 1, as shown below. In some embodiments, the antibody that disrupts or blocks RGMb is a bispecific antibody.

In some embodiments, the composition comprises an agent that disrupts PD-L1 or PD-1, e.g., cemiplimab (REGN2810), nivolumab (BMS-936558, MDX-1106, ONO-4538), pembrolizumab (MK-3475, SCH 900475), SHR1210, sintilimab (IBI308), spartalizumab (PDR001), tislelizumab (BGB-A317), pidilizumab, BCD-100, toripalimab (JS001), PF-06801591, AB122, AK105, AMG 404, BCD-100, BI 754091, F520, HLX10, HX008, JTX-4014, LZM009, MEDIO680, MGA012, Sym021, TSR-042, PSB205, MGD019, MGD013, AK104, XmAb20717, R07121661, CX-188, atezolizumab (MPDL3280A, RG7446, R05541267), durvalumab (MEDI4736, MEDI-4736), avelumab (MSB0010718C), FS118, BCD-135, BGB-A333, CBT-502, CK-301, CS1001, FAZ053, HLX20, KNO35, MDX-1105, MSB2311, SHR-1316, TG-1501, ZKAB001, INBRX-105, MCLA-145, KN046, M7824, or LY3415244.

As described in detail below, the pharmaceutical compositions disclosed herein may be specially formulated for administration in solid or liquid form, including those adapted for the following: (1) oral administration, for example, drenches (aqueous or non-aqueous solutions or suspensions), tablets, e.g., those targeted for buccal, sublingual, and systemic absorption, boluses, powders, granules, pastes for application to the tongue; or (2) parenteral administration, for example, by subcutaneous, intramuscular, intravenous, intrathecal, intracerebral or epidural injection as, for example, a sterile solution or suspension, or sustained-release formulation.

Methods of preparing these formulations or compositions include the step of bringing into association an agent described herein with the carrier and, optionally, one or more accessory ingredients. In general, the formulations are prepared by uniformly and intimately bringing into association an agent described herein with liquid carriers, or finely divided solid carriers, or both, and then, if necessary, shaping the product.

Pharmaceutical compositions suitable for parenteral administration comprise one or more agents described herein in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain sugars, alcohols, antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions include water, ethanol, dimethyl sulfoxide (DMSO), polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

Regardless of the route of administration selected, the agents provided herein, which may be used in a suitable hydrated form, and/or the pharmaceutical compositions disclosed herein, are formulated into pharmaceutically-acceptable dosage forms by conventional methods known to those of skill in the art.

As described in detail below, the pharmaceutical compositions and/or agents disclosed herein may be specially formulated for administration in solid or liquid form, including those adapted for the following: (1) oral administration, for example, drenches (aqueous or non-aqueous solutions or suspensions), tablets, e.g., those targeted for buccal, sublingual, and systemic absorption, boluses, powders, granules, pastes for application to the tongue; or (2) parenteral administration, for example, by subcutaneous, intramuscular, intravenous, intrathecal, intracerebral or epidural injection as, for example, a sterile solution or suspension, or sustained-release formulation. Methods of preparing pharmaceutical formulations or compositions include the step of bringing into association an agent described herein with the carrier and, optionally, one or more accessory ingredients. In general, the formulations are prepared by uniformly and intimately bringing into association an agent described herein with liquid carriers, or finely divided solid carriers, or both, and then, if necessary, shaping the product.

Pharmaceutical compositions suitable for parenteral administration comprise one or more agents described herein in combination with one or more pharmaceutically-acceptable sterile isotonic aqueous or non-aqueous solutions, dispersions, suspensions or emulsions, or sterile powders which may be reconstituted into sterile injectable solutions or dispersions just prior to use, which may contain sugars, alcohols, antioxidants, buffers, bacteriostats, solutes which render the formulation isotonic with the blood of the intended recipient or suspending or thickening agents.

Examples of suitable aqueous and non-aqueous carriers which may be employed in the pharmaceutical compositions include water, ethanol, dimethyl sulfoxide (DMSO), polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

### Additional Methods

The agents and/or compositions may be administered systemically or locally to the tumor present in the subject. In some embodiments, the agent or pharmaceutical composition is administered with an additional therapeutic agent. In some embodiments, the additional therapeutic agent is a chemotherapeutic agent. Exemplary chemotherapeutic agents include alkylating agents such as thiotepa and cyclophosphamide (Cytoxan™); alkyl sulfonates such as busulfan, improsulfan and piposulfan; aziridines such as benzodopa, carboquone, meturedopa, and uredopa; emylerumines and memylamelamines including alfretamine, triemylenemelamine, triethylenephosphoramide, triethylenethiophosphoramide, and trimemylolomelamine; acetogenins (especially bullatacin and bullatacinone); a camptothecin (including synthetic analogue topotecan); bryostatin; callystatin; CC-1065 (including its adozelesin, carzelesin and bizelesin synthetic analogues); cryptophycins (articularly cryptophycin 1 and cryptophycin 8); dolastatin; duocarmycin (including the synthetic analogues, KW-2189 and CBI-TMI); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, foremustine, lomustine, nimustine, ranimustine; antibiotics such as the enediyne antibiotics (e.g., calicheamicin, especially calicheamicin gammall and calicheamicin phili); dynemicin, including dynemicin A; bisphosphonates, such as clodronate; an esperamicin; as well as neocarzinostatin chromophore and related chromoprotein enediyne antibiotic chromomophores), aclacinomysins, actinomycin, authramycin, azaserine, bleomycins, cactinomycin, carabicin, carrninomycin, carzinophilin, chromomycins, dactinomycin, daunorubicin, detorubicin, 6-diazo-5-oxo-L-norleucine, doxorubicin (Adramycin™) (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as demopterin, methotrexate, pteropterin, trimetrexate; purine analogs such as fludarabine, 6-mercaptopurine, thiamiprine, thioguanine; pyrimidine analogues such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replinisher such as frolinic acid; aceglatone; aldophosphamide glycoside; aminolevulinic acid; eniluracil; amsacrine; hestrabucil; bisantrene; edatraxate; defofamine; demecolcine; diaziquone; elformthine; elliptinium acetate; an epothilone; etoglucid; gallium nitrate; hydroxyurea; lentinan; lonidamine; maytansinoids such as maytansine and ansamitocins; mitoguazone; mitoxantrone; mopidamol; nitracrine; pentostatin; phenamet; pirarubicin; losoxantrone; podophyllinic acid; 2-ethylhydrazide; procarbazine; PSK™; razoxane; rhizoxin; sizofiran; spirogermanium; tenuazonic acid; triaziquone; 2,2′,2″-tricUorotriemylamine; trichothecenes (especially T-2 toxin, verracurin A, roridin A and anguidine); urethane; vindesine; dacarbazine; mannomustine; mitobronitol; mitolactol; pipobroman; gacytosine; arabinoside (“Ara-C”); cyclophosphamide; thiopeta; taxoids, e.g., paclitaxel (Taxol™, Bristol Meyers Squibb Oncology, Princeton, N.J.) and docetaxel (Taxoteret™, Rhone-Poulenc Rorer, Antony, France); chlorambucil; gemcitabine (Gemzar™); 6-thioguanine; mercaptopurine; methotrexate; platinum analogs such as cisplatin and carboplatin; vinblastine; platinum; etoposide (VP-16); ifosfamide; mitroxantrone; vancristine; vinorelbine (Navelbine™); novantrone; teniposide; edatrexate; daunomycin; aminopterin; xeoloda; ibandronate; CPT-11; topoisomerase inhibitor RFS 2000; difluoromethylornithine (DMFO); retinoids such as retinoic acid; capecitabine; and pharmaceutically acceptable salts, acids or derivatives of any of the above. Also included in the definition of “chemotherapeutic agent” are anti-hormonal agents that act to regulate or inhibit hormone action on tumors such as anti-estrogens and selective estrogen receptor modulators (SERMs), including, for example, tamoxifen (including Nolvadex™), raloxifene, droloxifene, 4-hydroxytamoxifen, trioxifene, keoxifene, LY117018, onapristone, and toremifene (Fareston™); inhibitors of the enzyme aromatase, which regulates estrogen production in the adrenal glands, such as, for example, 4(5)-imidazoles, aminoglutethimide, megestrol acetate (Megace™), exemestane, formestane, fadrozole, vorozole (Rivisor™) letrozole (Femara™), and anastrozole (Arimidex™); and anti-androgens such as flutamide, nilutamide, bicalutamide, leuprohde, and goserelin; and pharmaceutically acceptable salts, acids or derivatives of any of the above. In some embodiments, the additional therapeutic agent is an immune checkpoint inhibitor. Immune Checkpoint inhibition broadly refers to inhibiting the checkpoints that cancer cells can produce to prevent or downregulate an immune response. Examples of immune checkpoint proteins are CTLA-4, PD-1, VISTA, B7-H2, B7-H3, PD-L1, B7-H4, B7-H6, ICOS, HVEM, CD160, gp49B, PIR-B, KIR family receptors, TIM-1, TIM-3, TIM-4, LAG-3, BTLA, SIRPalpha (CD47), CD48, 2B4 (CD244), B7.1, B7.2, ILT-2, ILT-4, TIGIT, HHLA2, butyrophilins, A2aR, and combinations thereof.

### Indications

In some aspects, provided herein are methods of treating a cancer by administering to a subject (e.g., to a tumor present in a subject or to the subject orally) the modified T cells described herein.

In some embodiments, the methods described herein may be used to treat any cancerous or pre-cancerous tumor. In some embodiments, the cancer includes a solid tumor. Cancers that may be treated by methods and compositions provided herein include, but are not limited to, cancer cells from the bladder, blood, bone, bone marrow, brain, breast (e.g., estrogen receptor (ER)-positive breast cancer, triple negative breast cancer, or HER2 positive breast cancer), colon, esophagus, gastrointestine, gum, head, kidney, liver, lung, nasopharynx, neck, ovary, prostate, skin, stomach, testis, tongue, or uterus. In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometrioid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; mammary paget's disease; acinar cell carcinoma; adenosquamous carcinoma; adenocarcinoma w/squamous metaplasia; malignant thymoma; malignant ovarian stromal tumor; malignant thecoma; malignant granulosa cell tumor; and malignant roblastoma; sertoli cell carcinoma; malignant leydig cell tumor; malignant lipid cell tumor; malignant paraganglioma; malignant extra-mammary paraganglioma; pheochromocytoma; glomangiosarcoma; malignant melanoma; amelanotic melanoma; superficial spreading melanoma; malignant melanoma in giant pigmented nevus; epithelioid cell melanoma; malignant blue nevus; sarcoma; fibrosarcoma; malignant fibrous histiocytoma; myxosarcoma; liposarcoma; leiomyosarcoma; rhabdomyosarcoma; embryonal rhabdomyosarcoma; alveolar rhabdomyosarcoma; stromal sarcoma; malignant mixed tumor; mullerian mixed tumor; nephroblastoma; hepatoblastoma; carcinosarcoma; malignant mesenchymoma; malignant brenner tumor; malignant phyllodes tumor; synovial sarcoma; malignant mesothelioma; dysgerminoma; embryonal carcinoma; malignant teratoma; malignant struma ovarii; choriocarcinoma; malignant mesonephroma; hemangiosarcoma; malignant hemangioendothelioma; kaposi's sarcoma; malignant hemangiopericytoma; lymphangiosarcoma; osteosarcoma; juxtacortical osteosarcoma; chondrosarcoma; malignant chondroblastoma; mesenchymal chondrosarcoma; giant cell tumor of bone; ewing's sarcoma; malignant odontogenic tumor; ameloblastic odontosarcoma; malignant ameloblastoma; ameloblastic fibrosarcoma; malignant pinealoma; chordoma; malignant glioma; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; malignant meningioma; neurofibrosarcoma; malignant neurilemmoma; malignant granular cell tumor; malignant lymphoma; Hodgkin's disease; Hodgkin's lymphoma; paragranuloma; small lymphocytic malignant lymphoma; diffuse large cell malignant lymphoma; follicular malignant lymphoma; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

In some embodiments, the subject has cancer. In some embodiments, the cancer comprises a solid tumor. In some embodiments, the tumor is an adenocarcinoma, an adrenal tumor, an anal tumor, a bile duct tumor, a bladder tumor, a bone tumor, a blood born tumor, a brain/CNS tumor, a breast tumor, a cervical tumor, a colorectal tumor, an endometrial tumor, an esophageal tumor, an Ewing tumor, an eye tumor, a gallbladder tumor, a gastrointestinal, a kidney tumor, a laryngeal or hypopharyngeal tumor, a liver tumor, a lung tumor, a mesothelioma tumor, a multiple myeloma tumor, a muscle tumor, a nasopharyngeal tumor, a neuroblastoma, an oral tumor, an osteosarcoma, an ovarian tumor, a pancreatic tumor, a penile tumor, a pituitary tumor, a primary tumor, a prostate tumor, a retinoblastoma, a Rhabdomyosarcoma, a salivary gland tumor, a soft tissue sarcoma, a melanoma, a metastatic tumor, a basal cell carcinoma, a Merkel cell tumor, a testicular tumor, a thymus tumor, a thyroid tumor, a uterine tumor, a vaginal tumor, a vulvar tumor, or a Wilms tumor.

### Exemplification

The effects of combined administration of an anti-RGMb mAb and anti-PD-L1 or anti-PD-1 in the MC38 tumor model in GF mice was investigated. Anti-RGMb (9D1 clone) attenuated tumor growth in GF mice when given with either anti-PD-L1 or anti-PD-1, in 10 contrast to the poor response to anti-RGMb, anti-PD-L1 or anti-PD-1 alone. Combination of anti-PD-L1 anti-RGMb equivalently promoted a robust anti-tumor response in GF mice with MC38 tumors as well as in wild-type mice with B16-OVA tumors. To determine whether the RGMb antibody exerts its anti-tumor effects by blocking pathway activity or by depleting cells, the following was compared: recombinant anti-RGMb 9D1 antibody with a wild-type (mouse IgG2a) or effectorless Fc (mouse IgG2a-LALA-PG) in which the Fc portion is not able to bind to the Fc receptor, and therefore cannot function to deplete RGMb positive cells. Both RGMb antibodies attenuated tumor growth in ABX+anti-PD-L1 treated mice, and significantly increased survival compared to anti-PD-L1 therapy alone, indicating that RGMb blockade is sufficient and depletion of RGMb positive cells is not part of the mechanism.

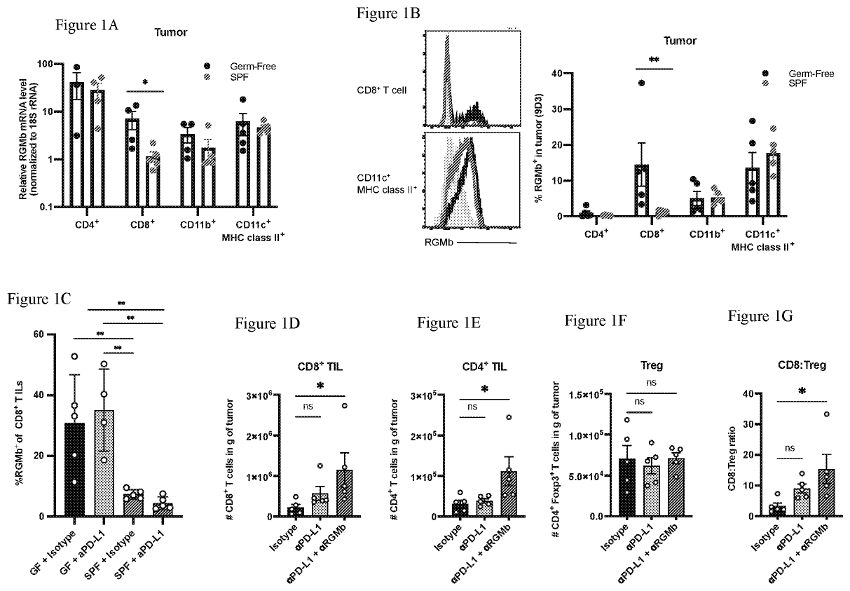

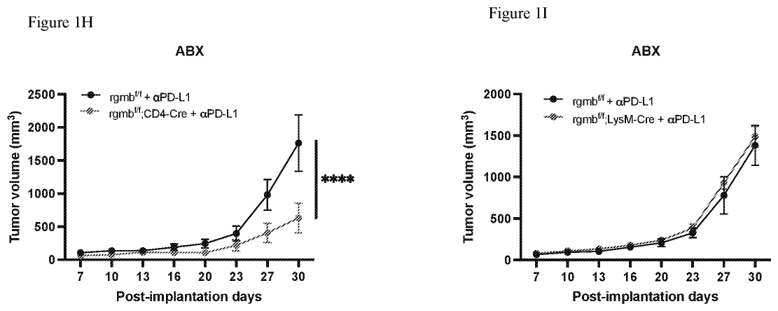

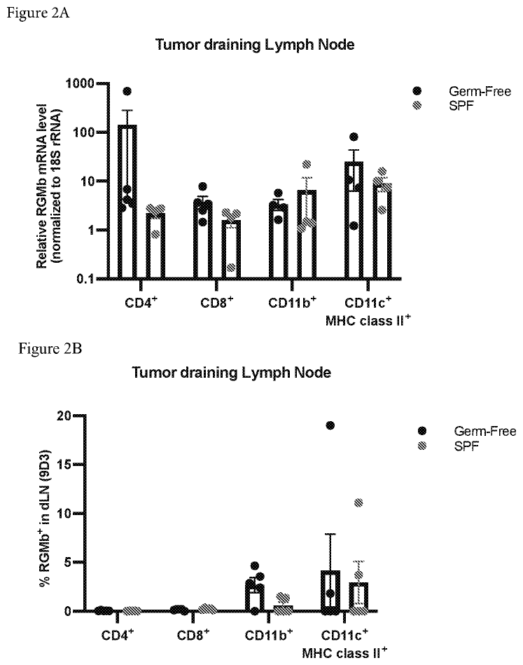

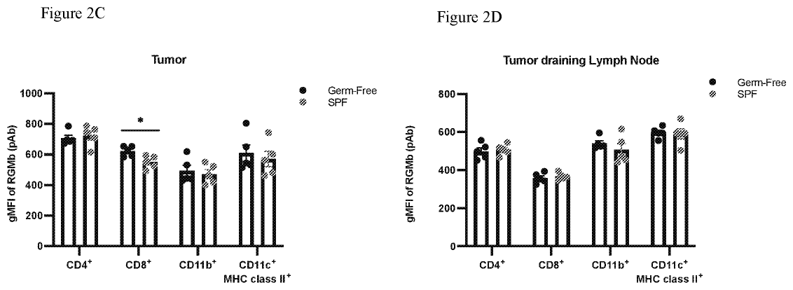

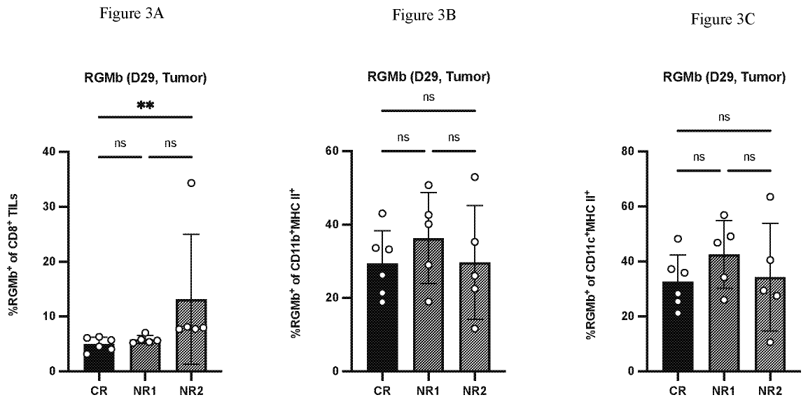

To further understand how the gut microbiota regulate the RGMb function, RGMb expression in MC38 tumor-bearing SPF was compared with GF mice. The transcript levels of RGMb in CD8⁺ tumor infiltrating T cells were 6.1-fold higher in GF mice compared to SPF mice (FIG. 1A). Similarly, RGMb protein expression, measured by a monoclonal antibody (9D3 clone) or a polyclonal antibody (FIG. 1B, FIG. 2), was significantly higher in CD8⁺ tumor infiltrating T cells from GF mice compared to SPF mice. Differences in RGMb expression in other cell subsets were not significant (FIG. 1B), suggesting that RGMb expression on T cells plays an important role in CD8+ T cell mediated anti-tumor immunity. Analysis of patient stool-colonized mice at day 29 pi revealed that NR2 tumors had significantly higher expression of RGMb on CD8⁺ T cells, but not on CD11b+ or CD1 c+ dendritic cells, suggesting that certain non-responder patients might have a microbiota that results in increased RGMb expression on T cells (FIG. 3). It was also found significantly fewer RGMb expressing intratumoral CD8⁺ T cells in SPF+anti-PD-L1 compared to GF+anti-PD-L1 mice (FIG. 1C). MC38 tumor cells expressing GFP showed high expression of PD-L1 as reported but undetectable RGMb expression when implanted in SPF or GF mice, suggesting RGMb expression on immune cells and not the tumor is responsible for influencing anti-tumor immunity (FIG. 4). Taken together, these findings demonstrate that 5 blockade of RGMb interactions can overcome microbiome-dependent resistance to anti-PD-1 or anti-PD-L1.

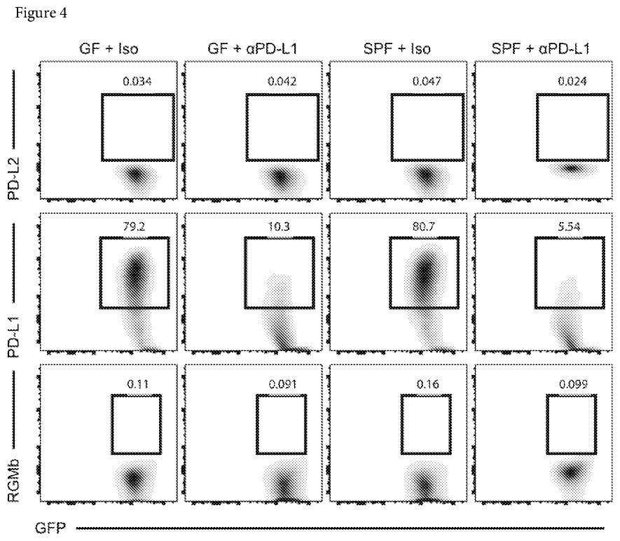

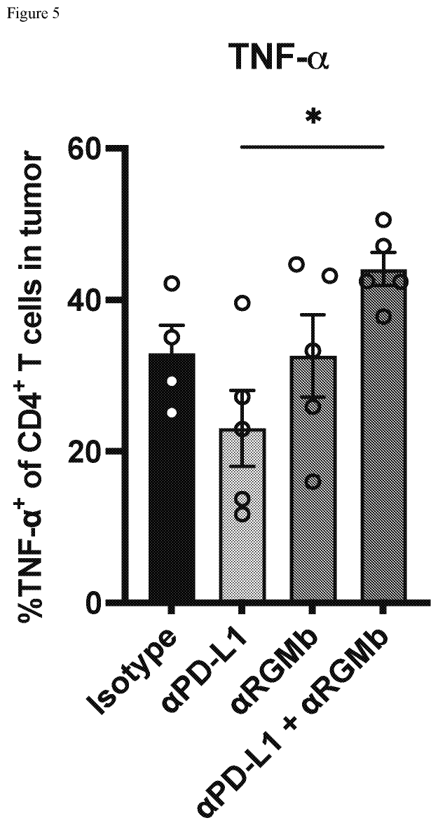

To determine how anti-RGMb treatment affected immune responses in GF mice, we analyzed immune cell subsets at day 18 after MC38 tumor implantation. Combined anti-PD-L1 and anti-RGMb therapy in GF mice led to a significant increase in the numbers of intratumoral CD8⁺ and CD4⁺ T cells, compared to the isotype control group. Anti-PD-L1 treatment alone did not significantly change the numbers of intratumoral CD8⁺ and CD4⁺ T cells in GF mice (FIG. 1D,E), consistent with other findings. The numbers of intratumoral Treg cells were similar in GF mice given anti-PD-L1, anti-PD-L1 plus anti-RGMb or isotype control antibody, consequently, the CD8⁺ to Treg cell ratio was increased in the tumors of GF mice treated with anti-PD-L1 and anti-RGMb (FIG. 1F,G). Even by day 11 p.i., anti-RGMb combined with anti-PD-L1 treatment significantly increased Tumor Necrosis Factor α (TNF-α) production by CD4⁺ T cells (FIG. 5), suggesting a potential role for RGMb in regulating pro-inflammatory cytokine expression by T cells. These data suggest that combined anti-PD-L1 and anti-RGMb treatment acts on the T cells to enhance anti-tumor immunity.

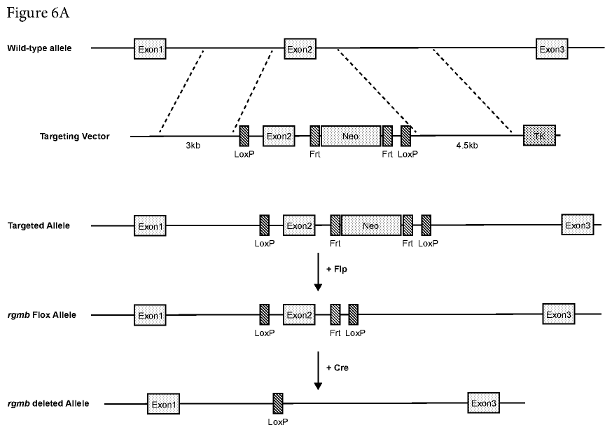

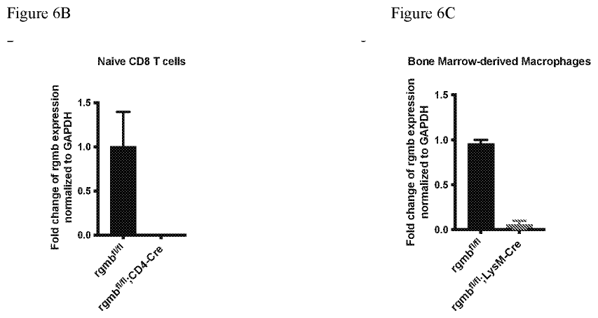

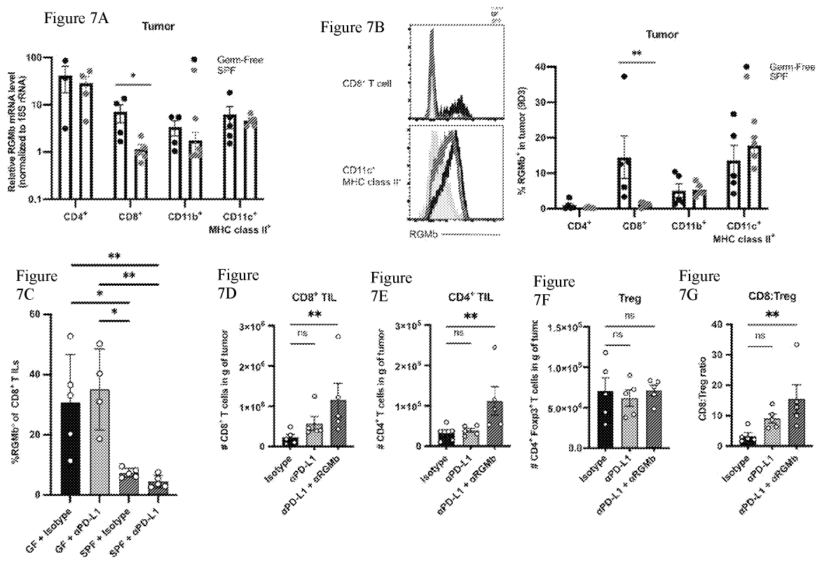

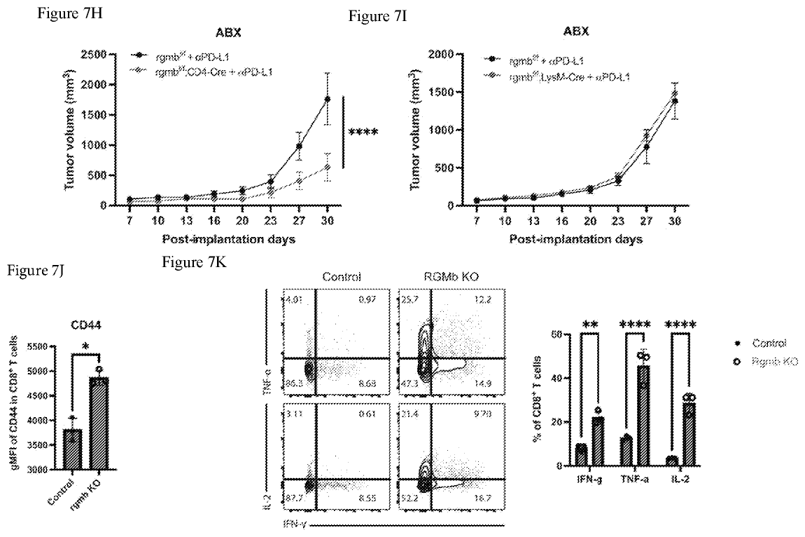

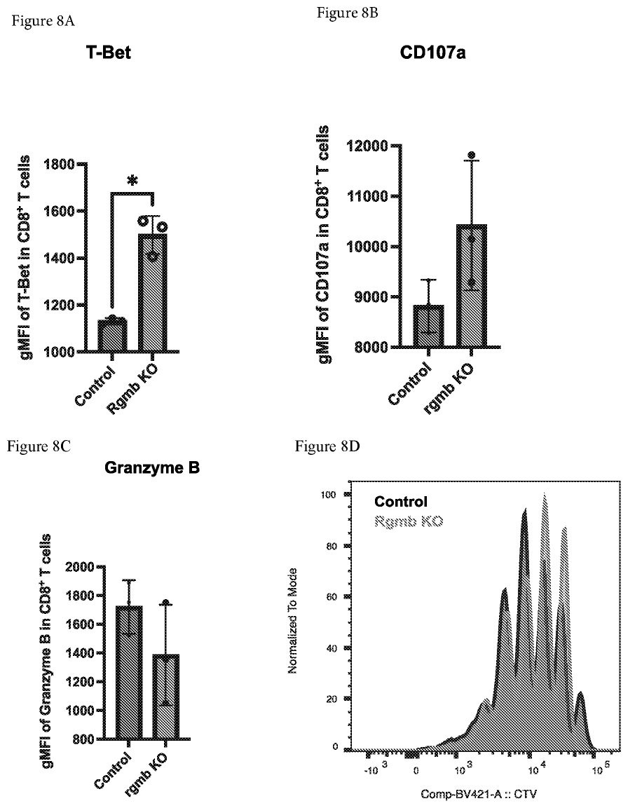

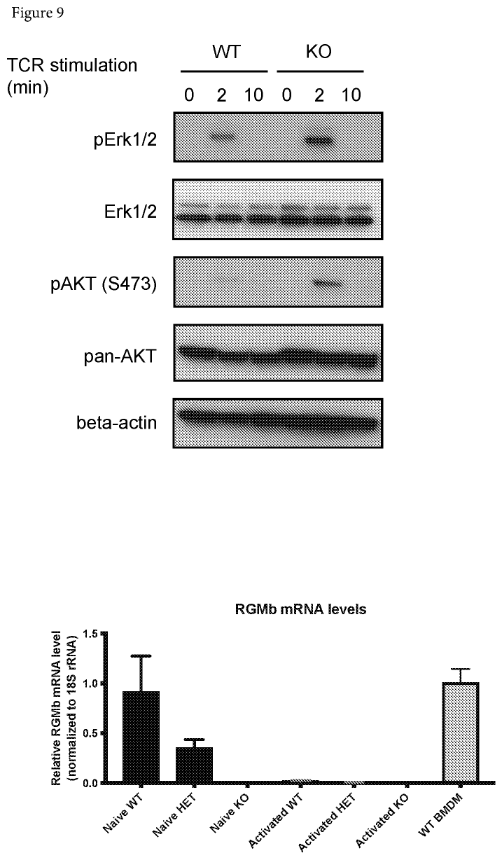

To directly investigate the function of RGMb on specific cell types in regulating anti-tumor immunity, RGMb conditional knockout mice (RGMb^{f/f}) were developed, and crossed with CD4-Cre to delete RGMb in T cells or with LysM-Cre mice to delete RGMb in macrophages and granulocytes (FIG. 6). Deletion of RGMb in T cells, but not macrophages and granulocytes, improved anti-tumor responses in ABX mice given anti-PD-L1 (FIG. 1H, I). These findings reveal a definitive and novel function for RGMb on T cells and provide a deeper mechanistic understanding of the role of RGMb in anti-tumor immunity.

The experimental data shown herein using RGMb conditional knockout mice lacking RGMb only on T cells reveals a novel and unanticipated role for RGMb on T cells in anti-tumor immunity, as most studies of RGMb on immune cells have focused on RGMb function on dendritic cells and myeloid cells.

In summary, shown herein is a novel mechanism by which the microbiome can promote response to PD-1 checkpoint blockade: by attenuating the expression of RGMb.

### Materials and Methods

### Mice

6-week old C57BL/6 female mice were purchased from Taconic Biosciences. $2m-deficient mice were obtained from The Jackson Laboratory. Germ-free mice were maintained in the germ-free facility at Harvard Medical School. All experimental mice were housed in specific pathogen-free conditions or germ-free isolators. RGMb conditional knockout mice were generated by homologous integration of a construct, which contains LoxP sites flanking exon 2 of rgmb gene and Neomycin cassette. Neo flanked by Frt sites were removed by breeding mice with germ-line transmission of the homologously integrated construct and a Flp delete strain. RGMb conditional knockout mice were further crossed with LysM-Cre (Jackson Laboratory, #004781) or CD4-Cre mice (Jackson Laboratory, #022071), to generate Macrophage-specific or T-cell specific conditional knockout mice, respectively. All mice were used in accordance with animal care guidelines from Harvard Medical School Standing Committee on Animals and the National Institutes of Health.

### Tumor Models and Antibody Treatment

Mice were anesthetized with 2.5% 2,2,2,-Tribromoethanol (Avertin, Sigma Aldrich Cat #T48402-25G) diluted in DPBS and injected subcutaneously in the abdominal flank with 2.5×10⁵ MC38 tumor cells or B16-OVA tumor cells, LLC-OVA, MB49 and Py8119-OVA cells. 2.5×10⁵ E0771 cells were injected into the mammary fat pad. Whenever GF or ABX mice were directly compared to SPF, HMB, or ABX/HMB, the same tumor cell harvest was implanted into all groups of mice on the same day. Mice were treated intraperitoneally with 100 μg of anti-PD-L1 antibody (clone 10F.9G2), anti-PD-1 antibody (clone RMP1-14), anti-RGMb antibody (clone 307.9D1), recombinant anti-RGMb antibody (clone 307.9D1 Fv with either wild-type mouse IgG2a Fc or L234A/L235A/P329G (LALA-PG) triple mutant Fc effector silent antibody (31), rat IgG2b isotype control (LTF-2, BioXCell Cat #BE0090) or rat IgG2a isotype control (2A3 BioXCell Cat #BE0089), individually or in combination as indicated, on days 7, 10, 13, 16 post tumor implantation. Tumor volume was determined by the volume formula for an ellipsoid: 0.5 ×D×d², where D is the longer diameter and d is the shorter diameter. Mice were humanely euthanized when tumors were ulcerated or reached the volume of 2000 mm³.

### Tumor-Infiltrating Leukocyte Isolation

Tumors were harvested on post-implantation days 10-16, mechanically dissociated, and incubated in DPBS containing calcium, magnesium and 250 units/mL of Type 1 Collagenase (Worthington Biochemical Corporation) for 20 minutes at 37° C. with gentle rocking. After filtration, tumor-infiltrating lymphocytes were isolated by Percoll density gradient (40%/70%) centrifugation at 800×g for 20 minutes without brake. The interface of the Percoll layers were recovered for further analyses.

### Flow Cytometry and Cell Sorting

Primary mouse cells were isolated from draining lymph node, mesenteric lymph nodes and tumors. Single cell suspensions were prepared with a 70 μm cell strainer and incubated with TruStain FcX (BioLegend Cat #101319) to block Fc receptors prior to staining. Cells were stained with LIVE/DEAD™ Fixable Near-IR Dead Cell Stain Kit (Thermo Fisher Scientific Cat #L34975) according to the manufacturer's instructions. Surface antigen staining was performed in DPBS containing 1% FBS and 2 mM EDTA, followed by intracellular staining using the eBioscience™ FoxP3/Transcription Factor Staining Buffer Set (Thermo Fisher Scientific Cat #00-5523-00). For intracellular cytokine staining, isolated cells were stimulated with 50 ng/mL of Phorbol 12-myristate 13-acetate and 500 ng/mL of ionomycin for 5 hours in the presence of 1× GolgiPlug protein transport inhibitor (BD Biosciences Cat #555029) and 1× GolgiStop protein transport inhibitor (BD Biosciences Cat #554724) prior to intracellular staining. The following antibodies were used for staining procedures: BUV395 anti-mouse CD8$ clone H35-17.2 (BD Biosciences Cat #740278), Pacific Blue anti-mouse I-A/I-E clone M5/114.15.2 (BioLegend Cat #107620), BUV737 anti-mouse CD45.2 clone 104 (BD Biosciences, Cat #564880), Brilliant Violet 711 anti-mouse CD44 clone IM7 (BioLegend Cat #103057), Alexa Fluor 700 anti-mouse CD45 clone 30-F11 (BioLegend Cat #103128), BUV564 anti-mouse CD4 clone GK1.5 (BD Biosciences Cat #612923), PE-Cy7 anti-mouse IL-17A clone TC11-18H10.1 (BioLegend Cat #506922), BUV395 anti-mouse I-A/I-E clone 2G9 (BD Biosciences Cat #743876), BV510 anti-mouse CD366 clone 5D12/TIM-3 (BD Biosciences Cat #747625), PE-CF594 anti-mouse CD11b 30 clone M1/70 (Cat #562317), BUV496 anti-mouse CD3E clone 145-2C11 (BD Biosciences Cat #564661), Brilliant Violet 570 anti-mouse CD3E clone 17A2 (BioLegend Cat #100225), Brilliant Violet 570 anti-mouse TCR$ clone H57-597 (BioLegend Cat #109231), APC anti-mouse IFNγ clone XMG1.2 (BioLegend Cat #505810), PerCP-eFluor 710 anti-mouse CD274 clone MIH5 (Thermo Fisher Scientific Cat #46-5982-82), BUV737 anti-mouse CD80 clone 16-10A1 (BD Biosciences Cat #564670), Pe-Cy7 anti-mouse CD11c clone HL3 (BD Biosciences, Cat #558079), Brilliant Violet 510 anti-mouse CD1Ic clone HL3 (BD Biosciences Cat #562949), PerCP-Cy5.5 anti-mouse TNF-α, clone MP6-XT22 (BioLegend Cat #506322), FITC anti-human/mouse Granzyme B clone GB11 (BioLegend Cat #515403), PE-Texas Red anti-mouse CD4 clone RM4-5 (Thermos Fisher Scientific Cat #MCD0417), Brilliant Violet 605 anti-mouse CD279 clone 29F.1A12 (BioLegend Cat #135220), PE-Cy7 anti-mouse Galectin-9 clone RG9-35 (BioLegend Cat #136113), Brilliant Violet 510 anti-mouse CD40 clone 3/23 (BD Biosciences Cat #745041), PerCP-Cy5.5 anti-human/mouse/rat 10 CD278 clone C398.4A (BioLegend Cat #313518), PE-Cy7 anti-mouse CD252 clone RM134L (BioLegend Cat #108813), Biotinylated anti-mouse RGMb polyclonal antibody (R&D Systems Cat #BAF3597), APC streptavidin (BioLegend Cat #405243), Brilliant Violet 605 anti-mouse CD86 clone GL1 (BD Biosciences Cat #563055), PE anti-mouse CD275 (BioLegend Cat #107405), Brilliant Violet 421 anti-mouse CD273 clone TY25 (BioLegend 15 Cat #107219), PE anti-mouse CD223 clone eBioC9B7W (Thermo Fisher Scientific Cat #12-2231-82), PE-Cy7 T-bet clone 4B10 (BioLegend Cat #644823). Alexa Fluor 594 anti-RGMb clone 9D3 antibody was generated in Gordon Freeman's laboratory as previously described(32). Flow cytometry analyses were performed on a BD™ LSR II or BD FACSymphony™. For cell sorting, a BD FACSAria™ II was used. Data were analyzed using FlowJo software.

### Quantitative Real-Time PCR Analysis

Cells were sorted by FACS BD Aria II and lysed with RLT lysis buffer. RNA was extracted using an Qiagen RNeasy Plus Mini kit (Qiagen Cat #74236). cDNA was generated using a SuperScript™ VILO™ cNDA synthesis kit (Thermo Fisher Scientific Cat #11754250), and analyzed on a LightCycler 96 machine using Applied Biosystems™ TaqMan™ Fast Advanced Master Mix (Fisher scientific Cat #44-445-57) and Taqman probes (ThermoFisher Scientific Cat #4331182, Cat #4319413E).

### Statistical Analysis

Statistical analyses were performed using GraphPad Prism 9 software. Statistical significance was determined by p-value <0.05 by using an unpaired Student's t-test for comparing two groups or one-way ANOVA for comparisons with more than two groups. Two-way ANOVA for tumor growth curves with multiple groups, followed by Bonferroni's, Tukey's, or Sidak's multiple comparison test as indicated. All the data are represented as mean±S.E.M. p-values were denoted in figures as: not significant (n.s.), *p<0.05, **p<0.01, ***p<0.001, ****p<0.0001.

Exemplary RGMb Sequences
Table 1: Exemplary Protein RGMb sequences
>NP_001012779.2 RGMB [organism = Homo sapiens]
[GeneID = 285704] [isoform = 2]
(SEQ ID NO: 3)
MIRKKRKRSAPPGPCRSHGPRPATAPAPPPSPEPTRPAWTGMGLRAAPSSAAAAAAE
VEQRRSPGLCPPPLELLLLLLFSLGLLHAGDCQQPAQCRIQKCTTDFVSLTSHLNSAV
DGFDSEFCKALRAYAGCTQRTSKACRGNLVYHSAVLGISDLMSQRNCSKDGPTSST
NPEVTHDPCNYHSHAGAREHRRGDQNPPSYLFCGLFGDPHLRTFKDNFQTCKVEGA
WPLIDNNYLSVQVTNVPVVPGSSATATNKITIIFKAHHECTDQKVYQAVTDDLPAAF
VDGTTSGGDSDAKSLRIVERESGHYVEMHARYIGTTVFVRQVGRYLTLAIRMPEDL
AMSYEESQDLQLCVNGCPLSERIDDGQGQVSAILGHSLPRTSLVQAWPGYTLETAN
TQCHEKMPVKDIYFQSCVFDLLTTGDANFTAAAHSALEDVEALHPRKERWHIFPSS
GNGTPRGGSDLSVSLGLTCLILIVFL
>NP_001353437.1 RGMB [organism = Homo sapiens]
[GeneID = 285704] [isoform = 1 precursor]
(SEQ ID NO: 4)
MGLRAAPSSAAAAAAEVEQRRSPGLCPPPLELLLLLLFSLGLLHAGDCQQPAQCRIQ
KCTTDFVSLTSHLNSAVDGFDSEFCKALRAYAGCTQRTSKACRGNLVYHSAVLGIS
DLMSQRNCSKDGPTSSTNPEVTHDPCNYHSHAGAREHRRGDQNPPSYLFCGLFGDP
HLRTFKDNFQTCKVEGAWPLIDNNYLSVQVTNVPVVPGSSATATNKITIIFKAHHEC
TDQKVYQAVTDDLPAAFVDGTTSGGDSDAKSLRIVERESGHYVEMHARYIGTTVFV
RQVGRYLTLAIRMPEDLAMSYEESQDLQLCVNGCPLSERIDDGQGQVSAILGHSLPR
TSLVQAWPGYTLETANTQCHEKMPVKDIYFQSCVFDLLTTGDANFTAAAHSALEDV
EALHPRKERWHIFPSSGNGTPRGGSDLSVSLGLTCLILIVFL
>NP_001353438.1 RGMB [organism = Homo sapiens]
[GeneID = 285704] [isoform = 2]
(SEQ ID NO: 3)
MIRKKRKRSAPPGPCRSHGPRPATAPAPPPSPEPTRPAWTGMGLRAAPSSAAAAAAE
VEQRRSPGLCPPPLELLLLLLFSLGLLHAGDCQQPAQCRIQKCTTDFVSLTSHLNSAV
DGFDSEFCKALRAYAGCTQRTSKACRGNLVYHSAVLGISDLMSQRNCSKDGPTSST
NPEVTHDPCNYHSHAGAREHRRGDQNPPSYLFCGLFGDPHLRTFKDNFQTCKVEGA
WPLIDNNYLSVQVTNVPVVPGSSATATNKITIIFKAHHECTDQKVYQAVTDDLPAAF
VDGTTSGGDSDAKSLRIVERESGHYVEMHARYIGTTVFVRQVGRYLTLAIRMPEDL
AMSYEESQDLQLCVNGCPLSERIDDGQGQVSAILGHSLPRTSLVQAWPGYTLETAN
TQCHEKMPVKDIYFQSCVFDLLTTGDANFTAAAHSALEDVEALHPRKERWHIFPSS
GNGTPRGGSDLSVSLGLTCLILIVFL
>NP_001353439.1 RGMB [organism = Homo sapiens]
[GeneID = 285704] [isoform = 2]
(SEQ ID NO: 3)
MIRKKRKRSAPPGPCRSHGPRPATAPAPPPSPEPTRPAWTGMGLRAAPSSAAAAAAE
VEQRRSPGLCPPPLELLLLLLFSLGLLHAGDCQQPAQCRIQKCTTDFVSLTSHLNSAV
DGFDSEFCKALRAYAGCTQRTSKACRGNLVYHSAVLGISDLMSQRNCSKDGPTSST
NPEVTHDPCNYHSHAGAREHRRGDQNPPSYLFCGLFGDPHLRTFKDNFQTCKVEGA
WPLIDNNYLSVQVTNVPVVPGSSATATNKITIIFKAHHECTDQKVYQAVTDDLPAAF
VDGTTSGGDSDAKSLRIVERESGHYVEMHARYIGTTVFVRQVGRYLTLAIRMPEDL
AMSYEESQDLQLCVNGCPLSERIDDGQGQVSAILGHSLPRTSLVQAWPGYTLETAN
TQCHEKMPVKDIYFQSCVFDLLTTGDANFTAAAHSALEDVEALHPRKERWHIFPSS
GNGTPRGGSDLSVSLGLTCLILIVFL
>NP_001353440.1 RGMB [organism = Homo sapiens]
[GeneID = 285704] [isoform = 3]
(SEQ ID NO: 5)
MMKKRKRSAPPGPCRSHGPRPATAPAPPPSPEPTRPAWTGMGLRAAPSSAAAAAAE
VEQRRSPGLCPPPLELLLLLLFSLGLLHAGDCQQPAQCRIQKCTTDFVSLTSHLNSAV
DGFDSEFCKALRAYAGCTQRTSKACRGNLVYHSAVLGISDLMSQRNCSKDGPTSST
NPEVTHDPCNYHSHAGAREHRRGDQNPPSYLFCGLFGDPHLRTFKDNFQTCKVEGA
WPLIDNNYLSVQVTNVPVVPGSSATATNKITIIFKAHHECTDQKVYQAVTDDLPAAF
VDGTTSGGDSDAKSLRIVERESGHYVEMHARYIGTTVFVRQVGRYLTLAIRMPEDL
AMSYEESQDLQLCVNGCPLSERIDDGQGQVSAILGHSLPRTSLVQAWPGYTLETAN
TQCHEKMPVKDIYFQSCVFDLLTTGDANFTAAAHSALEDVEALHPRKERWHIFPSS
GNGTPRGGSDLSVSLGLTCLILIVFL
>XP_011541647.1 RGMB [organism = Homo sapiens]
[GeneID = 285704] [isoform = X2]
(SEQ ID NO: 3)
MIRKKRKRSAPPGPCRSHGPRPATAPAPPPSPEPTRPAWTGMGLRAAPSSAAAAAAE
VEQRRSPGLCPPPLELLLLLLFSLGLLHAGDCQQPAQCRIQKCTTDFVSLTSHLNSAV
DGFDSEFCKALRAYAGCTQRTSKACRGNLVYHSAVLGISDLMSQRNCSKDGPTSST
NPEVTHDPCNYHSHAGAREHRRGDQNPPSYLFCGLFGDPHLRTFKDNFQTCKVEGA
WPLIDNNYLSVQVTNVPVVPGSSATATNKITIIFKAHHECTDQKVYQAVTDDLPAAF
VDGTTSGGDSDAKSLRIVERESGHYVEMHARYIGTTVFVRQVGRYLTLAIRMPEDL
AMSYEESQDLQLCVNGCPLSERIDDGQGQVSAILGHSLPRTSLVQAWPGYTLETAN
TQCHEKMPVKDIYFQSCVFDLLTTGDANFTAAAHSALEDVEALHPRKERWHIFPSS
GNGTPRGGSDLSVSLGLTCLILIVFL
>XP_011541648.1 RGMB [organism = Homo sapiens]
[GeneID = 285704] [isoform = X4]
(SEQ ID NO: 6)
MPPNCDCQQPAQCRIQKCTTDFVSLTSHLNSAVDGFDSEFCKALRAYAGCTQRTSK
ACRGNLVYHSAVLGISDLMSQRNCSKDGPTSSTNPEVTHDPCNYHSHAGAREHRRG
DQNPPSYLFCGLFGDPHLRTFKDNFQTCKVEGAWPLIDNNYLSVQVTNVPVVPGSS
ATATNKITIIFKAHHECTDQKVYQAVTDDLPAAFVDGTTSGGDSDAKSLRIVERESG
HYVEMHARYIGTTVFVRQVGRYLTLAIRMPEDLAMSYEESQDLQLCVNGCPLSERI
DDGQGQVSAILGHSLPRTSLVQAWPGYTLETANTQCHEKMPVKDIYFQSCVFDLLT
TGDANFTAAAHSALEDVEALHPRKERWHIFPSSGNGTPRGGSDLSVSLGLTCLILIVF
L
>XP_047273080.1 RGMB [organism = Homo sapiens]
[GeneID = 285704] [isoform = X1]
(SEQ ID NO: 7)
MPVGRLHWFPEINGQEAHYLRALASGSQMLFAPAVLSGPACSEVQGPPDPAEPPPG
AKGLRMIRKKRKRSAPPGPCRSHGPRPATAPAPPPSPEPTRPAWTGMGLRAAPSSAA
AAAAEVEQRRSPGLCPPPLELLLLLLFSLGLLHAGDCQQPAQCRIQKCTTDFVSLTSH
LNSAVDGFDSEFCKALRAYAGCTQRTSKACRGNLVYHSAVLGISDLMSQRNCSKD
GPTSSTNPEVTHDPCNYHSHAGAREHRRGDQNPPSYLFCGLFGDPHLRTFKDNFQTC
KVEGAWPLIDNNYLSVQVTNVPVVPGSSATATNKITIIFKAHHECTDQKVYQAVTD
DLPAAFVDGTTSGGDSDAKSLRIVERESGHYVEMHARYIGTTVFVRQVGRYLTLAIR
MPEDLAMSYEESQDLQLCVNGCPLSERIDDGQGQVSAILGHSLPRTSLVQAWPGYT
LETANTQCHEKMPVKDIYFQSCVFDLLTTGDANFTAAAHSALEDVEALHPRKERW
HIFPSSGNGTPRGGSDLSVSLGLTCLILIVFL
>XP_047273081.1 RGMB [organism = Homo sapiens]
[GeneID = 285704] [isoform = X3]
(SEQ ID NO: 4)
MGLRAAPSSAAAAAAEVEQRRSPGLCPPPLELLLLLLFSLGLLHAGDCQQPAQCRIQ
KCTTDFVSLTSHLNSAVDGFDSEFCKALRAYAGCTQRTSKACRGNLVYHSAVLGIS
DLMSQRNCSKDGPTSSTNPEVTHDPCNYHSHAGAREHRRGDQNPPSYLFCGLFGDP
HLRTFKDNFQTCKVEGAWPLIDNNYLSVQVTNVPVVPGSSATATNKITIIFKAHHEC
TDQKVYQAVTDDLPAAFVDGTTSGGDSDAKSLRIVERESGHYVEMHARYIGTTVFV
RQVGRYLTLAIRMPEDLAMSYEESQDLQLCVNGCPLSERIDDGQGQVSAILGHSLPR
TSLVQAWPGYTLETANTQCHEKMPVKDIYFQSCVFDLLTTGDANFTAAAHSALEDV
EALHPRKERWHIFPSSGNGTPRGGSDLSVSLGLTCLILIVFL
Table 2: Exemplary RGMb RNA sequences
>NM_001366510.1 RGMB [organism = Homo sapiens] [GeneID = 285704]
[transcript = 4]
(SEQ ID NO: 8)
GTAACACACTTGCTCTGGGCCTTAGGTGTCTCTTTAAACCCTCTCTCCCCTCTTTATTTCCAAATCTTTA
TCTTTGGCTTTCTTAAGCTGTAAAGGCACACTGGCACCTTAGGTGGTCAGGTAAGATCGCTGGATTCCTT
ATTTCGGCGAAAGTCCAGCGGACCATTCCCGCCACTGTACCCATTTTAGAAACACTTGGAAGACCCTTAA
GGATCATTTACGGAGAGAAAGGTCCTGCGTGTTCCGAAGTTCAGGGGCCCCCTGATCCCGCTGAGCCCCC
TCCAGGAGCGAAAGGGTTAAGAATGATAAGGAAGAAGAGGAAGCGAAGCGCGCCCCCCGGCCCATGCCGC
AGCCACGGGCCCAGACCCGCCACGGCGCCCGCGCCGCCGCCCTCGCCGGAGCCCACGAGACCTGCATGGA
CGGGCATGGGCTTGAGAGCAGCACCTTCCAGCGCCGCCGCTGCCGCCGCCGAGGTTGAGCAGCGCCGCAG
CCCCGGGCTCTGCCCCCCGCCGCTGGAGCTGCTGCTGCTGCTGCTGTTCAGCCTCGGGCTGCTCCACGCA
GGTGACTGCCAACAGCCAGCCCAATGTCGAATCCAGAAATGCACCACGGACTTCGTGTCCCTGACTTCTC
ACCTGAACTCTGCCGTTGACGGCTTTGACTCTGAGTTTTGCAAGGCCTTGCGTGCCTATGCTGGCTGCAC
CCAGCGAACTTCAAAAGCCTGCCGTGGCAACCTGGTATACCATTCTGCCGTGTTGGGTATCAGTGACCTC
ATGAGCCAGAGGAATTGTTCCAAGGATGGACCCACATCCTCTACCAACCCCGAAGTGACCCATGATCCTT
GCAACTATCACAGCCACGCTGGAGCCAGGGAACACAGGAGAGGGGACCAGAACCCTCCCAGTTACCTTTT
TTGTGGCTTGTTTGGAGATCCTCACCTCAGAACTTTCAAGGATAACTTCCAAACATGCAAAGTAGAAGGG
GCCTGGCCACTCATAGATAATAATTATCTTTCAGTTCAAGTGACAAACGTACCTGTGGTCCCTGGATCCA
GTGCTACTGCTACAAATAAGATCACTATTATCTTCAAAGCCCACCATGAGTGTACAGATCAGAAAGTCTA
CCAAGCTGTGACAGATGACCTGCCGGCCGCCTTTGTGGATGGCACCACCAGTGGTGGGGACAGCGATGCC
AAGAGCCTGCGTATCGTGGAAAGGGAGAGTGGCCACTATGTGGAGATGCACGCCCGCTATATAGGGACCA
CAGTGTTTGTGCGGCAGGTGGGTCGCTACCTGACCCTTGCCATCCGTATGCCTGAAGACCTGGCCATGTC
CTACGAGGAGAGCCAGGACCTGCAGCTGTGCGTGAACGGCTGCCCCCTGAGTGAACGCATCGATGACGGG
CAGGGCCAGGTGTCTGCCATCCTGGGACACAGCCTGCCTCGCACCTCCTTGGTGCAGGCCTGGCCTGGCT
ACACACTGGAGACTGCCAACACTCAATGCCATGAGAAGATGCCAGTGAAGGACATCTATTTCCAGTCCTG
TGTCTTCGACCTGCTCACCACTGGTGATGCCAACTTTACTGCCGCAGCCCACAGTGCCTTGGAGGATGTG
GAGGCCCTGCACCCAAGGAAGGAACGCTGGCACATTTTCCCCAGCAGTGGCAATGGGACTCCCCGTGGAG
GCAGTGATTTGTCTGTCAGTCTAGGACTCACCTGCTTGATCCTTATCGTGTTTTTGTAGGGGTTGTCTTT
TGTTTTGGTTTTTTATTTTTTGTCTATAACAAAATTTTAAAATATATATTGTCATAATATATTGAGTAAA
AGAGTATATATGTATATACCATGTATATGACAGGATGTTTGTCCTGGGACACCCACCAGATTGTACATAC
TGTGTTTGGCTGTTTTCACATATGTTGGATGTAGTGTTCTTTGATTGTATCAATTTTGTTTTGCAGTTCT
GTGAAATGTTTTATAATGTCCCTGCCCAGGGACCTGTTAGAAAGCACTTTATTTTTTATATATTAAATAT
TTATGTGTGTGCTTGGTTGATATGTATAGTACATATACACAGACATCCATATGCAGCGTTTCCTTTGAAG
GTGACCAGTTGTTTGTAGCTATTCTTGGCTGTACCTTCCTGCCCTTTCCCATTGCTACTGATTTGCCACG
GTGTGCAGCTTTTACTCGCCACCTTCCGGTGGAGCTGCCTCGTTCCTTTGAACTATGCCCTCACCCTTCT
GCCCTCACTTGATTTGAAAGGGTCGTTAACTCTCCCTTACAGGTGCTTTGACTCTTAAACGCTGATCTTA
AGAAGCTCTCTTCATCTAAGAGCTGTTACTTTTTCAGAAGGGGGGGTATTATTGGTATTCTGATTACTCT
CAATTCTAATTGTTATATATTTGAGCCCATACAGTGTATTAGGTTGAACCATAGAAACTGCTATTCTCGT
AGGTCAAAAGGGTCTAGTGATGGAAGTTTTGTAGATAAGTACCAGGCATCTCAGTAACTCCTAGACTTTT
TCTCATCCCATGCCCCGTTTTAAATTGTCAGTTTTCCCTCTGACTCTTCTGTGTTAAAACATGAAACTAT
AAATTTAGTAATTATCATGCCTTGCTCTTTTTAATCTATATGACTGATGCAAGCCCCTCTTCTTAACCGT
TTCTTGGCTTTGAGCCCAGAAACACAGCTCTCCCTGTCTCCAACTCCAGTAAGCCCTCCTCAGCCTCACC
TTACGAATCCAAAGAACTGGGGTTTGTTAGGTTCTTTCTCTAATGTAGAGGCCCAGATCCCATCACAAAG
TTTTTCATTCTTCCTTGTCCACCATGATCTTCATCACAGTCTTTGATATGTCTGCATGCAAAGTGGAACA
GAGTTGGGCGGCAATGACAGAAGAGCTTCCTTGGCCTGACTCGGTGTGCGGCCACTTCGGCACTGCTTAA
TCCAGATATTCTTGTTAACTAAGCATTGTGCTTCCCAGGTGGTCTGAAGTCAGGTACTCTCTCTCTCAAC
ACCTGTAGTTGAATATGATTTGGTCAGTTGCTCGTTGTAACTTGGAGAAATTCCTATAAAGTAAGATCTC
CTTGCCTCTTCCATCCATTGTTGGCACCCCCTTGCAAAAGGAAAAGAACAGCAAAAGTCAGGAGCAGTAA
TCTGAGAAAGTTAACTCCAGGATAGGTAGGTTTCTATTGTTATAGCTAGATGTAAATCTTTAGTTCCAAG
AAGTGATAGAGTTTCTGCTTTAATAATTTGTTGATAAGTTTACATAAACAGAAATAAAAGATACTATCTT
TACCGTAGTAGTTCAGGCCAAGATTATGCTTAGTTTTAGTTCTCCAGGTAGTTACTTTTGCCATGTCCTA
TTGATCAGTGACACTGCCAGAGGCCCATACCGGCAAGAGGAAGAGGACGTCATTTTGTAAAGTTTAACTT
CTTAGCGAACTGATGTGCCACCCAGTCACAGAGTGGAGTTGTGAATTCATGTAGAGGTGGCAAACCTCTA
CCTTGTGTTGATGAGAGAATAATCTTGGGCAGTCTGGGAAAATAAGGAAGGCATCTCCTTCTTACTCATG
GAGATTCAACTATAGAGAGTTGAAACCTAAACCCGCCTTCCTTTTATAGAAGCTGGACTAGAGACGGACT
GACCATCAGCTCTGAACTGTGGCTTTTTTTGTTCACCTATGATGCCATGTACCAAATTCAGAAGCTATCG
TTAATAATTTGTTTTATAATTGAGTAGTACAAGCGAGGAAAAAATACGGAGGATAACCACTATTTTTGTG
CAAATAATATGAAAGTGAAGTAAAAGCAATAGAAGAAATTTCTATAGGATCTGGGTTTAGAGTGTGTATC
ATTAATAAATATACCTTTGCTCTTTTCAGGGAAAATAACAACCACCCTTACTGATAGTTGGGAAAAGAAG
ATTGGGTTATTTTGCCATATCATTTAGCTGGAAGTGACATTTAAAAGCACCCTGCATCACTAGTAATAGT
GTATTTTGCTATTCTGCCCTTGTAATCGGTGTCCCTGTAAAACAATCCCCACAGATTACTTTCAGAAATA
GATGTATTTCTCTACGTAAGGGCCAGGTTTATTTTCTCCTTTTTTGAGATTTCTAGAAAAAATGCTGCTT
GCACATGTTGGTTCTTGAAACCTTAGCTAGAAGAATTTCAGGTCATACCAACATGTGGATAGGCTATAGC
TGTTCAGAGGTCTCCTGGGGGAGCTTAAAACGGGGGAAACACTGGTTTTCACAGATGCTCCACATGGCTG
TCTTTAAAAGACTCAAAACTTTTTTTTGTCCTCTTTGTTATGCTTGGAAGCTCCCCCCCCCCCAACAGTG
TGTCGAGTCTTTGCAAAGAAACCTTTAGATGTGGTTCATAGATATATGAATACGTATCTGTGTAAAACAG
TGAGTGTGCAGTGTGTAAATACTTTAAATTATTATGCTAGAAAAATAAAGTTACATACCTTGCTGTGGAA
>NM_001366509.1 RGMB [organism = Homo sapiens] [GeneID = 285704]
[transcript = 3]
(SEQ ID NO: 9)
ATTCTGGAGTCGGAGAACTGGGAGAGCGGCCCCCGAGGCGGAGCCTCCCTCCCGCCCCGAGTCGCGCTGC
CCCCCACCTGGGGGAGAGGGGGGGGCGCCGCGCTGCCTTCCCTCCGCGCCTCGGCCGCGTGGCTTGCGGC
TTATTTTCCCAGCTGGCAAGCGTCGCGCTGCAGACAAGGGAATGCCTGTGGGAAGGCTGCACTGGTTTCC
TGAGATAAATGGACAGGAAGCGCATTACCTCCGCGCGCTGGCAAGCGGCTCCCAAATGCTTTTTGCACCT
GCCGTTTTGAGCGGTGAGAGCAGCGGCGGAGCCCGCAGGGATTGCGGGGGCCCGTTGTCTACGAGCGGGT
GATGAGCCCCCCGCAAGCCCCGGGCTCTCTCCCTCCGCGAGTGCATTGCTCACGCCAAGCTTTGGGACTC
TTCTCTCACACCCAGGTCCTGCGTGTTCCGAAGTTCAGGGGCCCCCTGATCCCGCTGAGCCCCCTCCAGG
AGCGAAAGGGTTAAGAATGATAAGGAAGAAGAGGAAGCGAAGCGCGCCCCCCGGCCCATGCCGCAGCCAC
GGGCCCAGACCCGCCACGGCGCCCGCGCCGCCGCCCTCGCCGGAGCCCACGAGACCTGCATGGACGGGCA
TGGGCTTGAGAGCAGCACCTTCCAGCGCCGCCGCTGCCGCCGCCGAGGTTGAGCAGCGCCGCAGCCCCGG
GCTCTGCCCCCCGCCGCTGGAGCTGCTGCTGCTGCTGCTGTTCAGCCTCGGGCTGCTCCACGCAGGTGAC
TGCCAACAGCCAGCCCAATGTCGAATCCAGAAATGCACCACGGACTTCGTGTCCCTGACTTCTCACCTGA
ACTCTGCCGTTGACGGCTTTGACTCTGAGTTTTGCAAGGCCTTGCGTGCCTATGCTGGCTGCACCCAGCG
AACTTCAAAAGCCTGCCGTGGCAACCTGGTATACCATTCTGCCGTGTTGGGTATCAGTGACCTCATGAGC
CAGAGGAATTGTTCCAAGGATGGACCCACATCCTCTACCAACCCCGAAGTGACCCATGATCCTTGCAACT
ATCACAGCCACGCTGGAGCCAGGGAACACAGGAGAGGGGACCAGAACCCTCCCAGTTACCTTTTTTGTGG
CTTGTTTGGAGATCCTCACCTCAGAACTTTCAAGGATAACTTCCAAACATGCAAAGTAGAAGGGGCCTGG
CCACTCATAGATAATAATTATCTTTCAGTTCAAGTGACAAACGTACCTGTGGTCCCTGGATCCAGTGCTA
CTGCTACAAATAAGATCACTATTATCTTCAAAGCCCACCATGAGTGTACAGATCAGAAAGTCTACCAAGC
TGTGACAGATGACCTGCCGGCCGCCTTTGTGGATGGCACCACCAGTGGTGGGGACAGCGATGCCAAGAGC
CTGCGTATCGTGGAAAGGGAGAGTGGCCACTATGTGGAGATGCACGCCCGCTATATAGGGACCACAGTGT
TTGTGCGGCAGGTGGGTCGCTACCTGACCCTTGCCATCCGTATGCCTGAAGACCTGGCCATGTCCTACGA
GGAGAGCCAGGACCTGCAGCTGTGCGTGAACGGCTGCCCCCTGAGTGAACGCATCGATGACGGGCAGGGC
CAGGTGTCTGCCATCCTGGGACACAGCCTGCCTCGCACCTCCTTGGTGCAGGCCTGGCCTGGCTACACAC
TGGAGACTGCCAACACTCAATGCCATGAGAAGATGCCAGTGAAGGACATCTATTTCCAGTCCTGTGTCTT
CGACCTGCTCACCACTGGTGATGCCAACTTTACTGCCGCAGCCCACAGTGCCTTGGAGGATGTGGAGGCC
CTGCACCCAAGGAAGGAACGCTGGCACATTTTCCCCAGCAGTGGCAATGGGACTCCCCGTGGAGGCAGTG
ATTTGTCTGTCAGTCTAGGACTCACCTGCTTGATCCTTATCGTGTTTTTGTAGGGGTTGTCTTTTGTTTT
GGTTTTTTATTTTTTGTCTATAACAAAATTTTAAAATATATATTGTCATAATATATTGAGTAAAAGAGTA
TATATGTATATACCATGTATATGACAGGATGTTTGTCCTGGGACACCCACCAGATTGTACATACTGTGTT
TGGCTGTTTTCACATATGTTGGATGTAGTGTTCTTTGATTGTATCAATTTTGTTTTGCAGTTCTGTGAAA
TGTTTTATAATGTCCCTGCCCAGGGACCTGTTAGAAAGCACTTTATTTTTTATATATTAAATATTTATGT
GTGTGCTTGGTTGATATGTATAGTACATATACACAGACATCCATATGCAGCGTTTCCTTTGAAGGTGACC
AGTTGTTTGTAGCTATTCTTGGCTGTACCTTCCTGCCCTTTCCCATTGCTACTGATTTGCCACGGTGTGC
AGCTTTTACTCGCCACCTTCCGGTGGAGCTGCCTCGTTCCTTTGAACTATGCCCTCACCCTTCTGCCCTC
ACTTGATTTGAAAGGGTCGTTAACTCTCCCTTACAGGTGCTTTGACTCTTAAACGCTGATCTTAAGAAGC
TCTCTTCATCTAAGAGCTGTTACTTTTTCAGAAGGGGGGGTATTATTGGTATTCTGATTACTCTCAATTC
TAATTGTTATATATTTGAGCCCATACAGTGTATTAGGTTGAACCATAGAAACTGCTATTCTCGTAGGTCA
AAAGGGTCTAGTGATGGAAGTTTTGTAGATAAGTACCAGGCATCTCAGTAACTCCTAGACTTTTTCTCAT
CCCATGCCCCGTTTTAAATTGTCAGTTTTCCCTCTGACTCTTCTGTGTTAAAACATGAAACTATAAATTT
AGTAATTATCATGCCTTGCTCTTTTTAATCTATATGACTGATGCAAGCCCCTCTTCTTAACCGTTTCTTG
GCTTTGAGCCCAGAAACACAGCTCTCCCTGTCTCCAACTCCAGTAAGCCCTCCTCAGCCTCACCTTACGA
ATCCAAAGAACTGGGGTTTGTTAGGTTCTTTCTCTAATGTAGAGGCCCAGATCCCATCACAAAGTTTTTC
ATTCTTCCTTGTCCACCATGATCTTCATCACAGTCTTTGATATGTCTGCATGCAAAGTGGAACAGAGTTG
GGCGGCAATGACAGAAGAGCTTCCTTGGCCTGACTCGGTGTGCGGCCACTTCGGCACTGCTTAATCCAGA
TATTCTTGTTAACTAAGCATTGTGCTTCCCAGGTGGTCTGAAGTCAGGTACTCTCTCTCTCAACACCTGT
AGTTGAATATGATTTGGTCAGTTGCTCGTTGTAACTTGGAGAAATTCCTATAAAGTAAGATCTCCTTGCC
TCTTCCATCCATTGTTGGCACCCCCTTGCAAAAGGAAAAGAACAGCAAAAGTCAGGAGCAGTAATCTGAG
AAAGTTAACTCCAGGATAGGTAGGTTTCTATTGTTATAGCTAGATGTAAATCTTTAGTTCCAAGAAGTGA
TAGAGTTTCTGCTTTAATAATTTGTTGATAAGTTTACATAAACAGAAATAAAAGATACTATCTTTACCGT
AGTAGTTCAGGCCAAGATTATGCTTAGTTTTAGTTCTCCAGGTAGTTACTTTTGCCATGTCCTATTGATC
AGTGACACTGCCAGAGGCCCATACCGGCAAGAGGAAGAGGACGTCATTTTGTAAAGTTTAACTTCTTAGC
GAACTGATGTGCCACCCAGTCACAGAGTGGAGTTGTGAATTCATGTAGAGGTGGCAAACCTCTACCTTGT
GTTGATGAGAGAATAATCTTGGGCAGTCTGGGAAAATAAGGAAGGCATCTCCTTCTTACTCATGGAGATT
CAACTATAGAGAGTTGAAACCTAAACCCGCCTTCCTTTTATAGAAGCTGGACTAGAGACGGACTGACCAT
CAGCTCTGAACTGTGGCTTTTTTTGTTCACCTATGATGCCATGTACCAAATTCAGAAGCTATCGTTAATA
ATTTGTTTTATAATTGAGTAGTACAAGCGAGGAAAAAATACGGAGGATAACCACTATTTTTGTGCAAATA
ATATGAAAGTGAAGTAAAAGCAATAGAAGAAATTTCTATAGGATCTGGGTTTAGAGTGTGTATCATTAAT
AAATATACCTTTGCTCTTTTCAGGGAAAATAACAACCACCCTTACTGATAGTTGGGAAAAGAAGATTGGG
TTATTTTGCCATATCATTTAGCTGGAAGTGACATTTAAAAGCACCCTGCATCACTAGTAATAGTGTATTT
TGCTATTCTGCCCTTGTAATCGGTGTCCCTGTAAAACAATCCCCACAGATTACTTTCAGAAATAGATGTA
TTTCTCTACGTAAGGGCCAGGTTTATTTTCTCCTTTTTTGAGATTTCTAGAAAAAATGCTGCTTGCACAT
GTTGGTTCTTGAAACCTTAGCTAGAAGAATTTCAGGTCATACCAACATGTGGATAGGCTATAGCTGTTCA
GAGGTCTCCTGGGGGAGCTTAAAACGGGGGAAACACTGGTTTTCACAGATGCTCCACATGGCTGTCTTTA
AAAGACTCAAAACTTTTTTTTGTCCTCTTTGTTATGCTTGGAAGCTCCCCCCCCCCCAACAGTGTGTCGA
GTCTTTGCAAAGAAACCTTTAGATGTGGTTCATAGATATATGAATACGTATCTGTGTAAAACAGTGAGTG
TGCAGTGTGTAAATACTTTAAATTATTATGCTAGAAAAATAAAGTTACATACCTTGCTGTGGAA
>XM_047417124.1 RGMB [organism = Homo sapiens] [GeneID = 285704]
[transcript = X1]
(SEQ ID NO: 10)
ATTCTGGAGTCGGAGAACTGGGAGAGCGGCCCCCGAGGCGGAGCCTCCCTCCCGCCCCGAGTCGCGCTGC
CCCCCACCTGGGGGAGAGGGGGGGGCGCCGCGCTGCCTTCCCTCCGCGCCTCGGCCGCGTGGCTTGCGGC
TTATTTTCCCAGCTGGCAAGCGTCGCGCTGCAGACAAGGGAATGCCTGTGGGAAGGCTGCACTGGTTTCC
TGAGATAAATGGACAGGAAGCGCATTACCTCCGCGCGCTGGCAAGCGGCTCCCAAATGCTTTTTGCACCT
GCCGTTTTGAGCGGTCCTGCGTGTTCCGAAGTTCAGGGGCCCCCTGATCCCGCTGAGCCCCCTCCAGGAG
CGAAAGGGTTAAGAATGATAAGGAAGAAGAGGAAGCGAAGCGCGCCCCCCGGCCCATGCCGCAGCCACGG
GCCCAGACCCGCCACGGCGCCCGCGCCGCCGCCCTCGCCGGAGCCCACGAGACCTGCATGGACGGGCATG
GGCTTGAGAGCAGCACCTTCCAGCGCCGCCGCTGCCGCCGCCGAGGTTGAGCAGCGCCGCAGCCCCGGGC
TCTGCCCCCCGCCGCTGGAGCTGCTGCTGCTGCTGCTGTTCAGCCTCGGGCTGCTCCACGCAGGTGACTG
CCAACAGCCAGCCCAATGTCGAATCCAGAAATGCACCACGGACTTCGTGTCCCTGACTTCTCACCTGAAC
TCTGCCGTTGACGGCTTTGACTCTGAGTTTTGCAAGGCCTTGCGTGCCTATGCTGGCTGCACCCAGCGAA
CTTCAAAAGCCTGCCGTGGCAACCTGGTATACCATTCTGCCGTGTTGGGTATCAGTGACCTCATGAGCCA
GAGGAATTGTTCCAAGGATGGACCCACATCCTCTACCAACCCCGAAGTGACCCATGATCCTTGCAACTAT
CACAGCCACGCTGGAGCCAGGGAACACAGGAGAGGGGACCAGAACCCTCCCAGTTACCTTTTTTGTGGCT
TGTTTGGAGATCCTCACCTCAGAACTTTCAAGGATAACTTCCAAACATGCAAAGTAGAAGGGGCCTGGCC
ACTCATAGATAATAATTATCTTTCAGTTCAAGTGACAAACGTACCTGTGGTCCCTGGATCCAGTGCTACT
GCTACAAATAAGATCACTATTATCTTCAAAGCCCACCATGAGTGTACAGATCAGAAAGTCTACCAAGCTG
TGACAGATGACCTGCCGGCCGCCTTTGTGGATGGCACCACCAGTGGTGGGGACAGCGATGCCAAGAGCCT
GCGTATCGTGGAAAGGGAGAGTGGCCACTATGTGGAGATGCACGCCCGCTATATAGGGACCACAGTGTTT
GTGCGGCAGGTGGGTCGCTACCTGACCCTTGCCATCCGTATGCCTGAAGACCTGGCCATGTCCTACGAGG
AGAGCCAGGACCTGCAGCTGTGCGTGAACGGCTGCCCCCTGAGTGAACGCATCGATGACGGGCAGGGCCA
GGTGTCTGCCATCCTGGGACACAGCCTGCCTCGCACCTCCTTGGTGCAGGCCTGGCCTGGCTACACACTG
GAGACTGCCAACACTCAATGCCATGAGAAGATGCCAGTGAAGGACATCTATTTCCAGTCCTGTGTCTTCG
ACCTGCTCACCACTGGTGATGCCAACTTTACTGCCGCAGCCCACAGTGCCTTGGAGGATGTGGAGGCCCT
GCACCCAAGGAAGGAACGCTGGCACATTTTCCCCAGCAGTGGCAATGGGACTCCCCGTGGAGGCAGTGAT
TTGTCTGTCAGTCTAGGACTCACCTGCTTGATCCTTATCGTGTTTTTGTAGGGGTTGTCTTTTGTTTTGG
TTTTTTATTTTTTGTCTATAACAAAATTTTAAAATATATATTGTCATAATATATTGAGTAAAAGAGTATA
TATGTATATACCATGTATATGACAGGATGTTTGTCCTGGGACACCCACCAGATTGTACATACTGTGTTTG
GCTGTTTTCACATATGTTGGATGTAGTGTTCTTTGATTGTATCAATTTTGTTTTGCAGTTCTGTGAAATG
TTTTATAATGTCCCTGCCCAGGGACCTGTTAGAAAGCACTTTATTTTTTATATATTAAATATTTATGTGT
GTGCTTGGTTGATATGTATAGTACATATACACAGACATCCATATGCAGCGTTTCCTTTGAAGGTGACCAG
TTGTTTGTAGCTATTCTTGGCTGTACCTTCCTGCCCTTTCCCATTGCTACTGATTTGCCACGGTGTGCAG
CTTTTACTCGCCACCTTCCGGTGGAGCTGCCTCGTTCCTTTGAACTATGCCCTCACCCTTCTGCCCTCAC
TTGATTTGAAAGGGTCGTTAACTCTCCCTTACAGGTGCTTTGACTCTTAAACGCTGATCTTAAGAAGCTC
TCTTCATCTAAGAGCTGTTACTTTTTCAGAAGGGGGGGTATTATTGGTATTCTGATTACTCTCAATTCTA
ATTGTTATATATTTGAGCCCATACAGTGTATTAGGTTGAACCATAGAAACTGCTATTCTCGTAGGTCAAA
AGGGTCTAGTGATGGAAGTTTTGTAGATAAGTACCAGGCATCTCAGTAACTCCTAGACTTTTTCTCATCC
CATGCCCCGTTTTAAATTGTCAGTTTTCCCTCTGACTCTTCTGTGTTAAAACATGAAACTATAAATTTAG
TAATTATCATGCCTTGCTCTTTTTAATCTATATGACTGATGCAAGCCCCTCTTCTTAACCGTTTCTTGGC
TTTGAGCCCAGAAACACAGCTCTCCCTGTCTCCAACTCCAGTAAGCCCTCCTCAGCCTCACCTTACGAAT
CCAAAGAACTGGGGTTTGTTAGGTTCTTTCTCTAATGTAGAGGCCCAGATCCCATCACAAAGTTTTTCAT
TCTTCCTTGTCCACCATGATCTTCATCACAGTCTTTGATATGTCTGCATGCAAAGTGGAACAGAGTTGGG
CGGCAATGACAGAAGAGCTTCCTTGGCCTGACTCGGTGTGCGGCCACTTCGGCACTGCTTAATCCAGATA
TTCTTGTTAACTAAGCATTGTGCTTCCCAGGTGGTCTGAAGTCAGGTACTCTCTCTCTCAACACCTGTAG
TTGAATATGATTTGGTCAGTTGCTCGTTGTAACTTGGAGAAATTCCTATAAAGTAAGATCTCCTTGCCTC
TTCCATCCATTGTTGGCACCCCCTTGCAAAAGGAAAAGAACAGCAAAAGTCAGGAGCAGTAATCTGAGAA
AGTTAACTCCAGGATAGGTAGGTTTCTATTGTTATAGCTAGATGTAAATCTTTAGTTCCAAGAAGTGATA
GAGTTTCTGCTTTAATAATTTGTTGATAAGTTTACATAAACAGAAATAAAAGATACTATCTTTACCGTAG
TAGTTCAGGCCAAGATTATGCTTAGTTTTAGTTCTCCAGGTAGTTACTTTTGCCATGTCCTATTGATCAG
TGACACTGCCAGAGGCCCATACCGGCAAGAGGAAGAGGACGTCATTTTGTAAAGTTTAACTTCTTAGCGA
ACTGATGTGCCACCCAGTCACAGAGTGGAGTTGTGAATTCATGTAGAGGTGGCAAACCTCTACCTTGTGT
TGATGAGAGAATAATCTTGGGCAGTCTGGGAAAATAAGGAAGGCATCTCCTTCTTACTCATGGAGATTCA
ACTATAGAGAGTTGAAACCTAAACCCGCCTTCCTTTTATAGAAGCTGGACTAGAGACGGACTGACCATCA
GCTCTGAACTGTGGCTTTTTTTGTTCACCTATGATGCCATGTACCAAATTCAGAAGCTATCGTTAATAAT
TTGTTTTATAATTGAGTAGTACAAGCGAGGAAAAAATACGGAGGATAACCACTATTTTTGTGCAAATAAT
ATGAAAGTGAAGTAAAAGCAATAGAAGAAATTTCTATAGGATCTGGGTTTAGAGTGTGTATCATTAATAA
ATATACCTTTGCTCTTTTCAGGGAAAATAACAACCACCCTTACTGATAGTTGGGAAAAGAAGATTGGGTT
ATTTTGCCATATCATTTAGCTGGAAGTGACATTTAAAAGCACCCTGCATCACTAGTAATAGTGTATTTTG
CTATTCTGCCCTTGTAATCGGTGTCCCTGTAAAACAATCCCCACAGATTACTTTCAGAAATAGATGTATT
TCTCTACGTAAGGGCCAGGTTTATTTTCTCCTTTTTTGAGATTTCTAGAAAAAATGCTGCTTGCACATGT
TGGTTCTTGAAACCTTAGCTAGAAGAATTTCAGGTCATACCAACATGTGGATAGGCTATAGCTGTTCAGA
GGTCTCCTGGGGGAGCTTAAAACGGGGGAAACACTGGTTTTCACAGATGCTCCACATGGCTGTCTTTAAA
AGACTCAAAACTTTTTTTTGTCCTCTTTGTTATGCTTGGAAGCTCCCCCCCCCCCAACAGTGTGTCGAGT
CTTTGCAAAGAAACCTTTAGATGTGGTTCATAGATATATGAATACGTATCTGTGTAAAACAGTGAGTGTG
CAGTGTGTAAATACTTTAAATTATTATGCTAGAAAAATAAAGTTACATACCTTGCTGTGGAA
>NM_001012761.3 RGMB [organism = Homo sapiens] [GeneID = 285704]
[transcript = 2]
(SEQ ID NO: 11)
ATTCTGGAGTCGGAGAACTGGGAGAGCGGCCCCCGAGGCGGAGCCTCCCTCCCGCCCCGAGTCGCGCTGC
CCCCCACCTGGGGGAGAGGGGGGGGCGCCGCGCTGCCTTCCCTCCGCGCCTCGGCCGCGTGGCTTGCGGC
TTATTTTCCCAGCTGGCAAGCGTCGCGCTGCAGACAAGGGAATGCCTGTGGTCCTGCGTGTTCCGAAGTT
CAGGGGCCCCCTGATCCCGCTGAGCCCCCTCCAGGAGCGAAAGGGTTAAGAATGATAAGGAAGAAGAGGA
AGCGAAGCGCGCCCCCCGGCCCATGCCGCAGCCACGGGCCCAGACCCGCCACGGCGCCCGCGCCGCCGCC
CTCGCCGGAGCCCACGAGACCTGCATGGACGGGCATGGGCTTGAGAGCAGCACCTTCCAGCGCCGCCGCT
GCCGCCGCCGAGGTTGAGCAGCGCCGCAGCCCCGGGCTCTGCCCCCCGCCGCTGGAGCTGCTGCTGCTGC
TGCTGTTCAGCCTCGGGCTGCTCCACGCAGGTGACTGCCAACAGCCAGCCCAATGTCGAATCCAGAAATG
CACCACGGACTTCGTGTCCCTGACTTCTCACCTGAACTCTGCCGTTGACGGCTTTGACTCTGAGTTTTGC
AAGGCCTTGCGTGCCTATGCTGGCTGCACCCAGCGAACTTCAAAAGCCTGCCGTGGCAACCTGGTATACC
ATTCTGCCGTGTTGGGTATCAGTGACCTCATGAGCCAGAGGAATTGTTCCAAGGATGGACCCACATCCTC
TACCAACCCCGAAGTGACCCATGATCCTTGCAACTATCACAGCCACGCTGGAGCCAGGGAACACAGGAGA
GGGGACCAGAACCCTCCCAGTTACCTTTTTTGTGGCTTGTTTGGAGATCCTCACCTCAGAACTTTCAAGG
ATAACTTCCAAACATGCAAAGTAGAAGGGGCCTGGCCACTCATAGATAATAATTATCTTTCAGTTCAAGT
GACAAACGTACCTGTGGTCCCTGGATCCAGTGCTACTGCTACAAATAAGATCACTATTATCTTCAAAGCC
CACCATGAGTGTACAGATCAGAAAGTCTACCAAGCTGTGACAGATGACCTGCCGGCCGCCTTTGTGGATG
GCACCACCAGTGGTGGGGACAGCGATGCCAAGAGCCTGCGTATCGTGGAAAGGGAGAGTGGCCACTATGT
GGAGATGCACGCCCGCTATATAGGGACCACAGTGTTTGTGCGGCAGGTGGGTCGCTACCTGACCCTTGCC
ATCCGTATGCCTGAAGACCTGGCCATGTCCTACGAGGAGAGCCAGGACCTGCAGCTGTGCGTGAACGGCT
GCCCCCTGAGTGAACGCATCGATGACGGGCAGGGCCAGGTGTCTGCCATCCTGGGACACAGCCTGCCTCG
CACCTCCTTGGTGCAGGCCTGGCCTGGCTACACACTGGAGACTGCCAACACTCAATGCCATGAGAAGATG
CCAGTGAAGGACATCTATTTCCAGTCCTGTGTCTTCGACCTGCTCACCACTGGTGATGCCAACTTTACTG
CCGCAGCCCACAGTGCCTTGGAGGATGTGGAGGCCCTGCACCCAAGGAAGGAACGCTGGCACATTTTCCC
CAGCAGTGGCAATGGGACTCCCCGTGGAGGCAGTGATTTGTCTGTCAGTCTAGGACTCACCTGCTTGATC
CTTATCGTGTTTTTGTAGGGGTTGTCTTTTGTTTTGGTTTTTTATTTTTTGTCTATAACAAAATTTTAAA
ATATATATTGTCATAATATATTGAGTAAAAGAGTATATATGTATATACCATGTATATGACAGGATGTTTG
TCCTGGGACACCCACCAGATTGTACATACTGTGTTTGGCTGTTTTCACATATGTTGGATGTAGTGTTCTT
TGATTGTATCAATTTTGTTTTGCAGTTCTGTGAAATGTTTTATAATGTCCCTGCCCAGGGACCTGTTAGA
AAGCACTTTATTTTTTATATATTAAATATTTATGTGTGTGCTTGGTTGATATGTATAGTACATATACACA
GACATCCATATGCAGCGTTTCCTTTGAAGGTGACCAGTTGTTTGTAGCTATTCTTGGCTGTACCTTCCTG
CCCTTTCCCATTGCTACTGATTTGCCACGGTGTGCAGCTTTTACTCGCCACCTTCCGGTGGAGCTGCCTC
GTTCCTTTGAACTATGCCCTCACCCTTCTGCCCTCACTTGATTTGAAAGGGTCGTTAACTCTCCCTTACA
GGTGCTTTGACTCTTAAACGCTGATCTTAAGAAGCTCTCTTCATCTAAGAGCTGTTACTTTTTCAGAAGG
GGGGGTATTATTGGTATTCTGATTACTCTCAATTCTAATTGTTATATATTTGAGCCCATACAGTGTATTA
GGTTGAACCATAGAAACTGCTATTCTCGTAGGTCAAAAGGGTCTAGTGATGGAAGTTTTGTAGATAAGTA
CCAGGCATCTCAGTAACTCCTAGACTTTTTCTCATCCCATGCCCCGTTTTAAATTGTCAGTTTTCCCTCT
GACTCTTCTGTGTTAAAACATGAAACTATAAATTTAGTAATTATCATGCCTTGCTCTTTTTAATCTATAT
GACTGATGCAAGCCCCTCTTCTTAACCGTTTCTTGGCTTTGAGCCCAGAAACACAGCTCTCCCTGTCTCC
AACTCCAGTAAGCCCTCCTCAGCCTCACCTTACGAATCCAAAGAACTGGGGTTTGTTAGGTTCTTTCTCT
AATGTAGAGGCCCAGATCCCATCACAAAGTTTTTCATTCTTCCTTGTCCACCATGATCTTCATCACAGTC
TTTGATATGTCTGCATGCAAAGTGGAACAGAGTTGGGCGGCAATGACAGAAGAGCTTCCTTGGCCTGACT
CGGTGTGCGGCCACTTCGGCACTGCTTAATCCAGATATTCTTGTTAACTAAGCATTGTGCTTCCCAGGTG
GTCTGAAGTCAGGTACTCTCTCTCTCAACACCTGTAGTTGAATATGATTTGGTCAGTTGCTCGTTGTAAC
TTGGAGAAATTCCTATAAAGTAAGATCTCCTTGCCTCTTCCATCCATTGTTGGCACCCCCTTGCAAAAGG
AAAAGAACAGCAAAAGTCAGGAGCAGTAATCTGAGAAAGTTAACTCCAGGATAGGTAGGTTTCTATTGTT
ATAGCTAGATGTAAATCTTTAGTTCCAAGAAGTGATAGAGTTTCTGCTTTAATAATTTGTTGATAAGTTT
ACATAAACAGAAATAAAAGATACTATCTTTACCGTAGTAGTTCAGGCCAAGATTATGCTTAGTTTTAGTT
CTCCAGGTAGTTACTTTTGCCATGTCCTATTGATCAGTGACACTGCCAGAGGCCCATACCGGCAAGAGGA
AGAGGACGTCATTTTGTAAAGTTTAACTTCTTAGCGAACTGATGTGCCACCCAGTCACAGAGTGGAGTTG
TGAATTCATGTAGAGGTGGCAAACCTCTACCTTGTGTTGATGAGAGAATAATCTTGGGCAGTCTGGGAAA
ATAAGGAAGGCATCTCCTTCTTACTCATGGAGATTCAACTATAGAGAGTTGAAACCTAAACCCGCCTTCC
TTTTATAGAAGCTGGACTAGAGACGGACTGACCATCAGCTCTGAACTGTGGCTTTTTTTGTTCACCTATG
ATGCCATGTACCAAATTCAGAAGCTATCGTTAATAATTTGTTTTATAATTGAGTAGTACAAGCGAGGAAA
AAATACGGAGGATAACCACTATTTTTGTGCAAATAATATGAAAGTGAAGTAAAAGCAATAGAAGAAATTT
CTATAGGATCTGGGTTTAGAGTGTGTATCATTAATAAATATACCTTTGCTCTTTTCAGGGAAAATAACAA
CCACCCTTACTGATAGTTGGGAAAAGAAGATTGGGTTATTTTGCCATATCATTTAGCTGGAAGTGACATT
TAAAAGCACCCTGCATCACTAGTAATAGTGTATTTTGCTATTCTGCCCTTGTAATCGGTGTCCCTGTAAA
ACAATCCCCACAGATTACTTTCAGAAATAGATGTATTTCTCTACGTAAGGGCCAGGTTTATTTTCTCCTT
TTTTGAGATTTCTAGAAAAAATGCTGCTTGCACATGTTGGTTCTTGAAACCTTAGCTAGAAGAATTTCAG
GTCATACCAACATGTGGATAGGCTATAGCTGTTCAGAGGTCTCCTGGGGGAGCTTAAAACGGGGGAAACA
CTGGTTTTCACAGATGCTCCACATGGCTGTCTTTAAAAGACTCAAAACTTTTTTTTGTCCTCTTTGTTAT
GCTTGGAAGCTCCCCCCCCCCCAACAGTGTGTCGAGTCTTTGCAAAGAAACCTTTAGATGTGGTTCATAG
ATATATGAATACGTATCTGTGTAAAACAGTGAGTGTGCAGTGTGTAAATACTTTAAATTATTATGCTAGA
AAAATAAAGTTACATACCTTGCTGTGGAA
>NM_001366511.1 RGMB [organism = Homo sapiens] [GeneID = 285704]
[transcript = 5]
(SEQ ID NO: 12)
ATTCTGGAGTCGGAGAACTGGGAGAGCGGCCCCCGAGGCGGAGCCTCCCTCCCGCCCCGAGTCGCGCTGC
CCCCCACCTGGGGGAGAGGGGGGGGCGCCGCGCTGCCTTCCCTCCGCGCCTCGGCCGCGTGGCTTGCGGC
TTATTTTCCCAGCTGGCAAGCGTCGCGCTGCAGACAAGGGAATGCCTGTGGTCCTGCGTGTTCCGAAGTT
CAGGGGCCCCCTGATCCCGCTGAGCCCCCTCCAGGAGCGAAAGGGTTAAGAATGATGAAGAAGAGGAAGC
GAAGCGCGCCCCCCGGCCCATGCCGCAGCCACGGGCCCAGACCCGCCACGGCGCCCGCGCCGCCGCCCTC
GCCGGAGCCCACGAGACCTGCATGGACGGGCATGGGCTTGAGAGCAGCACCTTCCAGCGCCGCCGCTGCC
GCCGCCGAGGTTGAGCAGCGCCGCAGCCCCGGGCTCTGCCCCCCGCCGCTGGAGCTGCTGCTGCTGCTGC
TGTTCAGCCTCGGGCTGCTCCACGCAGGTGACTGCCAACAGCCAGCCCAATGTCGAATCCAGAAATGCAC
CACGGACTTCGTGTCCCTGACTTCTCACCTGAACTCTGCCGTTGACGGCTTTGACTCTGAGTTTTGCAAG
GCCTTGCGTGCCTATGCTGGCTGCACCCAGCGAACTTCAAAAGCCTGCCGTGGCAACCTGGTATACCATT
CTGCCGTGTTGGGTATCAGTGACCTCATGAGCCAGAGGAATTGTTCCAAGGATGGACCCACATCCTCTAC
CAACCCCGAAGTGACCCATGATCCTTGCAACTATCACAGCCACGCTGGAGCCAGGGAACACAGGAGAGGG
GACCAGAACCCTCCCAGTTACCTTTTTTGTGGCTTGTTTGGAGATCCTCACCTCAGAACTTTCAAGGATA
ACTTCCAAACATGCAAAGTAGAAGGGGCCTGGCCACTCATAGATAATAATTATCTTTCAGTTCAAGTGAC
AAACGTACCTGTGGTCCCTGGATCCAGTGCTACTGCTACAAATAAGATCACTATTATCTTCAAAGCCCAC
CATGAGTGTACAGATCAGAAAGTCTACCAAGCTGTGACAGATGACCTGCCGGCCGCCTTTGTGGATGGCA
CCACCAGTGGTGGGGACAGCGATGCCAAGAGCCTGCGTATCGTGGAAAGGGAGAGTGGCCACTATGTGGA
GATGCACGCCCGCTATATAGGGACCACAGTGTTTGTGCGGCAGGTGGGTCGCTACCTGACCCTTGCCATC
CGTATGCCTGAAGACCTGGCCATGTCCTACGAGGAGAGCCAGGACCTGCAGCTGTGCGTGAACGGCTGCC
CCCTGAGTGAACGCATCGATGACGGGCAGGGCCAGGTGTCTGCCATCCTGGGACACAGCCTGCCTCGCAC
CTCCTTGGTGCAGGCCTGGCCTGGCTACACACTGGAGACTGCCAACACTCAATGCCATGAGAAGATGCCA
GTGAAGGACATCTATTTCCAGTCCTGTGTCTTCGACCTGCTCACCACTGGTGATGCCAACTTTACTGCCG
CAGCCCACAGTGCCTTGGAGGATGTGGAGGCCCTGCACCCAAGGAAGGAACGCTGGCACATTTTCCCCAG
CAGTGGCAATGGGACTCCCCGTGGAGGCAGTGATTTGTCTGTCAGTCTAGGACTCACCTGCTTGATCCTT
ATCGTGTTTTTGTAGGGGTTGTCTTTTGTTTTGGTTTTTTATTTTTTGTCTATAACAAAATTTTAAAATA
TATATTGTCATAATATATTGAGTAAAAGAGTATATATGTATATACCATGTATATGACAGGATGTTTGTCC
TGGGACACCCACCAGATTGTACATACTGTGTTTGGCTGTTTTCACATATGTTGGATGTAGTGTTCTTTGA
TTGTATCAATTTTGTTTTGCAGTTCTGTGAAATGTTTTATAATGTCCCTGCCCAGGGACCTGTTAGAAAG
CACTTTATTTTTTATATATTAAATATTTATGTGTGTGCTTGGTTGATATGTATAGTACATATACACAGAC
ATCCATATGCAGCGTTTCCTTTGAAGGTGACCAGTTGTTTGTAGCTATTCTTGGCTGTACCTTCCTGCCC
TTTCCCATTGCTACTGATTTGCCACGGTGTGCAGCTTTTACTCGCCACCTTCCGGTGGAGCTGCCTCGTT
CCTTTGAACTATGCCCTCACCCTTCTGCCCTCACTTGATTTGAAAGGGTCGTTAACTCTCCCTTACAGGT
GCTTTGACTCTTAAACGCTGATCTTAAGAAGCTCTCTTCATCTAAGAGCTGTTACTTTTTCAGAAGGGGG
GGTATTATTGGTATTCTGATTACTCTCAATTCTAATTGTTATATATTTGAGCCCATACAGTGTATTAGGT
TGAACCATAGAAACTGCTATTCTCGTAGGTCAAAAGGGTCTAGTGATGGAAGTTTTGTAGATAAGTACCA
GGCATCTCAGTAACTCCTAGACTTTTTCTCATCCCATGCCCCGTTTTAAATTGTCAGTTTTCCCTCTGAC
TCTTCTGTGTTAAAACATGAAACTATAAATTTAGTAATTATCATGCCTTGCTCTTTTTAATCTATATGAC
TGATGCAAGCCCCTCTTCTTAACCGTTTCTTGGCTTTGAGCCCAGAAACACAGCTCTCCCTGTCTCCAAC
TCCAGTAAGCCCTCCTCAGCCTCACCTTACGAATCCAAAGAACTGGGGTTTGTTAGGTTCTTTCTCTAAT
GTAGAGGCCCAGATCCCATCACAAAGTTTTTCATTCTTCCTTGTCCACCATGATCTTCATCACAGTCTTT
GATATGTCTGCATGCAAAGTGGAACAGAGTTGGGCGGCAATGACAGAAGAGCTTCCTTGGCCTGACTCGG
TGTGCGGCCACTTCGGCACTGCTTAATCCAGATATTCTTGTTAACTAAGCATTGTGCTTCCCAGGTGGTC
TGAAGTCAGGTACTCTCTCTCTCAACACCTGTAGTTGAATATGATTTGGTCAGTTGCTCGTTGTAACTTG
GAGAAATTCCTATAAAGTAAGATCTCCTTGCCTCTTCCATCCATTGTTGGCACCCCCTTGCAAAAGGAAA
AGAACAGCAAAAGTCAGGAGCAGTAATCTGAGAAAGTTAACTCCAGGATAGGTAGGTTTCTATTGTTATA
GCTAGATGTAAATCTTTAGTTCCAAGAAGTGATAGAGTTTCTGCTTTAATAATTTGTTGATAAGTTTACA
TAAACAGAAATAAAAGATACTATCTTTACCGTAGTAGTTCAGGCCAAGATTATGCTTAGTTTTAGTTCTC
CAGGTAGTTACTTTTGCCATGTCCTATTGATCAGTGACACTGCCAGAGGCCCATACCGGCAAGAGGAAGA
GGACGTCATTTTGTAAAGTTTAACTTCTTAGCGAACTGATGTGCCACCCAGTCACAGAGTGGAGTTGTGA
ATTCATGTAGAGGTGGCAAACCTCTACCTTGTGTTGATGAGAGAATAATCTTGGGCAGTCTGGGAAAATA
AGGAAGGCATCTCCTTCTTACTCATGGAGATTCAACTATAGAGAGTTGAAACCTAAACCCGCCTTCCTTT
TATAGAAGCTGGACTAGAGACGGACTGACCATCAGCTCTGAACTGTGGCTTTTTTTGTTCACCTATGATG
CCATGTACCAAATTCAGAAGCTATCGTTAATAATTTGTTTTATAATTGAGTAGTACAAGCGAGGAAAAAA
TACGGAGGATAACCACTATTTTTGTGCAAATAATATGAAAGTGAAGTAAAAGCAATAGAAGAAATTTCTA
TAGGATCTGGGTTTAGAGTGTGTATCATTAATAAATATACCTTTGCTCTTTTCAGGGAAAATAACAACCA
CCCTTACTGATAGTTGGGAAAAGAAGATTGGGTTATTTTGCCATATCATTTAGCTGGAAGTGACATTTAA
AAGCACCCTGCATCACTAGTAATAGTGTATTTTGCTATTCTGCCCTTGTAATCGGTGTCCCTGTAAAACA
ATCCCCACAGATTACTTTCAGAAATAGATGTATTTCTCTACGTAAGGGCCAGGTTTATTTTCTCCTTTTT
TGAGATTTCTAGAAAAAATGCTGCTTGCACATGTTGGTTCTTGAAACCTTAGCTAGAAGAATTTCAGGTC
ATACCAACATGTGGATAGGCTATAGCTGTTCAGAGGTCTCCTGGGGGAGCTTAAAACGGGGGAAACACTG
GTTTTCACAGATGCTCCACATGGCTGTCTTTAAAAGACTCAAAACTTTTTTTTGTCCTCTTTGTTATGCT
TGGAAGCTCCCCCCCCCCCAACAGTGTGTCGAGTCTTTGCAAAGAAACCTTTAGATGTGGTTCATAGATA
TATGAATACGTATCTGTGTAAAACAGTGAGTGTGCAGTGTGTAAATACTTTAAATTATTATGCTAGAAAA
ATAAAGTTACATACCTTGCTGTGGAA
>XM_047417125.1 RGMB [organism = Homo sapiens] [GeneID = 285704]
[transcript = X3]
(SEQ ID NO: 13)
GCCTTCCCTCCGCGCCTCGGCCGCGTGGCTTGCGGCTTATTTTCCCAGCTGGCAAGCGTCGCGCTGCAGA
CAAGGGAATGCCTGTGGTGAGTTGTTTATTTTGTTGAAGTTTGCGGTGGGCCCGGGGGACGTGGGGGGCG
AGCGGCAGGCAGCGGCCGGGACTGTTTTGTTTAGGTCTCCATCCACCTGTGGGGTATAAATCTGCCAGCC
TCAAGGCGGCGGGGGACCGGAGTGAGAAAGCTCTGACGCTCCCAAAAGCCCCTTTTCGCAACTTACAGAT
CTAGGACCACATTTGATTCTCCACGCCTGGAACCGGCTAGATCCTAGTTTACGCTCACCCCGCCCTCCAG
CCTCCCATCAACGCTTCCCGCACCCGCTGTGTGCAGGGCTGGCGCTCCGCAGAGCAAACAGCGCCCACGA
CCCAGGGACAGCTGGCCACCGAAGTCCCTCCCCGGCGCCCTTCCCCGTTCCTTGAGGATAGGGGAGCTGA
GGTTTTCATGTGAAGGGCGCCCATGAAATTCGTGGGAACTTGTTCTGGCGTCTTCTGGAGTGGAGTTCTT
TCAGGGGTTTCGGGTCTGCCCGGGACTACAAAATGGGATCCTGGGTTGGCTGGAGTTTCCCAGACTAGGC
TGGAGAGAGGTCGGCTGTTGGCTCGCGGGAGAGCCCAGAGGAACCCCTTGGGCGATGGCAGTCAAGACCG
GCGGCTGCGTTTGCCTGGAAGAAAAGCCTAAATACATTAGCGAGCTGGTAAAGCTTTTAAGGCCTTCTTG
GGAGCGAGTGGCTGGCTAATGAGAGGTTAATTTTGTTTCGAAGGGAAGGCTGCACTGGTTTCCTGAGATA
AATGGACAGGAAGCGCATTACCTCCGCGCGCTGGCAAGCGGCTCCCAAATGCTTTTTGCACCTGCCGTTT
TGAGCGGTGAGAGCAGCGGCGGAGCCCGCAGGGATTGCGGGGGCCCGTTGTCTACGAGCGGGTGATGAGC
CCCCCGCAAGCCCCGGGCTCTCTCCCTCCGCGAGTGCATTGCTCACGCCAAGCTTTGGGACTCTTCTCTC
ACACCCAGGTCCTGCGTGTTCCGAAGTTCAGGGGCCCCCTGATCCCGCTGAGCCCCCTCCAGGAGCGAAA
GGGTTAAGAATGATGTAAGTACAGGCACTTGATTTCTCAAGTCGCCCGCTTGGCCCTCTTCCAAGTTGTA
CTTTCCTTATTTTCTCTCCTTTCCTTTCCTTACTGTTCTAACGCTTTTTAATAAACTTTCACTCTTGCTC
TGAAAAAAAAAATGATGTAAGTACAAACCACGAGCTGCTTGACGCTGAGCTGTTGGACTCCGGGCTATTT
CAGCCGCTGCTTTGGGTACACAGTGGCCGGGGAGCAGCCGGGGAGCCCTTACTCTAGAAAGGATTGCACA
AATCGCAAGCTTAGGTGATCTGTTAAGCAGTCAGAGATTCTCCTGTTAGATTTCTGGAAATGGAGACATA
TTAACTGGAGAACAGTTAAAAACATCACGTATAGGCTCAGGAAATGAAACTGCATCTTGTTTAGAATCCA
TTAGTGTTTCTGGCATGTGTTTTAATTCTGTCCACTTGCACGTTCATGCCGTAGTATGCTTTGCTGGATT
CTCCGGATGTACTGTTCAGGTCTCCTCCTTCCTTTCTGAAAACAAAGAGAAAGGGAGAAAGAGTACCAGT
GGCAAGAAACAGGGTGGATGTGTGAATTACACAAGTCATCAGCCACTTCATGATTTTTTTTCTGATTAAA
ACATAAAACTTTTATAAGTCACCGATTTTTTTGTTTGTGTTCTAGCTCCCTCCCCGATTTTTCTTTGTAC
AATATAAAGTCGTTTGTAACTTGCAGCTCACGGGCTTGAGGAGTGGCTGCGGTATAAGCGACTATTCACT
ACGCTCTTGCTTTCCAAGCCAATTCCTGATCTTGTGAACAGTACGAATTTACCCGCGAGTGTGTCAACTG
ACCTATGTCTTGAAAATAGGAACCACCTTGCGGTTTACTTGTGAAAATTAGCATTTTCCTCTAGCGGCTG
AACACTTATGCAGCAATAAAAAGTGAATTGTTAGGCTTATAAATCTCGAGCTTGTCAAATTTTACAACAG
AGGAGCAATAAAGCACCTTATAGAGATGAAGTTTTTCCTCTGAAGAACAGTTCCGTTTAACGACACCAGG
ATTTGCAATGACTCGAGGACGCCCTCTATTGATAGGAAAATCTAGAGCACCCGTGTACTTATGGAACAGG
ATATAGGGGGAGGGGAGTAAAAAATCATAGAAAGTCACGAGTGGAAAGACCCAAACCAAACAGAAATTAT
TGATCCTTAACTATTGAGTATATGCTTTGAAGTATGATCAATAAAGTGAGATGCTGCTTTTATAGGGGTC
GAATTAAATCTTGGGTTAAGAGAAAAAAATGTAGTCTTTGTTAGCCAAATACTTTGAAAATCAGTATTAT
GTTTGATTACTTTTTTAAGATGCTTCTTAGTATGTCATGAACTGTATCTAATTATATGAAGTAATTGCAA
GATAGTAAATTTTTGCTCTAATAATACTTAGTCCGCAGCATTATCTAAAAATGTCATATAACTTCCTCCC
CAAAAGTCATTTCTCCAGGCAACTGTGAAGCTATTATTAAGCGTATTATGGATATACACAGGCTTGGATT
ATATTGTGGATAGTGAAAGGTGTGTTTACCCCTTAAGTCATCATAGTATGTAACAAGTAAGTAATGAAAA
GTTACAGTAAAAACAGATAGGCATCCTTCATTAGGGACCTAGTGGTTTGGGCAATTCAAAATGTGTCTAA
TAAGTGAGCTGGTGAGACTGAAGGTTTGCCCCTCAATTATCCTAGTCCATTGATTAGCTCATTTAAACAT
GTGTGGAATATCAGCATGTCCGTGGTTGATAAGAAATTCCTAATTAGAGATTCTGAGTAACATAGCGGGT
ACAGTTGCCAACTACGATTTGGATGTTAGTGGATTTTAAATGGCAACCCTGTGCTGTTTGGGGAAAGGCA
AAGGCGAGGGCGGTATCAAGTGTCTCTCAGCCTTGAGGTCATTCTTGAGATTAGATACCACTTCACACCT
ACCGATAACAAATTGGAATCTATCATCAGTCTCATAACTGCTTTTCTGTCTCCACTTGGCAGCCTCTTTC
TTTGTAGACAATTGTCTTTTTCCTCACTTTCGGACCCATCCGATCATAAAAGGAGAAAGTGAGCTGCCAT
TGAATTAATCCGTAGAAATTTGTGGAAAATTTAAATAATCTGTAGAAATTTGTGAAAAAGCCACTGAGCA
ACAACAAATCGTGGCATAATTACACTGCATAAAAACCCACCACTCTCATTAATTACACTGTAACCGAACG
TTGAAGTTTGCCCACTCTTAGACCTCTCCTTGCCGCTTTCAGGAAGGGAAATGTCAAACTAGAACACCTG
AGCCTCGCACAGCTGCCAGCGAATTCCGGCGGCAGTTCGCGGGTACCTTCCCAGCCCATCCTATCTGCTC
CGGACCGCGGGTGACATTCAGATTTCCAACACCGCCCCGAGCTTGCTTTACCAGACACCCTCAAACGACT
CGGAACGCGGTTCCGCAGATGTGATTGGCTGAAAGGGGACCTGCCGCGCGGCGCTCTGTAAAAGCGCCTG
CTTCCTCCAAGGAGGTGGAGCTGAAGCTGGCAGGGCCAGGGCAGGGAGGGGTAATTAAATTAAAAAAAAA
ACTTGGAGGGAAGATGAATCGGATCACATTAATTCAAGGAGAGGTCAAAGGCCGGTACCTTTGTAACGCC
GGCGAGAGTAAGACTTGCGGTCGCCCATTTCTGTTGGGCGCGAGGAAACAGTAAATCGCGGAAGGGGGTA
GAGGGGGAAGGAAAGGGAGCGCGAGGCAGCATGCGGCGCAGGAGAGAGGACTTGGAGTCGATGGACAGGG
AACTCGAATTACACTGAGCGGGGGGGGGAGAGGGCCCTCTGGGTCCTGCGATTGGCAGGCTGAGTCGGGC
TGACAAGGAAGAAGAGGAAGCGAAGCGCGCCCCCCGGCCCATGCCGCAGCCACGGGCCCAGACCCGCCAC
GGCGCCCGCGCCGCCGCCCTCGCCGGAGCCCACGAGACCTGCATGGACGGGCATGGGCTTGAGAGCAGCA
CCTTCCAGCGCCGCCGCTGCCGCCGCCGAGGTTGAGCAGCGCCGCAGCCCCGGGCTCTGCCCCCCGCCGC
TGGAGCTGCTGCTGCTGCTGCTGTTCAGCCTCGGGCTGCTCCACGCAGGTGACTGCCAACAGCCAGCCCA
ATGTCGAATCCAGAAATGCACCACGGACTTCGTGTCCCTGACTTCTCACCTGAACTCTGCCGTTGACGGC
TTTGACTCTGAGTTTTGCAAGGCCTTGCGTGCCTATGCTGGCTGCACCCAGCGAACTTCAAAAGCCTGCC
GTGGCAACCTGGTATACCATTCTGCCGTGTTGGGTATCAGTGACCTCATGAGCCAGAGGAATTGTTCCAA
GGATGGACCCACATCCTCTACCAACCCCGAAGTGACCCATGATCCTTGCAACTATCACAGCCACGCTGGA
GCCAGGGAACACAGGAGAGGGGACCAGAACCCTCCCAGTTACCTTTTTTGTGGCTTGTTTGGAGATCCTC
ACCTCAGAACTTTCAAGGATAACTTCCAAACATGCAAAGTAGAAGGGGCCTGGCCACTCATAGATAATAA
TTATCTTTCAGTTCAAGTGACAAACGTACCTGTGGTCCCTGGATCCAGTGCTACTGCTACAAATAAGATC
ACTATTATCTTCAAAGCCCACCATGAGTGTACAGATCAGAAAGTCTACCAAGCTGTGACAGATGACCTGC
CGGCCGCCTTTGTGGATGGCACCACCAGTGGTGGGGACAGCGATGCCAAGAGCCTGCGTATCGTGGAAAG
GGAGAGTGGCCACTATGTGGAGATGCACGCCCGCTATATAGGGACCACAGTGTTTGTGCGGCAGGTGGGT
CGCTACCTGACCCTTGCCATCCGTATGCCTGAAGACCTGGCCATGTCCTACGAGGAGAGCCAGGACCTGC
AGCTGTGCGTGAACGGCTGCCCCCTGAGTGAACGCATCGATGACGGGCAGGGCCAGGTGTCTGCCATCCT
GGGACACAGCCTGCCTCGCACCTCCTTGGTGCAGGCCTGGCCTGGCTACACACTGGAGACTGCCAACACT
CAATGCCATGAGAAGATGCCAGTGAAGGACATCTATTTCCAGTCCTGTGTCTTCGACCTGCTCACCACTG
GTGATGCCAACTTTACTGCCGCAGCCCACAGTGCCTTGGAGGATGTGGAGGCCCTGCACCCAAGGAAGGA
ACGCTGGCACATTTTCCCCAGCAGTGGCAATGGGACTCCCCGTGGAGGCAGTGATTTGTCTGTCAGTCTA
GGACTCACCTGCTTGATCCTTATCGTGTTTTTGTAGGGGTTGTCTTTTGTTTTGGTTTTTTATTTTTTGT
CTATAACAAAATTTTAAAATATATATTGTCATAATATATTGAGTAAAAGAGTATATATGTATATACCATG
TATATGACAGGATGTTTGTCCTGGGACACCCACCAGATTGTACATACTGTGTTTGGCTGTTTTCACATAT
GTTGGATGTAGTGTTCTTTGATTGTATCAATTTTGTTTTGCAGTTCTGTGAAATGTTTTATAATGTCCCT
GCCCAGGGACCTGTTAGAAAGCACTTTATTTTTTATATATTAAATATTTATGTGTGTGCTTGGTTGATAT
GTATAGTACATATACACAGACATCCATATGCAGCGTTTCCTTTGAAGGTGACCAGTTGTTTGTAGCTATT
CTTGGCTGTACCTTCCTGCCCTTTCCCATTGCTACTGATTTGCCACGGTGTGCAGCTTTTACTCGCCACC
TTCCGGTGGAGCTGCCTCGTTCCTTTGAACTATGCCCTCACCCTTCTGCCCTCACTTGATTTGAAAGGGT
CGTTAACTCTCCCTTACAGGTGCTTTGACTCTTAAACGCTGATCTTAAGAAGCTCTCTTCATCTAAGAGC
TGTTACTTTTTCAGAAGGGGGGGTATTATTGGTATTCTGATTACTCTCAATTCTAATTGTTATATATTTG
AGCCCATACAGTGTATTAGGTTGAACCATAGAAACTGCTATTCTCGTAGGTCAAAAGGGTCTAGTGATGG
AAGTTTTGTAGATAAGTACCAGGCATCTCAGTAACTCCTAGACTTTTTCTCATCCCATGCCCCGTTTTAA
ATTGTCAGTTTTCCCTCTGACTCTTCTGTGTTAAAACATGAAACTATAAATTTAGTAATTATCATGCCTT
GCTCTTTTTAATCTATATGACTGATGCAAGCCCCTCTTCTTAACCGTTTCTTGGCTTTGAGCCCAGAAAC
ACAGCTCTCCCTGTCTCCAACTCCAGTAAGCCCTCCTCAGCCTCACCTTACGAATCCAAAGAACTGGGGT
TTGTTAGGTTCTTTCTCTAATGTAGAGGCCCAGATCCCATCACAAAGTTTTTCATTCTTCCTTGTCCACC
ATGATCTTCATCACAGTCTTTGATATGTCTGCATGCAAAGTGGAACAGAGTTGGGCGGCAATGACAGAAG
AGCTTCCTTGGCCTGACTCGGTGTGCGGCCACTTCGGCACTGCTTAATCCAGATATTCTTGTTAACTAAG
CATTGTGCTTCCCAGGTGGTCTGAAGTCAGGTACTCTCTCTCTCAACACCTGTAGTTGAATATGATTTGG
TCAGTTGCTCGTTGTAACTTGGAGAAATTCCTATAAAGTAAGATCTCCTTGCCTCTTCCATCCATTGTTG
GCACCCCCTTGCAAAAGGAAAAGAACAGCAAAAGTCAGGAGCAGTAATCTGAGAAAGTTAACTCCAGGAT
AGGTAGGTTTCTATTGTTATAGCTAGATGTAAATCTTTAGTTCCAAGAAGTGATAGAGTTTCTGCTTTAA
TAATTTGTTGATAAGTTTACATAAACAGAAATAAAAGATACTATCTTTACCGTAGTAGTTCAGGCCAAGA
TTATGCTTAGTTTTAGTTCTCCAGGTAGTTACTTTTGCCATGTCCTATTGATCAGTGACACTGCCAGAGG
CCCATACCGGCAAGAGGAAGAGGACGTCATTTTGTAAAGTTTAACTTCTTAGCGAACTGATGTGCCACCC
AGTCACAGAGTGGAGTTGTGAATTCATGTAGAGGTGGCAAACCTCTACCTTGTGTTGATGAGAGAATAAT
CTTGGGCAGTCTGGGAAAATAAGGAAGGCATCTCCTTCTTACTCATGGAGATTCAACTATAGAGAGTTGA
AACCTAAACCCGCCTTCCTTTTATAGAAGCTGGACTAGAGACGGACTGACCATCAGCTCTGAACTGTGGC
TTTTTTTGTTCACCTATGATGCCATGTACCAAATTCAGAAGCTATCGTTAATAATTTGTTTTATAATTGA
GTAGTACAAGCGAGGAAAAAATACGGAGGATAACCACTATTTTTGTGCAAATAATATGAAAGTGAAGTAA
AAGCAATAGAAGAAATTTCTATAGGATCTGGGTTTAGAGTGTGTATCATTAATAAATATACCTTTGCTCT
TTTCAGGGAAAATAACAACCACCCTTACTGATAGTTGGGAAAAGAAGATTGGGTTATTTTGCCATATCAT
TTAGCTGGAAGTGACATTTAAAAGCACCCTGCATCACTAGTAATAGTGTATTTTGCTATTCTGCCCTTGT
AATCGGTGTCCCTGTAAAACAATCCCCACAGATTACTTTCAGAAATAGATGTATTTCTCTACGTAAGGGC
CAGGTTTATTTTCTCCTTTTTTGAGATTTCTAGAAAAAATGCTGCTTGCACATGTTGGTTCTTGAAACCT
TAGCTAGAAGAATTTCAGGTCATACCAACATGTGGATAGGCTATAGCTGTTCAGAGGTCTCCTGGGGGAG
CTTAAAACGGGGGAAACACTGGTTTTCACAGATGCTCCACATGGCTGTCTTTAAAAGACTCAAAACTTTT
TTTTGTCCTCTTTGTTATGCTTGGAAGCTCCCCCCCCCCCAACAGTGTGTCGAGTCTTTGCAAAGAAACC
TTTAGATGTGGTTCATAGATATATGAATACGTATCTGTGTAAAACAGTGAGTGTGCAGTGTGTAAATACT
TTAAATTATTATGCTAGAAAAATAAAGTTACATACCTTGCTGTGGAA
>XM_011543345.3 RGMB [organism = Homo sapiens] [GeneID = 285704]
[transcript = X2]
(SEQ ID NO: 14)
GCCTTCCCTCCGCGCCTCGGCCGCGTGGCTTGCGGCTTATTTTCCCAGCTGGCAAGCGTCGCGCTGCAGA
CAAGGGAATGCCTGTGGTGAGTTGTTTATTTTGTTGAAGTTTGCGGTGGGCCCGGGGGACGTGGGGGGCG
AGCGGCAGGCAGCGGCCGGGACTGTTTTGTTTAGGTCTCCATCCACCTGTGGGGTATAAATCTGCCAGCC
TCAAGGCGGCGGGGGACCGGAGTGAGAAAGCTCTGACGCTCCCAAAAGCCCCTTTTCGCAACTTACAGAT
CTAGGACCACATTTGATTCTCCACGCCTGGAACCGGCTAGATCCTAGTTTACGCTCACCCCGCCCTCCAG
CCTCCCATCAACGCTTCCCGCACCCGCTGTGTGCAGGGCTGGCGCTCCGCAGAGCAAACAGCGCCCACGA
CCCAGGGACAGCTGGCCACCGAAGTCCCTCCCCGGCGCCCTTCCCCGTTCCTTGAGGATAGGGGAGCTGA
GGTTTTCATGTGAAGGGCGCCCATGAAATTCGTGGGAACTTGTTCTGGCGTCTTCTGGAGTGGAGTTCTT
TCAGGGGTTTCGGGTCTGCCCGGGACTACAAAATGGGATCCTGGGTTGGCTGGAGTTTCCCAGACTAGGC
TGGAGAGAGGTCGGCTGTTGGCTCGCGGGAGAGCCCAGAGGAACCCCTTGGGCGATGGCAGTCAAGACCG
GCGGCTGCGTTTGCCTGGAAGAAAAGCCTAAATACATTAGCGAGCTGGTCCTGCGTGTTCCGAAGTTCAG
GGGCCCCCTGATCCCGCTGAGCCCCCTCCAGGAGCGAAAGGGTTAAGAATGATAAGGAAGAAGAGGAAGC
GAAGCGCGCCCCCCGGCCCATGCCGCAGCCACGGGCCCAGACCCGCCACGGCGCCCGCGCCGCCGCCCTC
GCCGGAGCCCACGAGACCTGCATGGACGGGCATGGGCTTGAGAGCAGCACCTTCCAGCGCCGCCGCTGCC
GCCGCCGAGGTTGAGCAGCGCCGCAGCCCCGGGCTCTGCCCCCCGCCGCTGGAGCTGCTGCTGCTGCTGC
TGTTCAGCCTCGGGCTGCTCCACGCAGGTGACTGCCAACAGCCAGCCCAATGTCGAATCCAGAAATGCAC
CACGGACTTCGTGTCCCTGACTTCTCACCTGAACTCTGCCGTTGACGGCTTTGACTCTGAGTTTTGCAAG
GCCTTGCGTGCCTATGCTGGCTGCACCCAGCGAACTTCAAAAGCCTGCCGTGGCAACCTGGTATACCATT
CTGCCGTGTTGGGTATCAGTGACCTCATGAGCCAGAGGAATTGTTCCAAGGATGGACCCACATCCTCTAC
CAACCCCGAAGTGACCCATGATCCTTGCAACTATCACAGCCACGCTGGAGCCAGGGAACACAGGAGAGGG
GACCAGAACCCTCCCAGTTACCTTTTTTGTGGCTTGTTTGGAGATCCTCACCTCAGAACTTTCAAGGATA
ACTTCCAAACATGCAAAGTAGAAGGGGCCTGGCCACTCATAGATAATAATTATCTTTCAGTTCAAGTGAC
AAACGTACCTGTGGTCCCTGGATCCAGTGCTACTGCTACAAATAAGATCACTATTATCTTCAAAGCCCAC
CATGAGTGTACAGATCAGAAAGTCTACCAAGCTGTGACAGATGACCTGCCGGCCGCCTTTGTGGATGGCA
CCACCAGTGGTGGGGACAGCGATGCCAAGAGCCTGCGTATCGTGGAAAGGGAGAGTGGCCACTATGTGGA
GATGCACGCCCGCTATATAGGGACCACAGTGTTTGTGCGGCAGGTGGGTCGCTACCTGACCCTTGCCATC
CGTATGCCTGAAGACCTGGCCATGTCCTACGAGGAGAGCCAGGACCTGCAGCTGTGCGTGAACGGCTGCC
CCCTGAGTGAACGCATCGATGACGGGCAGGGCCAGGTGTCTGCCATCCTGGGACACAGCCTGCCTCGCAC
CTCCTTGGTGCAGGCCTGGCCTGGCTACACACTGGAGACTGCCAACACTCAATGCCATGAGAAGATGCCA
GTGAAGGACATCTATTTCCAGTCCTGTGTCTTCGACCTGCTCACCACTGGTGATGCCAACTTTACTGCCG
CAGCCCACAGTGCCTTGGAGGATGTGGAGGCCCTGCACCCAAGGAAGGAACGCTGGCACATTTTCCCCAG
CAGTGGCAATGGGACTCCCCGTGGAGGCAGTGATTTGTCTGTCAGTCTAGGACTCACCTGCTTGATCCTT
ATCGTGTTTTTGTAGGGGTTGTCTTTTGTTTTGGTTTTTTATTTTTTGTCTATAACAAAATTTTAAAATA
TATATTGTCATAATATATTGAGTAAAAGAGTATATATGTATATACCATGTATATGACAGGATGTTTGTCC
TGGGACACCCACCAGATTGTACATACTGTGTTTGGCTGTTTTCACATATGTTGGATGTAGTGTTCTTTGA
TTGTATCAATTTTGTTTTGCAGTTCTGTGAAATGTTTTATAATGTCCCTGCCCAGGGACCTGTTAGAAAG
CACTTTATTTTTTATATATTAAATATTTATGTGTGTGCTTGGTTGATATGTATAGTACATATACACAGAC
ATCCATATGCAGCGTTTCCTTTGAAGGTGACCAGTTGTTTGTAGCTATTCTTGGCTGTACCTTCCTGCCC
TTTCCCATTGCTACTGATTTGCCACGGTGTGCAGCTTTTACTCGCCACCTTCCGGTGGAGCTGCCTCGTT
CCTTTGAACTATGCCCTCACCCTTCTGCCCTCACTTGATTTGAAAGGGTCGTTAACTCTCCCTTACAGGT
GCTTTGACTCTTAAACGCTGATCTTAAGAAGCTCTCTTCATCTAAGAGCTGTTACTTTTTCAGAAGGGGG
GGTATTATTGGTATTCTGATTACTCTCAATTCTAATTGTTATATATTTGAGCCCATACAGTGTATTAGGT
TGAACCATAGAAACTGCTATTCTCGTAGGTCAAAAGGGTCTAGTGATGGAAGTTTTGTAGATAAGTACCA
GGCATCTCAGTAACTCCTAGACTTTTTCTCATCCCATGCCCCGTTTTAAATTGTCAGTTTTCCCTCTGAC
TCTTCTGTGTTAAAACATGAAACTATAAATTTAGTAATTATCATGCCTTGCTCTTTTTAATCTATATGAC
TGATGCAAGCCCCTCTTCTTAACCGTTTCTTGGCTTTGAGCCCAGAAACACAGCTCTCCCTGTCTCCAAC
TCCAGTAAGCCCTCCTCAGCCTCACCTTACGAATCCAAAGAACTGGGGTTTGTTAGGTTCTTTCTCTAAT
GTAGAGGCCCAGATCCCATCACAAAGTTTTTCATTCTTCCTTGTCCACCATGATCTTCATCACAGTCTTT
GATATGTCTGCATGCAAAGTGGAACAGAGTTGGGCGGCAATGACAGAAGAGCTTCCTTGGCCTGACTCGG
TGTGCGGCCACTTCGGCACTGCTTAATCCAGATATTCTTGTTAACTAAGCATTGTGCTTCCCAGGTGGTC
TGAAGTCAGGTACTCTCTCTCTCAACACCTGTAGTTGAATATGATTTGGTCAGTTGCTCGTTGTAACTTG
GAGAAATTCCTATAAAGTAAGATCTCCTTGCCTCTTCCATCCATTGTTGGCACCCCCTTGCAAAAGGAAA
AGAACAGCAAAAGTCAGGAGCAGTAATCTGAGAAAGTTAACTCCAGGATAGGTAGGTTTCTATTGTTATA
GCTAGATGTAAATCTTTAGTTCCAAGAAGTGATAGAGTTTCTGCTTTAATAATTTGTTGATAAGTTTACA
TAAACAGAAATAAAAGATACTATCTTTACCGTAGTAGTTCAGGCCAAGATTATGCTTAGTTTTAGTTCTC
CAGGTAGTTACTTTTGCCATGTCCTATTGATCAGTGACACTGCCAGAGGCCCATACCGGCAAGAGGAAGA
GGACGTCATTTTGTAAAGTTTAACTTCTTAGCGAACTGATGTGCCACCCAGTCACAGAGTGGAGTTGTGA
ATTCATGTAGAGGTGGCAAACCTCTACCTTGTGTTGATGAGAGAATAATCTTGGGCAGTCTGGGAAAATA
AGGAAGGCATCTCCTTCTTACTCATGGAGATTCAACTATAGAGAGTTGAAACCTAAACCCGCCTTCCTTT
TATAGAAGCTGGACTAGAGACGGACTGACCATCAGCTCTGAACTGTGGCTTTTTTTGTTCACCTATGATG
CCATGTACCAAATTCAGAAGCTATCGTTAATAATTTGTTTTATAATTGAGTAGTACAAGCGAGGAAAAAA
TACGGAGGATAACCACTATTTTTGTGCAAATAATATGAAAGTGAAGTAAAAGCAATAGAAGAAATTTCTA
TAGGATCTGGGTTTAGAGTGTGTATCATTAATAAATATACCTTTGCTCTTTTCAGGGAAAATAACAACCA
CCCTTACTGATAGTTGGGAAAAGAAGATTGGGTTATTTTGCCATATCATTTAGCTGGAAGTGACATTTAA
AAGCACCCTGCATCACTAGTAATAGTGTATTTTGCTATTCTGCCCTTGTAATCGGTGTCCCTGTAAAACA
ATCCCCACAGATTACTTTCAGAAATAGATGTATTTCTCTACGTAAGGGCCAGGTTTATTTTCTCCTTTTT
TGAGATTTCTAGAAAAAATGCTGCTTGCACATGTTGGTTCTTGAAACCTTAGCTAGAAGAATTTCAGGTC
ATACCAACATGTGGATAGGCTATAGCTGTTCAGAGGTCTCCTGGGGGAGCTTAAAACGGGGGAAACACTG
GTTTTCACAGATGCTCCACATGGCTGTCTTTAAAAGACTCAAAACTTTTTTTTGTCCTCTTTGTTATGCT
TGGAAGCTCCCCCCCCCCCAACAGTGTGTCGAGTCTTTGCAAAGAAACCTTTAGATGTGGTTCATAGATA
TATGAATACGTATCTGTGTAAAACAGTGAGTGTGCAGTGTGTAAATACTTTAAATTATTATGCTAGAAAA
ATAAAGTTACATACCTTGCTGTGGAA
>XM_011543346.3 RGMB [organism = Homo sapiens] [GeneID = 285704]
[transcript = X4]
(SEQ ID NO: 15)
GCAGAGATATCCGGGCCGCCGGTGGGTGGTCGCTAGGGCTGGGCCAGCCTCTTGGAGGTCCACGCCCGCC
GAGCCCACGCTGGCTGGGGCCGGGGTGCCGGCGCGCTCGGGACTCGTCTCAGCAGTCGCTCACGGTCTTT
GTGTCTTCTCTTCCGCCCCTTTCCCTGCCTGCCGCCTCCGGCCGCCACGATGCCCCTGCGCCCCGCTGCT
GCCGCCGCGGACTGGCTGCGCCGGCTGCGCGCTGCTTGCTGCGGCGGTGGTGGCGCCCCATCTGCTACAC
GGGCCTGAAGAAGGAAGAAGAGGAAGCGAAGCGCGCCCCCCGGCCCATGCCGCAGCCACGGGCCCAGACC
CGCCACGGCGCCCGCGCCGCCGCCCTCGCCGGAGCCCACGAGACCTGCATGGACGGGCATGGGCTTGAGA
GCAGCACCTTCCAGCGCCGCCGCTGCCGCCGCCGAGGTTGAGCAGCGCCGCAGCCCCGGGCTCTGCCCCC
CGCCGCTGGAGCTGCTGCTGCTGCTGCTGTTCAGCCTCGGGCTGCTCCACGCAGGTAGGACGGGAGCGCG
CGAGGGGAGCCAGCCCCGGGGCCTAGGGCTTTGTTCCCAAATAGCCCCAGGTCTCGAAGGCGCTCGCCGG
GGCGGAAGGGCGGCGTGGCGCAACGGGAAAGGCCTCCCGAGCCCCTGACACGCACCTCTGTCGGGCTCCG
GGCCACAGTAACGCAGCCACCCACCGGGCGGCCGCCGGGCGCAGAGACGAGCCTTCTCTCCACCCTCCCC
GCCTTCGGCTCGGACCGCTGACAAATCTCCCCTCCGACAGCCGCTTATCAAACATTTAGCGGGCGGGGGC
CGCGCGCCCGAGACAAATGTTCCGGATGCTTTATTGCTCTCTGCGCTGGTGTTTACTGAAAGTCAGGCTC
GGATCAGGTTTCCGCCGTCGCAGCCGCCTCGGAGGGAGACAAGGAGCCTTGTGCTGCTTTTCTGGAGTTG
GGGGTGGCGGCGAGGCGAAGGAAGTACACCAGATGGAAAGATTTTTACCAGATTTTGCAAACCTGGGAGT
TAATCTCGAAGTAACGTCAACTTTTTTGAGCAGGGATTTCTTTCGTTTTGGCAAAAGCTGCGTTTGTTCA
GCGATCAGCCTCCCGGGAAAAGCTCGTGTTTTGTGACTAGCCGGGTGTCTTCGGGCTCCGTGAGGGGTTG
ACAGATAACCATGCTGCCTTCTGGAGGTCGGCGCTTCCCCAGCCTTGAGGCGTATGTTCCAAGGGAAGTA
AGCCTGCAAATGAGCCTTCTACCATGTGATACCTATTTTTCTTTTTAAAAAATCGTTTCATGAAAATCCA
GGACGCTCTTCACACTATTTTTTTCTAGGAATTTGCCTAGACAAAGAATTTTTTTTAATGCTAAGGTTGA
AAGGGTCATTTTCCTTTAGTCTTGAGAACAAACAGCATTAGATAAGTTTCTTTTCCTTCCAGACCTTTTT
TGTCCTGTATATATGAGACACTTCACAACGTTCTGGTCTGGAGAATCCTGAACTTCGATGCCACCCAACT
GTGACTGCCAACAGCCAGCCCAATGTCGAATCCAGAAATGCACCACGGACTTCGTGTCCCTGACTTCTCA
CCTGAACTCTGCCGTTGACGGCTTTGACTCTGAGTTTTGCAAGGCCTTGCGTGCCTATGCTGGCTGCACC
CAGCGAACTTCAAAAGCCTGCCGTGGCAACCTGGTATACCATTCTGCCGTGTTGGGTATCAGTGACCTCA
TGAGCCAGAGGAATTGTTCCAAGGATGGACCCACATCCTCTACCAACCCCGAAGTGACCCATGATCCTTG
CAACTATCACAGCCACGCTGGAGCCAGGGAACACAGGAGAGGGGACCAGAACCCTCCCAGTTACCTTTTT
TGTGGCTTGTTTGGAGATCCTCACCTCAGAACTTTCAAGGATAACTTCCAAACATGCAAAGTAGAAGGGG
CCTGGCCACTCATAGATAATAATTATCTTTCAGTTCAAGTGACAAACGTACCTGTGGTCCCTGGATCCAG
TGCTACTGCTACAAATAAGATCACTATTATCTTCAAAGCCCACCATGAGTGTACAGATCAGAAAGTCTAC
CAAGCTGTGACAGATGACCTGCCGGCCGCCTTTGTGGATGGCACCACCAGTGGTGGGGACAGCGATGCCA
AGAGCCTGCGTATCGTGGAAAGGGAGAGTGGCCACTATGTGGAGATGCACGCCCGCTATATAGGGACCAC
AGTGTTTGTGCGGCAGGTGGGTCGCTACCTGACCCTTGCCATCCGTATGCCTGAAGACCTGGCCATGTCC
TACGAGGAGAGCCAGGACCTGCAGCTGTGCGTGAACGGCTGCCCCCTGAGTGAACGCATCGATGACGGGC
AGGGCCAGGTGTCTGCCATCCTGGGACACAGCCTGCCTCGCACCTCCTTGGTGCAGGCCTGGCCTGGCTA
CACACTGGAGACTGCCAACACTCAATGCCATGAGAAGATGCCAGTGAAGGACATCTATTTCCAGTCCTGT
GTCTTCGACCTGCTCACCACTGGTGATGCCAACTTTACTGCCGCAGCCCACAGTGCCTTGGAGGATGTGG
AGGCCCTGCACCCAAGGAAGGAACGCTGGCACATTTTCCCCAGCAGTGGCAATGGGACTCCCCGTGGAGG
CAGTGATTTGTCTGTCAGTCTAGGACTCACCTGCTTGATCCTTATCGTGTTTTTGTAGGGGTTGTCTTTT
GTTTTGGTTTTTTATTTTTTGTCTATAACAAAATTTTAAAATATATATTGTCATAATATATTGAGTAAAA
GAGTATATATGTATATACCATGTATATGACAGGATGTTTGTCCTGGGACACCCACCAGATTGTACATACT
GTGTTTGGCTGTTTTCACATATGTTGGATGTAGTGTTCTTTGATTGTATCAATTTTGTTTTGCAGTTCTG
TGAAATGTTTTATAATGTCCCTGCCCAGGGACCTGTTAGAAAGCACTTTATTTTTTATATATTAAATATT
TATGTGTGTGCTTGGTTGATATGTATAGTACATATACACAGACATCCATATGCAGCGTTTCCTTTGAAGG
TGACCAGTTGTTTGTAGCTATTCTTGGCTGTACCTTCCTGCCCTTTCCCATTGCTACTGATTTGCCACGG
TGTGCAGCTTTTACTCGCCACCTTCCGGTGGAGCTGCCTCGTTCCTTTGAACTATGCCCTCACCCTTCTG
CCCTCACTTGATTTGAAAGGGTCGTTAACTCTCCCTTACAGGTGCTTTGACTCTTAAACGCTGATCTTAA
GAAGCTCTCTTCATCTAAGAGCTGTTACTTTTTCAGAAGGGGGGGTATTATTGGTATTCTGATTACTCTC
AATTCTAATTGTTATATATTTGAGCCCATACAGTGTATTAGGTTGAACCATAGAAACTGCTATTCTCGTA
GGTCAAAAGGGTCTAGTGATGGAAGTTTTGTAGATAAGTACCAGGCATCTCAGTAACTCCTAGACTTTTT
CTCATCCCATGCCCCGTTTTAAATTGTCAGTTTTCCCTCTGACTCTTCTGTGTTAAAACATGAAACTATA
AATTTAGTAATTATCATGCCTTGCTCTTTTTAATCTATATGACTGATGCAAGCCCCTCTTCTTAACCGTT
TCTTGGCTTTGAGCCCAGAAACACAGCTCTCCCTGTCTCCAACTCCAGTAAGCCCTCCTCAGCCTCACCT
TACGAATCCAAAGAACTGGGGTTTGTTAGGTTCTTTCTCTAATGTAGAGGCCCAGATCCCATCACAAAGT
TTTTCATTCTTCCTTGTCCACCATGATCTTCATCACAGTCTTTGATATGTCTGCATGCAAAGTGGAACAG
AGTTGGGCGGCAATGACAGAAGAGCTTCCTTGGCCTGACTCGGTGTGCGGCCACTTCGGCACTGCTTAAT
CCAGATATTCTTGTTAACTAAGCATTGTGCTTCCCAGGTGGTCTGAAGTCAGGTACTCTCTCTCTCAACA
CCTGTAGTTGAATATGATTTGGTCAGTTGCTCGTTGTAACTTGGAGAAATTCCTATAAAGTAAGATCTCC
TTGCCTCTTCCATCCATTGTTGGCACCCCCTTGCAAAAGGAAAAGAACAGCAAAAGTCAGGAGCAGTAAT
CTGAGAAAGTTAACTCCAGGATAGGTAGGTTTCTATTGTTATAGCTAGATGTAAATCTTTAGTTCCAAGA
AGTGATAGAGTTTCTGCTTTAATAATTTGTTGATAAGTTTACATAAACAGAAATAAAAGATACTATCTTT
ACCGTAGTAGTTCAGGCCAAGATTATGCTTAGTTTTAGTTCTCCAGGTAGTTACTTTTGCCATGTCCTAT
TGATCAGTGACACTGCCAGAGGCCCATACCGGCAAGAGGAAGAGGACGTCATTTTGTAAAGTTTAACTTC
TTAGCGAACTGATGTGCCACCCAGTCACAGAGTGGAGTTGTGAATTCATGTAGAGGTGGCAAACCTCTAC
CTTGTGTTGATGAGAGAATAATCTTGGGCAGTCTGGGAAAATAAGGAAGGCATCTCCTTCTTACTCATGG
AGATTCAACTATAGAGAGTTGAAACCTAAACCCGCCTTCCTTTTATAGAAGCTGGACTAGAGACGGACTG
ACCATCAGCTCTGAACTGTGGCTTTTTTTGTTCACCTATGATGCCATGTACCAAATTCAGAAGCTATCGT
TAATAATTTGTTTTATAATTGAGTAGTACAAGCGAGGAAAAAATACGGAGGATAACCACTATTTTTGTGC
AAATAATATGAAAGTGAAGTAAAAGCAATAGAAGAAATTTCTATAGGATCTGGGTTTAGAGTGTGTATCA
TTAATAAATATACCTTTGCTCTTTTCAGGGAAAATAACAACCACCCTTACTGATAGTTGGGAAAAGAAGA
TTGGGTTATTTTGCCATATCATTTAGCTGGAAGTGACATTTAAAAGCACCCTGCATCACTAGTAATAGTG
TATTTTGCTATTCTGCCCTTGTAATCGGTGTCCCTGTAAAACAATCCCCACAGATTACTTTCAGAAATAG
ATGTATTTCTCTACGTAAGGGCCAGGTTTATTTTCTCCTTTTTTGAGATTTCTAGAAAAAATGCTGCTTG
CACATGTTGGTTCTTGAAACCTTAGCTAGAAGAATTTCAGGTCATACCAACATGTGGATAGGCTATAGCT
GTTCAGAGGTCTCCTGGGGGAGCTTAAAACGGGGGAAACACTGGTTTTCACAGATGCTCCACATGGCTGT
CTTTAAAAGACTCAAAACTTTTTTTTGTCCTCTTTGTTATGCTTGGAAGCTCCCCCCCCCCCAACAGTGT
GTCGAGTCTTTGCAAAGAAACCTTTAGATGTGGTTCATAGATATATGAATACGTATCTGTGTAAAACAGT
GAGTGTGCAGTGTGTAAATACTTTAAATTATTATGCTAGAAAAATAAAGTTACATACCTTGCTGTGGAA
>NM_001366508.1 RGMB [organism = Homo sapiens] [GeneID = 285704]
[transcript = 1]
(SEQ ID NO: 16)
GCAGAGATATCCGGGCCGCCGGTGGGTGGTCGCTAGGGCTGGGCCAGCCTCTTGGAGGTCCACGCCCGCC
GAGCCCACGCTGGCTGGGGCCGGGGTGCCGGCGCGCTCGGGACTCGTCTCAGCAGTCGCTCACGGTCTTT
GTGTCTTCTCTTCCGCCCCTTTCCCTGCCTGCCGCCTCCGGCCGCCACGATGCCCCTGCGCCCCGCTGCT
GCCGCCGCGGACTGGCTGCGCCGGCTGCGCGCTGCTTGCTGCGGCGGTGGTGGCGCCCCATCTGCTACAC
GGGCCTGAAGAAGGAAGAAGAGGAAGCGAAGCGCGCCCCCCGGCCCATGCCGCAGCCACGGGCCCAGACC
CGCCACGGCGCCCGCGCCGCCGCCCTCGCCGGAGCCCACGAGACCTGCATGGACGGGCATGGGCTTGAGA
GCAGCACCTTCCAGCGCCGCCGCTGCCGCCGCCGAGGTTGAGCAGCGCCGCAGCCCCGGGCTCTGCCCCC
CGCCGCTGGAGCTGCTGCTGCTGCTGCTGTTCAGCCTCGGGCTGCTCCACGCAGGTGACTGCCAACAGCC
AGCCCAATGTCGAATCCAGAAATGCACCACGGACTTCGTGTCCCTGACTTCTCACCTGAACTCTGCCGTT
GACGGCTTTGACTCTGAGTTTTGCAAGGCCTTGCGTGCCTATGCTGGCTGCACCCAGCGAACTTCAAAAG
CCTGCCGTGGCAACCTGGTATACCATTCTGCCGTGTTGGGTATCAGTGACCTCATGAGCCAGAGGAATTG
TTCCAAGGATGGACCCACATCCTCTACCAACCCCGAAGTGACCCATGATCCTTGCAACTATCACAGCCAC
GCTGGAGCCAGGGAACACAGGAGAGGGGACCAGAACCCTCCCAGTTACCTTTTTTGTGGCTTGTTTGGAG
ATCCTCACCTCAGAACTTTCAAGGATAACTTCCAAACATGCAAAGTAGAAGGGGCCTGGCCACTCATAGA
TAATAATTATCTTTCAGTTCAAGTGACAAACGTACCTGTGGTCCCTGGATCCAGTGCTACTGCTACAAAT
AAGATCACTATTATCTTCAAAGCCCACCATGAGTGTACAGATCAGAAAGTCTACCAAGCTGTGACAGATG
ACCTGCCGGCCGCCTTTGTGGATGGCACCACCAGTGGTGGGGACAGCGATGCCAAGAGCCTGCGTATCGT
GGAAAGGGAGAGTGGCCACTATGTGGAGATGCACGCCCGCTATATAGGGACCACAGTGTTTGTGCGGCAG
GTGGGTCGCTACCTGACCCTTGCCATCCGTATGCCTGAAGACCTGGCCATGTCCTACGAGGAGAGCCAGG
ACCTGCAGCTGTGCGTGAACGGCTGCCCCCTGAGTGAACGCATCGATGACGGGCAGGGCCAGGTGTCTGC
CATCCTGGGACACAGCCTGCCTCGCACCTCCTTGGTGCAGGCCTGGCCTGGCTACACACTGGAGACTGCC
AACACTCAATGCCATGAGAAGATGCCAGTGAAGGACATCTATTTCCAGTCCTGTGTCTTCGACCTGCTCA
CCACTGGTGATGCCAACTTTACTGCCGCAGCCCACAGTGCCTTGGAGGATGTGGAGGCCCTGCACCCAAG
GAAGGAACGCTGGCACATTTTCCCCAGCAGTGGCAATGGGACTCCCCGTGGAGGCAGTGATTTGTCTGTC
AGTCTAGGACTCACCTGCTTGATCCTTATCGTGTTTTTGTAGGGGTTGTCTTTTGTTTTGGTTTTTTATT
TTTTGTCTATAACAAAATTTTAAAATATATATTGTCATAATATATTGAGTAAAAGAGTATATATGTATAT
ACCATGTATATGACAGGATGTTTGTCCTGGGACACCCACCAGATTGTACATACTGTGTTTGGCTGTTTTC
ACATATGTTGGATGTAGTGTTCTTTGATTGTATCAATTTTGTTTTGCAGTTCTGTGAAATGTTTTATAAT
GTCCCTGCCCAGGGACCTGTTAGAAAGCACTTTATTTTTTATATATTAAATATTTATGTGTGTGCTTGGT
TGATATGTATAGTACATATACACAGACATCCATATGCAGCGTTTCCTTTGAAGGTGACCAGTTGTTTGTA
GCTATTCTTGGCTGTACCTTCCTGCCCTTTCCCATTGCTACTGATTTGCCACGGTGTGCAGCTTTTACTC
GCCACCTTCCGGTGGAGCTGCCTCGTTCCTTTGAACTATGCCCTCACCCTTCTGCCCTCACTTGATTTGA
AAGGGTCGTTAACTCTCCCTTACAGGTGCTTTGACTCTTAAACGCTGATCTTAAGAAGCTCTCTTCATCT
AAGAGCTGTTACTTTTTCAGAAGGGGGGGTATTATTGGTATTCTGATTACTCTCAATTCTAATTGTTATA
TATTTGAGCCCATACAGTGTATTAGGTTGAACCATAGAAACTGCTATTCTCGTAGGTCAAAAGGGTCTAG
TGATGGAAGTTTTGTAGATAAGTACCAGGCATCTCAGTAACTCCTAGACTTTTTCTCATCCCATGCCCCG
TTTTAAATTGTCAGTTTTCCCTCTGACTCTTCTGTGTTAAAACATGAAACTATAAATTTAGTAATTATCA
TGCCTTGCTCTTTTTAATCTATATGACTGATGCAAGCCCCTCTTCTTAACCGTTTCTTGGCTTTGAGCCC
AGAAACACAGCTCTCCCTGTCTCCAACTCCAGTAAGCCCTCCTCAGCCTCACCTTACGAATCCAAAGAAC
TGGGGTTTGTTAGGTTCTTTCTCTAATGTAGAGGCCCAGATCCCATCACAAAGTTTTTCATTCTTCCTTG
TCCACCATGATCTTCATCACAGTCTTTGATATGTCTGCATGCAAAGTGGAACAGAGTTGGGCGGCAATGA
CAGAAGAGCTTCCTTGGCCTGACTCGGTGTGCGGCCACTTCGGCACTGCTTAATCCAGATATTCTTGTTA
ACTAAGCATTGTGCTTCCCAGGTGGTCTGAAGTCAGGTACTCTCTCTCTCAACACCTGTAGTTGAATATG
ATTTGGTCAGTTGCTCGTTGTAACTTGGAGAAATTCCTATAAAGTAAGATCTCCTTGCCTCTTCCATCCA
TTGTTGGCACCCCCTTGCAAAAGGAAAAGAACAGCAAAAGTCAGGAGCAGTAATCTGAGAAAGTTAACTC
CAGGATAGGTAGGTTTCTATTGTTATAGCTAGATGTAAATCTTTAGTTCCAAGAAGTGATAGAGTTTCTG
CTTTAATAATTTGTTGATAAGTTTACATAAACAGAAATAAAAGATACTATCTTTACCGTAGTAGTTCAGG
CCAAGATTATGCTTAGTTTTAGTTCTCCAGGTAGTTACTTTTGCCATGTCCTATTGATCAGTGACACTGC
CAGAGGCCCATACCGGCAAGAGGAAGAGGACGTCATTTTGTAAAGTTTAACTTCTTAGCGAACTGATGTG
CCACCCAGTCACAGAGTGGAGTTGTGAATTCATGTAGAGGTGGCAAACCTCTACCTTGTGTTGATGAGAG
AATAATCTTGGGCAGTCTGGGAAAATAAGGAAGGCATCTCCTTCTTACTCATGGAGATTCAACTATAGAG
AGTTGAAACCTAAACCCGCCTTCCTTTTATAGAAGCTGGACTAGAGACGGACTGACCATCAGCTCTGAAC
TGTGGCTTTTTTTGTTCACCTATGATGCCATGTACCAAATTCAGAAGCTATCGTTAATAATTTGTTTTAT
AATTGAGTAGTACAAGCGAGGAAAAAATACGGAGGATAACCACTATTTTTGTGCAAATAATATGAAAGTG
AAGTAAAAGCAATAGAAGAAATTTCTATAGGATCTGGGTTTAGAGTGTGTATCATTAATAAATATACCTT
TGCTCTTTTCAGGGAAAATAACAACCACCCTTACTGATAGTTGGGAAAAGAAGATTGGGTTATTTTGCCA
TATCATTTAGCTGGAAGTGACATTTAAAAGCACCCTGCATCACTAGTAATAGTGTATTTTGCTATTCTGC
CCTTGTAATCGGTGTCCCTGTAAAACAATCCCCACAGATTACTTTCAGAAATAGATGTATTTCTCTACGT
AAGGGCCAGGTTTATTTTCTCCTTTTTTGAGATTTCTAGAAAAAATGCTGCTTGCACATGTTGGTTCTTG
AAACCTTAGCTAGAAGAATTTCAGGTCATACCAACATGTGGATAGGCTATAGCTGTTCAGAGGTCTCCTG
GGGGAGCTTAAAACGGGGGAAACACTGGTTTTCACAGATGCTCCACATGGCTGTCTTTAAAAGACTCAAA
ACTTTTTTTTGTCCTCTTTGTTATGCTTGGAAGCTCCCCCCCCCCCAACAGTGTGTCGAGTCTTTGCAAA
GAAACCTTTAGATGTGGTTCATAGATATATGAATACGTATCTGTGTAAAACAGTGAGTGTGCAGTGTGTA
AATACTTTAAATTATTATGCTAGAAAAATAAAGTTACATACCTTGCTGTGGAA
Table 3: Exemplary RGMb genomic sequences
>NC_000005.10:98768632-98796494 RGMB [organism = Homo sapiens]
[GeneID = 285704] [chromosome = 5]
(SEQ ID NO: 17)
GTAACACACTTGCTCTGGGCCTTAGGTGTCTCTTTAAACCCTCTCTCCCCTCTTTATTTCCAAATCTTTA
TCTTTGGCTTTCTTAAGCTGTAAAGGCACACTGGCACCTTAGGTGGTCAGGTAAGATCGCTGGATTCCTT
ATTTCGGCGAAAGTCCAGCGGACCATTCCCGCCACTGTACCCATTTTAGAAACACTTGGAAGACCCTTAA
GGATCATTTACGGAGAGAAAGGTGTGCATTCTGCGGCTGCCGGCGGGCAGCTAGAGAACGTGGCGGGGGG
CAGCGCATCTGGCGCCTTGGCCGGGGCGCGGAGTGGGAACAGCCCGCACGCGTCCGCTGCCGCCCTGCCT
GGCGACGAGTGGCACAATCGCCCACCAGGAATCGGCTGGTGCCGCGGGTGGGCGAAGGCAGCGCCGCCGG
CCCGGAGGGCGGCGCGGAGGGTGGCGCTGGCGGCGTGAAAGGCGTCTTTGTGCAAGATTGAGTGTGGTGG
GGGGAAGGCGAAGAGAGAACTGATCCAGGACCTGGACGCGGGGGTCTGCGGCGCCCCTACGCCCCCACGC
CCCCAGCCGTCCGCAGTGCCGGAATGAGCCGGATAGCGCCGGCAGGGGGCAGTGTGGCGATATAGGGGTT
GCCGGGGGGAGAGGAGGGTCTCTGTTCACCTCTATTTGAGGCTGACTGGGGAGAAAGCTGCCTGGAGGAA
GCTGCTGGGGTGCGGGGGGCTGAGGGATTTGGCTCCCCGCCCTCTCCCCTAGATGGCGGAGAAATCGGGC
TAGCTGGAAGGGCGCAGCTCCTCTCCCAGAGCTCATTCTGGAGTCGGAGAACTGGGAGAGCGGCCCCCGA
GGCGGAGCCTCCCTCCCGCCCCGAGTCGCGCTGCCCCCCACCTGGGGGAGAGGGGGGGGCGCCGCGCTGC
CTTCCCTCCGCGCCTCGGCCGCGTGGCTTGCGGCTTATTTTCCCAGCTGGCAAGCGTCGCGCTGCAGACA
AGGGAATGCCTGTGGTGAGTTGTTTATTTTGTTGAAGTTTGCGGTGGGCCCGGGGGACGTGGGGGGCGAG
CGGCAGGCAGCGGCCGGGACTGTTTTGTTTAGGTCTCCATCCACCTGTGGGGTATAAATCTGCCAGCCTC
AAGGCGGCGGGGGACCGGAGTGAGAAAGCTCTGACGCTCCCAAAAGCCCCTTTTCGCAACTTACAGATCT
AGGACCACATTTGATTCTCCACGCCTGGAACCGGCTAGATCCTAGTTTACGCTCACCCCGCCCTCCAGCC
TCCCATCAACGCTTCCCGCACCCGCTGTGTGCAGGGCTGGCGCTCCGCAGAGCAAACAGCGCCCACGACC
CAGGGACAGCTGGCCACCGAAGTCCCTCCCCGGCGCCCTTCCCCGTTCCTTGAGGATAGGGGAGCTGAGG
TTTTCATGTGAAGGGCGCCCATGAAATTCGTGGGAACTTGTTCTGGCGTCTTCTGGAGTGGAGTTCTTTC
AGGGGTTTCGGGTCTGCCCGGGACTACAAAATGGGATCCTGGGTTGGCTGGAGTTTCCCAGACTAGGCTG
GAGAGAGGTCGGCTGTTGGCTCGCGGGAGAGCCCAGAGGAACCCCTTGGGCGATGGCAGTCAAGACCGGC
GGCTGCGTTTGCCTGGAAGAAAAGCCTAAATACATTAGCGAGCTGGTAAAGCTTTTAAGGCCTTCTTGGG
AGCGAGTGGCTGGCTAATGAGAGGTTAATTTTGTTTCGAAGGGAAGGCTGCACTGGTTTCCTGAGATAAA
TGGACAGGAAGCGCATTACCTCCGCGCGCTGGCAAGCGGCTCCCAAATGCTTTTTGCACCTGCCGTTTTG
AGCGGTGAGAGCAGCGGCGGAGCCCGCAGGGATTGCGGGGGCCCGTTGTCTACGAGCGGGTGATGAGCCC
CCCGCAAGCCCCGGGCTCTCTCCCTCCGCGAGTGCATTGCTCACGCCAAGCTTTGGGACTCTTCTCTCAC
ACCCAGGTCCTGCGTGTTCCGAAGTTCAGGGGCCCCCTGATCCCGCTGAGCCCCCTCCAGGAGCGAAAGG
GTTAAGAATGATGTAAGTACAGGCACTTGATTTCTCAAGTCGCCCGCTTGGCCCTCTTCCAAGTTGTACT
TTCCTTATTTTCTCTCCTTTCCTTTCCTTACTGTTCTAACGCTTTTTAATAAACTTTCACTCTTGCTCTG
AAAAAAAAAATGATGTAAGTACAAACCACGAGCTGCTTGACGCTGAGCTGTTGGACTCCGGGCTATTTCA
GCCGCTGCTTTGGGTACACAGTGGCCGGGGAGCAGCCGGGGAGCCCTTACTCTAGAAAGGATTGCACAAA
TCGCAAGCTTAGGTGATCTGTTAAGCAGTCAGAGATTCTCCTGTTAGATTTCTGGAAATGGAGACATATT
AACTGGAGAACAGTTAAAAACATCACGTATAGGCTCAGGAAATGAAACTGCATCTTGTTTAGAATCCATT
AGTGTTTCTGGCATGTGTTTTAATTCTGTCCACTTGCACGTTCATGCCGTAGTATGCTTTGCTGGATTCT
CCGGATGTACTGTTCAGGTCTCCTCCTTCCTTTCTGAAAACAAAGAGAAAGGGAGAAAGAGTACCAGTGG
CAAGAAACAGGGTGGATGTGTGAATTACACAAGTCATCAGCCACTTCATGATTTTTTTTCTGATTAAAAC
ATAAAACTTTTATAAGTCACCGATTTTTTTGTTTGTGTTCTAGCTCCCTCCCCGATTTTTCTTTGTACAA
TATAAAGTCGTTTGTAACTTGCAGCTCACGGGCTTGAGGAGTGGCTGCGGTATAAGCGACTATTCACTAC
GCTCTTGCTTTCCAAGCCAATTCCTGATCTTGTGAACAGTACGAATTTACCCGCGAGTGTGTCAACTGAC
CTATGTCTTGAAAATAGGAACCACCTTGCGGTTTACTTGTGAAAATTAGCATTTTCCTCTAGCGGCTGAA
CACTTATGCAGCAATAAAAAGTGAATTGTTAGGCTTATAAATCTCGAGCTTGTCAAATTTTACAACAGAG
GAGCAATAAAGCACCTTATAGAGATGAAGTTTTTCCTCTGAAGAACAGTTCCGTTTAACGACACCAGGAT
TTGCAATGACTCGAGGACGCCCTCTATTGATAGGAAAATCTAGAGCACCCGTGTACTTATGGAACAGGAT
ATAGGGGGAGGGGAGTAAAAAATCATAGAAAGTCACGAGTGGAAAGACCCAAACCAAACAGAAATTATTG
ATCCTTAACTATTGAGTATATGCTTTGAAGTATGATCAATAAAGTGAGATGCTGCTTTTATAGGGGTCGA
ATTAAATCTTGGGTTAAGAGAAAAAAATGTAGTCTTTGTTAGCCAAATACTTTGAAAATCAGTATTATGT
TTGATTACTTTTTTAAGATGCTTCTTAGTATGTCATGAACTGTATCTAATTATATGAAGTAATTGCAAGA
TAGTAAATTTTTGCTCTAATAATACTTAGTCCGCAGCATTATCTAAAAATGTCATATAACTTCCTCCCCA
AAAGTCATTTCTCCAGGCAACTGTGAAGCTATTATTAAGCGTATTATGGATATACACAGGCTTGGATTAT
ATTGTGGATAGTGAAAGGTGTGTTTACCCCTTAAGTCATCATAGTATGTAACAAGTAAGTAATGAAAAGT
TACAGTAAAAACAGATAGGCATCCTTCATTAGGGACCTAGTGGTTTGGGCAATTCAAAATGTGTCTAATA
AGTGAGCTGGTGAGACTGAAGGTTTGCCCCTCAATTATCCTAGTCCATTGATTAGCTCATTTAAACATGT
GTGGAATATCAGCATGTCCGTGGTTGATAAGAAATTCCTAATTAGAGATTCTGAGTAACATAGCGGGTAC
AGTTGCCAACTACGATTTGGATGTTAGTGGATTTTAAATGGCAACCCTGTGCTGTTTGGGGAAAGGCAAA
GGCGAGGGCGGTATCAAGTGTCTCTCAGCCTTGAGGTCATTCTTGAGATTAGATACCACTTCACACCTAC
CGATAACAAATTGGAATCTATCATCAGTCTCATAACTGCTTTTCTGTCTCCACTTGGCAGCCTCTTTCTT
TGTAGACAATTGTCTTTTTCCTCACTTTCGGACCCATCCGATCATAAAAGGAGAAAGTGAGCTGCCATTG
AATTAATCCGTAGAAATTTGTGGAAAATTTAAATAATCTGTAGAAATTTGTGAAAAAGCCACTGAGCAAC
AACAAATCGTGGCATAATTACACTGCATAAAAACCCACCACTCTCATTAATTACACTGTAACCGAACGTT
GAAGTTTGCCCACTCTTAGACCTCTCCTTGCCGCTTTCAGGAAGGGAAATGTCAAACTAGAACACCTGAG
CCTCGCACAGCTGCCAGCGAATTCCGGCGGCAGTTCGCGGGTACCTTCCCAGCCCATCCTATCTGCTCCG
GACCGCGGGTGACATTCAGATTTCCAACACCGCCCCGAGCTTGCTTTACCAGACACCCTCAAACGACTCG
GAACGCGGTTCCGCAGATGTGATTGGCTGAAAGGGGACCTGCCGCGCGGCGCTCTGTAAAAGCGCCTGCT
TCCTCCAAGGAGGTGGAGCTGAAGCTGGCAGGGCCAGGGCAGGGAGGGGTAATTAAATTAAAAAAAAAAC
TTGGAGGGAAGATGAATCGGATCACATTAATTCAAGGAGAGGTCAAAGGCCGGTACCTTTGTAACGCCGG
CGAGAGTAAGACTTGCGGTCGCCCATTTCTGTTGGGCGCGAGGAAACAGTAAATCGCGGAAGGGGGTAGA
GGGGGAAGGAAAGGGAGCGCGAGGCAGCATGCGGCGCAGGAGAGAGGACTTGGAGTCGATGGACAGGGAA
CTCGAATTACACTGAGCGGGCGGGGGAGAGGGCCCTCTGGGTCCTGCGATTGGCAGGCTGAGTCGGGCTG
ACGTGCGGTCACAACCAATGGGGAGGAAGGGAGGGACACGGCGGGGGATTATGGCGTCGTGGATTGGCGG
GCACAGGGCGGGGGGGGACGGCAGTGCGGGACTGAGATTGATCGACCGCGGGGGCTGCCGCGCAGAGATA
TCCGGGCCGCCGGTGGGTGGTCGCTAGGGCTGGGCCAGCCTCTTGGAGGTCCACGCCCGCCGAGCCCACG
CTGGCTGGGGCCGGGGTGCCGGCGCGCTCGGGACTCGTCTCAGCAGTCGCTCACGGTCTTTGTGTCTTCT
CTTCCGCCCCTTTCCCTGCCTGCCGCCTCCGGCCGCCACGATGCCCCTGCGCCCCGCTGCTGCCGCCGCG
GACTGGCTGCGCCGGCTGCGCGCTGCTTGCTGCGGCGGTGGTGGCGCCCCATCTGCTACACGGGCCTGAA
GAAGGAAGAAGAGGAAGCGAAGCGCGCCCCCCGGCCCATGCCGCAGCCACGGGCCCAGACCCGCCACGGC
GCCCGCGCCGCCGCCCTCGCCGGAGCCCACGAGACCTGCATGGACGGGCATGGGCTTGAGAGCAGCACCT
TCCAGCGCCGCCGCTGCCGCCGCCGAGGTTGAGCAGCGCCGCAGCCCCGGGCTCTGCCCCCCGCCGCTGG
AGCTGCTGCTGCTGCTGCTGTTCAGCCTCGGGCTGCTCCACGCAGGTAGGACGGGAGCGCGCGAGGGGAG
CCAGCCCCGGGGCCTAGGGCTTTGTTCCCAAATAGCCCCAGGTCTCGAAGGCGCTCGCCGGGGCGGAAGG
GCGGCGTGGCGCAACGGGAAAGGCCTCCCGAGCCCCTGACACGCACCTCTGTCGGGCTCCGGGCCACAGT
AACGCAGCCACCCACCGGGCGGCCGCCGGGCGCAGAGACGAGCCTTCTCTCCACCCTCCCCGCCTTCGGC
TCGGACCGCTGACAAATCTCCCCTCCGACAGCCGCTTATCAAACATTTAGCGGGCGGGGGCCGCGCGCCC
GAGACAAATGTTCCGGATGCTTTATTGCTCTCTGCGCTGGTGTTTACTGAAAGTCAGGCTCGGATCAGGT
TTCCGCCGTCGCAGCCGCCTCGGAGGGAGACAAGGAGCCTTGTGCTGCTTTTCTGGAGTTGGGGGTGGCG
GCGAGGCGAAGGAAGTACACCAGATGGAAAGATTTTTACCAGATTTTGCAAACCTGGGAGTTAATCTCGA
AGTAACGTCAACTTTTTTGAGCAGGGATTTCTTTCGTTTTGGCAAAAGCTGCGTTTGTTCAGCGATCAGC
CTCCCGGGAAAAGCTCGTGTTTTGTGACTAGCCGGGTGTCTTCGGGCTCCGTGAGGGGTTGACAGATAAC
CATGCTGCCTTCTGGAGGTCGGCGCTTCCCCAGCCTTGAGGCGTATGTTCCAAGGGAAGTAAGCCTGCAA
ATGAGCCTTCTACCATGTGATACCTATTTTTCTTTTTAAAAAATCGTTTCATGAAAATCCAGGACGCTCT
TCACACTATTTTTTTCTAGGAATTTGCCTAGACAAAGAATTTTTTTTAATGCTAAGGTTGAAAGGGTCAT
TTTCCTTTAGTCTTGAGAACAAACAGCATTAGATAAGTTTCTTTTCCTTCCAGACCTTTTTTGTCCTGTA
TATATGAGACACTTCACAACGTTCTGGTCTGGAGAATCCTGAACTTCGATGCCACCCAACTGTGCGGAGT
AATTTTTACTTATTAAACCATACCCGCCATGCTAAAAATCAGGGAGGTCTTTTAAGTAATGGGGGGGAAA
ATGTGACATCTGAGGAGACTTAATTTACTTTGCGTAGCTTTGCATTATGATGTAATAGCTGCTTTTTAAC
ACCGACAATTAGGGAAAAGACGGCTTAATAAAAAAGTGTAATAGAAAGTTTTAGGATGATTACGGCTTTC
AATTAGTAACCTAAGAACCTTAGAAAATAGTGTCAATCTGCATGTAGTTACGTGGAAAATTTTTCTTGAC
ATGTTAAAAGATATATTCAGGAGTGTTTGTGCGAAAAATATGTGTCCATAATACCAAAATGTAAGTTTCA
TGAAATGGAAATTGTGETAGGTGGGATTGTGTCCCCCTTGGCTGCTTGGATATTGTTTCCCATAGTTGCC
ATAGGATCCTGTGACAGAATTGGAATGTGGTCCCTGAAGGTTTCTTTGCTAGCCCGTGAAAGGCATACTA
AGTTGGCTTAAAGGTTTAGAAAAGTAGGGTGTCTTCGTTCAAGAATTTGGAATGTGAAGCTCCCAGTATC
TTCATGTGTCCTCATTTAAGTCACCATTAGATTCAGCCCAACAATGTGGTAAGCGATTTCTATGGACAAC
TTGTTGGAGTGAAGCGATTAATCCTGTTAAAGAAAAGAACGCTCCTTTTGAGAGATTTCTTTGAAGAGTG
GTGCTATTTAAATGCCCGGGTTGTTTCTCAGATAAGGGAACCCAGCAAGAGTGCCAGTGCTCTTTGCAGA
TGGACTACTATATGTTTTATGCCTGGATCCTTATCATCTGCCTCAGCACCCCATATGGCTCCACATTTCA
CTTAATTCAGCTTCAGAATACCTCATTTCTTTTATTATAAACTGGTGATTGTGCTGCAGATTCACACGTG
TAGTCCTGGTGTTAAAGAAACTGTCTTGAATGGTGAGGTCTTTATGTCTGATTCATTCAAAAATGATGAA
ATGAAAGCTCTAGACCCACAAATGCTTTAGCATGTTCAGTAACTGGTGTGAAAATACTGTCACACTGATC
ACAGATAATCTGACCCAAAAATAGAGAAATCTGTAGCTGCAGAGTTTTAATTCTGCCCTTGGGGAATGAA
AAGAGTATGAAAAGAGTACCTTTAGTTGCTTTATTTTATTTTATTTTTTTTAAGCAAACCTCTGCAATTC
CTGAATAGAGATCCAATATGGGAGTTACTTGCTTACTCATACACTTCATGGCCTTCTTGGCTACATCCTT
GAGATTGTTTCTGCTCCGCATTGTCTTGGCATTTTGACAGGAGGCTTACTTTACCGCCTATAACAGGCAA
AGTGTGCGTTGTCGTTATAAATTCATGAAGCCTGGATATTTCTGGGTGTATGCTACAAATTAGTCATTAT
ATAGAAGCCCACAAGGCAGCTGAAAATGCTGACCCAATCGCACTTTCTATTTAGCCCTCTTCGCTCCCAT
CATCTCTGTAGCAGAAGGCTGTTGGAAGCCTTAGCCAAAGCAAAAGAACCCCGGGAGATCTGTTTGATTT
GAGATTTGTTTAAGCAGTCAGCACCTACCTGCTATGAAAGAGGGAAAGGTTTAGGCCGGGCGCTGTGGCT
CACGCCTGTAATCCCAGCACTTTGGGAGACTGAGGCGGGCAGATCACCTGAGGTTGGGAGTTCAAGACCT
GCCTGACCAACATGGAGAAACCCTGTCTCTACTAAAAATACAAAATTAGCCGGGTGTGGTGGCGCGTGCC
TGTAATCCCAGCTACTAGGGAGGCTGAGGTAGGAGAATCGCTTGAACCTGGGAGGCGGAGGTTGCAGTGA
GCCGAGATCGCGCCACTGCTCTCCAGCCTGGGCAACAAGAGCAAGACTCCGTCTCAAAAAAAAAAAAAAG
GAAAGGAAAAGGTTTGGGGGGTTGCAAGGAAGGTATGAAAAGGGAGCTGGGAATCTAAATTCCACTTTTC
TTTCCTTCTGAATGGTTCCATCTGCATTTCCATACTGTTCGTTTGATATTTTATGACTATTTGAAATAAT
TATAAAGAAACGTATGCTCGGATAGATTTTATAGACCATAAAAAGAACAAATAGGCACTGCAAGTTTAGC
GGGAAGTTCTTGTGCTGTCATTTATCCGTAGTCTTAAGAGCTCTTCATTACTGAGACCTTGGCCCCATAT
TTATTTTCTTGCTTTCTGTGTATCTGATTTTAAAAGAAAAAAAATGCAATAGCTACTTAAGTCTTTTTTT
CAGCCATCAAGTACTGTCATTGCAGTTAGGAAATAAGAGGAGACGTGACCTGTGATGGAAGAACAACAAG
TTTTGAGGGAAGGCACCACATCCTGGCTTAATGATGCTAGCTGTTTTATGATTCTGCCAGAACAAGCCCT
TTAATATCCCTTTGCCTTGGATTTCCCATCTGCAAAATGGCTTTAACGCCTTTTGGAGTTGTTTAGTTGG
TTAATTTCTGTTTTGGTTTGATTTGAGTTCTAGTGAAAAATAGCTGAAAATTATTTGATCTGCTTTGGAA
AGAGAAATTCAGTAAATACTGGAGCTACTTTTGTCAGCTTTGGAATGAGCAAACATAAAGTAGATTCCAC
ACAAAGATGTTTTTGTAACTTTTTTTTTGAAAGTATGATTTGATGATTGCTGTAAATCCATAGCTCCAAC
ATATTAAAATGTTTTAATCTTTGACTCAGGTTAAAGTGAATACCAAGCCTAGACTATATAGTATTTTCCC
CTTTTTGACCCAAACACACTTTCTCTGTATTTAGCCCTCTTCCCTCCCATCATCTCTGTAGCAAAATGCT
GTTGGAAGCCTTAGCCAAAGCAGAAGAACCCAAGAAGATTTGAAATTTAAAATTTAAAATTTAAAATTTT
GCAAATTTAAAAGTGGTGACAGAGTTGTCTTCAGAAGTTGTCTTCTCCCACCTGTTTCTCCCAGCCAAAG
CCAGAAACAGAGCTTACAAGCTACTAAATTCAGATTTAAGAGCAGTTTACTAGTGAAAATGTTAAAATAG
CTTTCTAAGACAATATTAGGCTAAATTTTAAGTCTTTATCAGATGGAGTTCCCTAAATCTTGATATCATC
CTGAAGTTTATTGCATCCACATCTTGAAATAATTATATATTTAATAATAATCTACTGGCCCTGAATTTTT
TCCCCCATCCAGAATACAAATCATAAGATATTTACTGTCTGGAAAAGAATACAAAGTTCGTCTTAGAATA
GCTTCCTCATTTCTCAGATGAGGACCCTCACATTTAAAGAGATTAAGAGACGTCTCCATAGTTTAAAGAC
CTATAAAGATTTTATGTAACACAAATAATGTGTTACAGTTCATTTATATAAAATTTGCAGTTGATGACAA
AGAACAGTGTCTCTTTTCACTAAGTCTGTTGTTTCCATTTTATGGAACAGAATATTAAATTATTTGGGGA
CACACAACTGACCTAGTATTGGTAGTTTTTGTGCTTTAGGAGATTTTATAAATTTTTGTTTGAGAAACTA
GTGGTAAAAAGCATCATTTGGAAATTGATTTTTTTAAAACTAATAGGGCAGTGATACTTTGGTTTTTCAT
AATGTTTATATGAATTGTTTTTATTTAGTGTGCTTTTACTTGTGATCTGCTTGGGAGCTAGGATCCTAGG
GTAATGGAGATAGACAGTAAGGAAAAAGTAAATATATAGCTTGGTCAAGTGAATCTTGTTTTTTTTTTTC
TTAGAGCATTTACATTTTGTAGAAATTAGGAAATTGTGTCAGTTTATTTACAGTTTCTTGGTAAACGTGC
CTCAGGAGAAGTAGAATGACAGTACCTGGATTTTTTTTTTTCTTATTCTGTAGCCATACAGGTAAATCAA
GTAAAGGTGTTTTAACTAATTATACTATAATTTAGGATTTTACACCTTCCTGAAGTGGGGAGGTTTTAAC
CATGGTTTTGTGCCTAGTGCTGCTTTTCCAATGCTTTCTGAAGGAAGTTATGCAATTTTGGAAAATCAAA
ACAATGTTCATGTTTTTGGCGTAACCTTAGAGTAATCTTTTTCAAAACCTATGAACAAATAGCAACATTT
ATAAAATATTTTATTTACTATTCCCTTGTAATCTCAACCTGTGATCACTGGACTAGACTCATGTTCACTC
ACAGTGGCTCATGCTTTAAAGTTGTAAGTTAACTAATTGCATGCAGTTTTTCTTCCAGTGGCTTAGTTTG
AGAAAGACACAGGAACACAATGAAACCCTGTATCTGAAGGCTTCTTAAAATAGGAGTACAGTGTATAAAA
TAGAACAAACAATTCCCAAAGAACAATGTGATTTTCTCATGTAGCTCAGGAAGTTATGAAGAGTTTACAA
ATGTTTGGTCTTATCTTCTTTCCCATAGGTGACTGCCAACAGCCAGCCCAATGTCGAATCCAGAAATGCA
CCACGGACTTCGTGTCCCTGACTTCTCACCTGAACTCTGCCGTTGACGGCTTTGACTCTGAGTTTTGCAA
GGCCTTGCGTGCCTATGCTGGCTGCACCCAGCGAACTTCAAAAGCCTGCCGTGGCAACCTGGTATACCAT
TCTGCCGTGTTGGGTATCAGTGACCTCATGAGCCAGAGGAATTGTTCCAAGGATGGACCCACATCCTCTA
CCAACCCCGAAGTGACCCATGATCCTTGCAACTATCACAGCCACGCTGGAGCCAGGGAACACAGGAGAGG
GGACCAGAACCCTCCCAGTTACCTTTTTTGTGGCTTGTTTGGAGATCCTCACCTCAGAACTTTCAAGGAT
AACTTCCAAACATGCAAAGTAGAAGGGGCCTGGCCACTCATAGATAATAATTATCTTTCAGTTCAAGTGA
CAAACGTACCTGTGGTCCCTGGATCCAGTGCTACTGCTACAAATAAGGCAAGTATACCTTCTTTTTTCCC
TCTCCCTACTCAACTTTCAAAAGATGTTTGATTCTTCAGATAACTTTTGAAATGTGCTATAAAGGGCCTA
GTTTAAAAGGAACTTCTTTTGAAAAGCAATTAACAGTTGATAAAGGGTTAAATAAAAATTATCTAGTAAG
GAATTTCTTATTGGAATGTAAACGTGGTTCTAATTTTAAATAGACAGTGATATAAAGAATAAAAAGTAAA
CAGTGAAATTGAGTTCTCCAGGGAAAAGGCAGACCTGTTTAGTAAAAAAAGGATGCTTTTTTCAGTGATG
TCTTTTTTTGAGTGCATATGTGTGTGACTCTTGAAGAAATCCATGTTCAGATTTATCAGATGATTGAAGT
GGGTGTTCTGAATAAAGAAAGCTGTGAGGCCTGAGGCAGTGACGTATCAGGAAACATATTTTATTGGAGA
TTTGGAAGCTATAGTAAAACATAATGGCAATAAGCCAACTTCCCAGTGGTAAACCCACAGTGGTGGTTTA
GTTACTAACCTCTTGATGACCGAGGAGGTTAATAATTGGATATTGCAGAGCAGCAATATGTAACCTGTGT
GTAATCTCAGGGCCTCAGGTTAACAGTTTCAGTCAGAAGCTAAGAGAAACACTGACAAAATTTAGCTTAC
CATGACTAGCTGCCAGTTTATGTGGTCTGTGTTCGTTTGTTTTAAAGAAACTAATACTGTACAGAGATCT
TTGACACCAACTTCAAGGCACCAGTGTTTAGTGCAAGCAGGTAGAGAGGACCTATGAACTAGGTTTCTAA
TTGGCAGTCAGTGCTAATTCAGCCATTAGTAAGTAAAGTCACTTTTCCACTAAAATAGTGTCCCGACTAA
CCACCTCCTTCCGTGTCTTTTCCAACCACAAAACCACACACATAGACCATCTCATCTTACTTTATTTCTT
GCATTTATTGTATCCAAAAAAATTGGTTTAGTTTTTTAATGTAATTGACAAGACTCAAGCTTTTGAAATA
CTTACGACTGTTCTCTTGATTCCAGGACACCCTCCCCTGTGTCTAAACATGTGTGGTATACTTTTTAAAT
AAATTAAAGCTTATTTTACTTGGCATTTTGTGGAGGGAATTTTACAACACCTCGGGAGATATCTGCAGAC
ACCCTGGGGTGTCACAAATTTAAGACTAGAAGTCATTGCCCTAATCAAAGACGACTGTTACACTGGTCTA
GACATCTTAGGGGAACTCCAAAATGTTTATACTTCAACTCTCAGTAAAAGCTCAAGCTTGTCTTTTGCCT
TACCAAATTCCATATCTCAAAATAAACTTTTCTCCCTCTGGTTTTTGTATGTACTCAAATCATTTTTTTA
TAAATATCTCAGAATATGTATTTGCTATAAATTATATATAAAGTTATAGAATTATGAAGTTATAAGAGAA
ACGATGGCCCCAAATCCATCATTACTGCAGAGTCATATGGCACATGAAAGCTGGGAATAGTCTTTGGGTA
ACTGAAAGAAGGCAGTCCATTGAGAATGGAAAAAGTCATCAATTGTGCAAATAATTGAGGACTTGGCAGA
GTGACCTTCGAACAAAACTATAGAGATTAATCAGAGCTTGTAAATGGGGAAATACTCATTCTCTTCACAA
GAAGCAGTATTCAAATTTTATTCTTCATGCCTTCCATCCGGTCATGTCACTTCTGTTATTCAGTCTTTCA
GAATTCTATTGTTACTGATTATGGTGAAAATATGTGGGGGATAACCTTATGAATATTGCTATATATATGG
AACAGTTTACACTGAAGTATTTAAGATCATTTTATTTCAGTTGAATTCTGTTGTAATTTTTAGCAAATAA
ATAAAAAGGACAATCTTGTTTTCTCCCTCAGTTTGGAATAAGGATCAAAGGTTTTACCCATTCAAAAAAA
GTTGGAGAAAGTGGTCCTCTGACATAAATTCAAGTAGATGATGAGAATGCTTGGTCTTTAATTGCACTAT
TGGAAGGTATTTTCCCACATCTGGTGACCATGCTGTTCAGCAGCAAACTATTCTGTAGCCTCACTTCACA
ACCTCTAGGAGTTGTTGGGTTTGGTTCCCCATCCCCCTTCTCTTTGTAACTCTCTCCAAAGCCCCATAAG
CATCTGGAAGGTTGCTCTTGAAGACCTGTCTCAATACTAGTATGACGTGTGCTGGTGAAACCAAAGAAAG
GCCTAAATCTTACAATACGTTCCTTTTACCCAGCCAGGACCAAAATAAAACCTTTAAAAACCTTCAGCAC
AAAAAAGTGGTGGTGATAAAAATAAAAACAATAAGAGGCATAAAAGTGCTTATAAAATAAGTTCTAAACA
AGATCTGAAATTTTCCCTTAATGTCTACCACCATACTTTCTTTAAAAAATCTTTTGAAAAATTTTTTCAT
GGAAATCCCAGCTTTACTGAGAATTTAAGCAAGTGCATAGACCAGTTGTTGGGTAACCTAGTACTGTGCC
TTGTAGATACCAGATCATCCTTGTGTGGTCAAGTGTTTTTAATAACCCACGTGCTCAGTGCCACTAGATT
GAAAGACTGATGTCATTGGCTAGCTGGCCAGCTGTGATACTGAAATTTTCGGAAACATCCATTCATTTCG
CTCCCTTTAACACTGCACTGAAGCACTAGACCCCAGGGTATTTTGCTCAATGAGGCTACTAAGACAGCTT
CTCAGTCCTGGGAACCTGGCCTCTTGAGCAAGCCGTCTTCAAGCCCAACTTTGTGAGCTCTGCCTGTTCC
TAGGTACCCCCCTTTTCCTTATCACAAAAAGACAAAGTGGGGGAAAAATCTAAACACCATAGAATGAAAT
GAAGTCACCCGGGGCCATATATTAGCTAAGCCTTTTCTTTTTCTCTACATGGCAAGCAGTGAGTTCAAGG
CAGACATGCCTGCGGTGCTTAATGATGTTGCCTCATCTCCCCTGGTCTACTGTTTTCTTCCCCACCATGG
CCCTCTCCTCCCCTTCCTTGGGAAAGGTAGGGTTTGTTTTCCTGTTGGTCATGGTGTGCACAGGTATGCT
TTCTCAAGGCCTCTGTGTTTTTAGTTTTGCCCTGTGCCTGCCTCCCTCTCTTTGGAGGAACCTGTGCCAG
TTGGCTACCTTGCCAGGTAGATGTGTTTCTGAAGGGGAAGCCAGGGCTGGTTTTTCATCTCTCCACCCCG
GGGGCTGCAGGGGTGTCTGGCACACACACATACCTCGGCTGACATCCACATCAGCTCTGGGCCATCTTCC
CCACCTTTGGCTGTGAGAGGCGGCTACAGGCATGTAGGCACTCAGAATGAGATGAGAGGGACAGGCTCTG
AACTGGCAGAGTTAGCTACACTGAGAAGAAAGGCAGGTTTTGTTTTGCAGGAGGAAGTAGCCCACCCTTC
ACAGGCTCCAGAGAGGGCACATGTCAGTAGTTGTCCTGTGCCTCACACACTCAAGATAAACCAGATGATC
TGTTTTGAGGATGCTTGACTTCTTCCATGTTATTTTTGCCAACCTCTCAGTCTCAACTCTTGTTCATTCA
TTCTGTGGATCTTTATATCTCTGTTTAAGTGGGTTCAATAAGAATCTGTATATGCCATGATCACAGAAGG
TGATTGGGGAATAAATTTTTTTATGAAAGATTCTGCACTTTGCAAATGTATTCCCAATAGCATATATTAA
AATTGCCAGTCTCCAGGGAATTCAAAAGTATTCCTTTTTCAAAAACAATTCTGAAATTCATTTATTATTA
AGTTGCATACCTAAACAAACAATGGAAATGTTTTTAGTAATTATTTTTGTACAAGTAGTTTGAATGCCTT
CATTTTATTTTTCCTTTTGTTTTAATGTGGTTGTATCCTTTTTCACCACTCTTGCTTTCTTATTCTATCC
TTCCACCTCAGTAACACAAAATAAAAAGTAGGAGTGGAGACTGGGGATGACAACAGGTCCTACTGCAGAT
CTTCTGCTACACATTTTACTGTGCCTCACCCTTGAGTGAGAGAAGGTAGTTTACCTTAAGGCGTTTGAAT
TTGGGAAAGGGCTGTGCTCATCGTACTTGACCAAGCAAGATGACTGTTAGGCAGATGGACAGATGCTCTG
AGGTGCCTATCTTGTACGTTTTTGGTGCTGCCAACGGCCACCAGCTCCTGGCAATCCTATGGGCAGCACT
CATCGCCTGTCCAAAGCATAGTAGCATACCCATCATGCATTATGGATAGTCTAAATTCCATACTTACCCC
TGAATTTCAAATTCTGTAGCTACTTTCTTGGGAATTCTTGAAAATTTCCGCTGGTTGAAGATACTAGAGA
TGTAGAAAGACTTAGTTTGGTTTGCTTTTGGTTAATGTCTTCTTCAAACTCTGTAACCTACAGTTTTCCA
TTGTAAGAAACACCATTACCTTGTAGGCTCCTAGAGCATGAGGTAGGTCTGCTTTCTGGTGCCCCTTAGC
AGGGAGTTATTGTTCTACAACAGCATGTGGGCATGTAAACCAGTGTTCTGGGTCATTCTTTGTTGGTTAC
AAAATCAGAACCAGACATGCCTGCATTGCAACAGCTTTCAGTTGTCATTTTAAGTATATCTCTGCATAAG
GTTTGGAAATCTGTTCTCTGTTGACCCCAGACCTAGTACATTTATTGTAGCTAAGTTTTTGCAACCCATT
ATGTTTTTAGCATCTTTATGGAAACCATGTGAGCTACATAGTGTTTATCATTATTTTTCTGGACTAAGGG
ATATGGTTCTTCCATTGATAGGTCTGTGTTTTGAATCTATGGACACTGTTCTTGCATGCAGTGTTATAAA
GAGGGGGACGCATGGTATTTGTGGGGGAAAAATAGACCATTTTCCCTCCACAAGGAAACTGCAACTCTCT
TGGTGATACATTGTAACTAATTCTTATAATCTGAGATCGCTCTTTATTTTTGTTACAATTACATTGTTGC
ATGGGTAATTCTTGGCATCTCTTTGAGATTTGCTCCTTATTGTGACCCTCCTTGCTTTATGAAGATGCTA
AGAAGTTCTGACTGGTTGAGGCATATTGGGAACAGATGGAAAGGATTTTGCAGTTACACCTCCCTGCTTA
ATGTAATTATTTCCCTGATAATCATTCTCTGATAAATGTATGGTTAATTTGGTGCTGCATTTGAAACAGA
AGGTACTAAAGGGCCCACTGTGGGAGATGTAGAGGTGAATGCAGCCTTTGCCTCAATCTAAGCACATAAC
TAAAATTTGCAGCAGTTGGTGGGAAAGCACGGGGTTTGTGTGTAAACCCAGCATTTTCAGGGTTCTTTTC
ACATTGACAAGCTGGTTCCAATTGATGTTATTCATTTAGCAAGTTGGCAGGGGTGTCAAGAAGAGCCAGA
AGAGTGGGCTGCTACAAAGGCCTGTCCCACGCTAACACCCCTCCTTGCCAAATCATTGTCTTTACTCCCG
GGATTTGGCACAGTCTCTTTCCACCTCCCACAGTAAGAATGCTCACCTGCATCTGTATTTCCTCATCTGC
ATACAACCTAGCAGGGCCTAGAACCCTGGCTCCCACTTTTGAAAGACTGCTGATCTTTGATGTTATACCA
AAAAGCTTTCTAGAATTTGACTGTCATAGTCGCCAGTTCTTAATTAAATTCATTTTCCAGAGCCTCATCT
CATCTTTAAAATTCTCATAAAAGCATATGTTAGCTATTTTTACCTCCCTGAATTTTTTTATTATAGAAAC
AATATTGAAACATTAAAAGCCAGGTTTTCAAAAGAGCAGAAATCAGCTGTAGTTCCAGGTGGCAGTCTAA
TTATTTGTCCTTTTCTATTTATCATGCAGCTGTTGTGCATACTGGATTATAAGATCTTAGGCCCAGGAAG
TACTCAACTTTGCTTTATATCATTTCAGTAAATTGCCTGGAGGTGAAAAGGTTTCCAAGTGGATGTGGGG
AAAGGAAGCAGAGAAAAATGGGGAAATTGCTAGCTGTGTAATGAACCTATGTCTGAAAAATCATTTTGTA
AGATAAATGAGCTACCTGTTTATTCTTTATGCTATACTTTTCAAAGAAGATTTGCCTTTGTTTCATTCAT
ACACATTAAAAATTCTGGGTAATGAGTCTTTTACCATCTTTCTAATCTCCCTGACTGTGCCACTCCTTCT
GTCCCAGATGTGGTTTGGAAGGAGGAAGAGACCTTCAGAATGCCCCCCATTTGTAAGTGAAATTAACCCA
GCGCCTGTCCCTGTGTGAGCCAGGCAAGATTCCGTGAGGGTGCTATGGGGCTCTGCTGCTTTAAAACAAA
TCCACACTCTTCCATAGTTTAAACCAGAGGCATGAGGCCAAGTCTGATACACAGCTGAGAGTTAGAACTT
GTTTTTCTGTCACATTTCCAAATGTCTGCCTGGTTAAATTTTCTTTGAGTCAACATTTTGAGGGGAAGGG
GTGAGAAGGAAGCATTGGATGTTTGGTCCTTTTTTTGTTGGCTTCTATTAATGCTAACCAGTTTCTGCTC
GTGTGGAATGTGGCATATAAAAAGTTACCTACATTGTTGAACAATGAAAACTGCTACCTTATCCATTTTG
TTTTTATTTGTTTTGACTGCAGTAGAACTTGCTCTTCCACTGTTTCGAATTTTGTGTTCTTGTGGGTGGG
GTAACTTAAGGACTTTTCATACTGACAAGGAAGCTGGCCAACTTGTCAGTAAGTAATAAAAGAAAAGCTA
ACATACATCTAAGTTACAGACTTTAACTGGACAGCCTTGCAGTGAAATCACAGTGCTATTGGGTCTAGAA
AGAAGTTTAGGTGAAACTGGTTCTATGGAGAGAGGAGTTTTTTATGCCCCAGTTAACTGTTTACTTTAAG
AGTTGGAGCCAAAAGAACATGGGGGTTCTTTCATGTTGGGACACAGCATGTATACTGTGAATAGGATGAT
ATGACACCATCTAAAACTCACTTCTCATCTGCTCTGCATGTGCTCCTGAAATGGGGGTCAGTTTCCATTT
AAGACATGTGTGTGCATGCACAGATGTACATGCATGTGCTTTTACATGGTATCGAGGTCTTCCCCTCACC
CTACCAAGGAAAATGCAGACATAAAGATATTGTTTTGTAGGATTTCGGTTAACTCACTTCTTGAAATGCA
TGGTAACCATTCTAACCACTTCATTCCTGAGTTCTCATGGGTCTTTTCTTTTTCAGAGTGTGAGTGTATT
GTGAAGATCTTCAAGCCCTTTTACTTTTTTTGCTCACCTCCCAAAGCTGAAAAATGATCAAAGAGCCTGT
GGTTTTCTAATCCCATCGGTTTGGAGAACAGCTGTGCTGAACCTAGGCCCAGAGCAGGGCCTCGGGTCTG
TAGTCCTGCCACCTCTCTCTACCAGGAGAGGGAATGCCTGAAGGGAAGACCTGCTAGGACAGATTCGGCT
GAGAGTTACTGCCACCAGCCCTGGCTCTCACCTTTCAGAATATTTTTAGCCTCTAGTCTGAAGCAAGGTG
GCAGCCTAGGCCTATAATACTGTCCACACAGGCCTGCCTTCATATCTGGGCATATAGTGAGTTCCTACAC
TTGCTGGCCTCCTCACAATGTGGAGTCCGAACACAACAGACAAGCTAAGGGAGAGGTTCCATTTGGAGAT
TTTGGAGTGACCAAGACTTACAGGACAAAGGCCTTTCCTCATGCTCACCCTAATACAAGGAAGTTGGCGA
ATTTGCCTGTCTCTGTGGCTATTTATGCTTCCAGCATTCACTGTCTTAATTGTACAGAAATTACTCTTGG
GAAGCAGCATTTTTGTGCCTGTTCCTTTTATGTGTCTTCCTGGGCCAGCTTCCCTTTCATTCTCGTTTTA
GCCACTGAGCTTGATGCTGCTTCTCTAGACCACCCTAGCTGCCTTCAGCTCTGCGTGTCCTTCCCCTCTG
GTTTGGTTTCTTCATTCTCTATCTGCTGCCAAGTACCCCTTCAGCAGCCAAGGCAGCCCCTGTTTTTGCA
TGTATTTATTACCTACGGCAACACTGAAGAATAAGTTTGGTCCATTCCATTTCTCTGGATCCTGTAAGTG
CTCCAAGCTCTGTGGAAAAAGACATGAGGATAGAGAAACTGATACCTCTTTTTAAGGTAGCTGATTTTTT
AGTCTCATTCAAGCCCTAAAAGGAGTCCACAGTATGAGTTTTAATCTGCCACTCACCTCGTAAACACAGG
TACTGTCTTACTGTTAGTATCTTGTCCTAGTCCAATAAAATTGTTTAACAAAATGTATTTTTGTGGATCT
TAAGTTGAGCTTCCTGTAGATATTTGTTTTATAAGATATTTGATATGTAGGCTGAGTGGTGATTGTAAAA
TTACATCCCAGTTCATTGAGTTCATAGCTTCCTAACAGTTTTTTTCATAATTTTTAAAGGAAAATTATCA
ATGACATCAAGTATTTTCTAGTCCATTATTTTTTCTTAAATATGGCATATATAAAAACTCTCCTGGGTTT
TGCATGAAATTGTTCAGAAAGAGTTATTCTAAATCAATATACCTCTTTTCCCCCCTCGAGAGAGAGACAG
AGTTCTCCCCAGTTTTTATGTAACCAGGGGACAGTTGCCACCCCGCGTGGACAGAGTACCTCTGCTCTGG
CCAAAATCCACACTTCAGAATTACCCTGGCGGGTAGGGAGGAGTGGGACCAGGGAAGACACTTGAAGGAA
GGTGCTTTCAAAGGCCTCTACTTCCTTTAGGTGCTTCAGAGCATCCTAGTCTCTGACTTCGTAACATTTA
GAAAAATTGACCCTTGGAAGTAAAATTTGGGTTTTTTAGGAATGGAGAGAGAGCATGTATATGTTTTTCT
CCTTTGAAACATGTGCTCTTGTTGGAATGGTTTCTTTTTTGTTCACTGGGGAAGACTGTAGAGGCTGCAT
GCTGTTAACCATGTGTTAAGAGTAAAAGGGGCTGTGAGCACCGTGGGCTTATTCTCCCTTAGAGTCTTCT
ATTTTTCAACCTATCATTGCACCCAGATGATATGGCAAAGTAGACACATCAGCTAATTATATGAAATAAA
ATTCATCGACTTCTTTGACACTGCAGAATTTTAACCAGGTGCATATCAAAATTATGTTTTCATGTGTTTC
AAGCTAGTTTCTGTGCCTCTTCAGTTTAGTTTACTGACCAGTCTAGAGTCCAGAGATGAGAGCTCCTAAA
GTCTGGCTCTCATGTTTTGAGGATTCCAGAAAAGTCCTACACTCACAGTGCCAAATGGCCTGATTAAACA
CAGATAAATGTTTCTTCCAAAATATATTACGCACAAGTAGTCAAGGTGCACAGCCATTTGAGAAGTCTGC
TGTTTTAAAACCTGTTTGTGGCATTTTGCAAGGCTGCTGTGCTTTGGATAGATATCTCTGTACCTCTTGG
CGAGGCAGTACCTGTGTATTTCCCTCTTTGTTGGCTACCTAATTTTCATTTAATATGTTTTTTTTTTTTC
TCCCGGTGGAAAGGAGAGGCAGGTGCCTCTTAAAATTATTTCACAGCAAACCTTCGTAAGTTCTTATTGC
CTATTTCATGTAGAAAGGTTGATTTGTAGCTGCTGTCTTTCTCTTCCTAATAAACCTTGTGGGTGGTGAA
GATCTAGGAGAGTGAAACATTAAAAATGAATGAAGAGAACTGAAGTAGTTTGTCTAAGGTGGGGGTTGAG
AGGGGCTGGGTGGAGGAGGTTTAACAAAAGGCATTGATGTTTTAAATGCACAGAAATGCCATCTATCAAT
AATAAATGGTGTGGCATAGAACAGATGTGCCAAATTGAGTTATGACAGCATATATATTAGTTCATAGAGG
GGAAAAGTAAGTAGCATAGCTAACGTTTTTAAATTATCTGTGAGCTTTTGAAATACAATTTTATGTATTC
AGTTTTTACTTTCTAGTGGCTGCTAGCTGGAAATATTAAAAGTTTGGTGTTTTTTAAAATTCCAATTAAA
TTGTGCTTGTTTCTAAAGTAAGAATTAAGAATGAATAAATTTTGCTTTTCTGAAGAGGAAGACAGTCTGA
AATGAATTTCTTGCCCTTCATTGGCTGAGTACTTGCCTTTTAAAAGCTCATGTTGTAATTGTTATGAGGA
ATGAAAGTGCATTATAAAACTTAAGTCATGTGAAACTATTACCCAGTGTTTTCTAAATTTACATTGATTA
TATCTCTAGGTACCTGCTCAGTGCATATTTAAACTACAAGGGACTACTCAGGAAACCCACTCATGCTTTA
CAATCAATTCACCCCCCTCAGCATTTTGATTCAAGGGATAATTCTACACATATGCTCTGTGTGAGAATTA
CCTTGCTTGGTTGGGAAACAACAAGAGAAATATCCACTTGTCTCACAGCAAATCACTACTAGTAAATTGG
AAAGACAAATCTTTTAGGTTAACTAGGATATTTTGTAGGTGTAGATACTAGAGCAGAAAACAAGTTTTGA
GCTTCTAGATAGGTAGGAGTTATTGTTAATGTCACTGGCATAGCAGAGGACAATACTGTGGGAGGTGTTT
GAAATGAATCCTGAGTTCCTGAGTTGGAATTGGTGTGCTTAATGAAAAAAACAGCCGTGGTGAGACATAT
GGAACCATTCCACTCATCTCAATATGGACCCTTGCTTGCTTTTTGGAAACTTGTTTGGAATCGGTTATCT
TTGCTTTGTTGGCCCAACACAAGTAAATCCTCTTTGTTTTTCCTAAGGTGAACAAAAAAGTAGTTCTTGC
AGATAATATCAAGTTGGTTTAAATTTTAGGTTTGTTAGACTAGAAAGACGAATTATTTAGCCGTCATTGA
AGAGTTTAATCTTTGAGTAGATGTTACAGGTGAAAATGAGCATAGTGATTATATAAAAAGTCTATTACTA
ACTCTTATTTTAGCCATCTGTATTGTTGACTTTCAACTTTTTCAGGTGCAAAATTTTCTTGAATCAATCT
TTTCATCTCCAAGGGATCCCAGCATGCCTTAAAAAAAAACACTTGTCAACGGATTTTAGAGCCACCAAAT
AAATCTATTTTATGGTCCCGTTAGATATCTAGCTCAAAACCCCCATTCACTCTGAAAGGTCCTATGGTTG
GATGCACAACAAATTTCCTGAAATCATCTCTGATGCACCTCCTTTGGGTTGTTAAGAAGGTGTTGAAATG
TGAGAAGGCTGGTAGTGAAATCAAGTIGTGGCTCAGGGTAATTAGGAAGGGAGAAAACCCAGCATTTTCA
TAGTACATTGTCTGGCTTTTAGTTGCAGCTGCACACAGCGGATATGAATTTAACCGGTTGCTACAGTGCA
GCTGGTTCAGATCTGGCTTTATAACTGGCTGAAGTAAAATGAAATGGCTGGAAAATTCCAGTCAAGTAAC
ACCAGTGGGAACAAGTGGAATTAAATAGTGTGAGCAAATGCTGGCCGAGTTGGAGAAAAACTTAAGACAT
AAACTTGAGGGTGGTGGTGAGGACTTCGGGTGGGCAGGCGGGGGGAGGGGGGTGCATTTTCTGGTCTTTT
TCACAAGGGTCTATTGTTTCGATTAGTTTCCTTGCAGGAGGTAGAAGGTTTCCTCCATCCCGAGTAATTC
ATAGAGTGTTTGGCTTGTCTACCTTCCTCCTGACTGAAGTCACCTGATACTTTTTGTTTTTTCAAGAAGG
AAGAGAGAACCCTGTTGCCTCAGTTACTAGCAATGATACAATTCTCAAAATCTGGTCTTTTTTTGTTTCT
TTGAAATAGTTTCTCCATGTTGTGTGACACAGCAGCCCCTGTCTTATCATAGTTGTCTTCCCTCCACCAC
CTGTACCAGAGATGTTGGATATGTTGGAGGTGAAGGTGTGCAAGGTTTTTAACTAACTGTTCTAATTAAA
GGATTCTGCAGGAAAGAACATGGGTTTACAAAAGAGAAGCTTTTGTATTATTAGTAATTTTTTTTCTTTG
ATGAATTTATGTGCTTAGTTTGGAGAATCGAGAGTTGGCTGGGAAAAGATTTCTGAGGAGTTAAGGGACT
CTGGTGCTGTTTGGGAAAACATTTGCTGTAACTAGGTTTAGGGTATTCAGTCTCTGCCAGTGCTTAGGAG
CTATATTTGCCAGGTGATTTCCCTGTCGCAGGGATTAGCCAGTGATGTGAAACATTTGATTAAGGGACTA
ATAGAAATCTTTCCTGGGGTACATGAGTGGTGGCTGTTACTGGGGTGCCCCCTCCTCAACTCTTTCTCTC
TGTATTTCGAGTAAGTTTGTAGTGGTGAGTCATGGGATACAGTTTTTTAATCCTATGAAGAATCTTGGTA
GGAAGCAAAGTTCTGTTAGGTATATTGTTAATTAAGCCCTTTAAAGTTTCTGCATAATAACTCAGAAACG
AGCCTTTTTCTTTTCTCAGAACTATGCCATGACTCATCCACTTTGGAAGCAGACAAGATATAAATTATCA
ATAAAAATTGCCCATAATCTGGCCTGGCACAGTGGCTCATGCCTGTAATCCCAGCACTTTGGGAGATGAT
GGTGGGAGGATCGCTTGAGGCCAGGAGTTCAAGACCAGCCTGGGCAACGTAGTGAGGAACCCCCGACCTC
CACCATCCCCCCCGCCCCCCACCAACCCCCTCACTTCAAAGTTTTTTAAATCAGCCACGCATGGTGGTAT
ATGCCCTTAGTCCTGTGTACTCAGGAGGCTGAAGTGGGAAAGTCCTTTGAGCCCAGGAGTTGAACGTTGC
AGTGAGCTATGAATGTGCCACCGCACTCTAGCCTGGGCAAGAGAGCTGACCCTGTCACTTAAAAAAAAAA
AAAAAAAAAAACCGATATTCTGATAGCAACACTGCTCACTTGCTCACTCTTTCTCTTAGGAAAGTAAAAA
AGTTTTGTCTTGATGCACAGATTTATTTAAACTTTAGTTCTAAATATATAGGACTTTAAAAAATATTTTC
TGAAAGTGATATCCATTTTTGAGGTGCAAGCAGTTGGGTCCATTTTGGAGATTCCGCCTTAAGCCATTCA
GCTTCAAAATGGCGGGGAGCTCTTGCTACAGAGGGTTACCCCGTTCTGCTTCCTTCCCATTCTGTTAAAC
CTTGTACATGCTCCTTCCCACAGATCACTATTATCTTCAAAGCCCACCATGAGTGTACAGATCAGAAAGT
CTACCAAGCTGTGACAGATGACCTGCCGGCCGCCTTTGTGGATGGCACCACCAGTGGTGGGGACAGCGAT
GCCAAGAGCCTGCGTATCGTGGAAAGGGAGAGTGGCCACTATGTGGAGATGCACGCCCGCTATATAGGGA
CCACAGTGTTTGTGCGGCAGGTGGGTCGCTACCTGACCCTTGCCATCCGTATGCCTGAAGACCTGGCCAT
GTCCTACGAGGAGAGCCAGGACCTGCAGCTGTGCGTGAACGGCTGCCCCCTGAGTGAACGCATCGATGAC
GGGCAGGGCCAGGTGTCTGCCATCCTGGGACACAGCCTGCCTCGCACCTCCTTGGTGCAGGCCTGGCCTG
GCTACACACTGGAGACTGCCAACACTCAATGCCATGAGAAGATGCCAGTGAAGGACATCTATTTCCAGTC
CTGTGTCTTCGACCTGCTCACCACTGGTGATGCCAACTTTACTGCCGCAGCCCACAGTGCCTTGGAGGAT
GTGGAGGCCCTGCACCCAAGGAAGGAACGCTGGCACATTTTCCCCAGCAGTGGCAATGGGACTCCCCGTG
GAGGCAGTGATTTGTCTGTCAGTCTAGGACTCACCTGCTTGATCCTTATCGTGTTTTTGTAGGGGTTGTC
TTTTGTTTTGGTTTTTTATTTTTTGTCTATAACAAAATTTTAAAATATATATTGTCATAATATATTGAGT
AAAAGAGTATATATGTATATACCATGTATATGACAGGATGTTTGTCCTGGGACACCCACCAGATTGTACA
TACTGTGTTTGGCTGTTTTCACATATGTTGGATGTAGTGTTCTTTGATTGTATCAATTTTGTTTTGCAGT
TCTGTGAAATGTTTTATAATGTCCCTGCCCAGGGACCTGTTAGAAAGCACTTTATTTTTTATATATTAAA
TATTTATGTGTGTGCTTGGTTGATATGTATAGTACATATACACAGACATCCATATGCAGCGTTTCCTTTG
AAGGTGACCAGTTGTTTGTAGCTATTCTTGGCTGTACCTTCCTGCCCTTTCCCATTGCTACTGATTTGCC
ACGGTGTGCAGCTTTTACTCGCCACCTTCCGGTGGAGCTGCCTCGTTCCTTTGAACTATGCCCTCACCCT
TCTGCCCTCACTTGATTTGAAAGGGTCGTTAACTCTCCCTTACAGGTGCTTTGACTCTTAAACGCTGATC
TTAAGAAGCTCTCTTCATCTAAGAGCTGTTACTTTTTCAGAAGGGGGGGTATTATTGGTATTCTGATTAC
TCTCAATTCTAATTGTTATATATTTGAGCCCATACAGTGTATTAGGTTGAACCATAGAAACTGCTATTCT
CGTAGGTCAAAAGGGTCTAGTGATGGAAGTTTTGTAGATAAGTACCAGGCATCTCAGTAACTCCTAGACT
TTTTCTCATCCCATGCCCCGTTTTAAATTGTCAGTTTTCCCTCTGACTCTTCTGTGTTAAAACATGAAAC
TATAAATTTAGTAATTATCATGCCTTGCTCTTTTTAATCTATATGACTGATGCAAGCCCCTCTTCTTAAC
CGTTTCTTGGCTTTGAGCCCAGAAACACAGCTCTCCCTGTCTCCAACTCCAGTAAGCCCTCCTCAGCCTC
ACCTTACGAATCCAAAGAACTGGGGTTTGTTAGGTTCTTTCTCTAATGTAGAGGCCCAGATCCCATCACA
AAGTTTTTCATTCTTCCTTGTCCACCATGATCTTCATCACAGTCTTTGATATGTCTGCATGCAAAGTGGA
ACAGAGTTGGGCGGCAATGACAGAAGAGCTTCCTTGGCCTGACTCGGTGTGCGGCCACTTCGGCACTGCT
TAATCCAGATATTCTTGTTAACTAAGCATTGTGCTTCCCAGGTGGTCTGAAGTCAGGTACTCTCTCTCTC
AACACCTGTAGTTGAATATGATTTGGTCAGTTGCTCGTTGTAACTTGGAGAAATTCCTATAAAGTAAGAT
CTCCTTGCCTCTTCCATCCATTGTTGGCACCCCCTTGCAAAAGGAAAAGAACAGCAAAAGTCAGGAGCAG
TAATCTGAGAAAGTTAACTCCAGGATAGGTAGGTTTCTATTGTTATAGCTAGATGTAAATCTTTAGTTCC
AAGAAGTGATAGAGTTTCTGCTTTAATAATTTGTTGATAAGTTTACATAAACAGAAATAAAAGATACTAT
CTTTACCGTAGTAGTTCAGGCCAAGATTATGCTTAGTTTTAGTTCTCCAGGTAGTTACTTTTGCCATGTC
CTATTGATCAGTGACACTGCCAGAGGCCCATACCGGCAAGAGGAAGAGGACGTCATTTTGTAAAGTTTAA
CTTCTTAGCGAACTGATGTGCCACCCAGTCACAGAGTGGAGTTGTGAATTCATGTAGAGGTGGCAAACCT
CTACCTTGTGTTGATGAGAGAATAATCTTGGGCAGTCTGGGAAAATAAGGAAGGCATCTCCTTCTTACTC
ATGGAGATTCAACTATAGAGAGTTGAAACCTAAACCCGCCTTCCTTTTATAGAAGCTGGACTAGAGACGG
ACTGACCATCAGCTCTGAACTGTGGCTTTTTTTGTTCACCTATGATGCCATGTACCAAATTCAGAAGCTA
TCGTTAATAATTTGTTTTATAATTGAGTAGTACAAGCGAGGAAAAAATACGGAGGATAACCACTATTTTT
GTGCAAATAATATGAAAGTGAAGTAAAAGCAATAGAAGAAATTTCTATAGGATCTGGGTTTAGAGTGTGT
ATCATTAATAAATATACCTTTGCTCTTTTCAGGGAAAATAACAACCACCCTTACTGATAGTTGGGAAAAG
AAGATTGGGTTATTTTGCCATATCATTTAGCTGGAAGTGACATTTAAAAGCACCCTGCATCACTAGTAAT
AGTGTATTTTGCTATTCTGCCCTTGTAATCGGTGTCCCTGTAAAACAATCCCCACAGATTACTTTCAGAA
ATAGATGTATTTCTCTACGTAAGGGCCAGGTTTATTTTCTCCTTTTTTGAGATTTCTAGAAAAAATGCTG
CTTGCACATGTTGGTTCTTGAAACCTTAGCTAGAAGAATTTCAGGTCATACCAACATGTGGATAGGCTAT
AGCTGTTCAGAGGTCTCCTGGGGGAGCTTAAAACGGGGGAAACACTGGTTTTCACAGATGCTCCACATGG
CTGTCTTTAAAAGACTCAAAACTTTTTTTTGTCCTCTTTGTTATGCTTGGAAGCTCCCCCCCCCCCAACA
GTGTGTCGAGTCTTTGCAAAGAAACCTTTAGATGTGGTTCATAGATATATGAATACGTATCTGTGTAAAA
CAGTGAGTGTGCAGTGTGTAAATACTTTAAATTATTATGCTAGAAAAATAAAGTTACATACCTTGCTGTG
GAA
>NC_060929.1:99271925-99299789 RGMB [organism = Homo sapiens]
[GeneID = 285704] [chromosome = 5]
(SEQ ID NO: 18)
GTAACACACTTGCTCTGGGCCTTAGGTGTCTCTTTAAACCCTCTCTCCCCTCTTTATTTCCAAATCTTTA
TCTTTGGCTTTCTTAAGCTGTAAAGGCACACTGGCACCTTAGGTGGTCAGGTAAGATCGCTGGATTCCTT
ATTTCGGCGAAAGTCCAGCGGACCATTCCCGCCACTGTACCCATTTTAGAAACACTTGGAAGACCCTTAA
GGATCATTTACGGAGAGAAAGGTGTGCATTCTGCGGCTGCCGGCGGGCAGCTAGAGAACGTGGCGGGGGG
CAGCGCATCTGGCGCCTTGGCCGGGGCGCGGAGTGGGAACAGCCCGCACGCGTCCGCTGCCGCCCTGCCT
GGCGACGAGTGGCACAATCGCCCACCAGGAATCGGCTGGTGCCGCGGGTGGGCGAAGGCAGCGCCGCCGG
CCCGGAGGGCGGCGCGGAGGGTGGCGCTGGCGGCGTGAAAGGCGTCTTTGTGCAAGATTGAGTGTGGTGG
GGGGAAGGCGAAGAGAGAACTGATCCAGGACCTGGACGCGGGGGTCTGCGGCGCCCCTACGCCCCCACGC
CCCCAGCCGTCCGCAGTGCCGGAATGAGCCGGATAGCGCCGGCAGGGGGCAGTGTGGCGATATAGGGGTT
GCCGGGGGGAGGGGAGGGTCTCTGTTCACCTCTATTTGAGGCTGACTGGGGAGAAAGCTGCCTGGAGGAA
GCTGCTGGGGTGCGGGGGGCTGAGGGATTTGGCTCCCCGCCCTCTCCCCTAGATGGCGGAGAAATCGGGC
TAGCTGGAAGGGCGCAGCTCCTCTCCCAGAGCTCATTCTGGAGTCGGAGAACTGGGAGAGCGGCCCCCGA
GGCGGAGCCTCCCTCCCGCCCCGAGTCGCGCTGCCCCCCACCTGGGGGAGAGGGGGGGGCGCCGCGCTGC
CTTCCCTCCGCGCCTCGGCCGCGTGGCTTGCGGCTTATTTTCCCAGCTGGCAAGCGTCGCGCTGCAGACA
AGGGAATGCCTGTGGTGAGTTGTTTATTTTGTTGAAGTTTGCGGTGGGCCCGGGGGACGTGGGGGGCGAG
CGGCAGGCAGCGGCCGGGACTGTTTTGTTTAGGTCTCCATCCACCTGTGTGGTATAAATCTGCCAGCCTC
AAGGCGGCGGGGGACCGGAGTGAGAAAGCTCTGACGCTCCCAAAAGCCCCTTTTCGCAACTTACAGATCT
AGGACCACATTTGATTCTCCACGCCTGGAACCGGCTAGATCCTAGTTTACGCTCACCCCGCCCTCCAGCC
TCCCATCAACGCTTCCCGCACCCGCTGTGTGCAGGGCTGGCGCTCCGCAGAGCAAACAGCGCCCACGACC
CAGGGACAGCTGGCCACCGAAGTCCCTCCCCGGCGCCCTTCCCCGTTCCTTGAGGATAGGGGAGCTGAGG
TTTTCATGTGAAGGGCGCCCATGAAATTCGTGGGAACTTGTTCTGGCGTCTTCTGGAGTGGAGTTCTTTC
AGGGGTTTCGGGTCTGCCCGGGACTACAAAATGGGATCCTGGGTTGGCTGGAGTTTCCCAGACTAGGCTG
GAGAGAGGTCGGCTGTTGGCTCGCGGGAGAGCCCAGAGGAACCCCTTGGGCGATGGCAGTCAAGACCGGC
GGCTGCGTTTGCCTGGAAGAAAAGCCTAAATACATTAGCGAGCTGGTAAAGCTTTTAAGGCCTTCTTGGG
AGCGAGTGGATGGCTAATGAGAGGTTAATTTTGTTTCGAAGGGAAGGCTGCACTGGTTTCCTGAGATAAA
TGGACAGGAAGCGCATTACCTCCGCGCGCTGGCAAGCGGCTCCCAAATGCTTTTTGCACCTGCCGTTTTG
AGCGGTGAGAGCAGCGGCGGAGCCCGCAGGGATTGCGGGGGCCCGTTGTCTACGAGCGGGTGATGAGCCC
CCCGCAAGCCCCGGGCTCTCTCCCTCCGCGAGTGCATTGCTCACGCCAAGCTTTGGGACTCTTCTCTCAC
ACCCAGGTCCTGCGTGTTCCGAAGTTCAGGGGCCCCCTGATCCCGCTGAGCCCCCTCCAGGAGCGAAAGG
GTTAAGAATGATGTAAGTACAGGCACTTGATTTCTCAAGTCGCCCGCTTGGCCCTCTTCCAAGTTGTACT
TTCCTTATTTTCTCTCCTTTCCTTTCCTTACTGTTCTAACGCTTTTTAATAAACTTTCACTCTTGCTCTG
AAAAAAAAAATGATGTAAGTACAAACCACGAGCTGCTTGACGCTGAGCTGTTGGACTCCGGGCTATTTCA
GCCGCTGCTTTGGGTACACAGTGGCCGGGGAGCAGCCGGGGAGCCCTTACTCTAGAAAGGATTGCACAAA
TCGCAAGCTTAGGTGATCTGTTAAGCAGTCAGAGATTCTCCTGTTAGATTTCTGGAAATGGAGACATATT
AACTGGAGAACAGTTAAAAACATCACGTATAGGCTCAGGAAATGAAACTGCATCTTGTTTAGAATCCATT
AGTGTTTCTGGCATGTGTTTTAATTCTGTCCACTTGCACGTTCATGCCGTAGTATGCTTTGCTGGATTCT
CCGGATGTACTGTTCAGGTCTCCTCCTTCCTTTCTGAAAACAAAGAGAAAGGGAGAAAGAGTACCAGTGG
CAAGAAACAGGGTGGATGTGTGAATTACACAAGTCATCAGCCACTTCATGATTTTTTTTCTGATTAAAAC
ATAAAACTTTTATAAGTCACCGATTTTTTTGTTTGTGTTCTAGCTCCCTCCCCGATTTTTCTTTGTACAA
TATAAAGTCGTTTGTAACTTGCAGCTCACGGGCTTGAGGAGTGGCTGCGGTATAAGCGACTATTCACTAC
GCTCTTGCTTTCCAAGCCAATTCCTGATCTTGTGAACAGTACGAATTTACCCGCGAGTGTGTCAACTGAC
CTATGTCTTGAAAATAGGAACCACCTTGCGGTTTACTTGTGAAAATTAGCATTTTCCTCTAGCGGCTGAA
CACTTATGCAGCAATAAAAAGTGAATTGTTAGGCTTATAAATCTCGAGCTTGTCAAATTTTACAACAGAG
GAGCAATAAAGCACCTTATAGAGATGAAGTTTTTCCTCTGAAGAACAGTTCCGTTTAACGACACCAGGAT
TTGCAATGACTCGAGGACGCCCTCTATTGATAGGAAAATCTAGAGCACCCGTGTACTTATGGAACAGGAT
ATAGGGGGAGGGGAGTAAAAAATCATAGAAAGTCACGAGTGGAAAGACCCAAACCAAACAGAAATTATTG
ATCCTTAACTATTGAGTATATGCTTTGAAGTATGATCAATAAAGTGAGATGCTGCTTTTATAGGGGTCGA
ATTAAATCTTGGGTTAAGAGAAAAAAATGTAGTCTTTGTTAGCCAAATACTTTGAAAATCAGTATTATGT
TTGATTACTTTTTTAAGATGCTTCTTAGTATGTCATGAACTGTATCTAATTATATGAAGTAATTGCAAGA
TAGTAAATTTTTGCTCTAATAATACTTAGTCCGCAGCATTATCTAAAAATGTCATATAACTTCCTCCCCA
AAAGTCATTTCTCCAGGCAACTGTGAAGCTATTATTAAGCGTATTATGGATATACACAGGCTTGGATTAT
ATTGTGGATAGTGAAAGGTGTGTTTACCCCTTAAGTCATCATAGTATGTAACAAGTAAGTAATGAAAAGT
TACAGTAAAAACAGATAGGCATCCTTCATTAGGGACCTAGTGGTTTGGGCAATTCAAAATGTGTCTAATA
AGTGAGCTGGTGAGACTGAAGGTTTGCCCCTCAATTATCCTAGTCCATTGATTAGCTCATTTAAACATGT
GTGGAATATCAGCATGTCCGTGGTTGATAAGAAATTCCTAATTAGAGATTCTGAGTAACATAGCGGGTAC
AGTTGCCAACTACGATTTGGATGTTAGTGGATTTTAAATGGCAACCCTGTGCTGTTTGGGGAAAGGCAAA
GGCGAGGGCGGTATCAAGTGTCTCTCAGCCTTGAGGTCATTCTTGAGATTAGATACCACTTCACACCTAC
CGATAACAAATTGGAATCTATCATCAGTCTCATAACTGCTTTTCTGTCTCCACTTGGCAGCCTCTTTCTT
TGTAGACAATTGTCTTTTTCCTCACTTTCGGACCCATCCGATCATAAAAGGAGAAAGTGAGCTGCCATTG
AATTAATCCGTAGAAATTTGTGGAAAATTTAAATAATCTGTAGAAATTTGTGAAAAAGCCACTGAGCAAC
AACAAATCGTGGCATAATTACACTGCATAAAAACCCACCACTCTCATTAATTACACTGTAACCGAACGTT
GAAGTTTGCCCACTCTTAGACCTCTCCTTGCCGCTTTCAGGAAGGGAAATGTCAAACTAGAACACCTGAG
CCTCGCACAGCTGCCAGCGAATTCCGGCGGCAGTTCGCGGGTACCTTCCCAGCCCATCCTATCTGCTCCG
GACCGCGGGTGACATTCAGATTTCCAACACCGCCCCGAGCTTGCTTTACCAGACACCCTCAAACGACTCG
GAACGCGGTTCCGCAGATGTGATTGGCTGAAAGGGGACCTGCCGCGCGGCGCTCTGTAAAAGCGCCTGCT
TCCTCCAAGGAGGTGGAGCTGAAGCTGGCAGGGCCAGGGCAGGGAGGGGTAATTAAATTAAAAAAAAAAC
TTGGAGGGAAGATGAATCGGATCACATTAATTCAAGGAGAGGTCAAAGGCCGGTACCTTTGTAACGCCGG
CGAGAGTAAGACTTGCGGTCGCCCATTTCTGTTGGGCGCGAGGAAACAGTAAATCGCGGAAGGGGGTAGA
GGGGGAAGGAAAGGGAGCGCGAGGCAGCATGCGGCGCAGGAGAGAGGACTTGGAGTCGATGGACAGGGAA
CTCGAATTACACTGAGCGGGCGGGGGAGAGGGCCCTCTGGGTCCTGCGATTGGCAGGCTGAGTCGGGCTG
ACGTGCGGTCACAACCAATGGGGAGGAAGGGAGGGACACGGCGGGGGATTATGGCGTCGTGGATTGGCGG
GCACAGGGCGGGGGGGGACGGCAGTGCGGGACTGAGATTGATCGACCGCGGGGGCTGCCGCGCAGAGATA
TCCGGGCCGCCGGTGGGTGGTCGCTAGGGCTGGGCCAGCCTCTTGGAGGTCCACGCCCGCCGAGCCCACG
CTGGCTGGGGCCGGGGTGCCGGCGCGCTCGGGACTCGTCTCAGCAGTCGCTCACGGTCTTTGTGTCTTCT
CTTCCGCCCCTTTCCCTGCCTGCCGCCTCCGGCCGCCACGATGCCCCTGCGCCCCGCTGCTGCCGCCGCG
GACTGGCTGCGCCGGCTGCGCGCTGCTTGCTGCGGCGGTGGTGGCGCCCCATCTGCTACACGGGCCTGAA
GAAGGAAGAAGAGGAAGCGAAGCGCGCCCCCCGGCCCATGCCGCAGCCACGGGCCCAGACCCGCCACGGC
GCCCGCGCCGCCGCCCTCGCCGGAGCCCACGAGACCTGCATGGACGGGCATGGGCTTGAGAGCAGCACCT
TCCAGCGCCGCCGCTGCCGCCGCCGAGGTTGAACAGCGCCGCCGCCCCGGGCTCTGCCCCCCGCCGCTGG
AGCTGCTGCTGCTGCTGCTGTTCAGCCTCGGGCTGCTCCACGCAGGTAGGACGGGAGCGCGCGAGGGGAG
CCAGCCCCGGGGCCTAGGGCTTTGTTCCCAAATAGCCCCAGGTCTCGAAGGCGCTCGCCGGGGCGGAAGG
GCGGCGTGGCGCAACGGGAAAGGCCTCCCGAGCCCCTGACACGCACCTCTGTCGGGCTCCGGGCCACCGT
AACGCAGCCACCCACCGGGCGGCCGCCGGGCGCAGAGACGAGCCTTCTCTCCACCCTCCCCGCCTTCGGC
TCGGACCGCTGACAAATCTCCCCTCCGACAGCCGCTTATCAAACATTTAGCGGGCGGGGGCCGCGCGCCC
GAGACAAATGTTCCGGATGCTTTATTGCTCTCTGCGCTGGTGTTTACTGAAAGTCAGGCTCGGATCAGGT
TTCCGCCGTCGCAGCCGCCTCGGAGGGAGACAAGGAGCCTTGTGCTGCTTTTCTGGAGTTGGGGGTGGCG
GCGAGGCGAAGGAAGTACACCAGATGGAAAGATTTTTACCAGATTTTGCAAACCTGGGAGTTAATCTCGA
AGTAACGTCAACTTTTTTGAGCAGGGATTTCTTTCGTTTTGGCAAAAGCTGCGTTTGTTCAGCGATCAGC
CTCCCGGGAAAAGCTCGTGTTTTGTGACTAGCCGGGTGTCTTCGGGCTCCGTGAGGGGTTGACAGATAAC
CATGCTGCCTTCTGGAGGTCGGCGCTTCCCCAGCCTTGAGGCGTATGTTCCAAGGGAAGTAAGCCTGCAA
ATGAGCCTTCTACCATGTGATACCTATTTTTCTTTTTAAAAAATCGTTTCATGAAAATCCAGGACGCTCT
TCACACTATTTTTTTCTAGGAATTTGCCTAGACAAAGAATTTTTTTTAATGCTAAGGTTGAAAGGGTCAT
TTTCCTTTAGTCTTGAGAACAAACAGCATTAGATAAGTTTCTTTTCCTTCCAGACCTTTTTTGTCCTGTA
TATATGAGACACTTCACAACGTTCTGGTCTGGAGAATCCTGAACTTCGATGCCACCCAACTGTGCGGAGT
AATTTTTACTTATTAAACCATACCCGCCATGCTAAAAATCAGGGAGGTCTTTTAAGTAATGGGGGGGAAA
ATGTGACATCTGAGGAGACTTAATTTACTTTGCGTAGCTTTGCATTATGATGTAATAGCTGCTTTTTAAC
ACCGACAATTAGGGAAAAGACGGCTTAATAAAAAAGTGTAATAGAAAGTTTTAGGATGATTACGGCTTTC
AATTAGTAACCTAAGAACCTTAGAAAATAGTGTCAATCTGCATGTAGTTACGTGGAAAATTTTTCTTGAC
ATGTTAAAAGATATATTCAGGAGTGTTTGTGCGAAAAATATGTGTCCATAATACCAAAATGTAAGTTTCA
TGAAATGGAAATTGTGCTAGGTGGGATTGTGTCCCCCTTGGCTGCTTGGATATTGTTTCCCATAGTTGCC
ATAGGATCCTGTGACAGAATTGGAATGTGGTCCCTGAAGGTTTCTTTGCTAGCCCGTGAAAGGCATACTA
AGTTGGCTTAAAGGTTTAGAAAAGTAGGGTGTCTTCGTTCAAGAATTTGGAATGTGAAGCTCCCAGTATC
TTCATGTGTCCTCATTTAAGTCACCATTAGATTCAGCCCAACAATGTGGTAAGCGATTTCTATGGACAAC
TTGTTGGAGTGAAGCGATTAATCCTGTTAAAGAAAAGAACGCTCCTTTTGAGAGATTTCTTTGAAGAGTG
GTGCTATTTAAATGCCCGGGTTGTTTCTCAGATAAGGGAACCCAGCAAGAGTGCCAGTGCTCTTTGCAGA
TGGACTACTATATGTTTTATGCCTGGATCCTTATCATCTGCCTCAGCACCCCATATGGCTCCACATTTCA
CTTAATTCAGCTTCAGAATACCTCATTTCTTTTATTATAAACTGGTGATTGTGCTGCAGATTCACACGTG
TAGTCCTGGTGTTAAAGAAACTGTCTTGAATGGTGAGGTCTTTATGTCTGATTCATTCAAAAATGATGAA
ATGAAAGCTCTAGACCCACAAATGCTTTAGCATGTTCAGTAACTGGTGTGATAATACTGTCACACTGATC
ACAGATAATCTGACCCAAAAATAGAGAAATCTGTAGCTGCAGAGTTTTAATTCTGCCCTTGGGGAATGAA
AAGAGTATGAAAAGAGTACCTTTAGTTGCTTTATTTTATTTTATTTTTTTTAAGCAAACCTCTGCAATTC
CTGAATAGAGATCCAATATGGGAGTTACTTGCTTACTCATACACTTCATGGCCTTCTTGGCTACATCCTT
GAGATTGTTTCTGCTCCGCATTGTCTTGGCATTTTGACAGGAGGCTTACTTTACCGCCTATAACAGGCAA
AGTGTGCGTTGTCGTTATAAATTCATGAAGCCTGGATATTTCTGGGTGTATGCTACAAATTAGTCATTAT
ATAGAAGCCCACAAGGCAGCTGAAAATGCTGACCCAATCGCACTTTCTATTTAGCCCTCTTCGCTCCCAT
CATCTCTGTAGCAGAAGGCTGTTGGAAGCCTTAGCCAAAGCAAAAGAACCCCGGGAGATCTGTTTGATTT
GAGATTTGTTTAAGCAGTCAGCACCTACCTGCTATGAAAGAGGGAAAGGTTTAGGCCGGGCGCTGTGGCT
CACGCCTGTAATCCCAGCACTTTGGGAGACTGAGGCGGGCAGATCACCTGAGGTTGGGAGTTCAAGACCT
GCCTGACCAACATGGAGAAACCCTGTCTCTACTAAAAATACAAAATTAGCCGGGTGTGGTGGCGCGTGCC
TGTAATCCCAGCTACTAGGGAGGCTGAGGTAGGAGAATCGCTTGAACCTGGGAGGCGGAGGTTGCAGTGA
GCCGAGATCGCGCCACTGCTCTCCAGCCTGGGCAACAAGAGCAAGACTCCGTCTCAAAAAAAAAAAAAAG
GAAAGGAAAAGGTTTGGGGGGTTGCAAGGAAGGTATGAAAAGGGAGCTGGGAATCTAAATTCCACTTTTC
TTTCCTTCTGAATGGTTCCATCTGCATTTCCATACTGTTCGTTTGATATTTTATGACTATTTGAAATAAT
TATAAAGAAACGTATGCTCGGATAGATTTTATAGACCATAAAAAGAACAAATAGGCACTGCAAGTTTAGC
GGGAAGTTCTTGTGCTGTCATTTATCCGTAGTCTTAAGAGCTCTTCATTACTGAGACCTTGGCCCCATAT
TTATTTTCTTGCTTTCTGTGTATCTGATTTTAAAAGAAAAAAAATGCAATAGCTACTTAAGTCTTTTTTT
CAGCCATCAAGTACTGTCATTGCAGTTAGGAAATAAGAGGAGACGTGACCTGTGATGGAAGAACAACAAG
TTTTGAGGGAAGGCACCACATCCTGGCTTAATGATGCTAGCTGTTTTATGATTCTGCCAGAACAAGCCCT
TTAATATCCCTTTGCCTTGGATTTCCCATCTGCAAAATGGCTTTAACGCCTTTTGGAGTTGTTTAGTTGG
TTAATTTCTGTTTTGGTTTGATTTGAGTTCTAGTGAAAAATAGCTGAAAATTATTTGATCTGCTTTGGAA
AGAGAAATTCAGTAAATACTGGAGCTACTTTTGTCAGCTTTGGAATGAGCAAACATAAAGTAGATTCCAC
ACAAAGATGTTTTTGTAACTTTTTTTTTGAAAGTATGATTTGATGATTGCTGTAAATCCATAGCTCCAAC
ATATTAAAATGTTTTAATCTTTGACTCAGGTTAAAGTGAATACCAAGCCTAGACTATATAGTATTTTCCC
CTTTTTGACCCAAACACACTTTCTCTGTATTTAGCCCTCTTCCCTCCCATCATCTCTGTAGCAAAATGCT
GTTGGAAGCCTTAGCCAAAGCAGAAGAACCCAAGAAGATTTGAAATTTAAAATTTAAAATTTAAAATTTT
GCAAATTTAAAAGTGGTGACAGAGTTGTCTTCAGAAGTTGTCTTCTCCCACCTGTTTCTCCCAGCCAAAG
CCAGAAACAGAGCTTACAAGCTACTAAATTCAGATTTAAGAGCAGTTTACTAGTGAAAATGTTAAAATAG
CTTTCTAAGACAATATTAGGCTAAATTTTAAGTCTTTATCAGATGGAGTTCCCTAAATCTTGATATCATC
CTGAAGTTTATTGCATCCACATCTTGAAATAATTATATATTTAATAATAATCTACTGGCCCTGAATTTTT
TCCCCCATCCAGAATACAAATCATAAGATATTTACTGTCTGGAAAAGAATACAAAGTTCGTCTTAGAATA
GCTTTCTCATTTCTCAGATGAGGACCCTCACATTTAAAGAGATTAAGAGACGTCTCCATAGTTTAAAGAC
CTATAAAGATTTTATGTAACACAAATAATGTGTTACAGTTCATTTATATAAAATTTGCAGTTGATGACAA
AGAACAGTGTCTCTTTTCACTAAGTCTGTTGTTTCCATTTTATGGAACAGAATATTAAATTATTTGGGGA
CACACAACTGACCTAGTATTGGTAGTTTTTGTGCTTTAGGAGATTTTATAAATTTTTGTTTGAGAAACTA
GTGGTAAAAAGCATCATTTGGAAATTGATTTTTTTAAAACTAATAGGGCAGTGATACTTTGGTTTTTCAT
AATGTTTATATGAATTGTTTTTATTTAGTGTGCTTTTACTTGTGATCTGCTTGGGAGCTAGGATCCTAGG
GTAATGGAGATAGACAGTAAGGAAAAAGTAAATATATAGCTTGGTCAAGTGAATCTTGTTTTTTTTTTTC
TTAGAGCATTTACATTTTGTAGAAATTAGGAAATTGTGTCAGTTTATTTACAGTTTCTTGGTAAACGTGC
CTCAGGAGAAGTAGAATGACAGTACCTGGATTTTTTTTTTTCTTATTCTGTAGCCATACAGGTAAATCAA
GTAAAGGTGTTTTAACTAATTATACTATAATTTAGGATTTTACACCTTCCTGAAGTGGGGAGGTTTTAAC
CATGGTTTTGTGCCTAGTGCTGCTTTTCCAATGCTTTCTGAAGGAAGTTATGCAATTTTGGAAAATCAAA
ACAATGTTCATGTTTTTGGCGTAACCTTAGAGTAATCTTTTTCAAAACCTATGAACAAATAGCAACATTT
ATAAAATATTTTATTTACTATTCCCTTGTAATCTCAACCTGTGATCACTGGACTAGACTCATGTTCACTC
ACAGTGGCTCATGCTTTAAAGTTGTAAGTTAACTAATTGCATGCAGTTTTTCTTCCAGTGGCTTAGTTTG
AGAAAGACACAGGAACACAATGAAACCCTGTATCTGAAGGCTTCTTAAAATAGGAGTACAGTGTATAAAA
TAGAACAAACAATTCCCAAAGAACAATGTGATTTTCTCATGTAGCTCAGGAAGTTATGAAGAGTTTACAA
ATGTTTGGTCTTATCTTCTTTCCCATAGGTGACTGCCAACAGCCAGCCCAATGTCGAATCCAGAAATGCA
CCACGGACTTCGTGTCCCTGACTTCTCACCTGAACTCTGCCGTTGACGGCTTTGACTCTGAGTTTTGCAA
GGCCTTGCGTGCCTATGCTGGCTGCACCCAGCGAACTTCAAAAGCCTGCCGTGGCAACCTGGTATACCAT
TCTGCCGTGTTGGGTATCAGTGACCTCATGAGCCAGAGGAATTGTTCCAAGGATGGACCCACATCCTCTA
CCAACCCCGAAGTGACCCATGATCCTTGCAACTATCACAGCCACGCTGGAGCCAGGGAACACAGGAGAGG
GGACCAGAACCCTCCCAGTTACCTTTTTTGTGGCTTGTTTGGAGATCCTCACCTCAGAACTTTCAAGGAT
AACTTCCAAACATGCAAAGTAGAAGGGGCCTGGCCACTCATAGATAATAATTATCTTTCAGTTCAAGTGA
CAAACGTACCTGTGGTCCCTGGATCCAGTGCTACTGCTACAAATAAGGCAAGTATACCTTCTTTTTTCCC
TCTCCCTACTCAGCTTTCAAAAGATGTTTGATTCTTCAGATAACTTTTGAAATGTGCTATAAAGGGCCTA
GTTTAAAAGGAACTTCTTTTGAAAAGCAATTAACAGTTGATAAAGGATTAAATAAAAATTATCTAGTAAG
GAATTTCTTATTGGAATGTAAACGTGGTTCTAATTTTAAATAGACAGTGATATAAAGAATAAAAAGTAAA
CAGTGAAATTGAGTTCTCCAGGGAAAAGGCAGACCTGTTTAGTAAAAAAAGGATGCTTTTTTCAGTGATG
TCTTTTTTTGAGTGCATATGTGTGTGACTCTTGAAGAAATCCATGTTCAGATTTATCAGATGATTGAAGT
GGGTGTTCTGAATAAAGAAAGCTGTGAGGCCTGAGGCAGTGACGTATCAGGAAACATATTTTATTGGAGA
TTTGGAAGCTATAGTAAAACATAATGGCAATAAGCCAACTTCCCAGTGGTAAACCCACAGTGGTGGTTTA
GTTACTAACCTCTTGATGACCGAGGAGGTTAATAATTGGATATTGCAGAGCAGCAATATGTAACCTGTGT
GTAATCTCAGGGCCTCAGGTTAACAGTTTCAGTCAGAAGCTAAGAGAAACACTGACAAAATTTAGCTTAC
CATGACTAGCTGCCAGTTTATGTGGTCTGTGTTCGTTTGTTTTAAAGAAACTAATACTGTACAGAGATCT
TTGACACCAACTTCAAGGCACCAGTGTTTAGTGCAAGCAGGTAGAGAGGACCTATGAACTAGGTTTCTAA
TTGGCAGTCAGTGCTAATTCAGCCATTAGTAAGTAAAGTCACTTTTCCACTAAAATAGTGTCCCGACTAA
CCACCTCCTTCCGTGTCTTTTCCAACCACAAAACCACACACATAGACCATCTCATCTTACTTTATTTCTT
GCATTTATTGTATCCAAAAAAATTGGTTTAGTTTTTTAATGTAATTGACAAGACTCAAGCTTTTGAAATA
CTTACGACTGTTCTCTTGATTCCAGGACACCCTCCCCTGTGTCTAAACATGTGTGGTATACTTTTTAAAT
AAATTAAAGCTTATTTTACTTGGCATTTTGTGGAGGGAATTTTACAACACCTCGGGAGATATCTGCAGAC
ACCCTGGGGTGTCACAAATTTAAGACTAGAAGTCATTGCCCTAATCAAAGACGACTGTTACACTGGTCTA
GACATCTTAGGGGAACTCCAAAATGTTTATACTTCAACTCTCAGTAAAAGCTCAAGCTTGTCTTTTGCCT
TACCAAATTCCATATCTCAAAATAAACTTTTCTCCCTCTGGTTTTTGTATGTACTCAAATCATTTTTTTA
TAAATATCTCAGAATATGTATTTGCTATAAATTATATATAAAGTTATAGAATTATGAAGTTATAAGAGAA
ACGATGGCCCCAAATCCATCATTACTGCAGAGTCATATGGCACATGAAAGCTGGGAATAGTCTTTGGGTA
ACTGAAAGAAGGCAGTCCATTGAGAATGGAAAAAGTCATCAATTGTGCAAATAATTGAGGACTTGGCAGA
GTGACCTTCGAACAAAACTATAGAGATTAATCAGAGCTTGTAAATGGGGAAATACTCATTCTCTTCACAA
GAAGCAGTATTCAAATTTTATTCTTCATGCCTTCCATCCGGTCATGTCACTTCTGTTATTCAGTCTTTCA
GAATTCTATTGTTACTGATTATGGTGAAAATATGTGGGGGATAACCTTATGAATATTGCTATATATATGG
AACAGTTTACACTGAAGTATTTAAGATCATTTTATTTCAGTTGAATTCTGTTGTAATTTTTAGCAAATAA
ATAAAAAGGACAATCTTGTTTTCTCCCTCAGTTTGGAATAAGGATCAAAGGTTTTACCCATTCAAAAAAA
GTTGGAGAAAGTGGTCCTCTGACATAAATTCAAGTAGATGATGAGAATGCTTGGTCTTTAATTGCACTAT
TGGAAGGTATTTTCCCACATCTGGTGACCATGCTGTTCAGCAGCAAACTATTCTGTAGCCTCACTTCACA
ACCTCTAGGAGTTGTTGGGTTTGGTTCCCCATCCCCCTTCTCTTTGTAACTCTCTCCAAAGCCCCATAAG
CATCTGGAAGGTTGCTCTTGAAGACCTGTCTCAATACTAGTATGACGTGTGCTGGTGAAACCAAAGAAAG
GCCTAAATCTTACAATACGTTCCTTTTACCCAGCCAGGACCAAAATAAAACCTTTAAAAACCTTCAGCAC
AAAAAAGTGGTGGTGATAAAAATAAAAACAATAAGAGGCATAAAAGTGCTTATAAAATAAGTTCTAAACA
AGATCTGAAATTTTCCCTTAATGTCTACCACCATACTTTCTTTAAAAAATCTTTTGAAAAATTTTTTCAT
GGAAATCCCAGCTTTACTGAGAATTTAAGCAAGTGCATAGACCAGTTGTTGGGTAACCTAGTACTGTGCC
TTGTAGATACCAGATCATCCTTGTGTGGTCAAGTGTTTTTAATAACCCACGTGCTCAGTGCCACTAGATT
GAAAGACTGATGTCATTGGCTAGCTGGCCAGCTGTGATACTGAAATTTTCGGAAACATCCATTCATTTCG
CTCCCTTTAACACTGCACTGAAGCACTAGACCCCAGGGTATTTTGCTCAATGAGGCTACTAAGACAGCTT
CTCAGTCCTGGGAACCTGGCCTCTTGAGCAAGCCGTCTTCAAGCCCAACTTTGTGAGCTCTGCCTGTTCC
TAGGTACCCCCCTTTTCCTTATCACAAAAAGACAAAGTGGGGGAAAAATCTAAACACCATAGAATGAAAT
GAAGTCACCCGGGGCCATATATTAGCTAAGCCTTTTCTTTTTCTCTACATGGCAAGCAGTGAGTTCAAGG
CAGACATGCCTGCGGTGCTTAATGATGTTGCCTCATCTCCCCTGGTCTACTGTTTTCTTCCCCACCATGG
CCCTCTCCTCCCCTTCCTTGGGAAAGGTAGGGTTTGTTTTCCTGTTGGTCATGGTGTGCACAGGTATGCT
TTCTCAAGGCCTCTGTGTTTTTAGTTTTGCCCTGTGCCTGCCTCCCTCTCTTTGGAGGAACCTGTGCCAG
TTGGCTACCTTGCCAGGTAGATGTGTTTCTGAAGGGGAAGCCAGGGCTGGTTTTTCATCTCTCCACCCCG
GGGGCTGCAGGGGTGTCTGGCACACACACATACCTCGGCTGACATCCACATCAGCTCTGGGCCATCTTCC
CCACCTTTGGCTGTGAGAGGCGGCTACAGGCATGTAGGCACTCAGAATGAGATGAGAGGGACAGGCTCTG
AACTGGCAGAGTTAGCTACACTGAGAAGAAAGGCAGGTTTTGTTTTGCAGGAGGAAGTAGCCCACCCTTC
ACAGGCTCCAGAGAGGGCACATGTCAGTAGTTGTCCTGTGCCTCACACACTCAAGATAAACCAGATGATC
TGTTTTGAGGATGCTTGACTTCTTCCATGTTATTTTTGCCAACCTCTCAGTCTCAACTCTTGTTCATTCA
TTCTGTGGATCTTTATATCTCTGTTTAAGTGGGTTCAATAAGAATCTGTATATGCCATGATCACAGAAGG
TGATTGGGGAATAAATTTTTTTATGAAAGATTCTGCACTTTGCAAATGTATTCCCAATAGCATATATTAA
AATTGCCAGTCTCCAGGGAATTCAAAAGTATTCCTTTTTCAAAAACAATTCTGAAATTCATTTATTATTA
AGTTGCATACCTAAACAAACAATGGAAATGTTTTTAGTAATTATTTTTGTACAAGTAGTTTGAATGCCTT
CATTTTATTTTTCCTTTTGTTTTAATGTGGTTGTATCCTTTTTCACCACTCTTGCTTTCTTATTCTATCC
TTCCACCTCAGTAACACAAAATAAAAAGTAGGAGTGGAGACTGGGGATGACAACAGGTCCTACTGCAGAT
CTTCTGCTACACATTTTACTGTGCCTCACCCTTGAGTGAGAGAAGGTAGTTTACCTTAAGGCGTTTGAAT
TTGGGAAAGGGCTGTGCTCATCGTACTTGACCAAGCAAGATGACTGTTAGGCAGATGGGCAGATGCTCTG
AGGTGCCTATCTTGTACGTTTTTGGTGCTGCCAACGGCCACCAGCTCCTGGCAATCCTATGGGCAGCACT
CATCGCCTGTCCAAAGCATAGTAGCATACCCATCATGCATTATGGATAGTCTAAATTCCATACTTACCCC
TGAATTTCAAATTCTGTAGCTACTTTCTTGGGAATTCTTGAAAATTTCCGCTGGTTGAAGATACTAGAGA
TGTAGAAAGACTTAGTTTGGTTTGCTTTTGGTTAATGTCTTCTTCAAACTCTGTAACCTACAGTTTTCCA
TTGTAAGAAACACCATTACCTTGTAGGCTCCTAGAGCATGAGGTAGGTCTGCTTTCTGGTGCCCCTTAGC
AGGGAGTTATTGTTCTACAACAGCATGTGGGCATGTAAACCAGTGTTCTGGGTCATTCTTTGTTGGTTAC
AAAATCAGAACCAGACATGCCTGCATTGCAACAGCTTTCAGTTGTCATTTTAAGTATATCTCTGCATAAG
GTTTGGAAATCTGTTCTCTGTTGACCCCAGACCTAGTACATTTATTGTAGCTAAGTTTTTGCAACCCATT
ATGTTTTTAGCATCTTTATGGAAACCATGTGAGCTACATAGTGTTTATCATTATTTTTCTGGACTAAGGG
ATATGGTTCTTCCATTGATAGGTCTGTGTTTTGAATCTATGGACACTGTTCTTGCATGCAGTGTTATAAA
GAGGGGGACGCATGGTATTTGTGGGGGAAAAATAGACCATTTTCCCTCCACAAGGAAACTGCAACTCTCT
TGGTGATACATTGTAACTAATTCTTATAATCTGAGATCGCTCTTTATTTTTGTTACAATTACATTGTTGC
ATGGGTAATTCTTGGCATCTCTTTGAGATTTGCTCCTTATTGTGACCCTCCTTGCTTTATGAAGATGCTA
AGAAGTTCTGACTGGTTGAGGCATATTGGGAACAGATGGAAAGGATTTTGCAGTTACACCTCCCTGCTTA
ATGTAATTATTTCCCTGATAATCATTCTCTGATAAATGTATGGTTAATTTGGTGCTGCATTTGAAACAGA
AGGTACTAAAGGGCCCACTGTGGGAGATGTAGAGGTGAATGCAGCCTTTGCCTCAATCTAAGCACATAAC
TAAAATTTGCAGCAGTTGGTGGGAAAGCACGGGGTTTGTGTGTAAACCCAGCATTTTCAGGGTTCTTTTC
ACATTGACAAGCTGGTTCCAATTGATGTTATTCATTTAGCAAGTTGGCAGGGGTGTCAAGAAGAGCCAGA
AGAGTGGGCTGCTACAAAGGCCTGTCCCACGCTAACACCCCTCCTTGCCAAATCATTGTCTTTACTCCCG
GGATTTGGCACAGTCTCTTTCCACCTCCCACAGTAAGAATGCTCACCTGCATCTGTATTTCCTCATCTGC
ATACAACCTAGCAGGGCCTAGAACCCTGGCTCCCACTTTTGAAAGACTGCTGATCTTTGATGTTATACCA
AAAAGCTTTCTAGAATTTGACTGTCATAGTCGCCAGTTCTTAATTAAATTCATTTTCCAGAGCCTCATCT
CATCTTTAAAATTCTCATAAAAGCATATGTTAGCTATTTTTACCTCCCTGAATTTTTTTATTATAGAAAC
AATATTGAAACATTAAAAGCCAGGTTTTCAAAAGAGCAGAAATCAGCTGTAGTTCCAGGTGGCAGTCTAA
TTATTTGTCCTTTTCTATTTATCATGCAGCTGTTGTGCATACTGGATTATAAGATCTTAGGCCCAGGAAG
TACTCAACTTTGCTTTATATCATTTCAGTAAATTGCCTGGAGGTGAAAAGGTTTCCAAGTGGATGTGGGG
AAAGGAAGCAGAGAAAAATGGGGAAATTGCTAGCTGTGTAATGAACCTATGTCTGAAAAATCATTTTGTA
AGATAAATGAGCTACCTGTTTATTCTTTATGCTATACTTTTCAAAGAAGATTTGCCTTTGTTTCATTCAT
ACACATTAAAAATTCTGGGTAATGAGTCTTTTACCATCTTTCTAATCTCCCTGACTGTGCCACTCCTTCT
GTCCCAGATGTGGTTTGGAAGGAGGAAGAGACCTTCAGAATGCCCCCCATTTGTAAGTGAAATTAACCCA
GCGCCTGTCCCTGTGTGAGCCAGGCAAGATTCCGTGAGGGTGCTATGGGGCTCTGCTGCTTTAAAACAAA
TCCACACTCTTCCATAGTTTAAACCAGAGGCATGAGGCCAAGTCTGATACACAGCTGAGAGTTAGAACTT
GTTTTTCTGTCACATTTCCAAATGTCTGCCTGGTTAAATTTTCTTTGAGTCAACATTTTGAGGGGAAGGG
GTGAGAAGGAAGCATTGGATGTTTGGTCCTTTTTTTGTTGGCTTCTATTAATGCTAACCAGTTTCTGCTC
GTGTGGAATGTGGCATATAAAAAGTTACCTACATTGTTGAACAATGAAAACTGCTACCTTATCCATTTTG
TTTTTATTTGTTTTGACTGCAGTAGAACTTGCTCTTCCACTGTTTCGAATTTTGTGTTCTTGTGGGTGGG
GTAACTTAAGGACTTTTCATACTGACAAGGAAGCTGGCCAACTTGTCAGTAAGTAATAAAAGAAAAGCTA
ACATACATCTAAGTTACAGACTTTAACTGGACAGCCTTGCAGTGAAATCACAGTGCTATTGGGTCTAGAA
AGAAGTTTAGGTGAAACTGGTTCTATGGAGAGAGGAGTTTTTTATGCCCCAGTTAACTGTTTACTTTAAG
AGTTGGAGCCAAAAGAACATGGGGGTTCTTTCATGTTGGGACACAGCATGTATACTGTGAATAGGATGAT
ATGACACCATCTAAAACTCACTTCTCATCTGCTCTGCATGTGCTCCTGAAATGGGGGTCAGTTTCCATTT
AAGACATGTGTGTGCATGCACAGATGTACATGCATGTGCTTTTACATGGTATCGAGGTCTTCCCCTCACC
CTACCAAGGAAAATGCAGACATAAAGATATTGTTTTGTAGGATTTCGGTTAACTCACTTCTTGAAATGCA
TGGTAACCATTCTAACCACTTCATTCCTGAGTTCTCATGGGTCTTTTCTTTTTCAGAGTGTGAGTGTATT
GTGAAGATCTTCAAGCCCTTTTACTTTTTTTGCTCACCTCCCAAAGCTGAAAAATGATCAAAGAGCCTGT
GGTTTTCTAATCCCATCGGTTTGGAGAACAGCTGTGCTGAACCTAGGCCCAGAGCAGGGCCTCGGGTCTG
TAGTCCTGCCACCTCTCTCTACCAGGAGAGGGAATGCCTGAAGGGAAGACCTGCTAGGACAGATTCGGCT
GAGAGTTACTGCCACCAGCCCTGGCTCTCACCTTTCAGAATATTTTTAGCCTCTAGTCTGAAGCAAGGTG
GCAGCCTAGGCCTATAATACTGTCCACACAGGCCTGCCTTCATATCTGGGCATATAGTGAGTTCCTACAC
TTGCTGGCCTCCTCACAATGTGGAGTCCGAACACAACAGACAAGCTAAGGGAGAGGTTCCATTTGGAGAT
TTTGGAGTGACCAAGACTTACAGGACAAAGGCCTTTCCTCATGCTCACCCTAATACAAGGAAGTTGGCGA
ATTTGCCTGTCTCTGTGGCTATTTATGCTTCCAGCATTCACTGTCTTAATTGTACAGAAATTACTCTTGG
GAAGCAGCATTTTTGTGCCTGTTCCTTTTATGTGTCTTCCTGGGCCAGCTTCCCTTTCATTCTCGTTTTA
GCCACTGAGCTTGATGCTGCTTCTCTAGACCACCCTAGCTGCCTTCAGCTCTGCGTGTCCTTCCCCTCTG
GTTTGGTTTCTTCATTCTCTATCTGCTGCCAAGTACCCCTTCAGCAGCCAAGGCAGCCCCTGTTTTTGCA
TGTATTTATTACCTACGGCAACACTGAAGAATAAGTTTGGTCCATTCCATTTCTCTGGATCCTGTAAGTG
CTCCAAGCTCTGTGGAAAAAGACATGAGGATAGAGAAACTGATACCTCTTTTTAAGGTAGCTGATTTTTT
AGTCTCATTCAAGCCCTAAAAGGAGTCCACAGTATGAGTTTTAATCTGCCACTCACCTCGTAAACACAGG
TACTGTCTTACTGTTAGTATCTTGTCCTAGTCCAATAAAATTGTTTAACAAAATGTATTTTTGTGGATCT
TAAGTTGAGCTTCCTGTAGATATTTGTTTTATAAGATATTTGATATGTAGGCTGAGTGGTGATTGTAAAA
TTACATCCCAGTTCATTGAGTTCATAGCTTCCTAACAGTTTTTTTCATAATTTTTAAAGGAAAATTATCA
ATGACATCAAGTATTTTCTAGTCCATTATTTTTTCTTAAATATGGCATATATAAAAACTCTCCTGGGTTT
TGCATGAAATTGTTCAGAAAGAGTTATTCTAAATCAATATACCTCTTTTCCCCCCTCGAGAGAGAGACAG
AGTTCTCCCCAGTTTTTATGTAACCAGGGGACAGTTGCCACCCCGCGTGGACAGAGTACCTCTGCTCTGG
CCAAAATCCACACTTCAGAATTACCCTGGCGGGTAGGGAGGAGTGGGACCAGGGAAGACACTTGAAGGAA
GGTGCTTTCAAAGGCCTCTACTTCCTTTAGGTGCTTCAGAGCATCCTAGTCTCTGACTTCGTAACATTTA
GAAAAATTGACCCTTGGAAGTAAAATTTGGGTTTTTTAGGAATGGAGAGAGAGCATGTATATGTTTTTCT
CCTTTGAAACATGTGCTCTTGTTGGAATGGTTTCTTTTTTGTTCACTGGGGAAGACTGTAGAGGCTGCAT
GCTGTTAACCATGTGTTAAGAGTAAAAGGGGCTGTGAGCACCGTGGGCTTATTCTCCCTTAGAGTCTTCT
ATTTTTCAACCTATCATTGCACCCAGATGATATGGCAAAGTAGACACATCAGCTAATTATATGAAATAAA
ATTCATCGACTTCTTTGACACTGCAGAATTTTAACCAGGTGCATATCAAAATTATGTTTTCATGTGTTTC
AAGCTAGTTTCTGTGCCTCTTCAGTTTAGTTTACTGACCAGTCTAGAGTCCAGAGATGAGAGCTCCTAAA
GTCTGGCTCTCATGTTTTGAGGATTCCAGAAAAGTCCTACACTCACAGTGCCAAATGGCCTGATTAAACA
CAGATAAATGTTTCTTCCAAAATATATTACGCACAAGTAGTCAAGGTGCACAGCCATTTGAGAAGTCTGC
TGTTTTAAAACCTGTTTGTGGCATTTTGCAAGGCTGCTGTGCTTTGGATAGATATCTCTGTACCTCTTGG
CGAGGCAGTACCTGTGTATTTCCCTCTTTGTTGGCTACCTAATTTTCATTTAATATGTTTTTTTTTTTTC
TCCCGGTGGAAAGGAGAGGCAGGTGCCTCTTAAAATTATTTCACAGCAAACCTTCGTAAGTTCTTATTGC
CTATTTCATGTAGAAAGGTTGATTTGTAGCTGCTGTCTTTCTCTTCCTAATAAACCTTGTGGGTGGTGAA
GATCTAGGAGAGTGAAACATTAAAAATGAATGAAGAGAACTGAAGTAGTTTGTCTAAGGTGGGGGTTGAG
AGGGGCTGGGTGGAGGAGGTTTAACAAAAGGCATTGATGTTTTAAATGCACAGAAATGCCATCTATCAAT
AATAAATGGTGTGGCATAGAACAGATGTGCCAAATTGAGTTATGACAGCATATAGATTAGTTCATAGAGG
GGAAAAGTAAGTAGCATAGCTAACGTTTTTAAATTATCTGTGAGCTTTTGAAATACAATTTTATGTATTC
AGTTTTTACTTTCTAGTGGCTGCTAGCTGGAAATATTAAAAGTTTGGTGTTTTTTAAAATTCCAATTAAA
TTGTGCTTGTTTCTAAAGTAAGAATTAAGAATGAATAAATTTTGCTTTTCTGAAGAGGAAGACAGTCTGA
AATGAATTTCTTGCCCTTCATTGGCTGAGTACTTGCCTTTTAAAAGCTCATGTTGTAATTGTTATGAGGA
ATGAAAGTGCATTATAAAACTTAAGTCATGTGAAACTATTACCCAGTGTTTTCTAAATTTACATTGATTA
TATCTCTAGGTACCTGCTCAGTGCATATTTAAACTACAAGGGACTACTCAGGAAACCCACTCATGCTTTA
CAATCAATTCACCCCCCTCAGCATTTTGATTCAAGGGATAATTCTACACATATGCTCTGTGTGAGAATTA
CCTTGCTTGGTTGGGAAACAACAAGAGAAATATCCACTTGTCTCACAGCAAATCACTACTAGTAAATTGG
AAAGACAAATCTTTTAGGTTAACTAGGATATTTTGTAGGTGTAGATACTAGAGCAGAAAACAAGTTTTGA
GCTTCTAGATAGGTAGGAGTTATTGTTAATGTCACTGGCATAGCAGAGGACAATACTGTGGGAGGTGTTT
GAAATGAATCCTGAGTTCCTGAGTTGGAATTGGTGTGCTTAATGAAAAAAACAGCCGTGGTGAGACATAT
GGAACCATTCCACTCATCTCAATATGGACCCTTGCTTGCTTTTTGGAAACTTGTTTGGAATCGGTTATCT
TTGCTTTGTTGGCCCAACACAAGTAAATCCTCTTTGTTTTTCCTAAGGTGAACAAAAAAGTAGTTCTTGC
AGATAATATCAAGTTGGTTTAAATTTTAGGTTTGTTAGACTAGAAAGACGAATTATTTAGCCGTCATTGA
AGAGTTTAATCTTTGAGTAGATGTTACAGGTGAAAATGAGCATAGTGATTATATAAAAAGTCTATTACTA
ACTCTTATTTTAGCCATCTGTATTGTTGACTTTCAACTTTTTCAGGTGCAAAATTTTCTTGAATCAATCT
TTTCATCTCCAAGGGATCCCAGCATGCCTTAAAAAAAAACACTTGTCAACGGATTTTAGAGCCACCAAAT
AAATCTATTTTATGGTCCCGTTAGATATCTAGCTCAAAACCCCCATTCACTCTGAAAGGTCCTATGGTTG
GATGCACAACAAATTTCCTGAAATCATCTCTGATGCACCTCCTTTGGGTTGTTAAGAAGGTGTTGAAATG
TGAGAAGGCTGGTAGTGAAATCAAGTIGTGGCTCAGGGTAATTAGGAAGGGAGAAAACCCAGCATTTTCA
TAGTACATTGTCTGGCTTTTAGTTGCAGCTGCACACAGCGGATATGAATTTAACCGGTTGCTACAGTGCA
GCTGGTTCAGATCTGGCTTTATAACTGGCTGAAGTAAAATGAAATGGCTGGAAAATTCCAGTCAAGTAAC
ACCAGTGGGAACAAGTGGAATTAAATAGTGTGAGCAAATGCTGGCCGAGTTGGAGAAAAACTTAAGACAT
AAACTTGAGGGTGGTGGTGAGGACTTCGGGTGGGCAGGCGGGGGGAGGGGGGTGCATTTTCTGGTCTTTT
TCACAAGGGTCTATTGTTTCGATTAGTTTCCTTGCAGGAGGTAGAAGGTTTCCTCCATCCCGAGTAATTC
ATAGAGTGTTTGGCTTGTCTACCTTCCTCCTGACTGAAGTCACCTGATACTTTTTGTTTTTTCAAGAAGG
AAGAGAGAACCCTGTTGCCTCAGTTACTAGCAATGATACAATTCTCAAAATCTGGTCTTTTTTTGTTTCT
TTGAAATAGTTTCTCCATGTTGTGTGACACAGCAGCCCCTGTCTTATCATAGTTGTCTTCCCTCCACCAC
CTGTACCAGAGATGTTGGATATGTTGGAGGTGAAGGTGTGCAAGGTTTTTAACTAACTGTTCTAATTAAA
GGATTCTGCAGGAAAGAACATGGGTTTACAAAAGAGAAGCTTTTGTATTATTAGTAATTTTTTTTCTTTG
ATGAATTTATGTGCTTAGTTTGGAGAATCGAGAGTTGGCTGGGAAAAGATTTCTGAGGAGTTAAGGGACT
CTGGTGCTGTTTGGGAAAACATTTGCTGTAACTAGGTTTAGGGTATTCAGTCTCTGCCAGTGCTTAGGAG
CTATATTTGCCAGGTGATTTCCCTGTCGCAGGGATTAGCCAGTGATGTGAAACATTTGATTAAGGGACTA
ATAGAAATCTTTCCTGGGGTACATGAGTGGTGGCTGTTACTGGGGTGCCCCCTCCTCAACTCTTTCTCTC
TGTATTTCGAGTAAGTTTGTAGTGGTGAGTCATGGGATACAGTTTTTTAATCCTATGAAGAATCTTGGTA
GGAAGCAAAGTTCTGTTAGGTATATTGTTAATTAAGCCCTTTAAAGTTTCTGCATAATAACTCAGAAACG
AGCCTTTTTCTTTTCTCAGAACTATGCCATGACTCATCCACTTTGGAAGCAGACAAGATATAAATTATCA
ATAAAAATTGCCCATAATCTGGCCTGGCACAGTGGCTCATGCCTGTAATCCCAGCACTTTGGGAGATGAT
GGTGGGAGGATCGCTTGAGGCCAGGAGTTCAAGACCAGCCTGGGCAACGTAGTGAGGAACCCCCGACCTC
CACCATCCCCCCCGCCCCCCACCAACCCCCTCACTTCAAAGTTTTTTAAATCAGCCACGCATGGTGGTAT
ATGCCCTTAGTCCTGTGTACTCAGGAGGCTGAAGTGGGAAAGTCCTTTGAGCCCAGGAGTTGAACGTTGC
AGTGAGCTATGAATGTGCCACCGCACTCTAGCCTGGGCAAGAGAGCTGACCCTGTCACTTAAAAAAAAAA
AAAAAAAAAAACCGATATTCTGATAGCAACACTGCTCACTTGCTCACTCTTTCTCTTAGGAAAGTAAAAA
AGTTTTGTCTTGATGCACAGATTTATTTAAACTTTAGTTCTAAATATATAGGACTTTAAAAAATATTTTC
TGAAAGTGATATCCATTTTTGAGGTGCAAGCAGTTGGGTCCATTTTGGAGATTCCGCCTTAAGCCATTCA
GCTTCAAAATGGCGGGGAGCTCTTGCTACAGAGGGTTACCCCGTTCTGCTTCCTTCCCATTCTGTTAAAC
CTTGTACATGCTCCTTCCCACAGATCACTATTATCTTCAAAGCCCACCATGAGTGTACAGATCAGAAAGT
CTACCAAGCTGTGACAGATGACCTGCCGGCCGCCTTTGTGGATGGCACCACCAGTGGTGGGGACAGCGAT
GCCAAGAGCCTGCGTATCGTGGAAAGGGAGAGTGGCCACTATGTGGAGATGCACGCCCGCTATATAGGGA
CCACAGTGTTTGTGCGGCAGGTGGGTCGCTACCTGACCCTTGCCATCCGTATGCCTGAAGACCTGGCCAT
GTCCTACGAGGAGAGCCAGGACCTGCAGCTGTGCGTGAACGGCTGCCCCCTGAGTGAACGCATCGATGAC
GGGCAGGGCCAGGTGTCTGCCATCCTGGGACACAGCCTGCCTCGCACCTCCTTGGTGCAGGCCTGGCCTG
GCTACACACTGGAGACTGCCAACACTCAATGCCATGAGAAGATGCCAGTGAAGGACATCTATTTCCAGTC
CTGTGTCTTCGACCTGCTCACCACTGGTGATGCCAACTTTACTGCCGCAGCCCACAGTGCCTTGGAGGAT
GTGGAGGCCCTGCACCCAAGGAAGGAACGCTGGCACATTTTCCCCAGCAGTGGCAATGGGACTCCCCGTG
GAGGCAGTGATTTGTCTGTCAGTCTAGGACTCACCTGCTTGATCCTTATCGTGTTTTTGTAGGGGTTGTC
TTTTGTTTTGGTTTTTTATTTTTTGTCTATAACAAAATTTTAAAATATATATTGTCATAATATATTGAGT
AAAAGAGTATATATGTATATACCATGTATATGACAGGATGTTTGTCCTGGGACACCCACCAGATTGTACA
TACTGTGTTTGGCTGTTTTCACATATGTTGGATGTAGTGTTCTTTGATTGTATCAATTTTGTTTTGCAGT
TCTGTGAAATGTTTTATAATGTCCCTGCCCAGGGACCTGTTAGAAAGCACTTTATTTTTTATATATTAAA
TATTTATGTGTGTGCTTGGTTGATATGTATAGTACATATACACAGACATCCATATGCAGCGTTTCCTTTG
AAGGTGACCAGTTGTTTGTAGCTATTCTTGGCTGTACCTTCCTGCCCTTTCCCATTGCTACTGATTTGCC
ACGGTGTGCAGCTTTTACTCGCCACCTTCCGGTGGAGCTGCCTCGTTCCTTTGAACTATGCCCTCACCCT
TCTGCCCTCACTTGATTTGAAAGGGTCGTTAACTCTCCCTTACAGGTGCTTTGACTCTTAAACGCTGATC
TTAAGAAGCTCTCTTCATCTAAGAGCTGTTACTTTTTCAGAAGGGGGGGTATTATTGGTATTCTGATTAC
TCTCAATTCTAATTGTTATATATTTGAGCCCATACAGTGTATTAGGTTGAACCATAGAAACTGCTATTCT
CGTAGGTCAAAAGGGTCTAGTGATGGAAGTTTTGTAGATAAGTACCAGGCATCTCAGTAACTCCTAGACT
TTTTCTCATCCCATGCCCCGTTTTAAATTGTCAGTTTTCCCTCTGACTCTTCTGTGTTAAAACATGAAAC
TATAAATTTAGTAATTATCATGCCTTGCTCTTTTTAATCTATATGACTGATGCAAGCCCCTCTTCTTAAC
CGTTTCTTGGCTTTGAGCCCAGAAACACAGCTCTCCCTGTCTCCAACTCCAGTAAGCCCTCCTCAGCCTC
ACCTTACGAATCCAAAGAACTGGGGTTTGTTAGGTTCTTTCTCTAATGTAGAGGCCCAGATCCCATCACA
AAGTTTTTCATTCTTCCTTGTCCACGATGATCTTCATCACAGTCTTTGATATGTCTGCATGCAAAGTGGA
ACAGAGTTGGGCGGCAATGACAGAAGAGCTTCCTTGGCCTGACTCGGTGTGCGGCCACTTCGGCACTGCT
TAATCCAGATATTCTTGTTAACTAAGCATTGTGCTTCCCAGGTGGTCTGAAGTCAGGTACTCTCTCTCTC
AACACCTGTAGTTGAATATGATTTGGTCAGTTGCTCGTTGTAACTTGGAGAAATTCCTATAAAGTAAGAT
CTCCTTGCCTCTTCCATCCATTGTTGGCACCCCCTTGCAAAAGGAAAAGAACAGCAAAAGTCAGGAGCAG
TAATCTGAGAAAGTTAACTCCAGGATAGGTAGGTTTCTATTGTTATAGCTAGATGTAAATCTTTAGTTCC
AAGAAGTGATAGAGTTTCTGCTTTAATAATTTGTTGATAAGTTTACATAAACAGAAATAAAAGATACTAT
CTTTACCGTAGTAGTTCAGGCCAAGATTATGCTTAGTTTTAGTTCTCCAGGTAGTTACTTTTGCCATGTC
CTATTGATCAGTGACACTGCCAGAGGCCCATACCGGCAAGAGGAAGAGGACGTCATTTTGTAAAGTTTAA
CTTCTTAGCGAACTGATGTGCCACCCAGTCACAGAGTGGAGTTGTGAATTCATGTAGAGGTGGCAAACCT
CTACCTTGTGTTGATGAGAGAATAATCTTGGGCAGTCTGGGAAAATAAGGAAGGCATCTCCTTCTTACTC
ATGGAGATTCAACTATAGAGAGTTGAAACCTAAACCCGCCTTCCTTTTATAGAAGCTGGACTAGAGACGG
ACTGACCATCAGCTCTGAACTGTGGCTTTTTTTGTTCACCTATGATGCCATGTACCAAATTCAGAAGCTA
TCGTTAATAATTTGTTTTATAATTGAGTAGTACAAGCGAGGAAAAAATACGGAGGATAACCACTATTTTT
GTGCAAATAGTATGAAAGTGAAGTAAAAGCAATAGAAGAAATTTCTATAGGATCTGGGTTTAGAGTGTGT
ATCATTAATAAATATACCTTTGCTCTTTTCAGGGAAAATAACAACCACCCTTACTGATAGTTGGGAAAAG
AAGATTGGGTTATTTTGCCATATCATTTAGCTGGAAGTGACATTTAAAAGCACCCTGCATCACTAGTAAT
AGTGTATTTTGCTATTCTGCCCTTGTAATCGGTGTCCCTGTAAAACAATCCCCACAGATTACTTTCAGAA
ATAGATGTATTTCTCTACGTAAGGGCCAGGTTTATTTTCTCCTTTTTTGAGATTTCTAGAAAAAATGCTG
CTTGCACATGTTGGTTCTTGAAACCTTAGCTAGAAGAATTTCAGGTCATACCAACATGTGGATAGGCTAT
AGCTGTTCAGAGGTCTCCTGGGGGAGCTTAAAACGGGGGAAACACTGGTTTTCACAGATGCTCCACATGG
CTGTCTTTAAAAGACTCAAAACTTTTTTTTGTCCTCTTTGTTATGCTTGGAAGCTCCCCCCCCCCCCCCA
CAGTGTGTCGAGTCTTTGCAAAGAAACCTTTAGATGTGGTTCATAGATATATGAATACGTATCTGTGTAA
AACAGTGAGTGTGCAGTGTGTAAATACTTTAAATTATTATGCTAGAAAAATAAAGTTACATACCTTGCTG
TGGAA

### Incorporation by Reference

All publications, patents, and patent applications mentioned herein are hereby incorporated by reference in their entirety as if each individual publication, patent or patent application was specifically and individually indicated to be incorporated by reference. In case of conflict, the present application, including any definitions herein, will control.

### EQUIVALENTS

Those skilled in the art will recognize, or be able to ascertain using no more than routine experimentation, many equivalents to the specific embodiments are described herein. Such equivalents are intended to be encompassed by the following claims.

1. A composition comprising a population of modified T cells that comprise decreased expression or activity of RGMb.

2-4. (canceled)

5. The composition of claim 1, wherein the T cells have been modified ex vivo to inhibit the activity or expression of RGMb, wherein modifying comprises contacting the T cells with an RGMb agent that decreases the expression of or inhibits or disrupts the activity of RGMb in the T cells.

6. The composition of claim 5, wherein the RGMb agent is an antibody that specifically binds to an RGMb peptide, an RNA inhibitory molecule, or a gRNA complementary to a portion of a RGMb gene.

7-8. (canceled)

9. The composition of claim 1, wherein the T cells are cytotoxic T cells (CTLs), CAR-T cells or TCR-T cells.

10. The composition of claim 1, wherein the composition further comprises an agent that specifically binds to PD-1 or PD-L1.

11-14. (canceled)

15. The composition of claim 10, wherein the agent that specifically binds to PD-1 or PD-L1 is an antibody that blocks PD-1, and the antibody that blocks PD-1 is selected from cemiplimab (REGN2810), nivolumab (BMS-936558, MDX-1106, ONO-4538), pembrolizumab (MK-3475, SCH 900475), SHR1210, sintilimab (IBI308), spartalizumab (PDR001), tislelizumab (BGB-A317), pidilizumab, BCD-100, toripalimab (JS001), PF-06801591, AB122, AK105, AMG 404, BCD-100, BI 754091, F520, HLX10, HX008, JTX-4014, LZM009, MEDI0680, MGA012, Sym021, TSR-042, PSB205, MGD019, MGD013, AK104, XmAb20717, R07121661, and CX-188.

16-19. (canceled)

20. The composition of claim 10, wherein the agent that specifically binds to PD-1 or PD-L1 is an antibody that blocks PD-L1, and the antibody that blocks PD-L1 is selected from atezolizumab (MPDL3280A, RG7446, R05541267), durvalumab (MEDI4736, MEDI-4736), avelumab (MSB0010718C), FS118, BCD-135, BGB-A333, CBT-502, CK-301, CS1001, FAZ053, HLX20, KN035, MDX-1105, MSB2311, SHR-1316, TG-1501, ZKAB001, INBRX-105, MCLA-145, KN046, M7824, and LY3415244.

21. A method of treating or preventing cancer in a subject, comprising:
a) isolating a population of T cells ex vivo;
b) contacting the T cells with an RGMb agent that decreases the expression of or inhibits or disrupts the activity of RGMb in the T cells, and
b) administering to the subject T cells conjointly with an agent that specifically binds to PD-1 or PD-L1.

22. The method of claim 21, wherein the RGMb agent is an antibody that specifically binds to an RGMb peptide, an RNA inhibitory molecule, or a gRNA complementary to a portion of a RGMb gene.

23-24. (canceled)

25. A method of treating or preventing cancer in a subject, comprising administering to the subject T cells conjointly with an agent that specifically binds to PD-1 or PD-L1, wherein the T cells comprise decreased expression or activity of RGMb.

26. The method of claim 25, further comprising a step of modifying the T cells to inhibit the activity of RGMb, the expression of RGMb, or disrupt the activity of RGMb prior to administration to the subject, wherein modifying comprises contacting the T cells with an RGMb agent that decreases the expression of or inhibits or disrupts the activity of RGMb in the T cells.

27. (canceled)

28. The method of claim 26, wherein the RGMb agent is an antibody that specifically binds to an RGMb peptide, an RNA inhibitory molecule, or a gRNA complementary for a portion of the RGMb gene.

29-33. (canceled)

34. The method of claim 21, wherein the T cells are cytotoxic T cells (CTLs), CAR-T cells or TCR-T cells.

35. The method of claim 21, wherein the agent that specifically binds to PD-1 or PD-L1 is an antibody.

36-39. (canceled)

40. The method of claim 36, wherein the antibody that blocks PD-1 is selected from cemiplimab (REGN2810), nivolumab (BMS-936558, MDX-1106, ONO-4538), pembrolizumab (MK-3475, SCH 900475), SHR1210, sintilimab (IBI308), spartalizumab (PDR001), tislelizumab (BGB-A317), pidilizumab, BCD-100, toripalimab (JS001), PF-06801591, AB122, AK105, AMG 404, BCD-100, BI 754091, F520, HLX10, HX008, JTX-4014, LZM009, MEDI0680, MGA012, Sym021, TSR-042, PSB205, MGD019, MGD013, AK104, XmAb20717, R07121661, and CX-188.

41. The method of claim 21, wherein the agent that specifically binds to PD-1 or PD-L1 is an antibody that blocks PD-L1.

42-44. (canceled)

45. The method of claim 41, wherein the antibody that blocks PD-L1 is selected from atezolizumab (MPDL3280A, RG7446, R05541267), durvalumab (MEDI4736, MEDI-4736), avelumab (MSB0010718C), FS118, BCD-135, BGB-A333, CBT-502, CK-301, CS1001, FAZ053, HLX20, KN035, MDX-1105, MSB2311, SHR-1316, TG-1501, ZKAB001, INBRX-105, MCLA-145, KN046, M7824, and LY3415244.

46. The method of claim 21, wherein the subject is nonresponsive to immune checkpoint inhibitor therapy.

47. The method of claim 21, wherein the subject is nonresponsive to anti-PD-1 therapy or anti-PD-L1 therapy.

48. The method of claim 21, wherein the cancer is lung cancer, a breast cancer, a colon cancer, a cervical cancer, a pancreatic cancer, a renal cancer, a stomach cancer, a GI cancer, a liver cancer, a bone cancer, a hematological cancer, a neural tissue cancer, a melanoma, a thyroid cancer, a ovarian cancer, a testicular cancer, a prostate cancer, a cervical cancer, a vaginal cancer, or a bladder cancer.

49-58. (canceled)
